(19) United States
(12) Patent Application Publication
(10) Pub. No.: US 2025/0154524 A1
(43) Pub. Date: May 15, 2025

(54) EUKARYOTIC CELLS COMPRISING ADENOVIRUS-ASSOCIATED VIRUS POLYNUCLEOTIDES

(71) Applicant: Regeneron Pharmaceuticals, Inc., Tarrytown, NY (US)

(72) Inventors: Michael Goren, Tarrytown, NY (US); Yu Zhao, Willison Park, NY (US); Alexandros Strikoudis, New York, NY (US); Darya Burakov, Tarrytown, NY (US); Gang Chen, Yorktown Heights, NY (US)

(21) Appl. No.: 19/022,099

(22) Filed: Jan. 15, 2025

Related U.S. Application Data
(63) Continuation of application No. 18/047,341, filed on Oct. 18, 2022, now Pat. No. 12,234,472.
(60) Provisional application No. 63/256,730, filed on Oct. 18, 2021.

(51) Int. Cl. C12N 15/85; C07K 14/005; C12N 15/67

(52) U.S. Cl. CPC C12N 15/85; C07K 14/005; C12N 15/67; C12N 2830/42; C12N 2830/50; C12N 2840/203

(57) ABSTRACT

The present inventions provide eukaryotic cells, such as mammalian cells, that comprise adeno-associated virus (AAV) polynucleotides, including AAV capsid proteins (Cap), and are capable of expressing the polypeptides encoded by the AAV polynucleotides, and thereby are capable of producing AAV, including recombinant AAV. The eukaryotic cells also may comprise adenovirus (Ad) polynucleotides. The present inventions also provide methods of expressing AAV polynucleotides, as well as Ad polynucleotides, in eukaryotic cells, such as CHO cells, HEK 293 and BHK cells. The present inventions further provides other products and methods described herein.

This application claims priority to U.S. Application Ser. No. 63/256,730, filed Oct. 18, 2021, which is hereby incorporated by reference in its entirety.

### FIELD OF THE INVENTIONS

The present inventions provide eukaryotic cells that comprise adeno-associated virus (AAV) polynucleotides, including AAV capsid proteins (Cap). The cells are capable of expressing the polypeptides encoded by the AAV polynucleotides, and thereby are capable of producing AAV, including recombinant AAV. The eukaryotic cells also may comprise adenovirus (Ad) polynucleotides. The present inventions also provide methods of expressing AAV polynucleotides, as well as Ad polynucleotides, in eukaryotic cells. The present inventions also provide methods for producing recombinant adeno-associated virus utilizing eukaryotic cells that express AAV and Ad polypeptides encoded by polynucleotides, as well as recombinant AAV produced by these inventive methods. The present inventions further provide other products and methods described herein.

### REFERENCE TO ELECTRONIC SEQUENCE LISTING

The application contains a Sequence Listing, which has been submitted electronically in .XML format and is hereby incorporated by reference in its entirety. Said .XML copy, created on Oct. 5, 2022, is named “135975-61702.xml” and is 229,879 bytes in size. The sequence listing contained in this .XML file is part of the specification and is hereby incorporated by reference herein in its entirety.

### BACKGROUND OF THE INVENTIONS

Adeno-associated virus (AAV) is a non-enveloped, single-stranded DNA virus and is used as a gene delivery vector for both research and therapeutics. Weitzman and Linden, Adeno-Associated Virus Biology (chapter 1), Meth. Molec. Biol. 807: 1-23 (2011). Gene transfer vectors based on AAV have demonstrated promise for human gene therapy based on their safety profile and potential to achieve long-term efficacy in animal models. Wang et al., Nature, 18: 358-78 (2019). A major challenge for advancing AAV-based therapies into clinical development is the difficulty and cost of producing sufficient quantities of AAV through transient methodologies.

AAV has been produced in HEK 293, BHK, human amniotic (for example, epithelial cells such as HAEpiC) and SF9 lines. However, expression is transient due to the use of plasmid vectors containing the necessary AAV and helper virus genes. For example, recombinant AAV production in HEK 293 cells using adenovirus helper gene products utilizes adenovirus E2A, E4, VA RNA and AAV Rep and Cap, along with AAV inverted terminal repeats (ITR) flanking the polynucleotide of interest. The reliance on non-integrated plasmid vectors means that the requisite gene products will be lost over time and need to be continually re-established.

The AAV genome includes a capsid gene referred to as “Cap” or “CAP”. Cap in nature is translated to produce, via alternative start codons and transcript splicing, three size-variant structural proteins referred to as VP1 (about 90 kDa), VP2 (about 72 kDa) and VP3 (about 60 kDa). An AAV capsid contains 60 subunits total of the VP proteins. A ratio of 1:1:10 is considered the most typical ratio for VP1:VP2:VP3, which is a stoichiometry of 5 VP1 subunits:5 VP2 subunits:50 VP3 subunits. However, there can be variation. Wörner et al., Nature Communications 12:1642 (2021). AAV polynucleotides and proteins, including CAP, can be selected from any serotype.

Thus, there exists the need to develop improved cells and production methods that avoid the transient nature of non-integrated plasmid vectors.

### SUMMARY OF THE INVENTIONS

The present inventions provide stable eukaryotic cells, such as mammalian cells (for example, primate, rodent and canine cells), comprising integrated AAV polynucleotides and Ad polynucleotides. The protein and VA RNA products of the Ad polynucleotides act in a helper capacity. All AAV and Ad types are amenable for use according to the present inventions. The present inventions advantageously can employ site-specific integration into the cell genome, which refers to pre-selected genomic sites for exogenous DNA to be inserted into a cellular genome. Random insertion can be employed as well.

Herein described are polynucleotides, where each can comprise (i) a promoter, (ii) an intron, (iii) an internal ribosome entry site, (iv) a polynucleotide encoding Adeno-associated virus (AAV) Cap protein, and (v) a polyadenylation site. For example, the polynucleotide can be in a CHO cell and have the (i) promoter, (ii) intron, (iii) internal ribosome entry site and (iv) polynucleotide encoding AAV Cap protein can be operably linked. The polynucleotide can be integrated into a CHO cell genome, such as a CHO chromosome. Alternatively, the polynucleotide can be in a HEK 293 cell and have the (i) promoter, (ii) intron, (iii) internal ribosome entry site and (iv) polynucleotide encoding AAV Cap protein operably linked. The polynucleotide can be integrated into a HEK 293 cell genome, such as a HEK 293 cell chromosome. Another alternative is the polynucleotide can be in a BHK cell and have the (i) promoter, (ii) intron, (iii) internal ribosome entry site and (iv) polynucleotide encoding AAV Cap protein operably linked. The polynucleotide can be integrated into a BHK cell genome, such as a BHK cell chromosome. In yet another alternative is the polynucleotide can be in a human amniotic cell and have the (i) promoter, (ii) intron, (iii) internal ribosome entry site and (iv) polynucleotide encoding AAV Cap protein operably linked. The polynucleotide can be integrated into a human amniotic cell genome, such as a human amniotic cell chromosome. Additionally, the polynucleotide can be integrated into non-chromosomal locations as known by the person skilled in the art, such as episomes.

The polynucleotide can further comprise an operator. The promoter can be a CMV promoter and the operator can be a Tet operator. AAV proteins and polynucleotides, including CAP, can be selected from any serotype. When Cap is from serotype 5 (“Cap5”) and expressed in a CHO cell, the polynucleotide allows for production of AAV Cap5 VP2 and VP3 protein, wherein the amount of VP3 produced is greater than the amount of VP2 produced. Production of VP1 can be less than 1% the level of VP2 production. Production ratios can vary based upon experimental conditions and analytical techniques.

Additionally, polynucleotides are described, wherein each can comprise (i) a promoter, (ii) an intron, (iii) a first internal ribosome entry site, (iv) a first polynucleotide encoding AAV Cap protein, (v) a second internal ribosome entry site, (vi) a second polynucleotide encoding AAV Cap protein, and (vii) a polyadenylation site. For example, the polynucleotide can be in a CHO cell and the (i) promoter, (ii) intron, (iii) first internal ribosome entry site, (iv) first polynucleotide encoding AAV Cap protein, (v) a second internal ribosome entry site, and (vi) second polynucleotide encoding AAV Cap protein can be operably linked. The polynucleotide can be integrated into a CHO cell genome. Alternatively, the polynucleotide can be in a HEK 293 cell and the (i) promoter, (ii) intron, (iii) first internal ribosome entry site, (iv) first polynucleotide encoding AAV Cap protein, (v) a second internal ribosome entry site, and (vi) second polynucleotide encoding AAV Cap protein are operably linked. The polynucleotide can be integrated into a HEK 293 cell genome. In another alternative, the polynucleotide can be in a BHK cell and the (i) promoter, (ii) intron, (iii) first internal ribosome entry site, (iv) first polynucleotide encoding AAV Cap protein, (v) a second internal ribosome entry site, and (vi) second polynucleotide encoding AAV Cap protein are operably linked. The polynucleotide can be integrated into a BHK cell genome. In another alternative, the polynucleotide can be in a human amniotic cell and the (i) promoter, (ii) intron, (iii) first internal ribosome entry site, (iv) first polynucleotide encoding AAV Cap protein, (v) a second internal ribosome entry site, and (vi) second polynucleotide encoding AAV Cap protein are operably linked. The polynucleotide can be integrated into a human amniotic cell genome. The polynucleotide can further comprise an operator. The promoter can be a CMV promoter and the operator can be a Tet operator. AAV proteins and polynucleotides, including CAP, can be selected from any serotype. When Cap is from serotype 5 (“Cap5”) and expressed in a CHO cell, the polynucleotide allows for production of AAV Cap5 proteins VP1, VP2 and VP3. The amount of VP3 production can be greater than the amount of VP1 production and the amount of VP2 production. The amount of VP2 production can be greater than the amount of VP1 production. Production ratios can vary based upon experimental conditions and analytical techniques.

Moreover, there are described polynucleotides, wherein each polynucleotide can comprise (i) a promoter, (ii) an intron, (iii) a polynucleotide encoding AAV Cap protein, and (iv) a polyadenylation site, wherein the polynucleotide allows for production of AAV Cap VP1 protein when expressed. For example, the polynucleotide can be in a CHO cell and the (i) promoter, (ii) intron, and (iii) polynucleotide encoding AAV Cap protein can be operably linked. The polynucleotide can be integrated into a CHO cell genome. Alternatively, the polynucleotide can be in a HEK 293 cell and the (i) promoter, (ii) intron, and (iii) polynucleotide encoding AAV Cap protein can be operably linked. The polynucleotide can be integrated into a HEK 293 cell genome. In another alternative, the polynucleotide can be in a BHK cell and the (i) promoter, (ii) intron, and (iii) polynucleotide encoding AAV Cap protein can be operably linked. The polynucleotide can be integrated into a BHK cell genome. In still another alternative, the polynucleotide can be in a human amniotic cell and the (i) promoter, (ii) intron, and (iii) polynucleotide encoding AAV Cap protein can be operably linked. The polynucleotide can be integrated into a human amniotic cell genome. The polynucleotide can further comprise an operator. The promoter can be a CMV promoter and the operator can be a Tet operator.

Furthermore, there are described polynucleotides, wherein each can comprise (i) a promoter, (ii) an internal ribosome binding site, (iii) a polynucleotide encoding AAV Cap protein, and (iv) a polyadenylation site, wherein the polynucleotide allows for production of AAV VP1 Cap protein when expressed. The polynucleotide can be in a CHO cell and the (i) promoter, (ii) internal ribosome binding site, and (iii) polynucleotide encoding AAV Cap protein can be operably linked. The polynucleotide can be integrated into a CHO genome. Alternatively, the polynucleotide can be in a HEK 293 cell and the (i) promoter, (ii) internal ribosome binding site, and (iii) polynucleotide encoding AAV Cap protein can be operably linked. The polynucleotide can be integrated into a HEK 293 cell genome. In another alternative, the polynucleotide can be in a BHK cell and the (i) promoter, (ii) internal ribosome binding site, and (iii) polynucleotide encoding AAV Cap protein can be operably linked. The polynucleotide can be integrated into a BHK cell genome. In yet another alternative, the polynucleotide can be in a human amniotic cell and the (i) promoter, (ii) internal ribosome binding site, and (iii) polynucleotide encoding AAV Cap protein can be operably linked. The polynucleotide can be integrated into a human amniotic cell genome. The polynucleotide can comprise an operator. The promoter can be a CMV promoter and the operator can be a Tet operator.

Additionally, there are described eukaryotic cells, where each cell can comprise a polynucleotide comprising (i) a promoter, (ii) an intron, (iii) a first internal ribosome entry site, (iv) a first polynucleotide encoding AAV Cap protein, (v) a second internal ribosome entry site, (vi) a second polynucleotide encoding AAV Cap protein, and (vii) a polyadenylation site. The (i) promoter, (ii) intron, (iii) first internal ribosome entry site, (iv) first polynucleotide encoding AAV Cap protein, (v) a second internal ribosome entry site, and (vi) second polynucleotide encoding AAV Cap protein can be operably linked. The polynucleotide can be integrated into a cell genome. The cell can be a CHO cell, a HEK 293 cell, a BHK cell, a Human Amniotic Cell or other eukaryotic cell. The cell can further comprise an operator. The promoter can be a CMV promoter and the operator can be a Tet operator. The cell can further comprise: a polynucleotide encoding AAV Rep, a polynucleotide encoding Ad E1A, a polynucleotide encoding Ad E1B, a polynucleotide encoding Ad E2A or E2A orf, a polynucleotide encoding Ad E4 or E4 orf 6, a polynucleotide encoding VA RNA, and a polynucleotide encoding AAV ITRs and a protein of interest.

There are also described eukaryotic cells, where each cell can comprise (A) a first polynucleotide comprising (i) a promoter, (ii) an intron, (iii) an internal ribosome entry site, (iv) a polynucleotide encoding AAV Cap protein, and (v) a polyadenylation site; and (B) a second polynucleotide comprising (i) a promoter, (ii) an intron, (iii) a polynucleotide encoding AAV Cap protein, and (iv) a polyadenylation site. The (i) promoter, (ii) intron, (iii) internal ribosome entry site and (iv) polynucleotide encoding AAV Cap protein of (A) first polynucleotide can be operably linked, and wherein the (i) promoter, (ii) intron, and (iii) polynucleotide encoding AAV Cap protein of (B) second polynucleotide can be operably linked. The cell can have at least one polynucleotide integrated into a cell genome. The cell can be a CHO cell, a HEK 293 cell, a BHK cell, a human amniotic cell or other eukaryotic cell. The cell can further comprise an operator. The promoter can be a CMV promoter and the operator can be a Tet operator. The cell can further comprise a polynucleotide encoding AAV Rep, a polynucleotide encoding Ad E1A, a polynucleotide encoding Ad E1B, a polynucleotide encoding Ad E2A or E2A orf, a polynucleotide encoding E4 or E4 orf 6, a polynucleotide encoding VA RNA, and a polynucleotide encoding AAV ITRs and a protein of interest.

Additionally, there are described eukaryotic cells, where each cell can comprise: (A) a first polynucleotide comprising (i) a promoter, (ii) an intron, (iii) an internal ribosome entry site, (iv) a polynucleotide encoding AAV Cap protein, and (v) a polyadenylation site; and (B) a second polynucleotide comprising (i) a promoter, (ii) an internal ribosome entry site, (iii) a polynucleotide encoding AAV Cap protein, and (iv) a polyadenylation site. The cells can have the (i) promoter, (ii) intron, (iii) internal ribosome entry site and (iv) polynucleotide encoding AAV Cap protein of (A) first polynucleotide operably linked, and the (i) promoter, (ii) internal ribosome entry site, and (iii) polynucleotide encoding AAV Cap protein of (B) second polynucleotide operably linked. At least one polynucleotide can be integrated into a cell genome. The cell can be a CHO cell, a HEK 293 cell, a BHK cell, a human amniotic cell or other eukaryotic cell. The cell can further comprise an operator. The promoter can be a CMV promoter and the operator can be a Tet operator. The cell can further comprise: a polynucleotide encoding AAV Rep, a polynucleotide encoding Ad E1A, a polynucleotide encoding Ad E1B, a polynucleotide encoding Ad E2A or E2A orf, a polynucleotide encoding Ad E4 or E4 orf 6, a polynucleotide encoding VA RNA, and a polynucleotide encoding AAV ITRs and a protein of interest.

There also are described cell cultures comprising any of the above cells in any type of media, including growth media and maintenance media. Additionally, there are described methods of producing AAV proteins, including Cap proteins, and methods that can result in the production of recombinant AAV.

There are described methods of producing adeno-associated virus (AAV) Cap protein in cell culture, wherein a method comprises the steps of: providing eukaryotic cells, wherein a cell comprises a polynucleotide comprising (i) a promoter, (ii) an intron, (iii) a first internal ribosome entry site, (iv) a first polynucleotide encoding AAV Cap protein, (v) a second internal ribosome entry site, (vi) a second polynucleotide encoding AAV Cap protein, and (vii) a polyadenylation site; and culturing the cells in a culture medium to allow the cells to produce AAV Cap protein, wherein the polynucleotide allows for production of AAV Cap proteins VP1, VP2 and VP3. The (i) promoter, (ii) intron, (iii) first internal ribosome entry site, (iv) first polynucleotide encoding AAV Cap protein, (v) a second internal ribosome entry site, and (vi) second polynucleotide encoding AAV Cap protein can be operably linked. The polynucleotide can be integrated into a cell genome. The cell can be a CHO cell, a HEK 293 cell, a BHK cell, a human amniotic cell or other eukaryotic cell. The cell can further comprise an operator. The cell can further comprise: a polynucleotide encoding AAV Rep, a polynucleotide encoding Ad E1A, a polynucleotide encoding Ad E1B, a polynucleotide encoding Ad E2A or E2A orf, a polynucleotide encoding Ad E4 or E4 orf 6, a polynucleotide encoding VA RNA, and a polynucleotide encoding AAV ITRs and a protein of interest, wherein the cell can produce recombinant AAV.

Also described are methods of producing adeno-associated virus (AAV) Cap protein in cell culture, wherein a method comprises the steps of providing eukaryotic cells, where a cell comprises (a) a first polynucleotide comprising (i) a promoter, (ii) an intron, (iii) an internal ribosome entry site, (iv) a polynucleotide encoding AAV Cap protein, and (v) a polyadenylation site; and (b)
a second polynucleotide comprising (i) a promoter, (ii) an intron, (iii) a polynucleotide encoding AAV Cap protein, and (iv) a polyadenylation site; and culturing the cells in a culture medium to allow the cells to produce AAV Cap protein, wherein the polynucleotide allows for production of AAV Cap proteins VP1, VP2 and VP3. The (i) promoter, (ii) intron, (iii) internal ribosome entry site and (iv) polynucleotide encoding AAV Cap protein of (a) first polynucleotide can be operably linked, and the (i) promoter, (ii) intron, and (iii) polynucleotide encoding AAV Cap protein of (b) second polynucleotide can be operably linked. The polynucleotide can be integrated into a cell genome. The cell can be a CHO cell, a HEK 293 cell, a BHK cell, a human amniotic cell or other eukaryotic cell. The cell can further comprise an operator. The promoter can be a CMV promoter and the operator can be a Tet operator. The cell can further comprise: a polynucleotide encoding AAV Rep, a polynucleotide encoding Ad E1A, a polynucleotide encoding Ad E1B, a polynucleotide encoding Ad E2A or E2A orf, a polynucleotide encoding Ad E4 or E4 orf 6, a polynucleotide encoding VA RNA, and a polynucleotide encoding AAV ITRs and a protein of interest, wherein the cell can produce recombinant AAV.

There are also described methods of producing adeno-associated virus (AAV) Cap protein in cell culture, wherein a method comprises the steps of: providing eukaryotic cells, where a cell comprises (a) a first polynucleotide comprising (i) a promoter, (ii) an intron, (iii) an internal ribosome entry site, (iv) a polynucleotide encoding AAV Cap protein, and (v) a polyadenylation site; and (b) a second polynucleotide comprising (i) a promoter, (ii) an internal ribosome entry site, (iii) a polynucleotide encoding AAV Cap protein, and (iv) a polyadenylation site; and culturing the cells in a culture medium to allow the cells to produce AAV Cap protein, wherein the polynucleotide allows for production of AAV Cap proteins VP1, VP2 and VP3. The (i) promoter, (ii) intron, (iii) internal ribosome entry site and (iv) polynucleotide encoding AAV Cap protein of (a) first polynucleotide can be operably linked, and the (i) promoter, (ii) internal ribosome entry site, and (iii) polynucleotide encoding AAV Cap protein of (b) second polynucleotide can be operably linked. The polynucleotide can be integrated into a cell genome. The cell can be a CHO cell, a HEK 293 cell, a BHK cell, a human amniotic cell or other eukaryotic cell. The cell can further comprise an operator. The promoter can be a CMV promoter and the operator can be a Tet operator. The cell can further comprise: a polynucleotide encoding AAV Rep, a polynucleotide encoding Ad E1A, a polynucleotide encoding Ad E1B, a polynucleotide encoding Ad E2A or E2A orf, a polynucleotide encoding Ad E4 or E4 orf 6, a polynucleotide encoding VA RNA, and a polynucleotide encoding AAV ITRs and a protein of interest, wherein the cell can produce recombinant AAV.

### BRIEF DESCRIPTION OF THE FIGURES

FIG. 1 is a schematic diagram of a polynucleotide comprising a promoter, an intron, an internal ribosome entry site (IRES), a polynucleotide encoding AAV Cap protein and a polyadenylation site (poly A).

FIG. 2 is a schematic diagram of a polynucleotide comprising a promoter, an intron, two internal ribosome entry sites, two polynucleotides encoding AAV Cap protein and a polyadenylation site.

FIG. 3 is a schematic diagram of a polynucleotide comprising a promoter, an intron, a polynucleotide encoding AAV Cap protein and a polyadenylation site.

FIG. 4 is a schematic diagram of a polynucleotide comprising a promoter, an internal ribosome entry site, a polynucleotide encoding AAV Cap protein and a polyadenylation site.

FIG. 5 is a schematic diagram of a polynucleotide comprising a promoter with operator (Op), an intron, an internal ribosome entry site, a polynucleotide encoding AAV Cap protein and a polyadenylation site.

FIG. 6 is a schematic diagram of a polynucleotide comprising a promoter with operator (Op), an intron, two internal ribosome entry sites, two polynucleotides encoding AAV Cap protein and a polyadenylation site.

FIG. 7 is a schematic diagram of a polynucleotide comprising a promoter with operator (Op), an intron, a polynucleotide encoding AAV Cap protein and a polyadenylation site.

FIG. 8 is a schematic diagram of a polynucleotide comprising a promoter with operator (Op), an internal ribosome entry site, a polynucleotide encoding AAV Cap protein and a polyadenylation site.

### DETAILED DESCRIPTION OF THE INVENTIONS

Unless defined otherwise, all technical and scientific terms used herein have the same meaning as commonly understood by one of ordinary skill in the art to which these inventions belong.

### Definitions

The term “about” in the context of numerical values and ranges refers to values or ranges that approximate or are close to the recited values or ranges such that the inventions can perform, such as having a sought rate, amount, degree, increase, decrease, or extent of expression, concentration, or time, as is apparent from the teachings contained herein. Thus, this term encompasses values beyond those simply resulting from systematic error. For example, “about” can signify values either above or below the stated value in a range of approx. +/−10% or more or less depending on the ability to perform.

“Intron” is a section of DNA located between exons. An intron is removed to form a mature messenger RNA. Preferred introns are those that can affect the starting point of translation, and exemplars are the hCMV-IE intron (Human cytomegalovirus immediate early protein) and FMDV intron (Foot and Mouth Disease Virus). The globin gene intron also has been reportedly used for expression.

A “nucleic acid moiety” includes any arrangement of single stranded or double stranded nucleotide sequences. Nucleic acid moieties can include, but are not limited to, polynucleotides, promoters, enhancers, operators, repressors, transcription termination signals, ribosomal entry sites and polyadenylation signals.

A “DNA cassette” or “cassette” is a type of nucleic acid moiety that comprises at least a promoter, at least one open reading frame and optionally a polyadenylation signal, for example an SV40 polyadenylation signal. Other nucleic acid moieties, such as operators, also are optional. A DNA cassette thus is a polynucleotide that comprises two or more shorter polynucleotides.

“Operably linked” refers to one or more nucleotide sequences in functional relationships with one or more other nucleotide sequences. Such functional relationships can directly or indirectly control, cause, regulate, enhance, facilitate, permit, attenuate, repress or block an action or activity in accordance with the selected design. Exemplars include single-stranded or double-stranded nucleic acid moieties, and can comprise two or more nucleotide sequences arranged within a given moiety in such a way that sequence(s) can exert at least one functional effect on other(s). For example, a promoter operably linked to the coding region of a DNA polynucleotide sequence can facilitate transcription of the coding region. Other elements, such as enhancers, operators, repressors, transcription termination signals, ribosomal entry sites and polyadenylation signals also can be operably linked with a polynucleotide of interest to control its expression. Arrangements and spacing to achieve operable linkages can be ascertained by approaches available to the person skilled in the art, such as screening using western blots and RT-PCR.

“Operator” indicates a DNA sequence that is introduced in or near a polynucleotide sequence in such a way that the polynucleotide sequence may be regulated by the interaction of a molecule capable of binding to the operator and, as a result, prevent or allow transcription of the polynucleotide sequence, as the case may be. One skilled in the art will recognize that the operator must be located sufficiently in proximity to the promoter such that it is capable of controlling or influencing transcription by the promoter, which can be considered a type of operable linkage. The operator may be placed either downstream or upstream of the promoter. These include, but are not limited to, the operator region of the Lex A gene of E. coli, which binds the Lex A peptide and the lactose and 45 tryptophan operators, which bind the repressor proteins encoded by the Lad and trpR genes of E. coli. The bacteriophage operators from the lambda Pi and the phage P22 Mnt and Arc. Preferred operators are the Tet (tetracycline) operator and the Arc operator. Operators can have a native sequence or a mutant sequence. For example, mutant sequences of the Tet operator are disclosed in Wissmann et al., Nucleic Acids Res. 14: 4253-66 (1986).

The phrases “percent identity” or “% identical,” in their various grammatical forms, when describing a sequence is meant to include homologous sequences that display the recited identity along regions of contiguous homology, but the presence of gaps, deletions, or insertions that have no homolog in the compared sequence are not taken into account in calculating percent identity. As used herein, a “percent identity” or “% identical” determination between homologs would not include a comparison of sequences where the homolog has no homologous sequence to compare in an alignment. Thus, “percent identity” and “% identical” do not include penalties for gaps, deletions, and insertions.

A “homologous sequence” in the context of nucleic acid sequences refers to a sequence that is substantially homologous to a reference nucleic acid sequence. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding nucleotides are identical over a relevant stretch of residues. In some embodiments, the relevant stretch is a complete (i.e., full) sequence.

“Polynucleotide” includes a sequence of nucleotides covalently joined, and includes RNA and DNA. Oligonucleotides are considered shorter polynucleotides. Genes are DNA polynucleotides (polydeoxyribonucleic acid) that ultimately encode polypeptides, which are translated from RNA (polyribonucleic acid) that was typically transcribed from DNA. DNA polynucleotides also can encode RNA polynucleotides that is not translated, but rather function as RNA “products”. The type of polynucleotide (that is, DNA or RNA) is apparent from the context of the usage of the term. A polynucleotide referred to or identified by the polypeptide it encodes sets forth and covers all suitable sequences in accordance with codon degeneracy. Polynucleotides, including those disclosed herein, include percent identity sequences and homologous sequences when indicated.

“Polypeptide” or “peptide” refers to sequence(s) of amino acids covalently joined. Polypeptides include natural, semi-synthetic and synthetic proteins and protein fragments. “Polypeptide” and “protein” can be used interchangeably. Oligopeptides are considered shorter polypeptides.

“Protein of interest” or “polypeptide of interest” can have any amino acid sequence, and includes any protein, polypeptide, or peptide, and derivatives, components, domains, chains and fragments thereof. Included are, but not limited to, viral proteins, bacterial proteins, fungal proteins, plant proteins and animal (including human) proteins. Protein types can include, but are not limited to, antibodies, bi-specific antibodies, multi-specific antibodies, antibody chains (including heavy and light), antibody fragments, Fv fragments, Fc fragments, Fc-containing proteins, Fc-fusion proteins, receptor Fc-fusion proteins, receptors, receptor domains, trap and mini-trap proteins, enzymes, factors, repressors, activators, ligands, reporter proteins, selection proteins, protein hormones, protein toxins, structural proteins, storage proteins, transport proteins, neurotransmitters and contractile proteins. Derivatives, components, chains and fragments of the above also are included. The sequences can be natural, semi-synthetic or synthetic. Proteins of interest and polypeptides of interest are encoded by “genes of interest,” which also can be referred to as “polynucleotides of interest.” Where multiple genes (same or different) are integrated, they can be referred to as “first,” “second”, “third,” “fourth,” “fifth,” “sixth,” “seventh,” “eighth,” “ninth,” “tenth,” etc. as is apparent from the context of use.

“Promoter” indicates a DNA sequence that cause transcription of a DNA sequence to which it is operably linked, i.e., linked in such a way as to permit transcription of the nucleotide sequence of interest when the appropriate signals are present and repressors are absent. The expression of a polynucleotide of interest may be placed under control of any promoter or enhancer element known in the art. A eukaryotic promoter can be operably linked to a TATA Box. The TATA Box is typically located upstream of the transcription start site.

Useful promoters that may be used include, but are not limited to, the SV40 early promoter region, SV40 E/L (early late) promoter, the promoter contained in the 3′ long terminal repeat of Rous sarcoma virus, the regulatory sequences of the metallothionein gene, mouse or human cytomegalovirus major immediate early (CMV-MIE) promoter and other CMV promoters, including CMVmin promoters. Plant expression vectors comprising the nopaline synthetase promoter region, the cauliflower mosaic virus 35S RNA promoter, and the promoter of the photosynthetic enzyme ribulose biphosphate carboxylase; promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I; insulin; immunoglobulin; mouse mammary tumor virus; albumin; C.-feto protein; C.1-antitrypsin; 3-globin, and myosin light chain-2. Various forms of the CMV promoter can be used according to the inventions.

Minimal promoters, such as CMVmin promoters, can be truncated promoters or core promoters and are preferred for use in controlled expression systems. Minimal promoters and development approaches are widely known and disclosed in, for example, Saxena et al., Methods Molec. Biol. 1651:263-73 (2017); Ede et al., ACS Synth Biol. 5:395-404 (2016); Brown et al., Biotech Bioeng. 111:1638-47 (2014); Morita et al., Biotechniques 0:1-5 (2012); Lagrange et al., Genes Dev. 12:34-44 (1998). There are many CMVmin promoters described in the field.

“Reporter proteins” as used herein, refers to any protein capable of generating a detectable signal. Reporter proteins typically fluoresce, or catalyze a colorimetric or fluorescent reaction, and often are referred to as “fluorescent proteins” or “color proteins.” However, a reporter protein also can be non-enzymatic and non-fluorescent as long as it can be detected by another protein or moiety, such as a cell surface protein detected with a fluorescent ligand. A reporter protein also can be an inactive protein that is made functional through interaction with another protein that is fluorescent or catalyzes a reaction. Accordingly, any suitable reporter protein, as understood by one of skill in the art, could be used. In some aspects, the reporter protein may be selected from fluorescent protein, luciferase, alkaline phosphatase, β-galactosidase, β-lactamase, dihydrofolate reductase, ubiquitin, and variants thereof. Fluorescent proteins are useful for the recognition of gene cassettes that have or have not been successfully inserted and/or replaced, as the case may be. Fluid cytometry and fluorescence-activated cell sorting are suitable for detection. Examples of fluorescent proteins are well-known in the art, including, but not limited to Discosoma coral (DsRed), green fluorescent protein (GFP), enhanced green fluorescent protein (eGFP), cyano fluorescent protein (CFP), enhanced cyano fluorescent protein (eCFP), yellow fluorescent protein (YFP), enhanced yellow fluorescent protein (eYFP) and far-red fluorescent protein (e.g. mKate, mKate2, mPlum, mRaspberry or E2-crimson. See, for example, U.S. Pat. No. 9,816,110. Reporter proteins are encoded by polynucleotides, and are referred to herein as “reporter genes” or “reporter protein genes.” Reporters can be considered a type of marker. “Color” or “fluorescent,” in their various grammatical forms, also can be used the more specifically refer to a reporter protein or gene.

A “repressor protein”, also referred to as a “repressor,” is a protein that can bind to DNA in order to repressor transcription. Repressors are of eukaryotic and prokaryotic origin. Prokaryotic repressors are preferred. Examples of repressor families include: TetR, LysR, Lac, ArsR, IcIR, MerR, AsnC, MarR, DeoR, GntR and Crp families. Repressor proteins in the TetR family include: ArcR, Actil, AmeR, AmrR, ArpR, BpeR, EnvR, EthR, HemR, HydR, IfeR, LanK, LfrR, LmrA, MtrR, Pip, PqrA, QacR, RifQ, RmrR, SimReg2, SmeT, SrpR, TcmR, TetR, TtgR, TrgW, UrdK, VarR YdeS, ArpA., BarA, Aur1B, CaIR1, CprB, FarA, JadR*, JadR2, MphB, NonG, PhlF, TylQ, VanT, TarA, TyIP, BM1P1, Bm3R1, ButR, CampR, CamR, DhaR, KstR, LexA-like, AcnR, PaaRR, Psbl, Th1R, UidR, YDH1, Betl, McbR, MphR, PhaD, Q9ZF45, TtK, Yhgd, YixD, CasR, IcaR, LitR, LuxR, LuxT, OpaR, Orf2, SmcR, HapR, Ef0113, HlyllR, BarB, ScbR, MmfR, AmtR, PsrA andYjdC proteins See Ramos et al., Microbiol. Mol. Biol. Rev., 69: 326-56 (2005). Still other repressors include PurR, LacR, MetJ and PadR, Repressor proteins are encoded by genes referred to as “repressor genes” or “repressor protein genes.”

“Selectable” or “selection” marker proteins include proteins conferring certain traits, including but not limited to drug resistance or other selective advantages. Selection markers can give the cell receiving the selectable marker gene resistance towards a certain toxin, drug, antibiotic or other compound and permit the cell to produce protein and propagate in the presence of the toxin, drug, antibiotic or other compound, and are often referred to as “positive selectable markers.” Suitable examples of antibiotic resistance markers include, but are not limited to, proteins that impart resistance to various antibiotics, such as kanamycin, spectinomycin, neomycin, gentamycin (G418), ampicillin, tetracycline, chloramphenicol, puromycin, hygromycin, zeocin, and/or blasticidin. There are other selectable markers, often referred to as “negative selectable markers,” which cause a cell to stop propagating, stop protein production and/or are lethal to the cell in the presence of the negative selectable marker proteins. Thymidine kinase and certain fusion proteins can serve as negative selectable markers, including but not limited to GyrB-PKR. See White et al., Biotechniques, 50: 303-309 (May 2011). Selectable marker proteins and corresponding genes can be referred to generically as first (1), second (2), third (3), fourth (4), fifth (5), sixth (6), seventh (7), eighth (8), ninth (9), tenth (10), etc., as is apparent from the context of usage.

All numerical limits and ranges set forth herein include all numbers or values thereabout or there between of the numbers of the range or limit. The ranges and limits described herein expressly denominate and set forth all integers, decimals and fractional values defined and encompassed by the range or limit.

### DESCRIPTION

The inventions provide cells comprising AAV and optionally Ad polynucleotide sequences to allow production of recombinant AAV comprising a polynucleotide of interest, such as a gene or other sequence encoding a polypeptide of interest. The AAV and Ad polynucleotides provide the requisite structural and helper products required for AAV production.

AAV polynucleotides, and optionally Ad polynucleotides, can be integrated using a recombinase-mediated cassette exchange (RMCE), for example. “Stable” in the context of cell integration refers to a polynucleotide of interest, such as a gene, introduced into the genome of a cell and can be passed to subsequent generations of cells, and thereby can provide cell lines that are genetically homogeneous for a period of time.

Cells that are suitable for use with the inventions can be readily selected by those of skill in the art. In some embodiments the cell line is a eukaryotic cell line such as a yeast cell line, insect cell line (for example, Sf9 and Sf21 cells) or a mammalian cell line. Preferred mammalian cells include primate cells (including human), canine cells and rodent cells. Cells can be primary cells or immortalized cells. Suitable cells can be selected from Vero cells, COS cells, HEK 293 cells, HeLa cells, CHO cells, BHK cells, MDCK cells, amniotic cells (human), embryonic cells, cell lines transfected with viral genes, for example, AD5 E1, including but not limited to an immortalized human retinal cell transfected with an adenovirus gene, for example, a PER.C6 cell, or an NSO cell. In some embodiments, the cell is a Chinese hamster ovary (CHO) cell line. Some examples of CHO cells include, but are not limited to, CHO-ori, CHO-K1, CHO-s, CHO-DHB11, CHO-DXB11, CHO-K1 SV, and mutants and variants thereof. In other embodiments, the cell is a HEK293 cell. Some examples of HEK293 cells include, but are not limited, to HEK293, HEK293A, HEK293E, HEK293F, HEK293FT, HEK293FTM, HEK293H, HEK293MSR, HEK293S, HEK293SG, HEK293SGGD, HEK293T and mutants and variants thereof.

For hamster cells such as CHO and BHK, integration can be accomplished by inventions disclosed in U.S. Pat. No. 7,771,997 (“Stable Site 1”) and 9,816,110 (“Stable Site 2”), which are hereby incorporated by reference, including sequence information. Regeneron provides a suite of goods and services referred to as EESYR®. CHO cells with integrated sequences in Stable Site 1 and Stable Site 2 are disclosed in US 2019/0233544 A1, which is hereby incorporated by reference, including sequence information. Sequences set forth in these patents and Examples 14 and 15 can be used according to the inventions described and depicted herein. Additionally, an AAVS1-like region and the COSMC locus in hamster cells can be used according to the inventions.

Where human cells are employed, integration into adeno-associated virus integration site 1 (AAVS1) can be undertaken. See Lou et al., Human Gene Therapy Methods, 28: 124-38 (2017); Liu et al., BMC Research Note, 7:626 (2014). AAVS1 is reported to be located on chromosome 19. Other integration sites in human cells can be used as well, such as CCR5 and hROSA26.

Modification of cellular genomes can be undertaken with known approaches, such as Cre/Lox, Flp/Frt, transcription activator-like effector nuclease (TALEN), a TAL effector domain fusion protein, zinc finger nuclease (ZFN), a ZFN dimer, or a RNA-guided DNA endonuclease system, such as CRISPR/Cas9. See U.S. Pat. No. 9,816,110 at cols. 17-18. Integration using Bxb1 integrase in human, mouse and rat cells also can be undertaken. Russell et al., Biotechniques 40: 460-64 (2006).

To maximize stability and efficiency and facilitate integration and control of the inventions, Stable Integration Sites (SIS) can be created using Genomic Safe Harbors and the like in a wide variety of cell types and lines according to the teachings of U.S. Ser. No. 63/256,675. The descriptions (including examples) and figures providing methods and cells resulting from the methods of U.S. Ser. No. 63/256,675 are hereby incorporated by reference.

For production of recombinant AAV, the inventions provide for integration of AAV Cap to produce size variants VP1 (about 90 kDa), VP2 (about 72 kDa) and VP3 (about 60 kDa). The variants differ at their N-terminus.

Usually, recombinant AAV will contain a gene-of interest (GOI) flanked by AAV ITRs (inverted terminal repeats), For production of recombinant AAV, seven additional polynucleotides, namely adenovirus E1A, E1B, E4, E2A, VA RNA and AAV Rep and Cap, are typically employed for production.

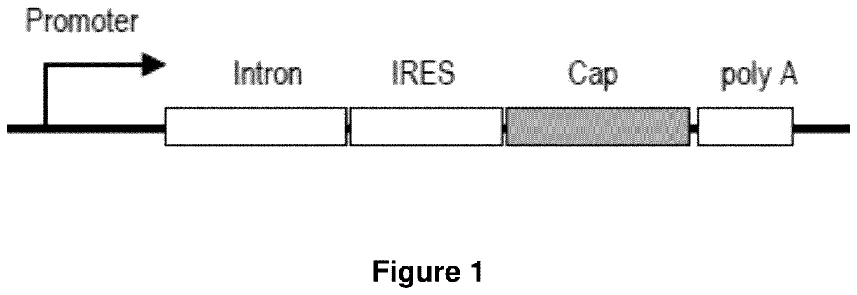

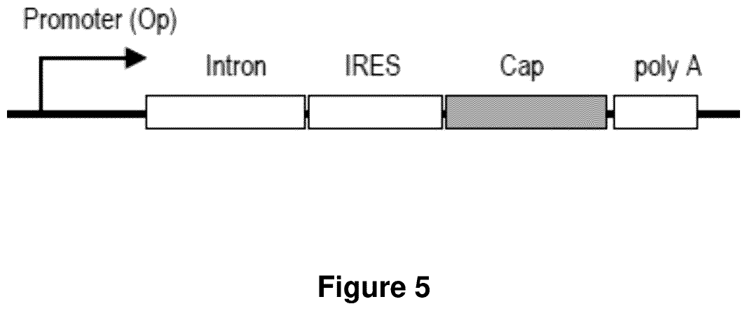

FIGS. 1-4 depict constructs that permit constitutive expression of Cap protein. FIGS. 5-8 depict constructs that permit controlled expression of Cap protein by inclusion of an operator downstream of the promoter. A preferred operator is the tetracycline operator (TetO), which binds the tetracycline repressor (TetR). Tetracycline, doxycycline and derivatives thereof can bind TetR so that TetR no longer binds TetO, and thus is permissive for transcription. An example of a CMV promoter and TetO is set forth in Example 13.

The inventions is further described by the following examples, which are illustrative of the many aspects of the invention, but do not limit the inventions in any manner.

### Example 1—CHO Cells

One or more Cap-containing polynucleotides according to FIGS. 1-8 are stably inserted into the CHO genome. A preferred promoter is the hCMV-IE promoter, and optionally a tet operator can be operably linked to the promoter for expression control. Optionally, an intron can be located 3′ of the promoter. A preferred intron is an hCMV-IE intron. AAV Cap, Rep and ITRs can be obtained from any AAV serotype. Preferred AAV serotypes are AAV2 and AAV5. AAV polynucleotide sequences are set forth in Example 11. Promoter, operator, IRES and intron sequences are set forth in Example 13.

AAV ITRs flanking a gene of interest, AAV Rep and Ad E1A, E1B, E2A (or E2A partial sequence (E2A orf)), E4 (or E4 partial sequence (E4 orf 6)) and VA RNA can be randomly integrated, site-specifically integrated or remain on a plasmid. Adenovirus polynucleotide sequences are available and are exemplified in Example 12. Adenovirus (Ad) proteins and polynucleotides can be selected from any serotype.

### Example 2—HEK 293 Cells

One or more Cap-containing polynucleotides according to FIGS. 1-8 are stably inserted into the HEK 293 genome. A preferred promoter is the hCMV-IE promoter, and optionally a tet operator can be operably linked to the promoter for expression control. Optionally, an intron can be located 3′ of the promoter. A preferred intron is an hCMV-IE intron. AAV Cap, Rep and ITRs can be obtained from any AAV serotype. Preferred AAV serotypes of AAV2 and AAV5. AAV polynucleotide sequences are set forth in Example 11.

AAV ITRs and Rep and Ad E1A, E1B, E2A (or E2A partial sequence (E2A orf)), E4 (or E4 partial sequence (E4 orf 6)) and VA RNA can be randomly integrated, site-specifically integrated or remain on a plasmid. Adenovirus polynucleotide sequences are available and are exemplified in Example 12.

### Example 3—BHK Cells

BHK cells are fibroblast cells from baby hamster kidneys. There are adherent BHK lines and BHK lines that can propagate in suspension. Wentz and Schugerl, Enzyme Microbial Tech. 14: 68-75 (1992).

One or more Cap-containing polynucleotides according to FIGS. 1-8 are stably inserted into the BHK genome. A preferred promoter is the hCMV-IE promoter, and optionally a tet operator can be operably linked to the promoter for expression control. Optionally, an intron can be located 3′ of the promoter. A preferred intron is an hCMV-IE intron. AAV Cap, Rep and ITRs can be obtained from any AAV serotype. Preferred AAV serotypes of AAV2 and AAV5. AAV polynucleotide sequences are set forth in Example 11.

AAV ITRs and Rep and Ad E1A, E1B, E2A (or E2A partial sequence (E2A orf)), E4 (or E4 partial sequence (E4 orf 6)) and VA RNA can be randomly integrated, site-specifically integrated or remain on a plasmid. Adenovirus polynucleotide sequences are available and are exemplified in Example 12.

### Example 4—Intron IRES CAP

Embodiments of this construct are depicted in FIGS. 1 (constitutive) and 5 (controllable). In a cell, both embodiments can primarily produce VP2 and VP3, with more VP3 being produced than VP2. In an experiment in CHO cells using Cap5, an average ratio of about 1:5.7 of VP2 to VP3 was observed by conducting densitometry analyses on western blots. Some VP1 also can be produced, but the level observed was typically less than 1% the level of VP2 that is produced. Accordingly, this construct can be used with a construct that will produce primarily VP1. See Examples 6 and 7. Production ratios can vary based upon experimental conditions and analytical techniques. A preferred IRES is the encephalomyocarditis virus (referred to as “EMCV” or “ECMV”) IRES.

### Example 5—Intron IRES CAP IRES CAP

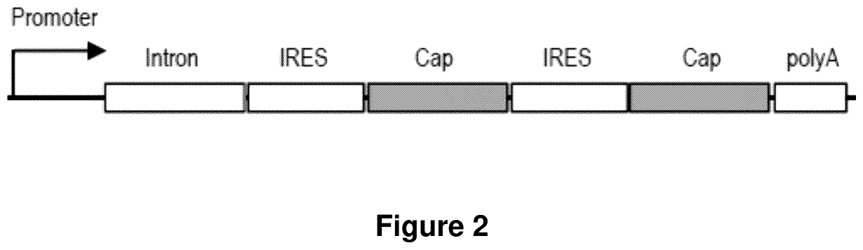

Embodiments of this construct are depicted in FIGS. 2 (constitutive) and 6 (controllable). This construct contains two IRES polynucleotides and two Cap polynucleotides. In a cell, both embodiments can produce a VP1, VP2 and VP3. The amount of VP3 production can be greater than the amount of VP1 production and the amount of VP2 production. The amount of VP2 production can be greater than the amount of VP1 production. In an experiment using CHO cells containing Cap5, an average ratio of about 1:2:9.3 of VP1 to VP2 to VP3 was observed by conducting densitometry analyses on western blots. Production ratios can vary based upon experimental conditions and analytical techniques. A preferred IRES is the encephalomyocarditis virus (referred to as “EMCV” or “ECMV”) IRES.

### Example 6—Intron CAP

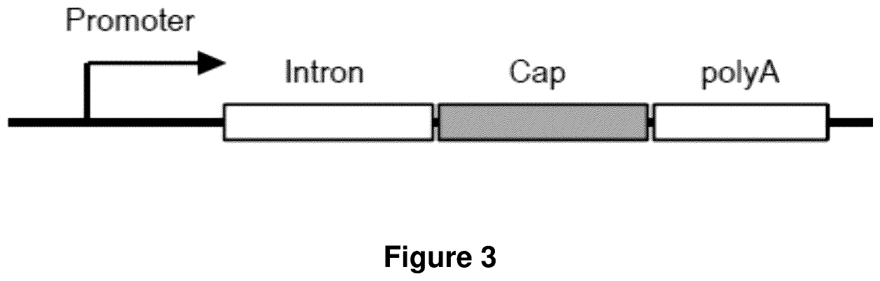

Embodiments of this construct are depicted in FIGS. 3 (constitutive) and 7 (controllable). This construct contains an Intron and Cap polynucleotide, and produces predominantly VP1, and can be used with the constructs of Example 4 to produce VP1, VP2 and VP3.

### Example 7—IRES CAP

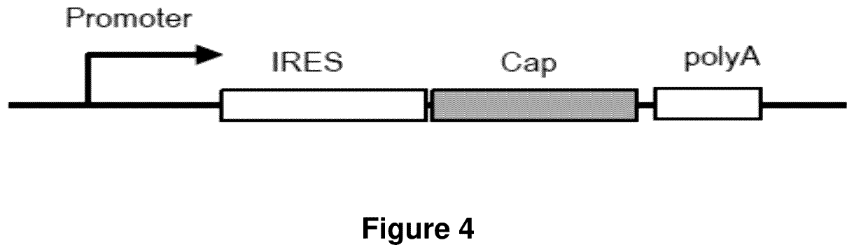

Embodiments of this construct are depicted in FIGS. 4 (constitutive) and 8 (controllable). This construct contains an Intron and Cap polynucleotide, and produces predominantly VP1, and can be used with the constructs of Example 4 to produce VP1, VP2 and VP3. A preferred IRES is the encephalomyocarditis virus (referred to as “EMCV” or “ECMV”) IRES.

### Example 8—CHO Cells Comprising Cap Constructs

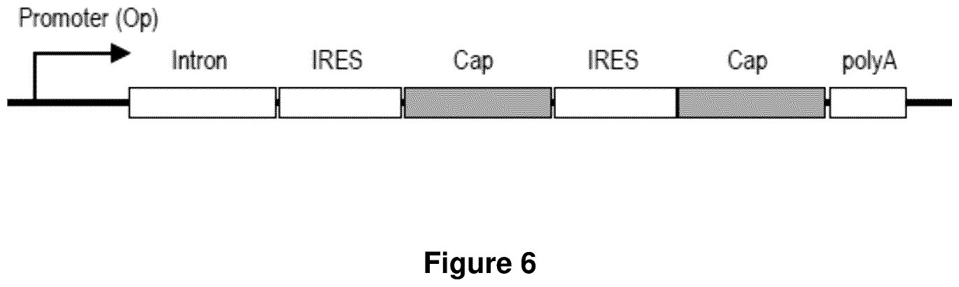

CHO cells of Example 1 can comprise an Intron IRES CAP IRES CAP polynucleotide of Example 5 (FIG. 2 or 6). In an experiment, CHO cells containing Cap5 expressed VP1:VP2:VP3 in an observed ratio of about 1:2:9.3 by conducting densitometry analyses on western blots.

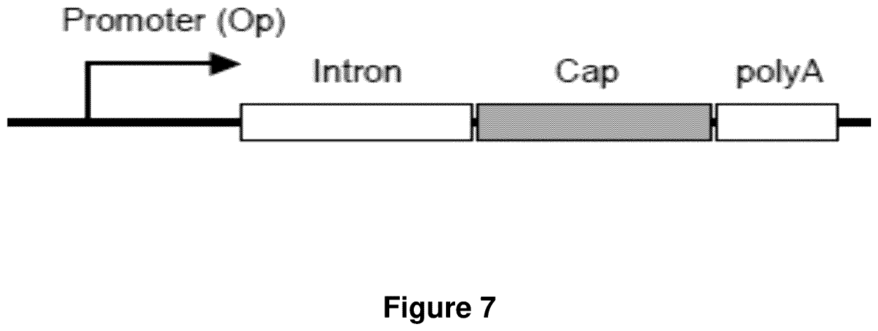

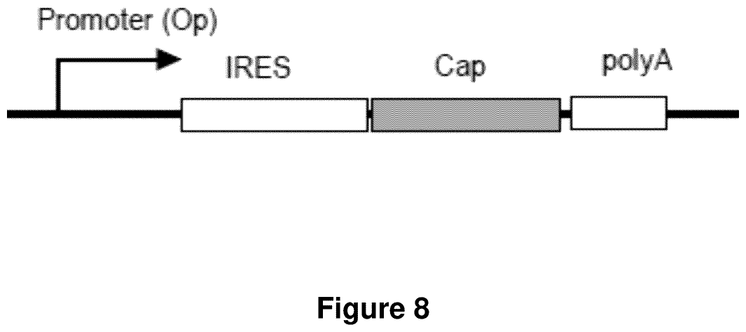

As an alternative, CHO cells of Example 1 can comprise CAP polynucleotides of Example 4 (FIG. 1 or 5) and Examples 6 (FIG. 3 or 7) or 6 (FIGS. 4 and 8). Such cells would express VP1, VP2 and VP3, although possibly at different ratios than according to Example 5.

### Example 9—HEK 293 Cells Comprising Cap Constructs

HEK 293 cells of Example 2 can comprise an Intron IRES CAP IRES CAP polynucleotide of Example 5 (FIG. 2 or 6).

As an alternative, HEK 293 cells of Example 2 can comprise CAP polynucleotides of Example 4 (FIG. 1 or 5) and Examples 6 (FIG. 3 or 7) or 7 (FIGS. 4 and 8). Such cells would express VP1, VP2 and VP3, although possibly at different ratios than according to Example 5.

### Example 10—BHK Cells Comprising Cap Constructs

BHK cells of Example 3 can comprise an Intron IRES CAP IRES CAP polynucleotide of Example 5 (FIG. 2 or 6).

As an alternative, BHK cells of Example 3 can comprise CAP polynucleotides of Example 4 (FIG. 1 or 5) and Examples 6 (FIG. 3 or 7) or 7 (FIGS. 4 and 8). Such cells would express VP1, VP2 and VP3, although possibly at different ratios than according to Example 5.

### Example 11—AAV Polynucleotide Sequences

AAV Rep, Cap and ITR sequences are known in the art. The present inventions are amenable to all AAV serotypes. AAV sequences from various AAV serotypes are set forth below. Many of these sequences are available from the National Center for Biotechnology Information (NCBI).

### AAV-1

### Full Genome: NC 002077

CapVP1:
(SEQ ID NO: 1)
ATGGCTGCCGATGGTTATCTTCCAGATTGGCTCGAGGACAACCTCTCTGAGGGCATTCGCGAGTGGTGGGACTTG
AAACCTGGAGCCCCGAAGCCCAAAGCCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGCGGGTT
CTCGAACCTCTCGGTCTGGTTGAGGAAGGCGCTAAGACGGCTCCTGGAAAGAAACGTCCGGTAGAGCAGTCGCCA
CAAGAGCCAGACTCCTCCTCGGGCATCGGCAAGACAGGCCAGCAGCCCGCTAAAAAGAGACTCAATTTTGGTCAG
ACTGGCGACTCAGAGTCAGTCCCCGATCCACAACCTCTCGGAGAACCTCCAGCAACCCCCGCTGCTGTGGGACCT
ACTACAATGGCTTCAGGCGGTGGCGCACCAATGGCAGACAATAACGAAGGCGCCGACGGAGTGGGTAATGCCTCA
GGAAATTGGCATTGCGATTCCACATGGCTGGGCGACAGAGTCATCACCACCAGCACCCGCACCTGGGCCTTGCCC
ACCTACAATAACCACCTCTACAAGCAAATCTCCAGTGCTTCAACGGGGGCCAGCAACGACAACCACTACTTCGGC
TACAGCACCCCCTGGGGGTATTTTGATTTCAACAGATTCCACTGCCACTTTTCACCACGTGACTGGCAGCGACTC
ATCAACAACAATTGGGGATTCCGGCCCAAGAGACTCAACTTCAAACTCTTCAACATCCAAGTCAAGGAGGTCACG
ACGAATGATGGCGTCACAACCATCGCTAATAACCTTACCAGCACGGTTCAAGTCTTCTCGGACTCGGAGTACCAG
CTTCCGTACGTCCTCGGCTCTGCGCACCAGGGCTGCCTCCCTCCGTTCCCGGCGGACGTGTTCATGATTCCGCAA
TACGGCTACCTGACGCTCAACAATGGCAGCCAAGCCGTGGGACGTTCATCCTTTTACTGCCTGGAATATTTCCCT
TCTCAGATGCTGAGAACGGGCAACAACTTTACCTTCAGCTACACCTTTGAGGAAGTGCCTTTCCACAGCAGCTAC
GCGCACAGCCAGAGCCTGGACCGGCTGATGAATCCTCTCATCGACCAATACCTGTATTACCTGAACAGAACTCAA
AATCAGTCCGGAAGTGCCCAAAACAAGGACTTGCTGTTTAGCCGTGGGTCTCCAGCTGGCATGTCTGTTCAGCCC
AAAAACTGGCTACCTGGACCCTGTTATCGGCAGCAGCGCGTTTCTAAAACAAAAACAGACAACAACAACAGCAAT
TTTACCTGGACTGGTGCTTCAAAATATAACCTCAATGGGCGTGAATCCATCATCAACCCTGGCACTGCTATGGCC
TCACACAAAGACGACGAAGACAAGTTCTTTCCCATGAGCGGTGTCATGATTTTTGGAAAAGAGAGCGCCGGAGCT
TCAAACACTGCATTGGACAATGTCATGATTACAGACGAAGAGGAAATTAAAGCCACTAACCCTGTGGCCACCGAA
AGATTTGGGACCGTGGCAGTCAATTTCCAGAGCAGCAGCACAGACCCTGCGACCGGAGATGTGCATGCTATGGGA
GCATTACCTGGCATGGTGTGGCAAGATAGAGACGTGTACCTGCAGGGTCCCATTTGGGCCAAAATTCCTCACACA
GATGGACACTTTCACCCGTCTCCTCTTATGGGCGGCTTTGGACTCAAGAACCCGCCTCCTCAGATCCTCATCAAA
AACACGCCTGTTCCTGCGAATCCTCCGGCGGAGTTTTCAGCTACAAAGTTTGCTTCATTCATCACCCAATACTCC
ACAGGACAAGTGAGTGTGGAAATTGAATGGGAGCTGCAGAAAGAAAACAGCAAGCGCTGGAATCCCGAAGTGCAG
TACACATCCAATTATGCAAAATCTGCCAACGTTGATTTTACTGTGGACAACAATGGACTTTATACTGAGCCTCGC
CCCATTGGCACCCGTTACCTTACCCGTCCCCTGTAA
Rep78:
(SEQ ID NO: 2)
ATGCCGGGCTTCTACGAGATCGTGATCAAGGTGCCGAGCGACCTGGACGAGCACCTGCCGGGCATTTCTGACTCG
TTTGTGAGCTGGGTGGCCGAGAAGGAATGGGAGCTGCCCCCGGATTCTGACATGGATCTGAATCTGATTGAGCAG
GCACCCCTGACCGTGGCCGAGAAGCTGCAGCGCGACTTCCTGGTCCAATGGCGCCGCGTGAGTAAGGCCCCGGAG
GCCCTCTTCTTTGTTCAGTTCGAGAAGGGCGAGTCCTACTTCCACCTCCATATTCTGGTGGAGACCACGGGGGTC
AAATCCATGGTGCTGGGCCGCTTCCTGAGTCAGATTAGGGACAAGCTGGTGCAGACCATCTACCGCGGGATCGAG
CCGACCCTGCCCAACTGGTTCGCGGTGACCAAGACGCGTAATGGCGCCGGAGGGGGGAACAAGGTGGTGGACGAG
TGCTACATCCCCAACTACCTCCTGCCCAAGACTCAGCCCGAGCTGCAGTGGGCGTGGACTAACATGGAGGAGTAT
ATAAGCGCCTGTTTGAACCTGGCCGAGCGCAAACGGCTCGTGGCGCAGCACCTGACCCACGTCAGCCAGACCCAG
GAGCAGAACAAGGAGAATCTGAACCCCAATTCTGACGCGCCTGTCATCCGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCTTCCAACTCGCGGTCCCAGATCAAGGCCGCTCTGGACAATGCCGGCAAGATCATGGCG
CTGACCAAATCCGCGCCCGACTACCTGGTAGGCCCCGCTCCGCCCGCGGACATTAAAACCAACCGCATCTACCGC
ATCCTGGAGCTGAACGGCTACGAACCTGCCTACGCCGGCTCCGTCTTTCTCGGCTGGGCCCAGAAAAGGTTCGGG
AAGCGCAACACCATCTGGCTGTTTGGGCCGGCCACCACGGGCAAGACCAACATCGCGGAAGCCATCGCCCACGCC
GTGCCCTTCTACGGCTGCGTCAACTGGACCAATGAGAACTTTCCCTTCAATGATTGCGTCGACAAGATGGTGATC
TGGTGGGAGGAGGGCAAGATGACGGCCAAGGTCGTGGAGTCCGCCAAGGCCATTCTCGGCGGCAGCAAGGTGCGC
GTGGACCAAAAGTGCAAGTCGTCCGCCCAGATCGACCCCACCCCCGTGATCGTCACCTCCAACACCAACATGTGC
GCCGTGATTGACGGGAACAGCACCACCTTCGAGCACCAGCAGCCGTTGCAGGACCGGATGTTCAAATTTGAACTC
ACCCGCCGTCTGGAGCATGACTTTGGCAAGGTGACAAAGCAGGAAGTCAAAGAGTTCTTCCGCTGGGCGCAGGAT
CACGTGACCGAGGTGGCGCATGAGTTCTACGTCAGAAAGGGTGGAGCCAACAAAAGACCCGCCCCCGATGACGCG
GATAAAAGCGAGCCCAAGCGGGCCTGCCCCTCAGTCGCGGATCCATCGACGTCAGACGCGGAAGGAGCTCCGGTG
GACTTTGCCGACAGGTACCAAAACAAATGTTCTCGTCACGCGGGCATGCTTCAGATGCTGTTTCCCTGCAAGACA
TGCGAGAGAATGAATCAGAATTTCAACATTTGCTTCACGCACGGGACGAGAGACTGTTCAGAGTGCTTCCCCGGC
GTGTCAGAATCTCAACCGGTCGTCAGAAAGAGGACGTATCGGAAACTCTGTGCCATTCATCATCTGCTGGGGCGG
GCTCCCGAGATTGCTTGCTCGGCCTGCGATCTGGTCAACGTGGACCTGGATGACTGTGTTTCTGAGCAATAA

### AAV-2

### Full Genome: NC 001401

Rep78:
(SEQ ID NO: 3)
ATGCCGGGGTTTTACGAGATTGTGATTAAGGTCCCCAGCGACCTTGACGAGCATCTGCCCGGCATTTCTGACAGC
TTTGTGAACTGGGTGGCCGAGAAGGAATGGGAGTTGCCGCCAGATTCTGACATGGATCTGAATCTGATTGAGCAG
GCACCCCTGACCGTGGCCGAGAAGCTGCAGCGCGACTTTCTGACGGAATGGCGCCGTGTGAGTAAGGCCCCGGAG
GCCCTTTTCTTTGTGCAATTTGAGAAGGGAGAGAGCTACTTCCACATGCACGTGCTCGTGGAAACCACCGGGGTG
AAATCCATGGTTTTGGGACGTTTCCTGAGTCAGATTCGCGAAAAACTGATTCAGAGAATTTACCGCGGGATCGAG
CCGACTTTGCCAAACTGGTTCGCGGTCACAAAGACCAGAAATGGCGCCGGAGGCGGGAACAAGGTGGTGGATGAG
TGCTACATCCCCAATTACTTGCTCCCCAAAACCCAGCCTGAGCTCCAGTGGGCGTGGACTAATATGGAACAGTAT
TTAAGCGCCTGTTTGAATCTCACGGAGCGTAAACGGTTGGTGGCGCAGCATCTGACGCACGTGTCGCAGACGCAG
GAGCAGAACAAAGAGAATCAGAATCCCAATTCTGATGCGCCGGTGATCAGATCAAAAACTTCAGCCAGGTACATG
GAGCTGGTCGGGTGGCTCGTGGACAAGGGGATTACCTCGGAGAAGCAGTGGATCCAGGAGGACCAGGCCTCATAC
ATCTCCTTCAATGCGGCCTCCAACTCGCGGTCCCAAATCAAGGCTGCCTTGGACAATGCGGGAAAGATTATGAGC
CTGACTAAAACCGCCCCCGACTACCTGGTGGGCCAGCAGCCCGTGGAGGACATTTCCAGCAATCGGATTTATAAA
ATTTTGGAACTAAACGGGTACGATCCCCAATATGCGGCTTCCGTCTTTCTGGGATGGGCCACGAAAAAGTTCGGC
AAGAGGAACACCATCTGGCTGTTTGGGCCTGCAACTACCGGGAAGACCAACATCGCGGAGGCCATAGCCCACACT
GTGCCCTTCTACGGGTGCGTAAACTGGACCAATGAGAACTTTCCCTTCAACGACTGTGTCGACAAGATGGTGATC
TGGTGGGAGGAGGGGAAGATGACCGCCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGATCGTCACCTCCAACACCAACATGTGC
GCCGTGATTGACGGGAACTCAACGACCTTCGAACACCAGCAGCCGTTGCAAGACCGGATGTTCAAATTTGAACTC
ACCCGCCGTCTGGATCATGACTTTGGGAAGGTCACCAAGCAGGAAGTCAAAGACTTTTTCCGGTGGGCAAAGGAT
CACGTGGTTGAGGTGGAGCATGAATTCTACGTCAAAAAGGGTGGAGCCAAGAAAAGACCCGCCCCCAGTGACGCA
GATATAAGTGAGCCCAAACGGGTGCGCGAGTCAGTTGCGCAGCCATCGACGTCAGACGCGGAAGCTTCGATCAAC
TACGCAGACAGGTACCAAAACAAATGTTCTCGTCACGTGGGCATGAATCTGATGCTGTTTCCCTGCAGACAATGC
GAGAGAATGAATCAGAATTCAAATATCTGCTTCACTCACGGACAGAAAGACTGTTTAGAGTGCTTTCCCGTGTCA
GAATCTCAACCCGTTTCTGTCGTCAAAAAGGCGTATCAGAAACTGTGCTACATTCATCATATCATGGGAAAGGTG
CCAGACGCTTGCACTGCCTGCGATCTGGTCAATGTGGATTTGGATGACTGCATCTTTGAACAATAA
Rep52:
(SEQ ID NO: 4)
ATGGAGCTGGTCGGGTGGCTCGTGGACAAGGGGATTACCTCGGAGAAGCAGTGGATCCAGGAGGACCAGGCCTCA
TACATCTCCTTCAATGCGGCCTCCAACTCGCGGTCCCAAATCAAGGCTGCCTTGGACAATGCGGGAAAGATTATG
AGCCTGACTAAAACCGCCCCCGACTACCTGGTGGGCCAGCAGCCCGTGGAGGACATTTCCAGCAATCGGATTTAT
AAAATTTTGGAACTAAACGGGTACGATCCCCAATATGCGGCTTCCGTCTTTCTGGGATGGGCCACGAAAAAGTTC
GGCAAGAGGAACACCATCTGGCTGTTTGGGCCTGCAACTACCGGGAAGACCAACATCGCGGAGGCCATAGCCCAC
ACTGTGCCCTTCTACGGGTGCGTAAACTGGACCAATGAGAACTTTCCCTTCAACGACTGTGTCGACAAGATGGTG
ATCTGGTGGGAGGAGGGGAAGATGACCGCCAAGGTCGTGGAGTCGGCCAAAGCCATTCTCGGAGGAAGCAAGGTG
CGCGTGGACCAGAAATGCAAGTCCTCGGCCCAGATAGACCCGACTCCCGTGATCGTCACCTCCAACACCAACATG
TGCGCCGTGATTGACGGGAACTCAACGACCTTCGAACACCAGCAGCCGTTGCAAGACCGGATGTTCAAATTTGAA
CTCACCCGCCGTCTGGATCATGACTTTGGGAAGGTCACCAAGCAGGAAGTCAAAGACTTTTTCCGGTGGGCAAAG
GATCACGTGGTTGAGGTGGAGCATGAATTCTACGTCAAAAAGGGTGGAGCCAAGAAAAGACCCGCCCCCAGTGAC
GCAGATATAAGTGAGCCCAAACGGGTGCGCGAGTCAGTTGCGCAGCCATCGACGTCAGACGCGGAAGCTTCGATC
AACTACGCAGACAGGTACCAAAACAAATGTTCTCGTCACGTGGGCATGAATCTGATGCTGTTTCCCTGCAGACAA
TGCGAGAGAATGAATCAGAATTCAAATATCTGCTTCACTCACGGACAGAAAGACTGTTTAGAGTGCTTTCCCGTG
TCAGAATCTCAACCCGTTTCTGTCGTCAAAAAGGCGTATCAGAAACTGTGCTACATTCATCATATCATGGGAAAG
GTGCCAGACGCTTGCACTGCCTGCGATCTGGTCAATGTGGATTTGGATGACTGCATCTTTGAACAATAA
CapVP1:
(SEQ ID NO: 5)
ATGGCTGCCGATGGTTATCTTCCAGATTGGCTCGAGGACACTCTCTCTGAAGGAATAAGACAGTGGTGGAAGCTC
AAACCTGGCCCACCACCACCAAAGCCCGCAGAGCGGCATAAGGACGACAGCAGGGGTCTTGTGCTTCCTGGGTAC
AAGTACCTCGGACCCTTCAACGGACTCGACAAGGGAGAGCCGGTCAACGAGGCAGACGCCGCGGCCCTCGAGCAC
GACAAAGCCTACGACCGGCAGCTCGACAGCGGAGACAACCCGTACCTCAAGTACAACCACGCCGACGCGGAGTTT
CAGGAGCGCCTTAAAGAAGATACGTCTTTTGGGGGCAACCTCGGACGAGCAGTCTTCCAGGCGAAAAAGAGGGTT
CTTGAACCTCTGGGCCTGGTTGAGGAACCTGTTAAGACGGCTCCGGGAAAAAAGAGGCCGGTAGAGCACTCTCCT
GTGGAGCCAGACTCCTCCTCGGGAACCGGAAAGGCGGGCCAGCAGCCTGCAAGAAAAAGATTGAATTTTGGTCAG
ACTGGAGACGCAGACTCAGTACCTGACCCCCAGCCTCTCGGACAGCCACCAGCAGCCCCCTCTGGTCTGGGAACT
AATACGATGGCTACAGGCAGTGGCGCACCAATGGCAGACAATAACGAGGGCGCCGACGGAGTGGGTAATTCCTCG
GGAAATTGGCATTGCGATTCCACATGGATGGGCGACAGAGTCATCACCACCAGCACCCGAACCTGGGCCCTGCCC
ACCTACAACAACCACCTCTACAAACAAATTTCCAGCCAATCAGGAGCCTCGAACGACAATCACTACTTTGGCTAC
AGCACCCCTTGGGGGTATTTTGACTTCAACAGATTCCACTGCCACTTTTCACCACGTGACTGGCAAAGACTCATC
AACAACAACTGGGGATTCCGACCCAAGAGACTCAACTTCAAGCTCTTTAACATTCAAGTCAAAGAGGTCACGCAG
AATGACGGTACGACGACGATTGCCAATAACCTTACCAGCACGGTTCAGGTGTTTACTGACTCGGAGTACCAGCTC
CCGTACGTCCTCGGCTCGGCGCATCAAGGATGCCTCCCGCCGTTCCCAGCAGACGTCTTCATGGTGCCACAGTAT
GGATACCTCACCCTGAACAACGGGAGTCAGGCAGTAGGACGCTCTTCATTTTACTGCCTGGAGTACTTTCCTTCT
CAGATGCTGCGTACCGGAAACAACTTTACCTTCAGCTACACTTTTGAGGACGTTCCTTTCCACAGCAGCTACGCT
CACAGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTCTAGG
AACTGGCTTCCTGGACCCTGTTACCGCCAGCAGCGAGTATCAAAGACATCTGCGGATAACAACAACAGTGAATAC
TCGTGGACTGGAGCTACCAAGTACCACCTCAATGGCAGAGACTCTCTGGTGAATCCGGGCCCGGCCATGGCAAGC
CACAAGGACGATGAAGAAAAGTTTTTTCCTCAGAGCGGGGTTCTCATCTTTGGGAAGCAAGGCTCAGAGAAAACA
AATGTGGACATTGAAAAGGTCATGATTACAGACGAAGAGGAAATCAGGACAACCAATCCCGTGGCTACGGAGCAG
TATGGTTCTGTATCTACCAACCTCCAGAGAGGCAACAGACAAGCAGCTACCGCAGATGTCAACACACAAGGCGTT
CTTCCAGGCATGGTCTGGCAGGACAGAGATGTGTACCTTCAGGGGCCCATCTGGGCAAAGATTCCACACACGGAC
GGACATTTTCACCCCTCTCCCCTCATGGGTGGATTCGGACTTAAACACCCTCCTCCACAGATTCTCATCAAGAAC
ACCCCGGTACCTGCGAATCCTTCGACCACCTTCAGTGCGGCAAAGTTTGCTTCCTTCATCACACAGTACTCCACG
GGACAGGTCAGCGTGGAGATCGAGTGGGAGCTGCAGAAGGAAAACAGCAAACGCTGGAATCCCGAAATTCAGTAC
ACTTCCAACTACAACAAGTCTGTTAATGTGGACTTTACTGTGGACACTAATGGCGTGTATTCAGAGCCTCGCCCC
ATTGGCACCAGATACCTGACTCGTAATCTGTAA
CapVP2:
(SEQ ID NO: 6)
ACGGCTCCGGGAAAAAAGAGGCCGGTAGAGCACTCTCCTGTGGAGCCAGACTCCTCCTCGGGAACCGGAAAGGCG
GGCCAGCAGCCTGCAAGAAAAAGATTGAATTTTGGTCAGACTGGAGACGCAGACTCAGTACCTGACCCCCAGCCT
CTCGGACAGCCACCAGCAGCCCCCTCTGGTCTGGGAACTAATACGATGGCTACAGGCAGTGGCGCACCAATGGCA
GACAATAACGAGGGCGCCGACGGAGTGGGTAATTCCTCGGGAAATTGGCATTGCGATTCCACATGGATGGGCGAC
AGAGTCATCACCACCAGCACCCGAACCTGGGCCCTGCCCACCTACAACAACCACCTCTACAAACAAATTTCCAGC
CAATCAGGAGCCTCGAACGACAATCACTACTTTGGCTACAGCACCCCTTGGGGGTATTTTGACTTCAACAGATTC
CACTGCCACTTTTCACCACGTGACTGGCAAAGACTCATCAACAACAACTGGGGATTCCGACCCAAGAGACTCAAC
TTCAAGCTCTTTAACATTCAAGTCAAAGAGGTCACGCAGAATGACGGTACGACGACGATTGCCAATAACCTTACC
AGCACGGTTCAGGTGTTTACTGACTCGGAGTACCAGCTCCCGTACGTCCTCGGCTCGGCGCATCAAGGATGCCTC
CCGCCGTTCCCAGCAGACGTCTTCATGGTGCCACAGTATGGATACCTCACCCTGAACAACGGGAGTCAGGCAGTA
GGACGCTCTTCATTTTACTGCCTGGAGTACTTTCCTTCTCAGATGCTGCGTACCGGAAACAACTTTACCTTCAGC
TACACTTTTGAGGACGTTCCTTTCCACAGCAGCTACGCTCACAGCCAGAGTCTGGACCGTCTCATGAATCCTCTC
ATCGACCAGTACCTGTATTACTTGAGCAGAACAAACACTCCAAGTGGAACCACCACGCAGTCAAGGCTTCAGTTT
TCTCAGGCCGGAGCGAGTGACATTCGGGACCAGTCTAGGAACTGGCTTCCTGGACCCTGTTACCGCCAGCAGCGA
GTATCAAAGACATCTGCGGATAACAACAACAGTGAATACTCGTGGACTGGAGCTACCAAGTACCACCTCAATGGC
AGAGACTCTCTGGTGAATCCGGGCCCGGCCATGGCAAGCCACAAGGACGATGAAGAAAAGTTTTTTCCTCAGAGC
GGGGTTCTCATCTTTGGGAAGCAAGGCTCAGAGAAAACAAATGTGGACATTGAAAAGGTCATGATTACAGACGAA
GAGGAAATCAGGACAACCAATCCCGTGGCTACGGAGCAGTATGGTTCTGTATCTACCAACCTCCAGAGAGGCAAC
AGACAAGCAGCTACCGCAGATGTCAACACACAAGGCGTTCTTCCAGGCATGGTCTGGCAGGACAGAGATGTGTAC
CTTCAGGGGCCCATCTGGGCAAAGATTCCACACACGGACGGACATTTTCACCCCTCTCCCCTCATGGGTGGATTC
GGACTTAAACACCCTCCTCCACAGATTCTCATCAAGAACACCCCGGTACCTGCGAATCCTTCGACCACCTTCAGT
GCGGCAAAGTTTGCTTCCTTCATCACACAGTACTCCACGGGACAGGTCAGCGTGGAGATCGAGTGGGAGCTGCAG
AAGGAAAACAGCAAACGCTGGAATCCCGAAATTCAGTACACTTCCAACTACAACAAGTCTGTTAATGTGGACTTT
ACTGTGGACACTAATGGCGTGTATTCAGAGCCTCGCCCCATTGGCACCAGATACCTGACTCGTAATCTGTAA
CapVP3:
(SEQ ID NO: 7)
ATGGCTACAGGCAGTGGCGCACCAATGGCAGACAATAACGAGGGCGCCGACGGAGTGGGTAATTCCTCGGGAAAT
TGGCATTGCGATTCCACATGGATGGGCGACAGAGTCATCACCACCAGCACCCGAACCTGGGCCCTGCCCACCTAC
AACAACCACCTCTACAAACAAATTTCCAGCCAATCAGGAGCCTCGAACGACAATCACTACTTTGGCTACAGCACC
CCTTGGGGGTATTTTGACTTCAACAGATTCCACTGCCACTTTTCACCACGTGACTGGCAAAGACTCATCAACAAC
AACTGGGGATTCCGACCCAAGAGACTCAACTTCAAGCTCTTTAACATTCAAGTCAAAGAGGTCACGCAGAATGAC
GGTACGACGACGATTGCCAATAACCTTACCAGCACGGTTCAGGTGTTTACTGACTCGGAGTACCAGCTCCCGTAC
GTCCTCGGCTCGGCGCATCAAGGATGCCTCCCGCCGTTCCCAGCAGACGTCTTCATGGTGCCACAGTATGGATAC
CTCACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCACAGC
CAGAGTCTGGACCGTCTCATGAATCCTCTCATCGACCAGTACCTGTATTACTTGAGCAGAACAAACACTCCAAGT
GGAACCACCACGCAGTCAAGGCTTCAGTTTTCTCAGGCCGGAGCGAGTGACATTCGGGACCAGTCTAGGAACTGG
CTTCCTGGACCCTGTTACCGCCAGCAGCGAGTATCAAAGACATCTGCGGATAACAACAACAGTGAATACTCGTGG
ACTGGAGCTACCAAGTACCACCTCAATGGCAGAGACTCTCTGGTGAATCCGGGCCCGGCCATGGCAAGCCACAAG
GACGATGAAGAAAAGTTTTTTCCTCAGAGCGGGGTTCTCATCTTTGGGAAGCAAGGCTCAGAGAAAACAAATGTG
GACATTGAAAAGGTCATGATTACAGACGAAGAGGAAATCAGGACAACCAATCCCGTGGCTACGGAGCAGTATGGT
TCTGTATCTACCAACCTCCAGAGAGGCAACAGACAAGCAGCTACCGCAGATGTCAACACACAAGGCGTTCTTCCA
GGCATGGTCTGGCAGGACAGAGATGTGTACCTTCAGGGGCCCATCTGGGCAAAGATTCCACACACGGACGGACAT
TTTCACCCCTCTCCCCTCATGGGTGGATTCGGACTTAAACACCCTCCTCCACAGATTCTCATCAAGAACACCCCG
GTACCTGCGAATCCTTCGACCACCTTCAGTGCGGCAAAGTTTGCTTCCTTCATCACACAGTACTCCACGGGACAG
GTCAGCGTGGAGATCGAGTGGGAGCTGCAGAAGGAAAACAGCAAACGCTGGAATCCCGAAATTCAGTACACTTCC
AACTACAACAAGTCTGTTAATGTGGACTTTACTGTGGACACTAATGGCGTGTATTCAGAGCCTCGCCCCATTGGC
ACCAGATACCTGACTCGTAATCTGTAA
CapAAP:
(SEQ ID NO: 8)
CTGGAGACGCAGACTCAGTACCTGACCCCCAGCCTCTCGGACAGCCACCAGCAGCCCCCTCTGGTCTGGGAACTA
ATACGATGGCTACAGGCAGTGGCGCACCAATGGCAGACAATAACGAGGGCGCCGACGGAGTGGGTAATTCCTCGG
GAAATTGGCATTGCGATTCCACATGGATGGGCGACAGAGTCATCACCACCAGCACCCGAACCTGGGCCCTGCCCA
CCTACAACAACCACCTCTACAAACAAATTTCCAGCCAATCAGGAGCCTCGAACGACAATCACTACTTTGGCTACA
GCACCCCTTGGGGGTATTTTGACTTCAACAGATTCCACTGCCACTTTTCACCACGTGACTGGCAAAGACTCATCA
ACAACAACTGGGGATTCCGACCCAAGAGACTCAACTTCAAGCTCTTTAACATTCAAGTCAAAGAGGTCACGCAGA
ATGACGGTACGACGACGATTGCCAATAACCTTACCAGCACGGTTCAGGTGTTTACTGACTCGGAGTACCAGCTCC
CGTACGTCCTCGGCTCGGCGCATCAAGGATGCCTCCCGCCGTTCCCAGCAGACGTCTTCATGGTGCCACAGTATG
GATACCTCACCCTGA

### AAV-3

### Full Genome: NC 001729

Rep78:
(SEQ ID NO: 9)
ATGCCGGGGTTCTACGAGATTGTCCTGAAGGTCCCGAGTGACCTGGACGAGCGCCTGCCGGGCATTTCTAACTCG
TTTGTTAACTGGGTGGCCGAGAAGGAATGGGACGTGCCGCCGGATTCTGACATGGATCCGAATCTGATTGAGCAG
GCACCCCTGACCGTGGCCGAAAAGCTTCAGCGCGAGTTCCTGGTGGAGTGGCGCCGCGTGAGTAAGGCCCCGGAG
GCCCTCTTTTTTGTCCAGTTCGAAAAGGGGGAGACCTACTTCCACCTGCACGTGCTGATTGAGACCATCGGGGTC
AAATCCATGGTGGTCGGCCGCTACGTGAGCCAGATTAAAGAGAAGCTGGTGACCCGCATCTACCGCGGGGTCGAG
CCGCAGCTTCCGAACTGGTTCGCGGTGACCAAAACGCGAAATGGCGCCGGGGGCGGGAACAAGGTGGTGGACGAC
TGCTACATCCCCAACTACCTGCTCCCCAAGACCCAGCCCGAGCTCCAGTGGGCGTGGACTAACATGGACCAGTAT
TTAAGCGCCTGTTTGAATCTCGCGGAGCGTAAACGGCTGGTGGCGCAGCATCTGACGCACGTGTCGCAGACGCAG
GAGCAGAACAAAGAGAATCAGAACCCCAATTCTGACGCGCCGGTCATCAGGTCAAAAACCTCAGCCAGGTACATG
GAGCTGGTCGGGTGGCTGGTGGACCGCGGGATCACGTCAGAAAAGCAATGGATTCAGGAGGACCAGGCCTCGTAC
ATCTCCTTCAACGCCGCCTCCAACTCGCGGTCCCAGATCAAGGCCGCGCTGGACAATGCCTCCAAGATCATGAGC
CTGACAAAGACGGCTCCGGACTACCTGGTGGGCAGCAACCCGCCGGAGGACATTACCAAAAATCGGATCTACCAA
ATCCTGGAGCTGAACGGGTACGATCCGCAGTACGCGGCCTCCGTCTTCCTGGGCTGGGCGCAAAAGAAGTTCGGG
AAGAGGAACACCATCTGGCTCTTTGGGCCGGCCACGACGGGTAAAACCAACATCGCGGAAGCCATCGCCCACGCC
GTGCCCTTCTACGGCTGCGTAAACTGGACCAATGAGAACTTTCCCTTCAACGATTGCGTCGACAAGATGGTGATC
TGGTGGGAGGAGGGCAAGATGACGGCCAAGGTCGTGGAGAGCGCCAAGGCCATTCTGGGCGGAAGCAAGGTGCGC
GTGGACCAAAAGTGCAAGTCATCGGCCCAGATCGAACCCACTCCCGTGATCGTCACCTCCAACACCAACATGTGC
GCCGTGATTGACGGGAACAGCACCACCTTCGAGCATCAGCAGCCGCTGCAGGACCGGATGTTTGAATTTGAACTT
ACCCGCCGTTTGGACCATGACTTTGGGAAGGTCACCAAACAGGAAGTAAAGGACTTTTTCCGGTGGGCTTCCGAT
CACGTGACTGACGTGGCTCATGAGTTCTACGTCAGAAAGGGTGGAGCTAAGAAACGCCCCGCCTCCAATGACGCG
GATGTAAGCGAGCCAAAACGGGAGTGCACGTCACTTGCGCAGCCGACAACGTCAGACGCGGAAGCACCGGCGGAC
TACGCGGACAGGTACCAAAACAAATGTTCTCGTCACGTGGGCATGAATCTGATGCTTTTTCCCTGTAAAACATGC
GAGAGAATGAATCAAATTTCCAATGTCTGTTTTACGCATGGTCAAAGAGACTGTGGGGAATGCTTCCCTGGAATG
TCAGAATCTCAACCCGTTTCTGTCGTCAAAAAGAAGACTTATCAGAAACTGTGTCCAATTCATCATATCCTGGGA
AGGGCACCCGAGATTGCCTGTTCGGCCTGCGATTTGGCCAATGTGGACTTGGATGACTGTGTTTCTGAGCAATAA
CapVP1:
(SEQ ID NO: 10)
ATGGCTGCTGACGGTTATCTTCCAGATTGGCTCGAGGACAACCTTTCTGAAGGCATTCGTGAGTGGTGGGCTCTG
AAACCTGGAGTCCCTCAACCCAAAGCGAACCAACAACACCAGGACAACCGTCGGGGTCTTGTGCTTCCGGGTTAC
AAATACCTCGGACCCGGTAACGGACTCGACAAAGGAGAGCCGGTCAACGAGGCGGACGCGGCAGCCCTCGAACAC
GACAAAGCTTACGACCAGCAGCTCAAGGCCGGTGACAACCCGTACCTCAAGTACAACCACGCCGACGCCGAGTTT
CAGGAGCGTCTTCAAGAAGATACGTCTTTTGGGGGCAACCTTGGCAGAGCAGTCTTCCAGGCCAAAAAGAGGATC
CTTGAGCCTCTTGGTCTGGTTGAGGAAGCAGCTAAAACGGCTCCTGGAAAGAAGGGGGCTGTAGATCAGTCTCCT
CAGGAACCGGACTCATCATCTGGTGTTGGCAAATCGGGCAAACAGCCTGCCAGAAAAAGACTAAATTTCGGTCAG
ACTGGAGACTCAGAGTCAGTCCCAGACCCTCAACCTCTCGGAGAACCACCAGCAGCCCCCACAAGTTTGGGATCT
AATACAATGGCTTCAGGCGGTGGCGCACCAATGGCAGACAATAACGAGGGTGCCGATGGAGTGGGTAATTCCTCA
GGAAATTGGCATTGCGATTCCCAATGGCTGGGCGACAGAGTCATCACCACCAGCACCAGAACCTGGGCCCTGCCC
ACTTACAACAACCATCTCTACAAGCAAATCTCCAGCCAATCAGGAGCTTCAAACGACAACCACTACTTTGGCTAC
AGCACCCCTTGGGGGTATTTTGACTTTAACAGATTCCACTGCCACTTCTCACCACGTGACTGGCAGCGACTCATT
AACAACAACTGGGGATTCCGGCCCAAGAAACTCAGCTTCAAGCTCTTCAACATCCAAGTTAGAGGGGTCACGCAG
AACGATGGCACGACGACTATTGCCAATAACCTTACCAGCACGGTTCAAGTGTTTACGGACTCGGAGTATCAGCTC
CCGTACGTGCTCGGGTCGGCGCACCAAGGCTGTCTCCCGCCGTTTCCAGCGGACGTCTTCATGGTCCCTCAGTAT
GGATACCTCACCCTGAACAACGGAAGTCAAGCGGTGGGACGCTCATCCTTTTACTGCCTGGAGTACTTCCCTTCG
CAGATGCTAAGGACTGGAAATAACTTCCAATTCAGCTATACCTTCGAGGATGTACCTTTTCACAGCAGCTACGCT
CACAGCCAGAGTTTGGATCGCTTGATGAATCCTCTTATTGATCAGTATCTGTACTACCTGAACAGAACGCAAGGA
ACAACCTCTGGAACAACCAACCAATCACGGCTGCTTTTTAGCCAGGCTGGGCCTCAGTCTATGTCTTTGCAGGCC
AGAAATTGGCTACCTGGGCCCTGCTACCGGCAACAGAGACTTTCAAAGACTGCTAACGACAACAACAACAGTAAC
TTTCCTTGGACAGCGGCCAGCAAATATCATCTCAATGGCCGCGACTCGCTGGTGAATCCAGGACCAGCTATGGCC
AGTCACAAGGACGATGAAGAAAAATTTTTCCCTATGCACGGCAATCTAATATTTGGCAAAGAAGGGACAACGGCA
AGTAACGCAGAATTAGATAATGTAATGATTACGGATGAAGAAGAGATTCGTACCACCAATCCTGTGGCAACAGAG
CAGTATGGAACTGTGGCAAATAACTTGCAGAGCTCAAATACAGCTCCCACGACTGGAACTGTCAATCATCAGGGG
GCCTTACCTGGCATGGTGTGGCAAGATCGTGACGTGTACCTTCAAGGACCTATCTGGGCAAAGATTCCTCACACG
GATGGACACTTTCATCCTTCTCCTCTGATGGGAGGCTTTGGACTGAAACATCCGCCTCCTCAAATCATGATCAAA
AATACTCCGGTACCGGCAAATCCTCCGACGACTTTCAGCCCGGCCAAGTTTGCTTCATTTATCACTCAGTACTCC
ACTGGACAGGTCAGCGTGGAAATTGAGTGGGAGCTACAGAAAGAAAACAGCAAACGTTGGAATCCAGAGATTCAG
TACACTTCCAACTACAACAAGTCTGTTAATGTGGACTTTACTGTAGACACTAATGGTGTTTATAGTGAACCTCGC
CCTATTGGAACCCGGTATCTCACACGAAACTTGTGA

### AAV-4

### Full Genome: NC_001829

Rep78:
(SEQ ID NO: 11)
ATGCCGGGGTTCTACGAGATCGTGCTGAAGGTGCCCAGCGACCTGGACGAGCACCTGCCCGGCATTTCTGACTCT
TTTGTGAGCTGGGTGGCCGAGAAGGAATGGGAGCTGCCGCCGGATTCTGACATGGACTTGAATCTGATTGAGCAG
GCACCCCTGACCGTGGCCGAAAAGCTGCAACGCGAGTTCCTGGTCGAGTGGCGCCGCGTGAGTAAGGCCCCGGAG
GCCCTCTTCTTTGTCCAGTTCGAGAAGGGGGACAGCTACTTCCACCTGCACATCCTGGTGGAGACCGTGGGCGTC
AAATCCATGGTGGTGGGCCGCTACGTGAGCCAGATTAAAGAGAAGCTGGTGACCCGCATCTACCGCGGGGTCGAG
CCGCAGCTTCCGAACTGGTTCGCGGTGACCAAGACGCGTAATGGCGCCGGAGGCGGGAACAAGGTGGTGGACGAC
TGCTACATCCCCAACTACCTGCTCCCCAAGACCCAGCCCGAGCTCCAGTGGGCGTGGACTAACATGGACCAGTAT
ATAAGCGCCTGTTTGAATCTCGCGGAGCGTAAACGGCTGGTGGCGCAGCATCTGACGCACGTGTCGCAGACGCAG
GAGCAGAACAAGGAAAACCAGAACCCCAATTCTGACGCGCCGGTCATCAGGTCAAAAACCTCCGCCAGGTACATG
GAGCTGGTCGGGTGGCTGGTGGACCGCGGGATCACGTCAGAAAAGCAATGGATCCAGGAGGACCAGGCGTCCTAC
ATCTCCTTCAACGCCGCCTCCAACTCGCGGTCACAAATCAAGGCCGCGCTGGACAATGCCTCCAAAATCATGAGC
CTGACAAAGACGGCTCCGGACTACCTGGTGGGCCAGAACCCGCCGGAGGACATTTCCAGCAACCGCATCTACCGA
ATCCTCGAGATGAACGGGTACGATCCGCAGTACGCGGCCTCCGTCTTCCTGGGCTGGGCGCAAAAGAAGTTCGGG
AAGAGGAACACCATCTGGCTCTTTGGGCCGGCCACGACGGGTAAAACCAACATCGCGGAAGCCATCGCCCACGCC
GTGCCCTTCTACGGCTGCGTGAACTGGACCAATGAGAACTTTCCGTTCAACGATTGCGTCGACAAGATGGTGATC
TGGTGGGAGGAGGGCAAGATGACGGCCAAGGTCGTAGAGAGCGCCAAGGCCATCCTGGGCGGAAGCAAGGTGCGC
GTGGACCAAAAGTGCAAGTCATCGGCCCAGATCGACCCAACTCCCGTGATCGTCACCTCCAACACCAACATGTGC
GCGGTCATCGACGGAAACTCGACCACCTTCGAGCACCAACAACCACTCCAGGACCGGATGTTCAAGTTCGAGCTC
ACCAAGCGCCTGGAGCACGACTTTGGCAAGGTCACCAAGCAGGAAGTCAAAGACTTTTTCCGGTGGGCGTCAGAT
CACGTGACCGAGGTGACTCACGAGTTTTACGTCAGAAAGGGTGGAGCTAGAAAGAGGCCCGCCCCCAATGACGCA
GATATAAGTGAGCCCAAGCGGGCCTGTCCGTCAGTTGCGCAGCCATCGACGTCAGACGCGGAAGCTCCGGTGGAC
TACGCGGACAGGTACCAAAACAAATGTTCTCGTCACGTGGGTATGAATCTGATGCTTTTTCCCTGCCGGCAATGC
GAGAGAATGAATCAGAATGTGGACATTTGCTTCACGCACGGGGTCATGGACTGTGCCGAGTGCTTCCCCGTGTCA
GAATCTCAACCCGTGTCTGTCGTCAGAAAGCGGACGTATCAGAAACTGTGTCCGATTCATCACATCATGGGGAGG
GCGCCCGAGGTGGCCTGCTCGGCCTGCGAACTGGCCAATGTGGACTTGGATGACTGTGACATGGAACAATAA
CapVP1:
(SEQ ID NO: 12)
ATGACTGACGGTTACCTTCCAGATTGGCTAGAGGACAACCTCTCTGAAGGCGTTCGAGAGTGGTGGGCGCTGCAA
CCTGGAGCCCCTAAACCCAAGGCAAATCAACAACATCAGGACAACGCTCGGGGTCTTGTGCTTCCGGGTTACAAA
TACCTCGGACCCGGCAACGGACTCGACAAGGGGGAACCCGTCAACGCAGCGGACGCGGCAGCCCTCGAGCACGAC
AAGGCCTACGACCAGCAGCTCAAGGCCGGTGACAACCCCTACCTCAAGTACAACCACGCCGACGCGGAGTTCCAG
CAGCGGCTTCAGGGCGACACATCGTTTGGGGGCAACCTCGGCAGAGCAGTCTTCCAGGCCAAAAAGAGGGTTCTT
GAACCTCTTGGTCTGGTTGAGCAAGCGGGTGAGACGGCTCCTGGAAAGAAGAGACCGTTGATTGAATCCCCCCAG
CAGCCCGACTCCTCCACGGGTATCGGCAAAAAAGGCAAGCAGCCGGCTAAAAAGAAGCTCGTTTTCGAAGACGAA
ACTGGAGCAGGCGACGGACCCCCTGAGGGATCAACTTCCGGAGCCATGTCTGATGACAGTGAGATGCGTGCAGCA
GCTGGCGGAGCTGCAGTCGAGGGCGGACAAGGTGCCGATGGAGTGGGTAATGCCTCGGGTGATTGGCATTGCGAT
TCCACCTGGTCTGAGGGCCACGTCACGACCACCAGCACCAGAACCTGGGTCTTGCCCACCTACAACAACCACCTC
TACAAGCGACTCGGAGAGAGCCTGCAGTCCAACACCTACAACGGATTCTCCACCCCCTGGGGATACTTTGACTTC
AACCGCTTCCACTGCCACTTCTCACCACGTGACTGGCAGCGACTCATCAACAACAACTGGGGCATGCGACCCAAA
GCCATGCGGGTCAAAATCTTCAACATCCAGGTCAAGGAGGTCACGACGTCGAACGGCGAGACAACGGTGGCTAAT
AACCTTACCAGCACGGTTCAGATCTTTGCGGACTCGTCGTACGAACTGCCGTACGTGATGGATGCGGGTCAAGAG
GGCAGCCTGCCTCCTTTTCCCAACGACGTCTTTATGGTGCCCCAGTACGGCTACTGTGGACTGGTGACCGGCAAC
ACTTCGCAGCAACAGACTGACAGAAATGCCTTCTACTGCCTGGAGTACTTTCCTTCGCAGATGCTGCGGACTGGC
AACAACTTTGAAATTACGTACAGTTTTGAGAAGGTGCCTTTCCACTCGATGTACGCGCACAGCCAGAGCCTGGAC
CGGCTGATGAACCCTCTCATCGACCAGTACCTGTGGGGACTGCAATCGACCACCACCGGAACCACCCTGAATGCC
GGGACTGCCACCACCAACTTTACCAAGCTGCGGCCTACCAACTTTTCCAACTTTAAAAAGAACTGGCTGCCCGGG
CCTTCAATCAAGCAGCAGGGCTTCTCAAAGACTGCCAATCAAAACTACAAGATCCCTGCCACCGGGTCAGACAGT
CTCATCAAATACGAGACGCACAGCACTCTGGACGGAAGATGGAGTGCCCTGACCCCCGGACCTCCAATGGCCACG
GCTGGACCTGCGGACAGCAAGTTCAGCAACAGCCAGCTCATCTTTGCGGGGCCTAAACAGAACGGCAACACGGCC
ACCGTACCCGGGACTCTGATCTTCACCTCTGAGGAGGAGCTGGCAGCCACCAACGCCACCGATACGGACATGTGG
GGCAACCTACCTGGCGGTGACCAGAGCAACAGCAACCTGCCGACCGTGGACAGACTGACAGCCTTGGGAGCCGTG
CCTGGAATGGTCTGGCAAAACAGAGACATTTACTACCAGGGTCCCATTTGGGCCAAGATTCCTCATACCGATGGA
CACTTTCACCCCTCACCGCTGATTGGTGGGTTTGGGCTGAAACACCCGCCTCCTCAAATTTTTATCAAGAACACC
CCGGTACCTGCGAATCCTGCAACGACCTTCAGCTCTACTCCGGTAAACTCCTTCATTACTCAGTACAGCACTGGC
CAGGTGTCGGTGCAGATTGACTGGGAGATCCAGAAGGAGCGGTCCAAACGCTGGAACCCCGAGGTCCAGTTTACC
TCCAACTACGGACAGCAAAACTCTCTGTTGTGGGCTCCCGATGCGGCTGGGAAATACACTGAGCCTAGGGCTATC
GGTACCCGCTACCTCACCCACCACCTGTAA

### AAV-5

### Full Genome: NC 006152

Rep78:
(SEQ ID NO: 13)
ATGGCTACCTTCTATGAAGTCATTGTTCGCGTCCCATTTGACGTGGAGGAACATCTGCCTGGAATTTCTGACAGC
TTTGTGGACTGGGTAACTGGTCAAATTTGGGAGCTGCCTCCAGAGTCAGATTTAAATTTGACTCTGGTTGAACAG
CCTCAGTTGACGGTGGCTGATAGAATTCGCCGCGTGTTCCTGTACGAGTGGAACAAATTTTCCAAGCAGGAGTCC
AAATTCTTTGTGCAGTTTGAAAAGGGATCTGAATATTTTCATCTGCACACGCTTGTGGAGACCTCCGGCATCTCT
TCCATGGTCCTCGGCCGCTACGTGAGTCAGATTCGCGCCCAGCTGGTGAAAGTGGTCTTCCAGGGAATTGAACCC
CAGATCAACGACTGGGTCGCCATCACCAAGGTAAAGAAGGGCGGAGCCAATAAGGTGGTGGATTCTGGGTATATT
CCCGCCTACCTGCTGCCGAAGGTCCAACCGGAGCTTCAGTGGGCGTGGACAAACCTGGACGAGTATAAATTGGCC
GCCCTGAATCTGGAGGAGCGCAAACGGCTCGTCGCGCAGTTTCTGGCAGAATCCTCGCAGCGCTCGCAGGAGGCG
GCTTCGCAGCGTGAGTTCTCGGCTGACCCGGTCATCAAAAGCAAGACTTCCCAGAAATACATGGCGCTCGTCAAC
TGGCTCGTGGAGCACGGCATCACTTCCGAGAAGCAGTGGATCCAGGAAAATCAGGAGAGCTACCTCTCCTTCAAC
TCCACCGGCAACTCTCGGAGCCAGATCAAGGCCGCGCTCGACAACGCGACCAAAATTATGAGTCTGACAAAAAGC
GCGGTGGACTACCTCGTGGGGAGCTCCGTTCCCGAGGACATTTCAAAAAACAGAATCTGGCAAATTTTTGAGATG
AATGGCTACGACCCGGCCTACGCGGGATCCATCCTCTACGGCTGGTGTCAGCGCTCCTTCAACAAGAGGAACACC
GTCTGGCTCTACGGACCCGCCACGACCGGCAAGACCAACATCGCGGAGGCCATCGCCCACACTGTGCCCTTTTAC
GGCTGCGTGAACTGGACCAATGAAAACTTTCCCTTTAATGACTGTGTGGACAAAATGCTCATTTGGTGGGAGGAG
GGAAAGATGACCAACAAGGTGGTTGAATCCGCCAAGGCCATCCTGGGGGGCTCAAAGGTGCGGGTCGATCAGAAA
TGTAAATCCTCTGTTCAAATTGATTCTACCCCTGTCATTGTAACTTCCAATACAAACATGTGTGTGGTGGTGGAT
GGGAATTCCACGACCTTTGAACACCAGCAGCCGCTGGAGGACCGCATGTTCAAATTTGAACTGACTAAGCGGCTC
CCGCCAGATTTTGGCAAGATTACTAAGCAGGAAGTCAAGGACTTTTTTGCTTGGGCAAAGGTCAATCAGGTGCCG
GTGACTCACGAGTTTAAAGTTCCCAGGGAATTGGCGGGAACTAAAGGGGCGGAGAAATCTCTAAAACGCCCACTG
GGTGACGTCACCAATACTAGCTATAAAAGTCTGGAGAAGCGGGCCAGGCTCTCATTTGTTCCCGAGACGCCTCGC
AGTTCAGACGTGACTGTTGATCCCGCTCCTCTGCGACCGCTCAATTGGAATTCAAGGTATGATTGCAAATGTGAC
TATCATGCTCAATTTGACAACATTTCTAACAAATGTGATGAATGTGAATATTTGAATCGGGGCAAAAATGGATGT
ATCTGTCACAATGTAACTCACTGTCAAATTTGTCATGGGATTCCCCCCTGGGAAAAGGAAAACTTGTCAGATTTT
GGGGATTTTGACGATGCCAATAAAGAACAGTAA
CapVP1:
(SEQ ID NO: 14)
ATGTCTTTTGTTGATCACCCTCCAGATTGGTTGGAAGAAGTTGGTGAAGGTCTTCGCGAGTTTTTGGGCCTTGAA
GCGGGCCCACCGAAACCAAAACCCAATCAGCAGCATCAAGATCAAGCCCGTGGTCTTGTGCTGCCTGGTTATAAC
TATCTCGGACCCGGAAACGGTCTCGATCGAGGAGAGCCTGTCAACAGGGCAGACGAGGTCGCGCGAGAGCACGAC
ATCTCGTACAACGAGCAGCTTGAGGCGGGAGACAACCCCTACCTCAAGTACAACCACGCGGACGCCGAGTTTCAG
GAGAAGCTCGCCGACGACACATCCTTCGGGGGAAACCTCGGAAAGGCAGTCTTTCAGGCCAAGAAAAGGGTTCTC
GAACCTTTTGGCCTGGTTGAAGAGGGTGCTAAGACGGCCCCTACCGGAAAGCGGATAGACGACCACTTTCCAAAA
AGAAAGAAGGCTCGGACCGAAGAGGACTCCAAGCCTTCCACCTCGTCAGACGCCGAAGCTGGACCCAGCGGATCC
CAGCAGCTGCAAATCCCAGCCCAACCAGCCTCAAGTTTGGGAGCTGATACAATGTCTGCGGGAGGTGGCGGCCCA
TTGGGCGACAATAACCAAGGTGCCGATGGAGTGGGCAATGCCTCGGGAGATTGGCATTGCGATTCCACGTGGATG
GGGGACAGAGTCGTCACCAAGTCCACCCGAACCTGGGTGCTGCCCAGCTACAACAACCACCAGTACCGAGAGATC
AAAAGCGGCTCCGTCGACGGAAGCAACGCCAACGCCTACTTTGGATACAGCACCCCCTGGGGGTACTTTGACTTT
AACCGCTTCCACAGCCACTGGAGCCCCCGAGACTGGCAAAGACTCATCAACAACTACTGGGGCTTCAGACCCCGG
TCCCTCAGAGTCAAAATCTTCAACATTCAAGTCAAAGAGGTCACGGTGCAGGACTCCACCACCACCATCGCCAAC
AACCTCACCTCCACCGTCCAAGTGTTTACGGACGACGACTACCAGCTGCCCTACGTCGTCGGCAACGGGACCGAG
GGATGCCTGCCGGCCTTCCCTCCGCAGGTCTTTACGCTGCCGCAGTACGGTTACGCGACGCTGAACCGCGACAAC
ACAGAAAATCCCACCGAGAGGAGCAGCTTCTTCTGCCTAGAGTACTTTCCCAGCAAGATGCTGAGAACGGGCAAC
AACTTTGAGTTTACCTACAACTTTGAGGAGGTGCCCTTCCACTCCAGCTTCGCTCCCAGTCAGAACCTGTTCAAG
CTGGCCAACCCGCTGGTGGACCAGTACTTGTACCGCTTCGTGAGCACAAATAACACTGGCGGAGTCCAGTTCAAC
AAGAACCTGGCCGGGAGATACGCCAACACCTACAAAAACTGGTTCCCGGGGCCCATGGGCCGAACCCAGGGCTGG
AACCTGGGCTCCGGGGTCAACCGCGCCAGTGTCAGCGCCTTCGCCACGACCAATAGGATGGAGCTCGAGGGCGCG
AGTTACCAGGTGCCCCCGCAGCCGAACGGCATGACCAACAACCTCCAGGGCAGCAACACCTATGCCCTGGAGAAC
ACTATGATCTTCAACAGCCAGCCGGCGAACCCGGGCACCACCGCCACGTACCTCGAGGGCAACATGCTCATCACC
AGCGAGAGCGAGACGCAGCCGGTGAACCGCGTGGCGTACAACGTCGGCGGGCAGATGGCCACCAACAACCAGAGC
TCCACCACTGCCCCCGCGACCGGCACGTACAACCTCCAGGAAATCGTGCCCGGCAGCGTGTGGATGGAGAGGGAC
GTGTACCTCCAAGGACCCATCTGGGCCAAGATCCCAGAGACGGGGGCGCACTTTCACCCCTCTCCGGCCATGGGC
GGATTCGGACTCAAACACCCACCGCCCATGATGCTCATCAAGAACACGCCTGTGCCCGGAAATATCACCAGCTTC
TCGGACGTGCCCGTCAGCAGCTTCATCACCCAGTACAGCACCGGGCAGGTCACCGTGGAGATGGAGTGGGAGCTC
AAGAAGGAAAACTCCAAGAGGTGGAACCCAGAGATCCAGTACACAAACAACTACAACGACCCCCAGTTTGTGGAC
TTTGCCCCGGACAGCACCGGGGAATACAGAACCACCAGACCTATCGGAACCCGATACCTTACCCGACCCCTTTAA

### AAv-6

### Full Genome: AF028704

Rep78:
(SEQ ID NO: 15)
ATGCCGGGGTTTTACGAGATTGTGATTAAGGTCCCCAGCGACCTTGACGAGCATCTGCCCGGCATTTCTGACAGC
TTTGTGAACTGGGTGGCCGAGAAGGAATGGGAGTTGCCGCCAGATTCTGACATGGATCTGAATCTGATTGAGCAG
GCACCCCTGACCGTGGCCGAGAAGCTGCAGCGCGACTTCCTGGTCCAGTGGCGCCGCGTGAGTAAGGCCCCGGAG
GCCCTCTTCTTTGTTCAGTTCGAGAAGGGCGAGTCCTACTTCCACCTCCATATTCTGGTGGAGACCACGGGGGTC
AAATCCATGGTGCTGGGCCGCTTCCTGAGTCAGATTAGGGACAAGCTGGTGCAGACCATCTACCGCGGGATCGAG
CCGACCCTGCCCAACTGGTTCGCGGTGACCAAGACGCGTAATGGCGCCGGAGGGGGGAACAAGGTGGTGGACGAG
TGCTACATCCCCAACTACCTCCTGCCCAAGACTCAGCCCGAGCTGCAGTGGGCGTGGACTAACATGGAGGAGTAT
ATAAGCGCGTGTTTAAACCTGGCCGAGCGCAAACGGCTCGTGGCGCACGACCTGACCCACGTCAGCCAGACCCAG
GAGCAGAACAAGGAGAATCTGAACCCCAATTCTGACGCGCCTGTCATCCGGTCAAAAACCTCCGCACGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCTCTGGACAATGCCGGCAAGATCATGGCG
CTGACCAAATCCGCGCCCGACTACCTGGTAGGCCCCGCTCCGCCCGCCGACATTAAAACCAACCGCATTTACCGC
ATCCTGGAGCTGAACGGCTACGACCCTGCCTACGCCGGCTCCGTCTTTCTCGGCTGGGCCCAGAAAAGGTTCGGA
AAACGCAACACCATCTGGCTGTTTGGGCCGGCCACCACGGGCAAGACCAACATCGCGGAAGCCATCGCCCACGCC
GTGCCCTTCTACGGCTGCGTCAACTGGACCAATGAGAACTTTCCCTTCAACGATTGCGTCGACAAGATGGTGATC
TGGTGGGAGGAGGGCAAGATGACGGCCAAGGTCGTGGAGTCCGCCAAGGCCATTCTCGGCGGCAGCAAGGTGCGC
GTGGACCAAAAGTGCAAGTCGTCCGCCCAGATCGATCCCACCCCCGTGATCGTCACCTCCAACACCAACATGTGC
GCCGTGATTGACGGGAACAGCACCACCTTCGAGCACCAGCAGCCGTTGCAGGACCGGATGTTCAAATTTGAACTC
ACCCGCCGTCTGGAGCATGACTTTGGCAAGGTGACAAAGCAGGAAGTCAAAGAGTTCTTCCGCTGGGCGCAGGAT
CACGTGACCGAGGTGGCGCATGAGTTCTACGTCAGAAAGGGTGGAGCCAACAAGAGACCCGCCCCCGATGACGCG
GATAAAAGCGAGCCCAAGCGGGCCTGCCCCTCAGTCGCGGATCCATCGACGTCAGACGCGGAAGGAGCTCCGGTG
GACTTTGCCGACAGGTACCAAAACAAATGTTCTCGTCACGCGGGCATGCTTCAGATGCTGTTTCCCTGCAAAACA
TGCGAGAGAATGAATCAGAATTTCAACATTTGCTTCACGCACGGGACCAGAGACTGTTCAGAATGTTTCCCCGGC
GTGTCAGAATCTCAACCGGTCGTCAGAAAGAGGACGTATCGGAAACTCTGTGCCATTCATCATCTGCTGGGGCGG
GCTCCCGAGATTGCTTGCTCGGCCTGCGATCTGGTCAACGTGGATCTGGATGACTGTGTTTCTGAGCAATAA
CapVP1:
(SEQ ID NO: 16)
ATGGCTGCCGATGGTTATCTTCCAGATTGGCTCGAGGACAACCTCTCTGAGGGCATTCGCGAGTGGTGGGACTTG
AAACCTGGAGCCCCGAAACCCAAAGCCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGCGGCGGATGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGAGGGTT
CTCGAACCTTTTGGTCTGGTTGAGGAAGGTGCTAAGACGGCTCCTGGAAAGAAACGTCCGGTAGAGCAGTCGCCA
CAAGAGCCAGACTCCTCCTCGGGCATTGGCAAGACAGGCCAGCAGCCCGCTAAAAAGAGACTCAATTTTGGTCAG
ACTGGCGACTCAGAGTCAGTCCCCGACCCACAACCTCTCGGAGAACCTCCAGCAACCCCCGCTGCTGTGGGACCT
ACTACAATGGCTTCAGGCGGTGGCGCACCAATGGCAGACAATAACGAAGGCGCCGACGGAGTGGGTAATGCCTCA
GGAAATTGGCATTGCGATTCCACATGGCTGGGCGACAGAGTCATCACCACCAGCACCCGAACATGGGCCTTGCCC
ACCTATAACAACCACCTCTACAAGCAAATCTCCAGTGCTTCAACGGGGGCCAGCAACGACAACCACTACTTCGGC
TACAGCACCCCCTGGGGGTATTTTGATTTCAACAGATTCCACTGCCATTTCTCACCACGTGACTGGCAGCGACTC
ATCAACAACAATTGGGGATTCCGGCCCAAGAGACTCAACTTCAAGCTCTTCAACATCCAAGTCAAGGAGGTCACG
ACGAATGATGGCGTCACGACCATCGCTAATAACCTTACCAGCACGGTTCAAGTCTTCTCGGACTCGGAGTACCAG
TTGCCGTACGTCCTCGGCTCTGCGCACCAGGGCTGCCTCCCTCCGTTCCCGGCGGACGTGTTCATGATTCCGCAG
TACGGCTACCTAACGCTCAACAATGGCAGCCAGGCAGTGGGACGGTCATCCTTTTACTGCCTGGAATATTTCCCA
TCGCAGATGCTGAGAACGGGCAATAACTTTACCTTCAGCTACACCTTCGAGGACGTGCCTTTCCACAGCAGCTAC
GCGCACAGCCAGAGCCTGGACCGGCTGATGAATCCTCTCATCGACCAGTACCTGTATTACCTGAACAGAACTCAG
AATCAGTCCGGAAGTGCCCAAAACAAGGACTTGCTGTTTAGCCGGGGGTCTCCAGCTGGCATGTCTGTTCAGCCC
AAAAACTGGCTACCTGGACCCTGTTACCGGCAGCAGCGCGTTTCTAAAACAAAAACAGACAACAACAACAGCAAC
TTTACCTGGACTGGTGCTTCAAAATATAACCTTAATGGGCGTGAATCTATAATCAACCCTGGCACTGCTATGGCC
TCACACAAAGACGACAAAGACAAGTTCTTTCCCATGAGCGGTGTCATGATTTTTGGAAAGGAGAGCGCCGGAGCT
TCAAACACTGCATTGGACAATGTCATGATCACAGACGAAGAGGAAATCAAAGCCACTAACCCCGTGGCCACCGAA
AGATTTGGGACTGTGGCAGTCAATCTCCAGAGCAGCAGCACAGACCCTGCGACCGGAGATGTGCATGTTATGGGA
GCCTTACCTGGAATGGTGTGGCAAGACAGAGACGTATACCTGCAGGGTCCTATTTGGGCCAAAATTCCTCACACG
GATGGACACTTTCACCCGTCTCCTCTCATGGGCGGCTTTGGACTTAAGCACCCGCCTCCTCAGATCCTCATCAAA
AACACGCCTGTTCCTGCGAATCCTCCGGCAGAGTTTTCGGCTACAAAGTTTGCTTCATTCATCACCCAGTATTCC
ACAGGACAAGTGAGCGTGGAGATTGAATGGGAGCTGCAGAAAGAAAACAGCAAACGCTGGAATCCCGAAGTGCAG
TATACATCTAACTATGCAAAATCTGCCAACGTTGATTTCACTGTGGACAACAATGGACTTTATACTGAGCCTCGC
CCCATTGGCACCCGTTACCTCACCCGTCCCCTGTAA

### AAV-7

### Full Genome: NC_006260

Rep78:
(SEQ ID NO: 17)
ATGCCGGGTTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCTGAATCTGATCGAGCAG
GCACCCCTGACCGTGGCCGAGAAGCTGCAGCGCGACTTCCTGGTCCAATGGCGCCGCGTGAGTAAGGCCCCGGAG
GCCCTGTTCTTTGTTCAGTTCGAGAAGGGCGAGAGCTACTTCCACCTTCACGTTCTGGTGGAGACCACGGGGGTC
AAGTCCATGGTGCTAGGCCGCTTCCTGAGTCAGATTCGGGAGAAGCTGGTCCAGACCATCTACCGCGGGGTCGAG
CCCACGCTGCCCAACTGGTTCGCGGTGACCAAGACGCGTAATGGCGCCGGCGGGGGGAACAAGGTGGTGGACGAG
TGCTACATCCCCAACTACCTCCTGCCCAAGACCCAGCCCGAGCTGCAGTGGGCGTGGACTAACATGGAGGAGTAT
ATAAGCGCGTGTTTGAACCTGGCCGAACGCAAACGGCTCGTGGCGCAGCACCTGACCCACGTCAGCCAGACGCAG
GAGCAGAACAAGGAGAATCTGAACCCCAATTCTGACGCGCCCGTGATCAGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCGCTGGACAATGCCGGCAAGATCATGGCG
CTGACCAAATCCGCGCCCGACTACCTGGTGGGGCCCTCGCTGCCCGCGGACATTAAAACCAACCGCATCTACCGC
ATCCTGGAGCTGAACGGGTACGATCCTGCCTACGCCGGCTCCGTCTTTCTCGGCTGGGCCCAGAAAAAGTTCGGG
AAGCGCAACACCATCTGGCTGTTTGGGCCCGCCACCACCGGCAAGACCAACATTGCGGAAGCCATCGCCCACGCC
GTGCCCTTCTACGGCTGCGTCAACTGGACCAATGAGAACTTTCCCTTCAACGATTGCGTCGACAAGATGGTGATC
TGGTGGGAGGAGGGCAAGATGACGGCCAAGGTCGTGGAGTCCGCCAAGGCCATTCTCGGCGGCAGCAAGGTGCGC
GTGGACCAAAAGTGCAAGTCGTCCGCCCAGATCGACCCCACCCCCGTGATCGTCACCTCCAACACCAACATGTGC
GCCGTGATTGACGGGAACAGCACCACCTTCGAGCACCAGCAGCCGTTGCAGGACCGGATGTTCAAATTTGAACTC
ACCCGCCGTCTGGAGCACGACTTTGGCAAGGTGACGAAGCAGGAAGTCAAAGAGTTCTTCCGCTGGGCCAGTGAT
CACGTGACCGAGGTGGCGCATGAGTTCTACGTCAGAAAGGGCGGAGCCAGCAAAAGACCCGCCCCCGATGACGCG
GATATAAGCGAGCCCAAGCGGGCCTGCCCCTCAGTCGCGGATCCATCGACGTCAGACGCGGAAGGAGCTCCGGTG
GACTTTGCCGACAGGTACCAAAACAAATGTTCTCGTCACGCGGGCATGATTCAGATGCTGTTTCCCTGCAAAACG
TGCGAGAGAATGAATCAGAATTTCAACATTTGCTTCACACACGGGGTCAGAGACTGTTTAGAGTGTTTCCCCGGC
GTGTCAGAATCTCAACCGGTCGTCAGAAAAAAGACGTATCGGAAACTCTGCGCGATTCATCATCTGCTGGGGCGG
GCGCCCGAGATTGCTTGCTCGGCCTGCGACCTGGTCAACGTGGACCTGGACGACTGCGTTTCTGAGCAATAA
CapVP1:
(SEQ ID NO: 18)
ATGGCTGCCGATGGTTATCTTCCAGATTGGCTCGAGGACAACCTCTCTGAGGGCATTCGCGAGTGGTGGGACCTG
AAACCTGGAGCCCCGAAACCCAAAGCCAACCAGCAAAAGCAGGACAACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCATTTGGGGGCAACCTCGGGCGAGCAGTCTTCCAGGCCAAGAAGCGGGTT
CTCGAACCTCTCGGTCTGGTTGAGGAAGGCGCTAAGACGGCTCCTGCAAAGAAGAGACCGGTAGAGCCGTCACCT
CAGCGTTCCCCCGACTCCTCCACGGGCATCGGCAAGAAAGGCCAGCAGCCCGCCAGAAAGAGACTCAATTTCGGT
CAGACTGGCGACTCAGAGTCAGTCCCCGACCCTCAACCTCTCGGAGAACCTCCAGCAGCGCCCTCTAGTGTGGGA
TCTGGTACAGTGGCTGCAGGCGGTGGCGCACCAATGGCAGACAATAACGAAGGTGCCGACGGAGTGGGTAATGCC
TCAGGAAATTGGCATTGCGATTCCACATGGCTGGGCGACAGAGTCATTACCACCAGCACCCGAACCTGGGCCCTG
CCCACCTACAACAACCACCTCTACAAGCAAATCTCCAGTGAAACTGCAGGTAGTACCAACGACAACACCTACTTC
GGCTACAGCACCCCCTGGGGGTATTTTGACTTTAACAGATTCCACTGCCACTTCTCACCACGTGACTGGCAGCGA
CTCATCAACAACAACTGGGGATTCCGGCCCAAGAAGCTGCGGTTCAAGCTCTTCAACATCCAGGTCAAGGAGGTC
ACGACGAATGACGGCGTTACGACCATCGCTAATAACCTTACCAGCACGATTCAGGTATTCTCGGACTCGGAATAC
CAGCTGCCGTACGTCCTCGGCTCTGCGCACCAGGGCTGCCTGCCTCCGTTCCCGGCGGACGTCTTCATGATTCCT
CAGTACGGCTACCTGACTCTCAACAATGGCAGTCAGTCTGTGGGACGTTCCTCCTTCTACTGCCTGGAGTACTTC
CCCTCTCAGATGCTGAGAACGGGCAACAACTTTGAGTTCAGCTACAGCTTCGAGGACGTGCCTTTCCACAGCAGC
TACGCACACAGCCAGAGCCTGGACCGGCTGATGAATCCCCTCATCGACCAGTACTTGTACTACCTGGCCAGAACA
CAGAGTAACCCAGGAGGCACAGCTGGCAATCGGGAACTGCAGTTTTACCAGGGCGGGCCTTCAACTATGGCCGAA
CAAGCCAAGAATTGGTTACCTGGACCTTGCTTCCGGCAACAAAGAGTCTCCAAAACGCTGGATCAAAACAACAAC
AGCAACTTTGCTTGGACTGGTGCCACCAAATATCACCTGAACGGCAGAAACTCGTTGGTTAATCCCGGCGTCGCC
ATGGCAACTCACAAGGACGACGAGGACCGCTTTTTCCCATCCAGCGGAGTCCTGATTTTTGGAAAAACTGGAGCA
ACTAACAAAACTACATTGGAAAATGTGTTAATGACAAATGAAGAAGAAATTCGTCCTACTAATCCTGTAGCCACG
GAAGAATACGGGATAGTCAGCAGCAACTTACAAGCGGCTAATACTGCAGCCCAGACACAAGTTGTCAACAACCAG
GGAGCCTTACCTGGCATGGTCTGGCAGAACCGGGACGTGTACCTGCAGGGTCCCATCTGGGCCAAGATTCCTCAC
ACGGATGGCAACTTTCACCCGTCTCCTTTGATGGGCGGCTTTGGACTTAAACATCCGCCTCCTCAGATCCTGATC
AAGAACACTCCCGTTCCCGCTAATCCTCCGGAGGTGTTTACTCCTGCCAAGTTTGCTTCGTTCATCACACAGTAC
AGCACCGGACAAGTCAGCGTGGAAATCGAGTGGGAGCTGCAGAAGGAAAACAGCAAGCGCTGGAACCCGGAGATT
CAGTACACCTCCAACTTTGAAAAGCAGACTGGTGTGGACTTTGCCGTTGACAGCCAGGGTGTTTACTCTGAGCCT
CGCCCTATTGGCACTCGTTACCTCACCCGTAATCTGTAA

### AAV-8

### Full Genome: NC_006261

Rep78:
(SEQ ID NO: 19)
ATGCCGGGCTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCGGAATCTGATCGAGCAG
GCACCCCTGACCGTGGCCGAGAAGCTGCAGCGCGACTTCCTGGTCCAATGGCGCCGCGTGAGTAAGGCCCCGGAG
GCCCTCTTCTTTGTTCAGTTCGAGAAGGGCGAGAGCTACTTTCACCTGCACGTTCTGGTCGAGACCACGGGGGTC
AAGTCCATGGTGCTAGGCCGCTTCCTGAGTCAGATTCGGGAAAAGCTTGGTCCAGACCATCTACCCGCGGGGTCG
AGCCCCACCTTGCCCAACTGGTTCGCGGTGACCAAAGACGCGGTAATGGCGCCGGCGGGGGGGAACAAGGTGGTG
GACGAGTGCTACATCCCCAACTACCTCCTGCCCAAGACTCAGCCCGAGCTGCAGTGGGCGTGGACTAACATGGAG
GAGTATATAAGCGCGTGCTTGAACCTGGCCGAGCGCAAACGGCTCGTGGCGCAGCACCTGACCCACGTCAGCCAG
ACGCAGGAGCAGAACAAGGAGAATCTGAACCCCAATTCTGACGCGCCCGTGATCAGGTCAAAAACCTCCGCGCGC
TATATGGAGCTGGTCGGGTGGCTGGTGGACCGGGGCATCACCTCCGAGAAGCAGTGGATCCAGGAGGACCAGGCC
TCGTACATCTCCTTCAACGCCGCCTCCAACTCGCGGTCCCAGATCAAGGCCGCGCTGGACAATGCCGGCAAGATC
ATGGCGCTGACCAAATCCGCGCCCGACTACCTGGTGGGGCCCTCGCTGCCCGCGGACATTACCCAGAACCGCATC
TACCGCATCCTCGCTCTCAACGGCTACGACCCTGCCTACGCCGGCTCCGTCTTTCTCGGCTGGGCTCAGAAAAAG
TTCGGGAAACGCAACACCATCTGGCTGTTTGGACCCGCCACCACCGGCAAGACCAACATTGCGGAAGCCATCGCC
CACGCCGTGCCCTTCTACGGCTGCGTCAACTGGACCAATGAGAACTTTCCCTTCAATGATTGCGTCGACAAGATG
GTGATCTGGTGGGAGGAGGGCAAGATGACGGCCAAGGTCGTGGAGTCCGCCAAGGCCATTCTCGGCGGCAGCAAG
GTGCGCGTGGACCAAAAGTGCAAGTCGTCCGCCCAGATCGACCCCACCCCCGTGATCGTCACCTCCAACACCAAC
ATGTGCGCCGTGATTGACGGGAACAGCACCACCTTCGAGCACCAGCAGCCTCTCCAGGACCGGATGTTTAAGTTC
GAACTCACCCGCCGTCTGGAGCACGACTTTGGCAAGGTGACAAAGCAGGAAGTCAAAGAGTTCTTCCGCTGGGCC
AGTGATCACGTGACCGAGGTGGCGCATGAGTTTTACGTCAGAAAGGGCGGAGCCAGCAAAAGACCCGCCCCCGAT
GACGCGGATAAAAGCGAGCCCAAGCGGGCCTGCCCCTCAGTCGCGGATCCATCGACGTCAGACGCGGAAGGAGCT
CCGGTGGACTTTGCCGACAGGTACCAAAACAAATGTTCTCGTCACGCGGGCATGCTTCAGATGCTGTTTCCCTGC
AAAACGTGCGAGAGAATGAATCAGAATTTCAACATTTGCTTCACACACGGGGTCAGAGACTGCTCAGAGTGTTTC
CCCGGCGTGTCAGAATCTCAACCGGTCGTCAGAAAGAGGACGTATCGGAAACTCTGTGCGATTCATCATCTGCTG
GGGCGGGCTCCCGAGATTGCTTGCTCGGCCTGCGATCTGGTCAACGTGGACCTGGATGACTGTGTTTCTGAGCAA
TAA
CapVP1:
(SEQ ID NO: 20)
ATGGCTGCCGATGGTTATCTTCCAGATTGGCTCGAGGACAACCTCTCTGAGGGCATTCGCGAGTGGTGGGCGCTG
AAACCTGGAGCCCCGAAGCCCAAAGCCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTGCAGGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGCGGGTT
CTCGAACCTCTCGGTCTGGTTGAGGAAGGCGCTAAGACGGCTCCTGGAAAGAAGAGACCGGTAGAGCCATCACCC
CAGCGTTCTCCAGACTCCTCTACGGGCATCGGCAAGAAAGGCCAACAGCCCGCCAGAAAAAGACTCAATTTTGGT
CAGACTGGCGACTCAGAGTCAGTTCCAGACCCTCAACCTCTCGGAGAACCTCCAGCAGCGCCCTCTGGTGTGGGA
CCTAATACAATGGCTGCAGGCGGTGGCGCACCAATGGCAGACAATAACGAAGGCGCCGACGGAGTGGGTAGTTCC
TCGGGAAATTGGCATTGCGATTCCACATGGCTGGGCGACAGAGTCATCACCACCAGCACCCGAACCTGGGCCCTG
CCCACCTACAACAACCACCTCTACAAGCAAATCTCCAACGGGACATCGGGAGGAGCCACCAACGACAACACCTAC
TTCGGCTACAGCACCCCCTGGGGGTATTTTGACTTTAACAGATTCCACTGCCACTTTTCACCACGTGACTGGCAG
CGACTCATCAACAACAACTGGGGATTCCGGCCCAAGAGACTCAGCTTCAAGCTCTTCAACATCCAGGTCAAGGAG
GTCACGCAGAATGAAGGCACCAAGACCATCGCCAATAACCTCACCAGCACCATCCAGGTGTTTACGGACTCGGAG
TACCAGCTGCCGTACGTTCTCGGCTCTGCCCACCAGGGCTGCCTGCCTCCGTTCCCGGCGGACGTGTTCATGATT
CCCCAGTACGGCTACCTAACACTCAACAACGGTAGTCAGGCCGTGGGACGCTCCTCCTTCTACTGCCTGGAATAC
TTTCCTTCGCAGATGCTGAGAACCGGCAACAACTTCCAGTTTACTTACACCTTCGAGGACGTGCCTTTCCACAGC
AGCTACGCCCACAGCCAGAGCTTGGACCGGCTGATGAATCCTCTGATTGACCAGTACCTGTACTACTTGTCTCGG
ACTCAAACAACAGGAGGCACGGCAAATACGCAGACTCTGGGCTTCAGCCAAGGTGGGCCTAATACAATGGCCAAT
CAGGCAAAGAACTGGCTGCCAGGACCCTGTTACCGCCAACAACGCGTCTCAACGACAACCGGGCAAAACAACAAT
AGCAACTTTGCCTGGACTGCTGGGACCAAATACCATCTGAATGGAAGAAATTCATTGGCTAATCCTGGCATCGCT
ATGGCAACACACAAAGACGACGAGGAGCGTTTTTTTCCCAGTAACGGGATCCTGATTTTTGGCAAACAAAATGCT
GCCAGAGACAATGCGGATTACAGCGATGTCATGCTCACCAGCGAGGAAGAAATCAAAACCACTAACCCTGTGGCT
ACAGAGGAATACGGTATCGTGGCAGATAACTTGCAGCAGCAAAACACGGCTCCTCAAATTGGAACTGTCAACAGC
CAGGGGGCCTTACCCGGTATGGTCTGGCAGAACCGGGACGTGTACCTGCAGGGTCCCATCTGGGCCAAGATTCCT
CACACGGACGGCAACTTCCACCCGTCTCCGCTGATGGGCGGCTTTGGCCTGAAACATCCTCCGCCTCAGATCCTG
ATCAAGAACACGCCTGTACCTGCGGATCCTCCGACCACCTTCAACCAGTCAAAGCTGAACTCTTTCATCACGCAA
TACAGCACCGGACAGGTCAGCGTGGAAATTGAATGGGAGCTGCAGAAGGAAAACAGCAAGCGCTGGAACCCCGAG
ATCCAGTACACCTCCAACTACTACAAATCTACAAGTGTGGACTTTGCTGTTAATACAGAAGGCGTGTACTCTGAA
CCCCGCCCCATTGGCACCCGTTACCTCACCCGTAATCTGTAA

### AAV-9

### Cap Only: AY530579

CapVP1:
(SEQ ID NO: 21)
ATGGCTGCCGATGGTTATCTTCCAGATTGGCTCGAGGACAACCTTAGTGAAGGAATTCGCGAGTGGTGGGCTTTG
AAACCTGGAGCCCCTCAACCCAAGGCAAATCAACAACATCAAGACAACGCTCGAGGTCTTGTGCTTCCGGGTTAC
AAATACCTTGGACCCGGCAACGGACTCGACAAGGGGGAGCCGGTCAACGCAGCAGACGCGGCGGCCCTCGAGCAC
GACAAGGCCTACGACCAGCAGCTCAAGGCCGGAGACAACCCGTACCTCAAGTACAACCACGCCGACGCCGAGTTC
CAGGAGCGGCTCAAAGAAGATACGTCTTTTGGGGGCAACCTCGGGCGAGCAGTCTTCCAGGCCAAAAAGAGGCTT
CTTGAACCTCTTGGTCTGGTTGAGGAAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGCTAAAAAGAGACTCAATTTCGGTCAG
ACTGGCGACACAGAGTCAGTCCCAGACCCTCAACCAATCGGAGAACCTCCCGCAGCCCCCTCAGGTGTGGGATCT
CTTACAATGGCTTCAGGTGGTGGCGCACCAGTGGCAGACAATAACGAAGGTGCCGATGGAGTGGGTAGTTCCTCG
GGAAATTGGCATTGCGATTCCCAATGGCTGGGGGACAGAGTCATCACCACCAGCACCCGAACCTGGGCCCTGCCC
ACCTACAACAATCACCTCTACAAGCAAATCTCCAACAGCACATCTGGAGGATCTTCAAATGACAACGCCTACTTC
GGCTACAGCACCCCCTGGGGGTATTTTGACTTCAACAGATTCCACTGCCACTTCTCACCACGTGACTGGCAGCGA
CTCATCAACAACAACTGGGGATTCCGGCCTAAGCGACTCAACTTCAAGCTCTTCAACATTCAGGTCAAAGAGGTT
ACGGACAACAATGGAGTCAAGACCATCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTTCCCAGCGGACGTTTTCATGATTCCT
CAGTACGGGTATCTGACGCTTAATGATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTTTGAGAACGTACCTTTCCATAGCAGC
TACGCTCACAGCCAAAGCCTGGACCGACTAATGAATCCACTCATCGACCAATACTTGTACTATCTCTCAAAGACT
ATTAACGGTTCTGGACAGAATCAACAAACGCTAAAATTCAGTGTGGCCGGACCCAGCAACATGGCTGTCCAGGGA
AGAAACTACATACCTGGACCCAGCTACCGACAACAACGTGTCTCAACCACTGTGACTCAAAACAACAACAGCGAA
TTTGCTTGGCCTGGAGCTTCTTCTTGGGCTCTCAATGGACGTAATAGCTTGATGAATCCTGGACCTGCTATGGCC
AGCCACAAAGAAGGAGAGGACCGTTTCTTTCCTTTGTCTGGATCTTTAATTTTTGGCAAACAAGGAACTGGAAGA
GACAACGTGGATGCGGACAAAGTCATGATAACCAACGAAGAAGAAATTAAAACTACTAACCCGGTAGCAACGGAG
TCCTATGGACAAGTGGCCACAAACCACCAGAGTGCCCAAGCACAGGCGCAGACCGGCTGGGTTCAAAACCAAGGA
ATACTTCCGGGTATGGTTTGGCAGGACAGAGATGTGTACCTGCAAGGACCCATTTGGGCCAAAATTCCTCACACG
GACGGCAACTTTCACCCTTCTCCGCTGATGGGAGGGTTTGGAATGAAGCACCCGCCTCCTCAGATCCTCATCAAA
AACACACCTGTACCTGCGGATCCTCCAACGGCCTTCAACAAGGACAAGCTGAACTCTTTCATCACCCAGTATTCT
ACTGGCCAAGTCAGCGTGGAGATCGAGTGGGAGCTGCAGAAGGAAAACAGCAAGCGCTGGAACCCGGAGATCCAG
TACACTTCCAACTATTACAAGTCTAATAATGTTGAATTTGCTGTTAATACTGAAGGTGTATATAGTGAACCCCGC
CCCATTGGCACCAGATACCTGACTCGTAATCTGTAA

### AAV-10

### Partial Genome: AY631965

Rep78:
(SEQ ID NO: 22)
ATGCCGGGCTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCGGAATCTGATCGAGCAG
GCACCCCTGACCGTGGCCGAGAAGCTGCAGCGCGACTTCCTGGTCCACTGGCGCCGCGTGAGTAAGGCCCCGGAG
GCCCTCTTCTTTGTTCAGTTCGAGAAGGGCGAGTCCTACTTTCACCTGCACGTTCTGGTCGAGACCACGGGGGTC
AAGTCCATGGTCCTGGGCCGCTTCCTGAGTCAGATCAGAGACAGGCTGGTGCAGACCATCTACCGCGGGGTAGAG
CCCACGCTGCCCAACTGGTTCGCGGTGACCAAGACGCGAAATGGCGCCGGCGGGGGGAACAAGGTGGTGGACGAG
TGCTACATCCCCAACTACCTCCTGCCCAAGACGCAGCCCGAGCTGCAGTGGGCGTGGACTAACATGGAGGAGTAT
ATAAGCGCGTGTCTGAACCTCGCGGAGCGTAAACGGCTCGTGGCGCAGCACCTGACCCACGTCAGCCAGACGCAG
GAGCAGAACAAGGAGAATCTGAACCCGAATTCTGACGCGCCCGTGATCAGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCGCTGGACAATGCCGGAAAGATCATGGCG
CTGACCAAATCCGCGCCCGACTACCTGGTAGGCCCGTCCTTACCCGCGGACATTAAGGCCAACCGCATCTACCGC
ATCCTGGAGCTCAACGGCTACGACCCCGCCTACGCCGGCTCCGTCTTCCTGGGCTGGGCGCAGAAAAAGTTCGGT
AAAAGGAATACAATTTGGCTGTTCGGGCCCGCCACCACCGGCAAGACCAACATCGCGGAAGCCATCGCCCACGCC
GTGCCCTTCTACGGCTGCGTCAACTGGACCAATGAGAACTTTCCCTTCAACGATTGCGTCGACAAGATGGTGATC
TGGTGGGAGGAGGGCAAGATGACCGCCAAGGTCGTGGAGTCCGCCAAGGCCATTCTGGGCGGAAGCAAGGTGCGC
GTCGACCAAAAGTGCAAGTCCTCGGCCCAGATCGACCCCACGCCCGTGATCGTCACCTCCAACACCAACATGTGC
GCCGTGATCGACGGGAACAGCACCACCTTCGAGCACCAGCAGCCCCTGCAGGACCGCATGTTCAAGTTCGAGCTC
ACCCGCCGTCTGGAGCACGACTTTGGCAAGGTGACCAAGCAGGAAGTCAAAGAGTTCTTCCGCTGGGCTCAGGAT
CACGTGACTGAGGTGACGCATGAGTTCTACGTCAGAAAGGGCGGAGCCACCAAAAGACCCGCCCCCAGTGACGCG
GATATAAGCGAGCCCAAGCGGGCCTGCCCCTCAGTTGCGGAGCCATCGACGTCAGACGCGGAAGCACCGGTGGAC
TTTGCGGACAGGTACCAAAACAAATGTTCTCGTCACGCGGGCATGCTTCAGATGCTGTTTCCCTGCAAGACATGC
GAGAGAATGAATCAGAATTTCAACGTCTGCTTCACGCACGGGGTCAGAGACTGCTCAGAGTGCTTCCCCGGCGCG
TCAGAATCTCAACCTGTCGTCAGAAAAAAGACGTATCAGAAACTGTGCGCGATTCATCATCTGCTGGGGGGGCA
CCCGAGATTGCGTGTTCGGCCTGCGATCTCGTCAACGTGGACTTGGATGACTGTGTTTCTGAGCAATAA
CapVP1:
(SEQ ID NO: 23)
ATGGCTGCTGACGGTTATCTTCCAGATTGGCTCGAGGACAACCTCTCTGAGGGCATTCGCGAGTGGTGGGACCTG
AAACCTGGAGCCCCCAAGCCCAAGGCCAACCAGCAGAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGCGGGTT
CTCGAACCTCTCGGTCTGGTTGAGGAAGCTGCTAAGACGGCTCCTGGAAAGAAGAGACCGGTAGAACCGTCACCT
CAGCGTTCCCCCGACTCCTCCACGGGCATCGGCAAGAAAGGCCAGCAGCCCGCTAAAAAGAGACTGAACTTTGGG
CAGACTGGCGAGTCAGAGTCAGTCCCCGACCCTCAACCAATCGGAGAACCACCAGCAGGCCCCTCTGGTCTGGGA
TCTGGTACAATGGCTGCAGGCGGTGGCGCTCCAATGGCAGACAATAACGAAGGCGCCGACGGAGTGGGTAGTTCC
TCAGGAAATTGGCATTGCGATTCCACATGGCTGGGCGACAGAGTCATCACCACCAGCACCCGAACCTGGGCCCTG
CCCACCTACAACAACCACCTCTACAAGCAAATCTCCAACGGGACATCGGGAGGAAGCACCAACGACAACACCTAC
TTCGGCTACAGCACCCCCTGGGGGTATTTTGACTTCAACAGATTCCACTGCCACTTCTCACCACGTGACTGGCAG
CGACTCATCAACAACAACTGGGGATTCCGGCCAAAAAGACTCAGCTTCAAGCTCTTCAACATCCAGGTCAAGGAG
GTCACGCAGAATGAAGGCACCAAGACCATCGCCAATAACCTTACCAGCACGATTCAGGTATTTACGGACTCGGAA
TACCAGCTGCCGTACGTCCTCGGCTCCGCGCACCAGGGCTGCCTGCCTCCGTTCCCGGCGGATGTCTTCATGATT
CCCCAGTACGGCTACCTGACACTGAACAATGGAAGTCAAGCCGTAGGCCGTTCCTCCTTCTACTGCCTGGAATAT
TTTCCATCTCAAATGCTGCGAACTGGAAACAATTTTGAATTCAGCTACACCTTCGAGGACGTGCCTTTCCACAGC
AGCTACGCACACAGCCAGAGCTTGGACCGACTGATGAATCCTCTCATTGACCAGTACCTGTACTACTTATCCAGA
ACTCAGTCCACAGGAGGAACTCAAGGTACCCAGCAATTGTTATTTTCTCAAGCTGGGCCTGCAAACATGTCGGCT
CAGGCCAAGAACTGGCTGCCTGGACCTTGCTACCGGCAGCAGCGAGTCTCCACGACACTGTCGCAAAACAACAAC
AGCAACTTTGCTTGGACTGGTGCCACCAAATATCACCTGAACGGAAGAGACTCTCTGGTGAATCCCGGTGTCGCC
ATGGCAACCCACAAGGACGACGAGGAACGCTTCTTCCCGTCGAGCGGAGTCCTGATGTTTGGAAAACAGGGTGCT
GGAAGAGACAATGTGGACTACAGCAGCGTTATGCTAACAAGCGAAGAAGAAATTAAAACCACTAACCCTGTAGCC
ACAGAACAATACGGCGTGGTGGCTGACAACTTGCAGCAAGCCAATACAGGGCCTATTGTGGGAAATGTCAACAGC
CAAGGAGCCTTACCTGGCATGGTCTGGCAGAACCGAGACGTGTACCTGCAGGGTCCCATCTGGGCCAAGATTCCT
CACACGGACGGCAACTTTCACCCGTCTCCTCTGATGGGCGGCTTTGGACTTAAACACCCGCCTCCACAGATCCTG
ATCAAGAACACGCCGGTACCTGCGGATCCTCCAACAACGTTCAGCCAGGCGAAATTGGCTTCCTTCATCACGCAG
TACAGCACCGGACAGGTCAGCGTGGAAATCGAGTGGGAGCTGCAGAAGGAGAACAGCAAACGCTGGAACCCAGAG
ATTCAGTACACTTCAAACTACTACAAATCTACAAATGTGGACTTTGCTGTCAATACAGAGGGAACTTATTCTGAG
CCTCGCCCCATTGGTACTCGTTATCTGACACGTAATCTGTAA

### AAV-11

### Partial Genome: AY631966

Rep78:
(SEQ ID NO: 24)
ATGCCGGGCTTCTACGAGATCGTGATCAAGGTGCCGAGCGACCTGGACGAGCACCTGCCGGGCATTTCTGACTCG
TTTGTGAACTGGGTGGCCGAGAAGGAATGGGAGCTGCCCCCGGATTCTGACATGGATCGGAATCTGATCGAGCAG
GCACCCCTGACCGTGGCCGAGAAGCTGCAGCGCGACTTCCTGGTCCACTGGCGCCGCGTGAGTAAGGCCCCGGAG
GCCCTCTTCTTTGTTCAGTTCGAGAAGGGCGAGTCCTACTTCCACCTCCACGTTCTCGTCGAGACCACGGGGGTC
AAGTCCATGGTCCTGGGCCGCTTCCTGAGTCAGATCAGAGACAGGCTGGTGCAGACCATCTACCGCGGGGTCGAG
CCCACGCTGCCCAACTGGTTCGCGGTGACCAAGACGCGAAATGGCGCCGGCGGGGGGAACAAGGTGGTGGACGAG
TGCTACATCCCCAACTACCTCCTGCCCAAGACCCAGCCCGAGCTGCAGTGGGCGTGGACTAACATGGAGGAGTAT
ATAAGCGCGTGTCTAAACCTCGCGGAGCGTAAACGGCTCGTGGCGCAGCACCTGACCCACGTCAGCCAGACGCAG
GAGCAGAACAAGGAGAATCTGAACCCGAATTCTGACGCGCCCGTGATCAGGTCAAAAACCTCCGCGCGCTACATG
GAGCTGGTCGGGTGGCTGGTGGACCGGGGCATCACCTCCGAGAAGCAGTGGATCCAGGAGGACCAGGCCTCGTAC
ATCTCCTTCAACGCCGCCTCCAACTCGCGGTCCCAGATCAAGGCCGCGCTGGACAATGCCGGAAAGATCATGGCG
CTGACCAAATCCGCGCCCGACTACCTGGTAGGCCCGTCCTTACCCGCGGACATTAAGGCCAACCGCATCTACCGC
ATCCTGGAGCTCAACGGCTACGACCCCGCCTACGCCGGCTCCGTCTTCCTGGGCTGGGCGCAGAAAAAGTTCGGT
AAACGCAACACCATCTGGCTGTTTGGGCCCGCCACCACCGGCAAGACCAACATCGCGGAAGCCATAGCCCACGCC
GTGCCCTTCTACGGCTGCGTGAACTGGACCAATGAGAACTTTCCCTTCAACGATTGCGTCGACAAGATGGTGATC
TGGTGGGAGGAGGGCAAGATGACCGCCAAGGTCGTGGAGTCCGCCAAGGCCATTCTGGGCGGAAGCAAGGTGCGC
GTGGACCAAAAGTGCAAGTCCTCGGCCCAGATCGACCCCACGCCCGTGATCGTCACCTCCAACACCAACATGTGC
GCCGTGATCGACGGGAACAGCACCACCTTCGAGCACCAGCAGCCGCTGCAGGACCGCATGTTCAAGTTCGAGCTC
ACCCGCCGTCTGGAGCACGACTTTGGCAAGGTGACCAAGCAGGAAGTCAAAGAGTTCTTCCGCTGGGCTCAGGAT
CACGTGACTGAGGTGGCGCATGAGTTCTACGTCAGAAAGGGCGGAGCCACCAAAAGACCCGCCCCCAGTGACGCG
GATATAAGCGAGCCCAAGCGGGCCTGCCCCTCAGTTCCGGAGCCATCGACGTCAGACGCGGAAGCACCGGTGGAC
TTTGCGGACAGGTACCAAAACAAATGTTCTCGTCACGCGGGCATGCTTCAGATGCTGTTTCCCTGCAAGACATGC
GAGAGAATGAATCAGAATTTCAACGTCTGCTTCACGCACGGGGTCAGAGACTGCTCAGAGTGCTTCCCCGGCGCG
TCAGAATCTCAACCCGTCGTCAGAAAAAAGACGTATCAGAAACTGTGCGCGATTCATCATCTGCTGGGGGGGGCA
CCCGAGATTGCGTGTTCGGCCTGCGATCTCGTCAACGTGGACTTGGATGACTGTGTTTCTGAGCAATAA
CapVP1:
(SEQ ID NO: 25)
ATGGCTGCTGACGGTTATCTTCCAGATTGGCTCGAGGACAACCTCTCTGAGGGCATTCGCGAGTGGTGGGACCTG
AAACCTGGAGCCCCGAAGCCCAAGGCCAACCAGCAGAAGCAGGACGACGGCCGGGGTCTGGTGCTTCCTGGCTAC
AAGTACCTCGGACCCTTCAACGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAACCACGCCGACGCCGAGTTT
CAGGAGCGTCTGCAAGAAGATACGTCTTTTGGGGGCAACCTCGGGCGAGCAGTCTTCCAGGCCAAGAAGAGGGTA
CTCGAACCTCTGGGCCTGGTTGAAGAAGGTGCTAAAACGGCTCCTGGAAAGAAGAGACCGTTAGAGTCACCACAA
GAGCCCGACTCCTCCTCGGGCATCGGCAAAAAAGGCAAACAACCAGCCAGAAAGAGGCTCAACTTTGAAGAGGAC
ACTGGAGCCGGAGACGGACCCCCTGAAGGATCAGATACCAGCGCCATGTCTTCAGACATTGAAATGCGTGCAGCA
CCGGGCGGAAATGCTGTCGATGCGGGACAAGGTTCCGATGGAGTGGGTAATGCCTCGGGTGATTGGCATTGCGAT
TCCACCTGGTCTGAGGGCAAGGTCACAACAACCTCGACCAGAACCTGGGTCTTGCCCACCTACAACAACCACTTG
TACCTGCGTCTCGGAACAACATCAAGCAGCAACACCTACAACGGATTCTCCACCCCCTGGGGATATTTTGACTTC
AACAGATTCCACTGTCACTTCTCACCACGTGACTGGCAAAGACTCATCAACAACAACTGGGGACTACGACCAAAA
GCCATGCGCGTTAAAATCTTCAATATCCAAGTTAAGGAGGTCACAACGTCGAACGGCGAGACTACGGTCGCTAAT
AACCTTACCAGCACGGTTCAGATATTTGCGGACTCGTCGTATGAGCTCCCGTACGTGATGGACGCTGGACAAGAG
GGGAGCCTGCCTCCTTTCCCCAATGACGTGTTCATGGTGCCTCAATATGGCTACTGTGGCATCGTGACTGGCGAG
AATCAGAACCAAACGGACAGAAACGCTTTCTACTGCCTGGAGTATTTTCCTTCGCAAATGTTGAGAACTGGCAAC
AACTTTGAAATGGCTTACAACTTTGAGAAGGTGCCGTTCCACTCAATGTATGCTCACAGCCAGAGCCTGGACAGA
CTGATGAATCCCCTCCTGGACCAGTACCTGTGGCACTTACAGTCGACTACCTCTGGAGAGACTCTGAATCAAGGC
AATGCAGCAACCACATTTGGAAAAATCAGGAGTGGAGACTTTGCCTTTTACAGAAAGAACTGGCTGCCTGGGCCT
TGTGTTAAACAGCAGAGATTCTCAAAAACTGCCAGTCAAAATTACAAGATTCCTGCCAGCGGGGGCAACGCTCTG
TTAAAGTATGACACCCACTATACCTTAAACAACCGCTGGAGCAACATCGCGCCCGGACCTCCAATGGCCACAGCC
GGACCTTCGGATGGGGACTTCAGTAACGCCCAGCTTATATTCCCTGGACCATCTGTTACCGGAAATACAACAACT
TCAGCCAACAATCTGTTGTTTACATCAGAAGAAGAAATTGCTGCCACCAACCCAAGAGACACGGACATGTTTGGC
CAGATTGCTGACAATAATCAGAATGCTACAACTGCTCCCATAACCGGCAACGTGACTGCTATGGGAGTGCTGCCT
GGCATGGTGTGGCAAAACAGAGACATTTACTACCAAGGGCCAATTTGGGCCAAGATCCCACACGCGGACGGACAT
TTTCATCCTTCACCGCTGATTGGTGGGTTTGGACTGAAACACCCGCCTCCCCAGATATTCATCAAGAACACTCCC
GTACCTGCCAATCCTGCGACAACCTTCACTGCAGCCAGAGTGGACTCTTTCATCACACAATACAGCACCGGCCAG
GTCGCTGTTCAGATTGAATGGGAAATTGAAAAGGAACGCTCCAAACGCTGGAATCCTGAAGTGCAGTTTACTTCA
AACTATGGGAACCAGTCTTCTATGTTGTGGGCTCCTGATACAACTGGGAAGTATACAGAGCCGCGGGTTATTGGC
TCTCGTTATTTGACTAATCATTTGTAA

### AAV-12

### Partial Genome: DQ813647

Rep78:
(SEQ ID NO: 26)
ATGCCGGGGTTCTACGAGGTGGTGATCAAGGTGCCCAGCGACCTGGACGAGCACCTGCCCGGCATTTCTGACTCC
TTTGTGAACTGGGTGGCCGAGAAGGAATGGGAGTTGCCCCCGGATTCTGACATGGATCAGAATCTGATTGAGCAG
GCACCCCTGACCGTGGCCGAGAAGCTGCAGCGCGAGTTCCTGGTGGAATGGCGCCGAGTGAGTAAATTTCTGGAG
GCCAAGTTTTTTGTGCAGTTTGAAAAGGGGGACTCGTACTTTCATTTGCATATTCTGATTGAAATTACCGGCGTG
AAATCCATGGTGGTGGGCCGCTACGTGAGTCAGATTAGGGATAAACTGATCCAGCGCATCTACCGCGGGGTCGAG
CCCCAGCTGCCCAACTGGTTCGCGGTCACAAAGACCCGAAATGGCGCCGGAGGCGGGAACAAGGTGGTGGACGAG
TGCTACATCCCCAACTACCTGCTCCCCAAGGTCCAGCCCGAGCTTCAGTGGGCGTGGACTAACATGGAGGAGTAT
ATAAGCGCCTGTTTGAACCTCGCGGAGCGTAAACGGCTCGTGGCGCAGCACCTGACGCACGTCTCCCAGACCCAG
GAGGGCGACAAGGAGAATCTGAACCCGAATTCTGACGCGCCGGTGATCCGGTCAAAAACCTCCGCCAGGTACATG
GAGCTGGTCGGGTGGCTGGTGGACAAGGGCATCACGTCCGAGAAGCAGTGGATCCAGGAGGACCAGGCCTCGTAC
ATCTCCTTCAACGCGGCCTCCAACTCCCGGTCGCAGATCAAGGCGGCCCTGGACAATGCCTCCAAAATCATGAGC
CTCACCAAAACGGCTCCGGACTATCTCATCGGGCAGCAGCCCGTGGGGGACATTACCACCAACCGGATCTACAAA
ATCCTGGAACTGAACGGGTACGACCCCCAGTACGCCGCCTCCGTCTTTCTCGGCTGGGCCCAGAAAAAGTTTGGA
AAGCGCAACACCATCTGGCTGTTTGGGCCCGCCACCACCGGCAAGACCAACATCGCGGAAGCCATCGCCCACGCG
GTCCCCTTCTACGGCTGCGTCAACTGGACCAATGAGAACTTTCCCTTCAACGACTGCGTCGACAAAATGGTGATT
TGGTGGGAGGAGGGCAAGATGACCGCCAAGGTCGTAGAGTCCGCCAAGGCCATTCTGGGCGGCAGCAAGGTGCGC
GTGGACCAAAAATGCAAGGCCTCTGCGCAGATCGACCCCACCCCCGTGATCGTCACCTCCAACACCAACATGTGC
GCCGTGATTGACGGGAACAGCACCACCTTCGAGCACCAGCAGCCCCTGCAGGACCGGATGTTCAAGTTTGAACTC
ACCCGCCGCCTCGACCACGACTTTGGCAAGGTCACCAAGCAGGAAGTCAAGGACTTTTTCCGGTGGGCGGCTGAT
CACGTGACTGACGTGGCTCATGAGTTTTACGTCACAAAGGGTGGAGCTAAGAAAAGGCCCGCCCCCTCTGACGAG
GATATAAGCGAGCCCAAGCGGCCGCGCGTGTCATTTGCGCAGCCGGAGACGTCAGACGCGGAAGCTCCCGGAGAC
TTCGCCGACAGGTACCAAAACAAATGTTCTCGTCACGCGGGTATGCTGCAGATGCTCTTTCCCTGCAAGACGTGC
GAGAGAATGAATCAGAATTCCAACGTCTGCTTCACGCACGGTCAGAAAGATTGCGGGGAGTGCTTTCCCGGGTCA
GAATCTCAACCGGTTTCTGTCGTCAGAAAAACGTATCAGAAACTGTGCATCCTTCATCAGCTCCGGGGGGCACCC
GAGATCGCCTGCTCTGCTTGCGACCAACTCAACCCCGATTTGGACGATTGCCAATTTGAGCAATAA
CapVP1:
(SEQ ID NO: 27)
ATGGCTGCTGACGGTTATCTTCCAGATTGGCTCGAGGACAACCTCTCTGAAGGCATTCGCGAGTGGTGGGCGCTG
AAACCTGGAGCTCCACAACCCAAGGCCAACCAACAGCATCAGGACAACGGCAGGGGTCTTGTGCTTCCTGGGTAC
AAGTACCTCGGACCCTTCAACGGACTCGACAAGGGAGAGCCGGTCAACGAGGCAGACGCCGCGGCCCTCGAGCAC
GACAAGGCCTACGACAAGCAGCTCGAGCAGGGGGACAACCCGTATCTCAAGTACAACCACGCCGACGCCGAGTTC
CAGCAGCGCTTGGCGACCGACACCTCTTTTGGGGGCAACCTCGGGCGAGCAGTCTTCCAGGCCAAAAAGAGGATT
CTCGAGCCTCTGGGTCTGGTTGAAGAGGGCGTTAAAACGGCTCCTGGAAAGAAACGCCCATTAGAAAAGACTCCA
AATCGGCCGACCAACCCGGACTCTGGGAAGGCCCCGGCCAAGAAAAAGCAAAAAGACGGCGAACCAGCCGACTCT
GCTAGAAGGACACTCGACTTTGAAGACTCTGGAGCAGGAGACGGACCCCCTGAGGGATCATCTTCCGGAGAAATG
TCTCATGATGCTGAGATGCGTGCGGCGCCAGGCGGAAATGCTGTCGAGGCGGGACAAGGTGCCGATGGAGTGGGT
AATGCCTCCGGTGATTGGCATTGCGATTCCACCTGGTCAGAGGGCCGAGTCACCACCACCAGCACCCGAACCTGG
GTCCTACCCACGTACAACAACCACCTGTACCTGCGAATCGGAACAACGGCCAACAGCAACACCTACAACGGATTC
TCCACCCCCTGGGGATACTTTGACTTTAACCGCTTCCACTGCCACTTTTCCCCACGCGACTGGCAGCGACTCATC
AACAACAACTGGGGACTCAGGCCGAAATCGATGCGTGTTAAAATCTTCAACATACAGGTCAAGGAGGTCACGACG
TCAAACGGCGAGACTACGGTCGCTAATAACCTTACCAGCACGGTTCAGATCTTTGCGGATTCGACGTATGAACTC
CCATACGTGATGGACGCCGGTCAGGAGGGGAGCTTTCCTCCGTTTCCCAACGACGTCTTTATGGTTCCCCAATAC
GGATACTGCGGAGTTGTCACTGGAAAAAACCAGAACCAGACAGACAGAAATGCCTTTTACTGCCTGGAATACTTT
CCATCCCAAATGCTAAGAACTGGCAACAATTTTGAAGTCAGTTACCAATTTGAAAAAGTTCCTTTCCATTCAATG
TACGCGCACAGCCAGAGCCTGGACAGAATGATGAATCCTTTACTGGATCAGTACCTGTGGCATCTGCAATCGACC
ACTACCGGAAATTCCCTTAATCAAGGAACAGCTACCACCACGTACGGGAAAATTACCACTGGAGACTTTGCCTAC
TACAGGAAAAACTGGTTGCCTGGAGCCTGCATTAAACAACAAAAATTTTCAAAGAATGCCAATCAAAACTACAAG
ATTCCCGCCAGCGGGGGAGACGCCCTTTTAAAGTATGACACGCATACCACTCTAAATGGGCGATGGAGTAACATG
GCTCCTGGACCTCCAATGGCAACCGCAGGTGCCGGGGACTCGGATTTTAGCAACAGCCAGCTGATCTTTGCCGGA
CCCAATCCGAGCGGTAACACGACCACATCTTCAAACAATTTGTTGTTTACCTCAGAAGAGGAGATTGCCACAACA
AACCCACGAGACACGGACATGTTTGGACAGATTGCAGATAATAATCAAAATGCCACCACCGCCCCTCACATCGCT
AACCTGGACGCTATGGGAATTGTTCCCGGAATGGTCTGGCAAAACAGAGACATCTACTACCAGGGCCCTATTTGG
GCCAAGGTCCCTCACACGGACGGACACTTTCACCCTTCGCCGCTGATGGGAGGATTTGGACTGAAACACCCGCCT
CCACAGATTTTCATCAAAAACACCCCCGTACCCGCCAATCCCAATACTACCTTTAGCGCTGCAAGGATTAATTCT
TTTCTGACGCAGTACAGCACCGGACAAGTTGCCGTTCAGATCGACTGGGAAATTCAGAAGGAGCATTCCAAACGC
TGGAATCCCGAAGTTCAATTTACTTCAAACTACGGCACTCAAAATTCTATGCTGTGGGCTCCCGACAATGCTGGC
AACTACCACGAACTCCGGGCTATTGGGTCCCGTTTCCTCACCCACCACTTGTAA

### AAV-13

### Partial Genome: EU285562

Rep78:
(SEQ ID NO: 28)
ATGCCGGGATTCTACGAGATTGTCCTGAAGGTGCCCAGCGACCTGGACGAGCACCTGCCTGGCATTTCTGACTCT
TTTGTAAACTGGGTGGCGGAGAAGGAATGGGAGCTGCCGCCGGATTCTGACATGGATCTGAATCTGATTGAGCAG
GCACCCCTAACCGTGGCCGAAAAGCTGCAACGCGAATTCCTGGTCGAGTGGCGCCGCGTGAGTAAGGCCCCGGAG
GCCCTCTTCTTTGTTCAGTTCGAGAAGGGGGACAGCTACTTCCACCTACACATTCTGGTGGAGACCGTGGGCGTG
AAATCCATGGTGGTGGGCCGCTACGTGAGCCAGATTAAAGAGAAGCTGGTGACCCGCATCTACCGCGGGGTCGAG
CCGCAGCTTCCGAACTGGTTCGCGGTGACCAAGACGCGTAATGGCGCCGGAGGCGGGAACAAGGTGGTGGACGAC
TGCTACATCCCCAACTACCTGCTCCCCAAGACCCAGCCCGAGCTCCAGTGGGCGTGGACTAATATGGACCAGTAT
TTAAGCGCCTGTTTGAATCTCGCGGAGCGTAAACGGCTGGTGGCGCAGCATCTGACGCACGTGTCGCAGACGCAG
GAGCAGAACAAAGAGAACCAGAATCCCAATTCTGACGCGCCGGTGATCAGATCAAAAACCTCCGCGAGGTACATG
GAGCTGGTCGGGTGGCTGGTGGACCGCGGGATCACGTCAGAAAAGCAATGGATCCAGGAGGACCAGGCCTCTTAC
ATCTCCTTCAACGCCGCCTCCAACTCGCGGTCACAAATCAAGGCCGCACTGGACAATGCCTCCAAATTTATGAGC
CTGACAAAAACGGCTCCGGACTACCTGGTGGGAAACAACCCGCCGGAGGACATTACCAGCAACCGGATCTACAAA
ATCCTCGAGATGAACGGGTACGATCCGCAGTACGCGGCCTCCGTCTTCCTGGGCTGGGCGCAAAAGAAGTTCGGG
AAGAGGAACACCATCTGGCTCTTTGGGCCGGCCACGACGGGTAAAACCAACATCGCTGAAGCTATCGCCCACGCC
GTGCCCTTTTACGGCTGCGTGAACTGGACCAATGAGAACTTTCCGTTCAACGATTGCGTCGACAAGATGGTGATC
TGGTGGGAGGAGGGCAAGATGACGGCCAAGGTCGTGGAGTCCGCCAAGGCCATTCTGGGCGGAAGCAAGGTGCGC
GTGGACCAAAAGTGCAAGTCATCGGCCCAGATCGACCCAACTCCCGTCATCGTCACCTCCAACACCAACATGTGC
GCGGTCATCGACGGAAATTCCACCACCTTCGAGCACCAACAACCACTCCAAGACCGGATGTTCAAGTTCGAGCTC
ACCAAGCGCCTGGAGCACGACTTTGGCAAGGTCACCAAGCAGGAAGTCAAGGACTTTTTCCGGTGGGCGTCAGAT
CACGTGACTGAGGTGTCTCACGAGTTTTACGTCAGAAAGGGTGGAGCTAGAAAGAGGCCCGCCCCCAATGACGCA
GATATAAGTGAGCCCAAGCGGGCCTGTCCGTCAGTTGCGCAGCCATCGACGTCAGACGCGGAAGCTCCGGTGGAC
TACGCGGACAGGTACCAAAACAAATGTTCTCGTCACGTGGGCATGAATCTGATGCTTTTTCCCTGCCGGCAATGC
GAGAGAATGAATCAGAATGTGGACATTTGCTTCACGCACGGGGTCATGGACTGTGCCGAGTGCTTCCCCGTGTCA
GAATCTCAACCCGTGTCTGTCGTCAGAAAGCGGACATATCAGAAACTGTGTCCGATTCATCACATCATGGGGAGG
GCGCCCGAGGTGGCTTGTTCGGCCTGCGATCTGGCCAATGTGGACTTGGATGACTGTGACATGGAGCAATAA
CapVP1:
(SEQ ID NO: 29)
ATGACTGACGGTTACCTTCCAGATTGGCTAGAGGACAACCTCTCTGAAGGCGTTCGAGAGTGGTGGGCGCTGCAA
CCTGGAGCCCCTAAACCCAAGGCAAATCAACAACATCAGGACAACGCTCGGGGTCTTGTGCTTCCGGGTTACAAA
TACCTCGGACCCGGCAACGGACTTGACAAGGGGGAACCCGTCAACGCAGCGGACGCGGCAGCCCTCGAACACGAC
AAGGCCTACGACCAGCAGCTCAAGGCCGGTGACAACCCCTACCTCAAGTACAACCACGCCGACGCCGAGTTTCAG
GAGCGTCTTCAAGAAGATACGTCTTTTGGGGGCAACCTCGGACGAGCAGTCTTCCAGGCCAAAAAGAGGATCCTT
GAGCCTCTGGGTCTGGTTGAGGAAGCGGCTAAGACGGCTCCTGGAAAAAAGAGACCTGTAGAGCAATCTCCAGCA
GAACCGGACTCCTCTTCGGGCATCGGCAAATCAGGCCAGCAGCCCGCTAGAAAAAGACTGAATTTTGGTCAGACT
GGCGACACAGAGTCAGTCCCAGACCCTCAACCACTCGGACAACCTCCCGCAGCCCCCTCTGGTGTGGGATCTACT
ACAATGGCTTCAGGCGGTGGCGCACCAATGGCAGACAATAACGAGGGTGCCGATGGAGTGGGTAATTCCTCAGGA
AATTGGCATTGCGATTCCCAATGGCTGGGCGACAGAGTCATCACCACCAGCACCCGCACCTGGGCCCTGCCCACC
TACAACAATCACCTCTACAAGCAAATCTCCAGCCAATCAGGAGCCACCAACGACAACCACTACTTTGGCTACAGC
ACCCCCTGGGGGTATTTTGACTTCAACAGATTCCACTGCCACTTTTCACCACGTGACTGGCAAAGACTCATCAAC
AACAACTGGGGATTCCGACCCAAGAGACTCAACTTCAAGCTCTTTAACATTCAAGTCAAAGAGGTCACGCAGAAT
GACGGTACGACGACGATTGCCAATAACCTTACCAGCACGGTTCAGGTGTTTACTGACTCCGAGTACCAGCTCCCG
TACGTCCTCGGCTCGGCGCATCAGGGATGCCTCCCGCCGTTCCCAGCAGACGTCTTCATGGTCCCACAGTATGGA
TACCTCACCCTGAACAACGGGAGTCAGGCGGTAGGACGCTCTTCCTTTTACTGCCTGGAGTACTTTCCTTCTCAG
ATGCTGCGTACTGGAAACAACTTTCAGTTTAGCTACACTTTTGAAGACGTGCCTTTCCACAGCAGCTACGCTCAC
AGCCAAAGTCTGGACCGTCTCATGAATCCTCTGATCGACCAGTACCTGTACTATCTGAACAGGACACAAACAGCC
AGTGGAACTCAGCAGTCTCGGCTACTGTTTAGCCAAGCTGGACCCACCAGTATGTCTCTTCAAGCTAAAAACTGG
CTGCCTGGACCTTGCTACAGACAGCAGCGTCTGTCAAAGCAGGCAAACGACAACAACAACAGCAACTTTCCCTGG
ACTGGTGCCACCAAATATCATCTGAATGGCCGGGACTCATTGGTGAACCCGGGCCCTGCTATGGCCAGTCACAAG
GATGACAAAGAAAAGTTTTTCCCCATGCATGGAACCCTGATATTTGGTAAAGAAGGAACAAATGCCAACAACGCG
GATTTGGAAAATGTCATGATTACAGATGAAGAAGAAATCCGCACCACCAATCCCGTGGCTACGGAGCAGTACGGG
ACTGTGTCAAATAATTTGCAAAACTCAAACGCTGGTCCAACTACTGGAACTGTCAATCACCAAGGAGCGTTACCT
GGTATGGTGTGGCAGGATCGAGACGTGTACCTGCAGGGACCCATTTGGGCCAAGATTCCTCACACCGATGGACAC
TTTCATCCTTCTCCACTGATGGGAGGTTTTGGGCTCAAACACCCGCCTCCTCAGATCATGATCAAAAACACTCCC
GTTCCAGCCAATCCTCCCACAAACTTTAGTGCGGCAAAGTTTGCTTCCTTCATCACACAGTACTCCACGGGGCAG
GTCAGCGTGGAGATCGAGTGGGAGCTGCAGAAGGAGAACAGCAAACGCTGGAATCCCGAAATTCAGTACACTTCC
AACTACAACAAATCTGTTAATGTGGACTTTACTGTGGACACTAATGGTGTGTATTCAGAGCCTCGCCCCATTGGC
ACCAGATACCTGACTCGTAATCTGTAA
ITR Sequence
(SEQ ID NO: 30)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCT
Rep2 Sequence - Contains Rep78 and Rep52 (start codon underlined
(SEQ ID NO: 31)
ATGCCGGGGTTTTACGAGATTGTGATTAAGGTCCCCAGCGACCTTGACGAGCATCTGCCCGGCATTTCTGACAGC
TTTGTGAACTGGGTGGCCGAGAAGGAATGGGAGTTGCCGCCAGATTCTGACATGGATCTGAATCTGATTGAGCAG
GCACCCCTGACCGTGGCCGAGAAGCTGCAGCGCGACTTTCTGACGGAATGGCGCCGTGTGAGTAAGGCCCCGGAG
GCCCTTTTCTTTGTGCAATTTGAGAAGGGAGAGAGCTACTTCCACATGCACGTGCTCGTGGAAACCACCGGGGTG
AAATCCATGGTTTTGGGACGTTTCCTGAGTCAGATTCGCGAAAAACTGATTCAGAGAATTTACCGCGGGATCGAG
CCGACTTTGCCAAACTGGTTCGCGGTCACAAAGACCAGAAATGGCGCCGGAGGCGGGAACAAGGTGGTGGATGAG
TGCTACATCCCCAATTACTTGCTCCCCAAAACCCAGCCTGAGCTCCAGTGGGCGTGGACTAATATGGAACAGTAT
TTAAGCGCCTGTTTGAATCTCACGGAGCGTAAACGGTTGGTGGCGCAGCATCTGACGCACGTGTCGCAGACGCAG
GAGCAGAACAAAGAGAATCAGAATCCCAATTCTGATGCGCCGGTGATCAGATCAAAAACTTCAGCCAGGTACATG
GAGCTGGTCGGGTGGCTCGTGGACAAGGGGATTACCTCGGAGAAGCAGTGGATCCAGGAGGACCAGGCCTCATAC
ATCTCCTTCAATGCGGCCTCCAACTCGCGGTCCCAAATCAAGGCTGCCTTGGACAATGCGGGAAAGATTATGAGC
CTGACTAAAACCGCCCCCGACTACCTGGTGGGCCAGCAGCCCGTGGAGGACATTTCCAGCAATCGGATTTATAAA
ATTTTGGAACTAAACGGGTACGATCCCCAATATGCGGCTTCCGTCTTTCTGGGATGGGCCACGAAAAAGTTCGGC
AAGAGGAACACCATCTGGCTGTTTGGGCCTGCAACTACCGGGAAGACCAACATCGCGGAGGCCATAGCCCACACT
GTGCCCTTCTACGGGTGCGTAAACTGGACCAATGAGAACTTTCCCTTCAACGACTGTGTCGACAAGATGGTGATC
TGGTGGGAGGAGGGGAAGATGACCGCCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGATCGTCACCTCCAACACCAACATGTGC
GCCGTGATTGACGGGAACTCAACGACCTTCGAACACCAGCAGCCGTTGCAAGACCGGATGTTCAAATTTGAACTC
ACCCGCCGTCTGGATCATGACTTTGGGAAGGTCACCAAGCAGGAAGTCAAAGACTTTTTCCGGTGGGCAAAGGAT
CACGTGGTTGAGGTGGAGCATGAATTCTACGTCAAAAAGGGTGGAGCCAAGAAAAGACCCGCCCCCAGTGACGCA
GATATAAGTGAGCCCAAACGGGTGCGCGAGTCAGTTGCGCAGCCATCGACGTCAGACGCGGAAGCTTCGATCAAC
TACGCAGACAGGTACCAAAACAAATGTTCTCGTCACGTGGGCATGAATCTGATGCTGTTTCCCTGCAGACAATGC
GAGAGAATGAATCAGAATTCAAATATCTGCTTCACTCACGGACAGAAAGACTGTTTAGAGTGCTTTCCCGTGTCA
GAATCTCAACCCGTTTCTGTCGTCAAAAAGGCGTATCAGAAACTGTGCTACATTCATCATATCATGGGAAAGGTG
CCAGACGCTTGCACTGCCTGCGATCTGGTCAATGTGGATTTGGATGACTGCATCTTTGAACAATAA
Cap2 Sequence - contains sequentially VP1, VP2, AAP, VP3 (start codons
underlined)
(SEQ ID NO: 32)
ATGGCTGCCGATGGTTATCTTCCAGATTGGCTCGAGGACACTCTCTCTGAAGGAATAAGACAGTGGTGGAAGCTC
AAACCTGGCCCACCACCACCAAAGCCCGCAGAGCGGCATAAGGACGACAGCAGGGGTCTTGTGCTTCCTGGGTAC
AAGTACCTCGGACCCTTCAACGGACTCGACAAGGGAGAGCCGGTCAACGAGGCAGACGCCGCGGCCCTCGAGCAC
GACAAAGCCTACGACCGGCAGCTCGACAGCGGAGACAACCCGTACCTCAAGTACAACCACGCCGACGCGGAGTTT
CAGGAGCGCCTTAAAGAAGATACGTCTTTTGGGGGCAACCTCGGACGAGCAGTCTTCCAGGCGAAAAAGAGGGTT
CTTGAACCTCTGGGCCTGGTTGAGGAACCTGTTAAGACGGCTCCGGGAAAAAAGAGGCCGGTAGAGCACTCTCCT
GTGGAGCCAGACTCCTCCTCGGGAACCGGAAAGGCGGGCCAGCAGCCTGCAAGAAAAAGATTGAATTTTGGTCAG
ACTGGAGACGCAGACTCAGTACCTGADDDDDAGCCTCTCGGACAGCCACCAGCAGCCCCCTCTGGTCTGGGAACT
AATACGATGGCTACAGGCAGTGGCGCACCAATGGCAGACAATAACGAGGGCGCCGACGGAGTGGGTAATTCCTCG
GGAAATTGGCATTGCGATTCCACATGGATGGGCGACAGAGTCATCACCACCAGCACCCGAACCTGGGCCCTGCCC
ACCTACAACAACCACCTCTACAAACAAATTTCCAGCCAATCAGGAGCCTCGAACGACAATCACTACTTTGGCTAC
AGCACCCCTTGGGGGTATTTTGACTTCAACAGATTCCACTGCCACTTTTCACCACGTGACTGGCAAAGACTCATC
AACAACAACTGGGGATTCCGACCCAAGAGACTCAACTTCAAGCTCTTTAACATTCAAGTCAAAGAGGTCACGCAG
AATGACGGTACGACGACGATTGCCAATAACCTTACCAGCACGGTTCAGGTGTTTACTGACTCGGAGTACCAGCTC
CCGTACGTCCTCGGCTCGGCGCATCAAGGATGCCTCCCGCCGTTCCCAGCAGACGTCTTCATGGTGCCACAGTAT
GGATACCTCACCCTGAACAACGGGAGTCAGGCAGTAGGACGCTCTTCATTTTACTGCCTGGAGTACTTTCCTTCT
CAGATGCTGCGTACCGGAAACAACTTTACCTTCAGCTACACTTTTGAGGACGTTCCTTTCCACAGCAGCTACGCT
CACAGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTCTAGG
AACTGGCTTCCTGGACCCTGTTACCGCCAGCAGCGAGTATCAAAGACATCTGCGGATAACAACAACAGTGAATAC
TCGTGGACTGGAGCTACCAAGTACCACCTCAATGGCAGAGACTCTCTGGTGAATCCGGGCCCGGCCATGGCAAGC
CACAAGGACGATGAAGAAAAGTTTTTTCCTCAGAGCGGGGTTCTCATCTTTGGGAAGCAAGGCTCAGAGAAAACA
AATGTGGACATTGAAAAGGTCATGATTACAGACGAAGAGGAAATCAGGACAACCAATCCCGTGGCTACGGAGCAG
TATGGTTCTGTATCTACCAACCTCCAGAGAGGCAACAGACAAGCAGCTACCGCAGATGTCAACACACAAGGCGTT
CTTCCAGGCATGGTCTGGCAGGACAGAGATGTGTACCTTCAGGGGCCCATCTGGGCAAAGATTCCACACACGGAC
GGACATTTTCACCCCTCTCCCCTCATGGGTGGATTCGGACTTAAACACCCTCCTCCACAGATTCTCATCAAGAAC
ACCCCGGTACCTGCGAATCCTTCGACCACCTTCAGTGCGGCAAAGTTTGCTTCCTTCATCACACAGTACTCCACG
GGACAGGTCAGCGTGGAGATCGAGTGGGAGCTGCAGAAGGAAAACAGCAAACGCTGGAATCCCGAAATTCAGTAC
ACTTCCAACTACAACAAGTCTGTTAATGTGGACTTTACTGTGGACACTAATGGCGTGTATTCAGAGCCTCGCCCC
ATTGGCACCAGATACCTGACTCGTAATCTGTAA
Cap5 Sequence - contains sequentially VP1, VP2, AAP, VP3 (start codons
underlined)
(SEQ ID NO: 33)
ATGGCTTTTGTTGATCACCCTCCAGATTGGTTGGAAGAAGTTGGTGAAGGTCTTCGCGAGTTTTTGGGCCTTGAA
GCGGGCCCACCGAAACCAAAACCCAATCAGCAGCATCAAGATCAAGCCCGTGGTCTTGTGCTGCCTGGTTATAAC
TATCTCGGACCCGGAAACGGTCTCGATCGAGGAGAGCCTGTCAACAGGGCAGACGAGGTCGCGCGAGAGCACGAC
ATCTCGTACAACGAGCAGCTTGAGGCGGGAGACAACCCCTACCTCAAGTACAACCACGCGGACGCCGAGTTTCAG
GAGAAGCTCGCCGACGACACATCCTTCGGGGGAAACCTCGGAAACGCAGTCTTTCAGGCCAAGAAAAGGGTTCTC
GAACCTTTTGGCCTGGTTGAAGAGGGTGCTAAGACGGCCCCTACCGGAAAGCGGATAGACGACCACTTTCCAAAA
AGAAAGAAGGCTCGGACCGAAGAGGACTCCAAGCCTTCCACCTCGTCAGACGCCGAAGCTGGACCCAGCGGATCC
CAGCAGCTGCAAATCCCAGCCCAACCAGCCTCAAGTTTGGGAGCTGATACAATGTCTGCGGGAGGTGGCGGCCCA
TTGGGCGACAATAACCAAGGTGCCGATGGAGTGGGCAATGCCTCGGGAGATTGGCATTGCGATTCCACGTGGATG
GGGGACAGAGTCGTCACCAAGTCCACCCGAACCTGGGTGCTGCCCAGCTACAACAACCACCAGTACCGAGAGATC
AAAAGCGGCTCCGTCGACGGAAGCAACGCCAACGCCTACTTTGGATACAGCACCCCCTGGGGGTACTTTGACTTT
AACCGCTTCCACAGCCACTGGAGCCCCCGAGACTGGCAAAGACTCATCAACAACTACTGGGGCTTCAGACCCCGG
TCCCTCAGAGTCAAAATCTTCAACATTCAAGTCAAAGAGGTCACGGTGCAGGACTCCACCACCACCATCGCCAAC
AACCTCACCTCCACCGTCCAAGTGTTTACGGACGACGACTACCAGCTGCCCTACGTCGTCGGCAACGGGACCGAG
GGATGCCTGCCGGCCTTCCCTCCGCAGGTCTTTACGCTGCCGCAGTACGGTTACGCGACGCTGAACCGCGACAAC
ACAGAAAATCCCACCGAGAGGAGCAGCTTCTTCTGCCTAGAGTACTTTCCCAGCAAGATGCTGAGAACGGGCAAC
AACTTTGAGTTTACCTACAACTTTGAGGAGGTGCCCTTCCACTCCAGCTTCGCTCCCAGTCAGAACCTCTTCAAG
CTGGCCAACCCGCTGGTGGACCAGTACTTGTACCGCTTCGTGAGCACAAATAACACTGGCGGAGTCCAGTTCAAC
AAGAACCTGGCCGGGAGATACGCCAACACCTACAAAAACTGGTTCCCGGGGCCCATGGGCCGAACCCAGGGCTGG
AACCTGGGCTCCGGGGTCAACCGCGCCAGTGTCAGCGCCTTCGCCACGACCAATAGGATGGAGCTCGAGGGCGCG
AGTTACCAGGTGCCCCCGCAGCCGAACGGCATGACCAACAACCTCCAGGGCAGCAACACCTATGCCCTGGAGAAC
ACTATGATCTTCAACAGCCAGCCGGCGAACCCGGGCACCACCGCCACGTACCTCGAGGGCAACATGCTCATCACC
AGCGAGAGCGAGACGCAGCCGGTGAACCGCGTGGCGTACAACGTCGGCGGGCAGATGGCCACCAACAACCAGAGC
TCCACCACTGCCCCCGCGACCGGCACGTACAACCTCCAGGAAATCGTGCCCGGCAGCGTGTGGATGGAGAGGGAC
GTGTACCTCCAAGGACCCATCTGGGCCAAGATCCCAGAGACGGGGGCGCACTTTCACCCCTCTCCGGCCATGGGC
GGATTCGGACTCAAACACCCACCGCCCATGATGCTCATCAAGAACACGCCTGTGCCCGGAAATATCACCAGCTTC
TCGGACGTGCCCGTCAGCAGCTTCATCACCCAGTACAGCACCGGGCAGGTCACCGTGGAGATGGAGTGGGAGCTC
AAGAAGGAAAACTCCAAGAGGTGGAACCCAGAGATCCAGTACACAAACAACTACAACGACCCCCAGTTTGTGGAC
TTTGCCCCGGACAGCACCGGGGAATACAGAAGCACCAGACCTATCGGAACCCGATACCTTACCCGACCCCTTTAA

### Example 12—Adenovirus Polynucleotide Sequences

Adenovirus (Ad) polynucleotides can be selected from any serotype, and representative polynucleotides are exemplified below.

E2A Full Sequence
(SEQ ID NO: 34)
CGACCGCACCCTGTGACGAAAGCCGCCCGCAAGCTGCGCCCCTGAGTTAGTCATCTGAACTTCGGCCTGGGCGT
CTCTGGGAAGTACCACAGTGGTGGGAGCGGGACTTTCCTGGTACACCAGGGCAGCGGGCCAACTACGGGGATTAA
GGTTATTACGAGGTGTGGTGGTAATAGCCGCCTGTTCGAGGAGAATTCGGTTTCGGTGGGCGCGGATTCCGTTGA
CCCGGGATATCATGTGGGGTCCCGCGCTCATGTAGTTTATTCGGGTTGAGTAGTCTTGGGCAGCTCCAGCCGCAA
GTCCCATTTGTGGCTGGTAACTCCACATGTAGGGCGTGGGAATTTCCTTGCTCATAATGGCGCTGACGACAGGTG
CTGGCGCCGGGTGTGGCCGCTGGAGATGACGTAGTTTTCGCGCTTAAATTTGAGAAAGGGCGCGAAACTAGTCCT
TAAGAGTCAGCGCGCAGTATTTGCTGAAGAGAGCCTCCGCGTCTTCCAGCGTGCGCCGAAGCTGATCTTCGCTTT
TGTGATACAGGCAGCTGCGGGTGAGGGAGCGCAGAGACCTGTTTTTTATTTTCAGCTCTTGTTCTTGGCCCCTGC
TTTGTTGAAATATAGCATACAGAGTGGGAAAAATCCTATTTCTAAGCTCGCGGGTCGATACGGGTTCGTTGGGCG
CCAGACGCAGCGCTCCTCCTCCTGCTGCTGCCGCCGCTGTGGATTTCTTGGGCTTTGTCAGAGTCTTGCTATCCG
GTCGCCTTTGCTTCTGTGTGACCGCTGCTGTTGCTGCCGCTGCCGCTGCCGCCGGTGCAGTAGGGGCTGTAGAGA
TGACGGTAGTAATGCAGGATGTTACGGGGGAAGGCCACGCCGTGATGGTAGAGAAGAAAGCGGCGGGCGAAGGAG
ATGTTGCCCCCACAGTCTTGCAAGCAAGCAACTATGGCGTTCTTGTGCCCGCGCCACGAGCGGTAGCCTTGGCGC
TGTTGTTGCTCTTGGGCTAACGGCGGCGGCTGCTTAGACTTACCGGCCCTGGTTCCAGTGGTGTCCCATCTACGG
TTGGGTCGGCGAACAGGCAGTGCCGGCGGCGCCTGAGGAGCGGAGGTTGTAGCGATGCTGGGAACGGTTGCCAAT
TTCTGGGGCGCCGGCGAGGGGAATGCGACCGAGGGTGACGGTGTTTCGTCTGACACCTCTTCGGCCTCGGAAGCT
TCGTCTAGGCTGTCCCAGTCTTCCATCATCTCCTCCTCCTCGTCCAAAACCTCCTCTGCCTGACTGTCCCAGTAT
TCCTCCTCGTCCGTGGGTGGCGGCGGCGGCAGCTGCAGCTTCTTTTTGGGTGCCATCCTGGGAAGCAAGGGCCCG
CGGCTGCTGATAGGGCTGCGGCGGCGGGGGGATTGGGTTGAGCTCCTCGCCGGACTGGGGGTCCAGGTAAACCCC
CCGTCCCTTTCGTAGCAGAAACTCTTGGCGGGCTTTGTTGATGGCTTGCAATTGGCCAAGGATGTGGCCCTGGGT
AATGACGCAGGCGGTAAGCTCCGCATTTGGCGGGCGGGATTGGTCTTCGTAGAACCTAATCTCGTGGGCGTGGTA
GTCCTCAGGTACAAATTTGCGAAGGTAAGCCGACGTCCACAGCCCCGGAGTGAGTTTCAACCCCGGAGCCGCGGA
CTTTTCGTCAGGCGAGGGACCCTGCAGCTCAAAGGTACCGATAATTTGACTTTCGCTAAGCAGTTGCGAATTGCA
GACCAGGGAGCGGTGCGGGGTGCATAGGTTGCAGCGACAGTGACACTCCAGTAGGCCGTCACCGCTCACGTCTTC
CATGATGTCGGAGTGGTAGGCAAGGTAGTTGGCTAGCTGCAGAAGGTAGCAGTGACCCCAAAGCGGCGGAGGGCA
TTCACGGTACTTAATGGGCACAAAGTCGCTAGGAAGCGCACAGCAGGTGGCGGGCAGAATTCCTGAACGCTCTAG
GATAAAGTTCCTAAAGTTTTGCAACATGCTTTGACTGGTGAAGTCTGGCAGACCCTGTTGCAGGGTTTTAAGCAG
GCGTTCGGGGAAGATAATGTCCGCCAGGTGCGCGGCCACGGAGCGCTCGTTGAAGGCCGTCCATAGGTCCTTCAA
GTTTTGCTTTAGCAGCTTCTGCAGCTCCTTTAGGTTGCGCTCCTCCAGGCATTGCTGCCACACGCCCATGGCCGT
TTGCCAGGTGTAGCACAGAAATAAGTAAACGCAGTCGCGGACGTAGTCGCGGCGCGCCTCGCCCTTGAGCGTGGA
ATGAAGCACGTTTTGCCCGAGGCGGTTTTCGTGCAAAATTCCAAGGTAGGAGACCAGGTTGCAGAGCTCCACGTT
GGAAATTTTGCAGGCCTGGCGCACGTAGCCCTGGCGAAAGGTGTAGTGCAACGTTTCCTCTAGCTTGCGCTGCAT
CTCCGGGTCAGCAAAGAACCGCTGCATGCACTCAAGCTCCACGGTAACAAGCACTGCGGCCATCATTAGCTTGCG
TCGCTCCTCCAAGTCGGCAGGCTCGCGCGTCTCAAGCCAGCGCGCCAGCTGCTCATCGCCAACTGCGGGTAGGCC
CTCCTCGGTTTGTTCTTGCAAGTTTGCATCCCTCTCCAGGGGTCGTGCACGGCGCACGATCAGCTCGCTCATGAC
TGTGCTCATAACCTTGGGGGGTAGGTTAAGTGCCGGGTAGGCAAAGTGGGTGACCTCGATGCTGCGTTTCAGCAC
GGCTAGGCGCGCGTTGTCACCCTCAAGTTCCACCAGCACTCCACAGTGACTTTCATTTTCGCTGTTTTCTTGTTG
CAGAGCGTTTGCCGCGCGTTTCTCGTCGCGTCCAAGACCCTCAAAGATTTTTGGCACTTCGTCGAGCGAGGCGAT
ATCAGGTATGACAGCGCCCTGCCGCAAGGCCAGCTGCTTGTCCGCTCGGCTGCGGTTGGCACGGCAGGATAGGGG
TATCTTGCAGTTTTGGAAAAAGATGTGATAGGTGGCAAGCACCTCTGGCACGGCAAATACGGGGTAGAAGTTGAG
GCGCGGGTTGGGCTCGCATGTGCCGTTTTCTTGGCGTTTGGGGGGTACGCGCGGTGAGAACAGGTGGCGTTCGTA
GGCAAGGCTGACATCCGCTATGGCGAGGGGCACATCGCTGCGCTCTTGCAACGCGTCGCAGATAATGGCGCACTG
GCGCTGCAGATGCTTCAACAGCACGTCGTCTCCCACATCTAGGTAGTCGCCATGCCTTTGGTCCCCCCGCCCGAC
TTGTTCCTCGTTTGCCTCTGCGTCGTCCTGGTCTTGCTTTTTATCCTCTGTTGGTACTGAGCGATCCTCGTCGTC
TTCGCTTACAAAACCTGGGTCCTGCTCGATAATCACTTCCTCCTCCTCAAGCGGGGGTGCCTCGACGGGGAAGGT
GGTAGGCGCGTTGGCGGCATCGGTGGAGGCGGTGGTGGCGAACTCAAAGGGGGCGGTTAGGCTGTCCTCCTTCTC
GACTGACTCCATGATCTTTTTCTGCCTATAGGAGAAGGAAATGGCCAGTCGGGAAGAGGAGCAGCGCGAAACCAC
CCCCGAGCGCGGACGCGGTGCGGCGCGACGTCCACCAACCATGGAGGACGTGTCGTCCCCGTCGCCGTCGCCGCC
GCCTCCCCGCGCGCCCCCAAAAAAGCGGCTGAGGCGGCGTCTCGAGTCCGAGGACGAAGAAGACTCGTCACAAGA
TGCGCTGGTGCCGCGCACACCCAGCCCGCGGCCATCGACCTCGACGGCGGATTTGGCCATTGCGTCCAAAAAGAA
AAAGAAGCGCCCCTCTCCCAAGCCCGAGCGCCCGCCATCCCCAGAGGTGATCGTGGACAGCGAGGAAGAAAGAGA
AGATGTGGCGCTACAAATGGTGGGTTTCAGCAACCCACCGGTGCTAATCAAGCACGGCAAGGGAGGTAAGCGCAC
GGTGCGGCGGCTGAATGAAGACGACCCAGTGGCGCGGGGTATGCGGACGCAAGAGGAAAAGGAAGAGTCCAGTGA
AGCGGAAAGTGAAAGCACGGTGATAAACCCGCTGAGCCTGCCGATCGTGTCTGCGTGGGAGAAGGGCATGGAGGC
TGCGCGCGCGTTGATGGACAAGTACCACGTGGATAACGATCTAAAGGCAAACTTCAAGCTACTGCCTGACCAAGT
GGAAGCTCTGGCGGCCGTATGCAAGACCTGGCTAAACGAGGAGCACCGCGGGTTGCAGCTGACCTTCACCAGCAA
CAAGACCTTTGTGACGATGATGGGGCGATTCCTGCAGGCGTACCTGCAGTCGTTTGCAGAGGTAACCTACAAGCA
CCACGAGCCCACGGGCTGCGCGTTGTGGCTGCACCGCTGCGCTGAGATCGAAGGCGAGCTTAAGTGTCTACACGG
GAGCATTATGATAAATAAGGAGCACGTGATTGAAATGGATGTGACGAGCGAAAACGGGCAGCGCGCGCTGAAGGA
GCAGTCTAGCAAGGCCAAGATCGTGAAGAACCGGTGGGGCCGAAATGTGGTGCAGATCTCCAACACCGACGCAAG
GTGCTGCGTGCATGACGCGGCCTGTCCGGCCAATCAGTTTTCCGGCAAGTCTTGCGGCATGTTCTTCTCTGAAGG
CGCAAAGGCTCAGGTGGCTTTTAAGCAGATCAAGGCTTTCATGCAGGCGCTGTATCCTAACGCCCAGACCGGGCA
CGGTCACCTTCTGATGCCACTACGGTGCGAGTGCAACTCAAAGCCTGGGCATGCACCCTTTTTGGGAAGGCAGCT
ACCAAAGTTGACTCCGTTCGCCCTGAGCAACGCGGAGGACCTGGACGCGGATCTGATCTCCGACAAGAGCGTGCT
GGCCAGCGTGCACCACCCGGCGCTGATAGTGTTCCAGTGCTGCAACCCTGTGTATCGCAACTCGCGCGCGCAGGG
CGGAGGCCCCAACTGCGACTTCAAGATATCGGCGCCCGACCTGCTAAACGCGTTGGTGATGGTGCGCAGCCTGTG
GAGTGAAAACTTCACCGAGCTGCCGCGGATGGTTGTGCCTGAGTTTAAGTGGAGCACTAAACACCAGTATCGCAA
CGTGTCCCTGCCAGTGGCGCATAGCGATGCGCGGCAGAACCCCTTTGATTTTTAAACGGCGCAGACGGCAAGGGT
GGGGGGTAAATAATCACCCGAGAGTGTACAAATAAAAACATTTGCCTTTATTGAAAGTGTCTCCTAGTACATTAT
TTTTACATGTTTTTCAAGTGACAAAAAGAAGTGGCGCTCCTAATCTGCGCACTGTGGCTGCGGAAGTAGGGCGAG
TGGCGCTCCAGGAAGCTGTAGAGCTGTTCCTGGTTGCGACGCAGGGTGGGCTGTACCTGGGGACTGTTAAGCATG
GAGTTGGGTACC
E2A ORF Sequence
(SEQ ID NO: 35)
ATGGCCAGTCGGGAAGAGGAGCAGCGCGAAACCACCCCCGAGCGCGGACGCGGTGCGGCGCGACGTCCACCAACC
ATGGAGGACGTGTCGTCCCCGTCGCCGTCGCCGCCGCCTCCCCGCGCGCCCCCAAAAAAGCGGCTGAGGCGGCGT
CTCGAGTCCGAGGACGAAGAAGACTCGTCACAAGATGCGCTGGTGCCGCGCACACCCAGCCCGCGGCCATCGACC
TCGACGGCGGATTTGGCCATTGCGTCCAAAAAGAAAAAGAAGCGCCCCTCTCCCAAGCCCGAGCGCCCGCCATCC
CCAGAGGTGATCGTGGACAGCGAGGAAGAAAGAGAAGATGTGGCGCTACAAATGGTGGGTTTCAGCAACCCACCG
GTGCTAATCAAGCACGGCAAGGGAGGTAAGCGCACGGTGCGGCGGCTGAATGAAGACGACCCAGTGGCGCGGGGT
ATGCGGACGCAAGAGGAAAAGGAAGAGTCCAGTGAAGCGGAAAGTGAAAGCACGGTGATAAACCCGCTGAGCCTG
CCGATCGTGTCTGCGTGGGAGAAGGGCATGGAGGCTGCGCGCGCGTTGATGGACAAGTACCACGTGGATAACGAT
CTAAAGGCAAACTTCAAGCTACTGCCTGACCAAGTGGAAGCTCTGGCGGCCGTATGCAAGACCTGGCTAAACGAG
GAGCACCGCGGGTTGCAGCTGACCTTCACCAGCAACAAGACCTTTGTGACGATGATGGGGCGATTCCTGCAGGCG
TACCTGCAGTCGTTTGCAGAGGTAACCTACAAGCACCACGAGCCCACGGGCTGCGCGTTGTGGCTGCACCGCTGC
GCTGAGATCGAAGGCGAGCTTAAGTGTCTACACGGGAGCATTATGATAAATAAGGAGCACGTGATTGAAATGGAT
GTGACGAGCGAAAACGGGCAGCGCGCGCTGAAGGAGCAGTCTAGCAAGGCCAAGATCGTGAAGAACCGGTGGGGC
CGAAATGTGGTGCAGATCTCCAACACCGACGCAAGGTGCTGCGTGCATGACGCGGCCTGTCCGGCCAATCAGTTT
TCCGGCAAGTCTTGCGGCATGTTCTTCTCTGAAGGCGCAAAGGCTCAGGTGGCTTTTAAGCAGATCAAGGCTTTC
ATGCAGGCGCTGTATCCTAACGCCCAGACCGGGCACGGTCACCTTCTGATGCCACTACGGTGCGAGTGCAACTCA
AAGCCTGGGCATGCACCCTTTTTGGGAAGGCAGCTACCAAAGTTGACTCCGTTCGCCCTGAGCAACGCGGAGGAC
CTGGACGCGGATCTGATCTCCGACAAGAGCGTGCTGGCCAGCGTGCACCACCCGGCGCTGATAGTGTTCCAGTGC
TGCAACCCTGTGTATCGCAACTCGCGCGCGCAGGGCGGAGGCCCCAACTGCGACTTCAAGATATCGGCGCCCGAC
CTGCTAAACGCGTTGGTGATGGTGCGCAGCCTGTGGAGTGAAAACTTCACCGAGCTGCCGCGGATGGTTGTGCCT
GAGTTTAAGTGGAGCACTAAACACCAGTATCGCAACGTGTCCCTGCCAGTGGCGCATAGCGATGCGCGGCAGAAC
CCCTTTGATTTTTAA
E4 Full Sequence
(SEQ ID NO: 36)
CCCGGGCGTTTTAGGGCGGAGTAACTTGCATGTATTGGGAATTGTAGTTTTTTTAAAATGGGAAGTGACGTATCG
TGGGAAAACGGAAGTGAAGATTTGAGGAAGTTGTGGGTTTTTTGGCTTTCGTTTCTGGGCGTAGGTTCGCGTGCG
GTTTTCTGGGTGTTTTTTGTGGACTTTAACCGTTACGTCATTTTTTAGTCCTATATATACTCGCTCTGTACTTGG
CCCTTTTTACACTGTGACTGATTGAGCTGGTGCCGTGTCGAGTGGTGTTTTTTAATAGGTTTTTTTACTGGTAAG
GCTGACTGTTATGGCTGCCGCTGTGGAAGCGCTGTATGTTGTTCTGGAGCGGGAGGGTGCTATTTTGCCTAGGCA
GGAGGGTTTTTCAGGTGTTTATGTGTTTTTCTCTCCTATTAATTTTGTTATACCTCCTATGGGGGCTGTAATGTT
GTCTCTACGCCTGCGGGTATGTATTCCCCCGGGCTATTTCGGTCGCTTTTTAGCACTGACCGATGTTAACCAACC
TGATGTGTTTACCGAGTCTTACATTATGACTCCGGACATGACCGAGGAACTGTCGGTGGTGCTTTTTAATCACGG
TGACCAGTTTTTTTACGGTCACGCCGGCATGGCCGTAGTCCGTCTTATGCTTATAAGGGTTGTTTTTCCTGTTGT
AAGACAGGCTTCTAATGTTTAAATGTTTTTTTTTTTGTTATTTTATTTTGTGTTTAATGCAGGAACCCGCAGACA
TGTTTGAGAGAAAAATGGTGTCTTTTTCTGTGGTGGTTCCGGAACTTACCTGCCTTTATCTGCATGAGCATGACT
ACGATGTGCTTGCTTTTTTGCGCGAGGCTTTGCCTGATTTTTTGAGCAGCACCTTGCATTTTATATCGCCGCCCA
TGCAACAAGCTTACATAGGGGCTACGCTGGTTAGCATAGCTCCGAGTATGCGTGTCATAATCAGTGTGGGTTCTT
TTGTCATGGTTCCTGGCGGGGAAGTGGCCGCGCTGGTCCGTGCAGACCTGCACGATTATGTTCAGCTGGCCCTGC
GAAGGGACCTACGGGATCGCGGTATTTTTGTTAATGTTCCGCTTTTGAATCTTATACAGGTCTGTGAGGAACCTG
AATTTTTGCAATCATGATTCGCTGCTTGAGGCTGAAGGTGGAGGGCGCTCTGGAGCAGATTTTTACAATGGCCGG
ACTTAATATTCGGGATTTGCTTAGAGACATATTGATAAGGTGGCGAGATGAAAATTATTTGGGCATGGTTGAAGG
TGCTGGAATGTTTATAGAGGAGATTCACCCTGAAGGGTTTAGCCTTTACGTCCACTTGGACGTGAGGGCAGTTTG
CCTTTTGGAAGCCATTGTGCAACATCTTACAAATGCCATTATCTGTTCTTTGGCTGTAGAGTTTGACCACGCCAC
CGGAGGGGAGCGCGTTCACTTAATAGATCTTCATTTTGAGGTTTTGGATAATCTTTTGGAATAAAAAAAAAAAAA
CATGGTTCTTCCAGCTCTTCCCGCTCCTCCCGTGTGTGACTCGCAGAACGAATGTGTAGGTTGGCTGGGTGTGGC
TTATTCTGCGGTGGTGGATGTTATCAGGGCAGCGGCGCATGAAGGAGTTTACATAGAACCCGAAGCCAGGGGGCG
CCTGGATGCTTTGAGAGAGTGGATATACTACAACTACTACACAGAGCGAGCTAAGCGACGAGACCGGAGACGCAG
ATCTGTTTGTCACGCCCGCACCTGGTTTTGCTTCAGGAAATATGACTACGTCCGGCGTTCCATTTGGCATGACAC
TACGACCAACACGATCTCGGTTGTCTCGGCGCACTCCGTACAGTAGGGATCGCCTACCTCCTTTTGAGACAGAGA
CCCGCGCTACCATACTGGAGGATCATCCGCTGCTGCCCGAATGTAACACTTTGACAATGCACAACGTGAGTTACG
TGCGAGGTCTTCCCTGCAGTGTGGGATTTACGCTGATTCAGGAATGGGTTGTTCCCTGGGATATGGTTCTGACGC
GGGAGGAGCTTGTAATCCTGAGGAAGTGTATGCACGTGTGCCTGTGTTGTGCCAACATTGATATCATGACGAGCA
TGATGATCCATGGTTACGAGTCCTGGGCTCTCCACTGTCATTGTTCCAGTCCCGGTTCCCTGCAGTGCATAGCCG
GCGGGCAGGTTTTGGCCAGCTGGTTTAGGATGGTGGTGGATGGCGCCATGTTTAATCAGAGGTTTATATGGTACC
GGGAGGTGGTGAATTACAACATGCCAAAAGAGGTAATGTTTATGTCCAGCGTGTTTATGAGGGGTCGCCACTTAA
TCTACCTGCGCTTGTGGTATGATGGCCACGTGGGTTCTGTGGTCCCCGCCATGAGCTTTGGATACAGCGCCTTGC
ACTGTGGGATTTTGAACAATATTGTGGTGCTGTGCTGCAGTTACTGTGCTGATTTAAGTGAGATCAGGGTGCGCT
GCTGTGCCCGGAGGACAAGGCGTCTCATGCTGCGGGCGGTGCGAATCATCGCTGAGGAGACCACTGCCATGTTGT
ATTCCTGCAGGACGGAGCGGCGGCGGCAGCAGTTTATTCGCGCGCTGCTGCAGCACCACCGCCCTATCCTGATGC
ACGATTATGACTCTACCCCCATGTAGGCGTGGACTTCCCCTTCGCCGCCCGTTGAGCAACCGCAAGTTGGACAGC
AGCCTGTGGCTCAGCAGCTGGACAGCGACATGAACTTAAGCGAGCTGCCCGGGGAGTTTATTAATATCACTGATG
AGCGTTTGGCTCGACAGGAAACCGTGTGGAATATAACACCTAAGAATATGTCTGTTACCCATGATATGATGCTTT
TTAAGGCCAGCCGGGGAGAAAGGACTGTGTACTCTGTGTGTTGGGAGGGAGGTGGCAGGTTGAATACTAGGGTTC
TGTGAGTTTGATTAAGGTACGGTGATCAATATAAGCTATGTGGTGGTGGGGCTATACTACTGAATGAAAAATGAC
TTGAAATTTTCTGCAATTGAAAAATAAACACGTTGAAACATAACATGCAACAGGTTCACGATTCTTTATTCCTGG
GCAATGTAGGAGAAGGTGTAAGAGTTGGTAGCAAAAGTTTCAGTGGTGTATTTTCCACTTTCCCAGGACCATGTA
AAAGACATAGAGTAAGTGCTTACCTCGCTAGTTTCTGTGGATTCACTAGAA
E4 Orf6 Sequence
(SEQ ID NO: 37)
ATGACTACGTCCGGCGTTCCATTTGGCATGACACTACGACCAACACGATCTCGGTTGTCTCGGCGCACTCCGTAC
AGTAGGGATCGCCTACCTCCTTTTGAGACAGAGACCCGCGCTACCATACTGGAGGATCATCCGCTGCTGCCCGAA
TGTAACACTTTGACAATGCACAACGTGAGTTACGTGCGAGGTCTTCCCTGCAGTGTGGGATTTACGCTGATTCAG
GAATGGGTTGTTCCCTGGGATATGGTTCTGACGCGGGAGGAGCTTGTAATCCTGAGGAAGTGTATGCACGTGTGC
CTGTGTTGTGCCAACATTGATATCATGACGAGCATGATGATCCATGGTTACGAGTCCTGGGCTCTCCACTGTCAT
TGTTCCAGTCCCGGTTCCCTGCAGTGCATAGCCGGCGGGCAGGTTTTGGCCAGCTGGTTTAGGATGGTGGTGGAT
GGCGCCATGTTTAATCAGAGGTTTATATGGTACCGGGAGGTGGTGAATTACAACATGCCAAAAGAGGTAATGTTT
ATGTCCAGCGTGTTTATGAGGGGTCGCCACTTAATCTACCTGCGCTTGTGGTATGATGGCCACGTGGGTTCTGTG
GTCCCCGCCATGAGCTTTGGATACAGCGCCTTGCACTGTGGGATTTTGAACAATATTGTGGTGCTGTGCTGCAGT
TACTGTGCTGATTTAAGTGAGATCAGGGTGCGCTGCTGTGCCCGGAGGACAAGGCGTCTCATGCTGCGGGCGGTG
CGAATCATCGCTGAGGAGACCACTGCCATGTTGTATTCCTGCAGGACGGAGCGGCGGCGGCAGCAGTTTATTCGC
GCGCTGCTGCAGCACCACCGCCCTATCCTGATGCACGATTATGACTCTACCCCCATGTAG
VA Sequence (VA transcripts I and II are underlined)
(SEQ ID NO: 38)
CGTAATCCGTAGATGTACCTGGACATCCAGGTGATGCCGGCGGCGGTGGTGGAGGCGCGCGGAAAGTCGCGGAC
GCGGTTCCAGATGTTGCGCAGCGGCAAAAAGTGCTCCATGGTCGGGACGCTCTGGCCGGTGAGGCGTGCGCAGTC
GTTGACGCTCTAGACCGTGCAAAAGGAGAGCCTGTAAGCGGGCACTCTTCCGTGGTCTGGTGGATAAATTCGCAA
GGGTATCATGGCGGACGACCGGGGTTCGAACCCCGGATCCGGCCGTCCGCCGTGATCCATGCGGTTACCGCCCGC
GTGTCGAACCCAGGTGTGCGACGTCAGACAACGGGGGAGCGCTCCTTTTGGCTTCCTTCCAGGCGCGGCGGCTGC
TGCGCTAGCTTTTTTGGCCACTGGCCGCGCGCGGCGTAAGCGGTTAGGCTGGAAAGCGAAAGCATTAAGTGGCTC
GCTCCCTGTAGCCGGAGGGTTATTTTCCAAGGGTTGAGTCGCAGGACCCCCGGTTCGAGTCTCGGGCCGGCCGGA
CTGCGGCGAACGGGGGTTTGCCTCCCCGTCATGCAAGACCCCGCTTGCAAATTCCTCCGGAAACAGGGACGAGCC
CCTTTTTTGCTTTTCCCAGATGCATCCGGTGCTGCGGCAGATGCGCCCCCCTCCTCAGCAGCGGCAAGAGCAAGA
GCAGCGGCAGACATGCAGGGCACCCTCCCCTTCTCCTACCGCGTCAGGAGGGGCAACATCCTACATCGA

Sequences for E1A and E1B are both contained within Accession AY339865.1

### Ad5 E1A

Two proteins can be transcribed, a 32 kDa protein (first accession number) and a 27 kDa protein (second accession number). These are both splice variants from the transcript:

Accession 1: AAQ19284.1
Accession 2: AAQ19285.1
(SEQ ID NO: 39)
ATGAGACATATTATCTGCCACGGAGGTGTTATTACCGAAGAAATGGCCGCCAGTCTTTTGGACCAGCTGATCGAA
GAGGTACTGGCTGATAATCTTCCACCTCCTAGCCATTTTGAACCACCTACCCTTCACGAACTGTATGATTTAGAC
GTGACGGCCCCCGAAGATCCCAACGAGGAGGCGGTTTCGCAGATTTTTCCCGACTCTGTAATGTTGGCGGTGCAG
GAAGGGATTGACTTACTCACTTTTCCGCCGGCGCCCGGTTCTCCGGAGCCGCCTCACCTTTCCCGGCAGCCCGAG
CAGCCGGAGCAGAGAGCCTTGGGTCCGGTTTCTATGCCAAACCTTGTACCGGAGGTGATCGATCTTACCTGCCAC
GAGGCTGGCTTTCCACCCAGTGACGACGAGGATGAAGAGGGTGAGGAGTTTGTGTTAGATTATGTGGAGCACCCC
GGGCACGGTTGCAGGTCTTGTCATTATCACCGGAGGAATACGGGGGACCCAGATATTATGTGTTCGCTTTGCTAT
ATGAGGACCTGTGGCATGTTTGTCTACAGTCCTGTGTCTGAACCTGAGCCTGAGCCCGAGCCAGAACCGGAGCCT
GCAAGACCTACCCGCCGTCCTAAAATGGCGCCTGCTATCCTGAGACGCCCGACATCACCTGTGTCTAGAGAATGC
AATAGTAGTACGGATAGCTGTGACTCCGGTCCTTCTAACACACCTCCTGAGATACACCCGGTGGTCCCGCTGTGC
CCCATTAAACCAGTTGCCGTGAGAGTTGGTGGGCGTCGCCAGGCTGTGGAATGTATCGAGGACTTGCTTAACGAG
CCTGGGCAACCTTTGGACTTGAGCTGTAAACGCCCCAGGCCATAA
(SEQ ID NO: 40)
ATGAGACATATTATCTGCCACGGAGGTGTTATTACCGAAGAAATGGCCGCCAGTCTTTTGGACCAGCTGATCGAA
GAGGTACTGGCTGATAATCTTCCACCTCCTAGCCATTTTGAACCACCTACCCTTCACGAACTGTATGATTTAGAC
GTGACGGCCCCCGAAGATCCCAACGAGGAGGCGGTTTCGCAGATTTTTCCCGACTCTGTAATGTTGGCGGTGCAG
GAAGGGATTGACTTACTCACTTTTCCGCCGGCGCCCGGTTCTCCGGAGCCGCCTCACCTTTCCCGGCAGCCCGAG
CAGCCGGAGCAGAGAGCCTTGGGTCCGGTTTCTATGCCAAACCTTGTACCGGAGGTGATCGATCTTACCTGCCAC
GAGGCTGGCTTTCCACCCAGTGACGACGAGGATGAAGAGGGTCCTGTGTCTGAACCTGAGCCTGAGCCCGAGCCA
GAACCGGAGCCTGCAAGACCTACCCGCCGTCCTAAAATGGCGCCTGCTATCCTGAGACGCCCGACATCACCTGTG
TCTAGAGAATGCAATAGTAGTACGGATAGCTGTGACTCCGGTCCTTCTAACACACCTCCTGAGATACACCCGGTG
GTCCCGCTGTGCCCCATTAAACCAGTTGCCGTGAGAGTTGGTGGGCGTCGCCAGGCTGTGGAATGTATCGAGGAC
TTGCTTAACGAGCCTGGGCAACCTTTGGACTTGAGCTGTAAACGCCCCAGGCCATAA

### Ad5 E1B_19K

Accession: AAQ19286.1
(SEQ ID NO: 41)
ATGGAGGCTTGGGAGTGTTTGGAAGATTTTTCTGCTGTGCGTAACTTGCTGGAACAGAGCTCTAACAGTACCTCT
TGGTTTTGGAGGTTTCTGTGGGGCTCATCCCAGGCAAAGTTAGTCTGCAGAATTAAGGAGGATTACAAGTGGGAA
TTTGAAGAGCTTTTGAAATCCTGTGGTGAGCTGTTTGATTCTTTGAATCTGGGTCACCAGGCGCTTTTCCAAGAG
AAGGTCATCAAGACTTTGGATTTTTCCACACCGGGGCGCGCTGCGGCTGCTGTTGCTTTTTTGAGTTTTATAAAG
GATAAATGGAGCGAAGAAACCCATCTGAGCGGGGGGTACCTGCTGGATTTTCTGGCCATGCATCTGTGGAGAGCG
GTTGTGAGACACAAGAATCGCCTGCTACTGTTGTCTTCCGTCCGCCCGGCGATAATACCGACGGAGGAGCAGCAG
CAGCAGCAGGAGGAAGCCAGGCGGCGGCGGCAGGAGCAGAGCCCATGGAACCCGAGAGCCGGCCTGGACCCTCGG
GAATGA

### Ad5 E1B_55K

Accession: AAQ19287.1
(SEQ ID NO: 42)
ATGGAGCGAAGAAACCCATCTGAGCGGGGGGTACCTGCTGGATTTTCTGGCCATGCATCTGTGGAGAGCGGTTGT
GAGACACAAGAATCGCCTGCTACTGTTGTCTTCCGTCCGCCCGGCGATAATACCGACGGAGGAGCAGCAGCAGCA
GCAGGAGGAAGCCAGGCGGCGGCGGCAGGAGCAGAGCCCATGGAACCCGAGAGCCGGCCTGGACCCTCGGGAATG
AATGTTGTACAGGTGGCTGAACTGTATCCAGAACTGAGACGCATTTTGACAATTACAGAGGATGGGCAGGGGCTA
AAGGGGGTAAAGAGGGAGCGGGGGGCTTGTGAGGCTACAGAGGAGGCTAGGAATCTAGCTTTTAGCTTAATGACC
AGACACCGTCCTGAGTGTATTACTTTTCAACAGATCAAGGATAATTGCGCTAATGAGCTTGATCTGCTGGCGCAG
AAGTATTCCATAGAGCAGCTGACCACTTACTGGCTGCAGCCAGGGGATGATTTTGAGGAGGCTATTAGGGTATAT
GCAAAGGTGGCACTTAGGCCAGATTGCAAGTACAAGATCAGCAAACTTGTAAATATCAGGAATTGTTGCTACATT
TCTGGGAACGGGGCCGAGGTGGAGATAGATACGGAGGATAGGGTGGCCTTTAGATGTAGCATGATAAATATGTGG
CCGGGGGTGCTTGGCATGGACGGGGTGGTTATTATGAATGTAAGGTTTACTGGCCCCAATTTTAGCGGTACGGTT
TTCCTGGCCAATACCAACCTTATCCTACACGGTGTAAGCTTCTATGGGTTTAACAATACCTGTGTGGAAGCCTGG
ACCGATGTAAGGGTTCGGGGCTGTGCCTTTTACTGCTGCTGGAAGGGGGTGGTGTGTCGCCCCAAAAGCAGGGCT
TCAATTAAGAAATGCCTCTTTGAAAGGTGTACCTTGGGTATCCTGTCTGAGGGTAACTCCAGGGTGCGCCACAAT
GTGGCCTCCGACTGTGGTTGCTTCATGCTAGTGAAAAGCGTGGCTGTGATTAAGCATAACATGGTATGTGGCAAC
TGCGAGGACAGGGCCTCTCAGATGCTGACCTGCTCGGACGGCAACTGTCACCTGCTGAAGACCATTCACGTAGCC
AGCCACTCTCGCAAGGCCTGGCCAGTGTTTGAGCATAACATACTGACCCGCTGTTCCTTGCATTTGGGTAACAGG
AGGGGGGTGTTCCTACCTTACCAATGCAATTTGAGTCACACTAAGATATTGCTTGAGCCCGAGAGCATGTCCAAG
GTGAACCTGAACGGGGTGTTTGACATGACCATGAAGATCTGGAAGGTGCTGAGGTACGATGAGACCCGCACCAGG
TGCAGACCCTGCGAGTGTGGCGGTAAACATATTAGGAACCAGCCTGTGATGCTGGATGTGACCGAGGAGCTGAGG
CCCGATCACTTGGTGCTGGCCTGCACCCGCGCTGAGTTTGGCTCTAGCGATGAAGATACAGATTGA

Sequences for E2A and E4A are both contained within Accession MN088492

### Ad5 E2A orf:

Accession: QHX41645.1
(SEQ ID NO: 43)
ATGGCCAGTCGGGAAGAGGAGCAGCGCGAAACCACCCCCGAGCGCGGACGCGGTGCGGCGCGACGTCCACCAACC
ATGGAGGACGTGTCGTCCCCGTCGCCGTCGCCGCCGCCTCCCCGCGCGCCCCCAAAAAAGCGGCTGAGGCGGCGT
CTCGAGTCCGAGGACGAAGAAGACTCGTCACAAGATGCGCTGGTGCCGCGCACACCCAGCCCGCGGCCATCGACC
TCGACGGCGGATTTGGCCATTGCGTCCAAAAAGAAAAAGAAGCGCCCCTCTCCCAAGCCCGAGCGCCCGCCATCC
CCAGAGGTGATCGTGGACAGCGAGGAAGAAAGAGAAGATGTGGCGCTACAAATGGTGGGTTTCAGCAACCCACCG
GTGCTAATCAAGCACGGCAAGGGAGGTAAGCGCACGGTGCGGCGGCTGAATGAAGACGACCCAGTGGCGCGGGGT
ATGCGGACGCAAGAGGAAAAGGAAGAGTCCAGTGAAGCGGAAAGTGAAAGCACGGTGATAAACCCGCTGAGCCTG
CCGATCGTGTCTGCGTGGGAGAAGGGCATGGAGGCTGCGCGCGCGTTGATGGACAAGTACCACGTGGATAACGAT
CTAAAGGCAAACTTCAAGCTACTGCCTGACCAAGTGGAAGCTCTGGCGGCCGTATGCAAGACCTGGCTAAACGAG
GAGCACCGCGGGTTGCAGCTGACCTTCACCAGCAACAAGACCTTTGTGACGATGATGGGGCGATTCCTGCAGGCG
TACCTGCAGTCGTTTGCAGAGGTAACCTACAAGCACCACGAGCCCACGGGCTGCGCGTTGTGGCTGCACCGCTGC
GCTGAGATCGAAGGCGAGCTTAAGTGTCTACACGGGAGCATTATGATAAATAAGGAGCACGTGATTGAAATGGAT
GTGACGAGCGAAAACGGGCAGCGCGCGCTGAAGGAGCAGTCTAGCAAGGCCAAGATCGTGAAGAACCGGTGGGGC
CGAAATGTGGTGCAGATCTCCAACACCGACGCAAGGTGCTGCGTGCATGACGCGGCCTGTCCGGCCAATCAGTTT
TCCGGCAAGTCTTGCGGCATGTTCTTCTCTGAAGGCGCAAAGGCTCAGGTGGCTTTTAAGCAGATCAAGGCTTTC
ATGCAGGCGCTGTATCCTAACGCCCAGACCGGGCACGGTCACCTTCTGATGCCACTACGGTGCGAGTGCAACTCA
AAGCCTGGGCATGCACCCTTTTTGGGAAGGCAGCTACCAAAGTTGACTCCGTTCGCCCTGAGCAACGCGGAGGAC
CTGGACGCGGATCTGATCTCCGACAAGAGCGTGCTGGCCAGCGTGCACCACCCGGCGCTGATAGTGTTCCAGTGC
TGCAACCCTGTGTATCGCAACTCGCGCGCGCAGGGCGGAGGCCCCAACTGCGACTTCAAGATATCGGCGCCCGAC
CTGCTAAACGCGTTGGTGATGGTGCGCAGCCTGTGGAGTGAAAACTTCACCGAGCTGCCGCGGATGGTTGTGCCT
GAGTTTAAGTGGAGCACTAAACACCAGTATCGCAACGTGTCCCTGCCAGTGGCGCATAGCGATGCGCGGCAGAAC
CCCTTTGATTTTTAA

### Ad5 E4A:

Two proteins are present in this ORF. The first is a splice variant contained within the ORF. The second is a non-spliced transcript present in the ORF. Accession 1: QHX41659.1

### Accession 2: QHX41660.1

(SEQ ID NO: 44)
ATGACTACGTCCGGCGTTCCATTTGGCATGACACTACGACCAACACGATCTCGGTTGTCTCGGCGCACTCCGTAC
AGTAGGGATCGCCTACCTCCTTTTGAGACAGAGACCCGCGCTACCATACTGGAGGATCATCCGCTGCTGCCCGAA
TGTAACACTTTGACAATGCACAACGCGTGGACTTCCCCTTCGCCGCCCGTTGAGCAACCGCAAGTTGGACAGCAG
CCTGTGGCTCAGCAGCTGGACAGCGACATGAACTTAAGCGAGCTGCCCGGGGAGTTTATTAATATCACTGATGAG
CGTTTGGCTCGACAGGAAACCGTGTGGAATATAACACCTAAGAATATGTCTGTTACCCATGATATGATGCTTTTT
AAGGCCAGCCGGGGAGAAAGGACTGTGTACTCTGTGTGTTGGGAGGGAGGTGGCAGGTTGAATACTAGGGTTCTG
TGA
(SEQ ID NO: 45)
ATGACTACGTCCGGCGTTCCATTTGGCATGACACTACGACCAACACGATCTCGGTTGTCTCGGCGCACTCCGTAC
AGTAGGGATCGCCTACCTCCTTTTGAGACAGAGACCCGCGCTACCATACTGGAGGATCATCCGCTGCTGCCCGAA
TGTAACACTTTGACAATGCACAACGTGAGTTACGTGCGAGGTCTTCCCTGCAGTGTGGGATTTACGCTGATTCAG
GAATGGGTTGTTCCCTGGGATATGGTTCTGACGCGGGAGGAGCTTGTAATCCTGAGGAAGTGTATGCACGTGTGC
CTGTGTTGTGCCAACATTGATATCATGACGAGCATGATGATCCATGGTTACGAGTCCTGGGCTCTCCACTGTCAT
TGTTCCAGTCCCGGTTCCCTGCAGTGCATAGCCGGCGGGCAGGTTTTGGCCAGCTGGTTTAGGATGGTGGTGGAT
GGCGCCATGTTTAATCAGAGGTTTATATGGTACCGGGAGGTGGTGAATTACAACATGCCAAAAGAGGTAATGTTT
ATGTCCAGCGTGTTTATGAGGGGTCGCCACTTAATCTACCTGCGCTTGTGGTATGATGGCCACGTGGGTTCTGTG
GTCCCCGCCATGAGCTTTGGATACAGCGCCTTGCACTGTGGGATTTTGAACAATATTGTGGTGCTGTGCTGCAGT
TACTGTGCTGATTTAAGTGAGATCAGGGTGCGCTGCTGTGCCCGGAGGACAAGGCGTCTCATGCTGCGGGCGGTG
CGAATCATCGCTGAGGAGACCACTGCCATGTTGTATTCCTGCAGGACGGAGCGGCGGCGGCAGCAGTTTATTCGC
GCGCTGCTGCAGCACCACCGCCCTATCCTGATGCACGATTATGACTCTACCCCCATGTAG

### Ad5 VA:

Accession: AF369965.1
(SEQ ID NO: 46)
TCGATGTAGGATGTTGCCCCTCCTGACGCGGTAGGAGAAGGGGAGGGTGCCCTGCATGTCTGCCGCTGCTCTTGC
TCTTGCCGCTGCTGAGGAGGGGGGCGCATCTGCCGCAGCACCGGATGCATCTGGGAAAAGCAAAAAAGGGGCTCG
TCCCTGTTTCCGGAGGAATTTGCAAGCGGGGTCTTGCATGACGGGGAGGCAAACCCCCGTTCGCCGCAGTCCGGC
CGGCCCGAGACTCGAACCGGGGGTCCTGCGACTCAACCCTTGGAAAATAACCCTCCGGCTACAGGGAGCGAGCCA
CTTAATGCTTTCGCTTTCCAGCCTAACCGCTTACGCCGCGCGCGGCCAGTGGCCAAAAAAGCTAGCGCAGCAGCC
GCCGCGCCTGGAAGGAAGCCAAAAGGAGCGCTCCCCCGTTGTCTGACGTCGCACACCTGGGTTCGACACGCGGGC
GGTAACCGCATGGATCACGGCGGACGGCCGGATCCGGGGTTCGAACCCCGGTCGTCCGCCATGATACCCTTGCGA
ATTTATCCACCAGACCACGGAAGAGTGCCCGCTTACAGGCTCTCCTTTTGCACGGTCTAGAGCGTCAACGACTGC
GCACGCCTCACCGGCCAGAGCGTCCCGACCATGGAGCACTTTTTGCCGCTGCGCAACATCTGGAACCGCGTCCGC
GACTTTCCGCGCGCCTCCACCACCGCCGCCGGCATCACCTGGATGTCCAGGTACATCTACGGATTACG

### Example 13—Promoter, Operator, IRES and Intron Sequences

CMV Promoter
(SEQ ID NO: 47)
TAGTTATTAATAGTAATCAATTACGGGGTCATTAGTTCATAGCCCATATATGGAGTTCCGCGTTACATAACTTAC
GGTAAATGGCCCGCCTGGCTGACCGCCCAACGACCCCCGCCCATTGACGTCAATAATGACGTATGTTCCCATAGT
AACGCCAATAGGGACTTTCCATTGACGTCAATGGGTGGAGTATTTACGGTAAACTGCCCACTTGGCAGTACATCA
AGTGTATCATATGCCAAGTACGCCCCCTATTGACGTCAATGACGGTAAATGGCCCGCCTGGCATTATGCCCAGTA
CATGACCTTATGGGACTTTCCTACTTGGCAGTACATCTACGTATTAGTCATCGCTATTACCATGGTGATGCGGTT
TTGGCAGTACATCAATGGGCGTGGATAGCGGTTTGACTCACGGGGATTTCCAAGTCTCCACCCCATTGACGTCAA
TGGGAGTTTGTTTTGGCACCAAAATCAACGGGACTTTCCAAAATGTCGTAACAACTCCGCCCCATTGACGCAAAT
GGGCGGTAGGCGTGTACGGTGGGAGGTCTATATAAGCAGAGCTCGTCGACGTTTAGTGAACCG
2xTet Operator Sequence
(SEQ ID NO: 48)
TCCCTATCAGTGATAGAGATCTCCCTATCAGTGATAGAGA
hCMV Intron Sequence
(SEQ ID NO: 49)
GTAAGTACCGCCTATAGAGTCTATAGGCCCACCCCCTTGGCTTCTTATGCATGCTATACTGTTTTTGGCTTGGGG
TCTATACACCCCCGCTTCCTCATGTTATAGGTGATGGTATAGCTTAGCCTATAGGTGTGGGTTATTGACCATTAT
TGACCACTCCCCTATTGGTGACGATACTTTCCATTACTAATCCATAACATGGCTCTTTGCCACAACTCTCTTTAT
TGGCTATATGCCAATACACTGTCCTTCAGAGACTGACACGGACTCTGTATTTTTACAGGATGGGGTCTCATTTAT
TATTTACAAATTCACATATACAACACCACCGTCCCCAGTGCCCGCAGTTTTTATTAAACATAACGTGGGATCTCC
ACGCGAATCTCGGGTACGTGTTCCGGACATGGTCTCTTCTCCGGTAGCGGCGGAGCTTCTACATCCGAGCCCTGC
TCCCATGCCTCCAGCGACTCATGGTCGCTCGGCAGCTCCTTGCTCCTAACAGTGGAGGCCAGACTTAGGCACAGC
ACGATGCCCACCACCACCAGTGTGCCGCACAAGGCCGTGGCGGTAGGGTATGTGTCTGAAAATGAGCTCGGGGAG
CGGGCTTGCACCGCTGACGCATTTGGAAGACTTAAGGCAGCGGCAGAAGAAGATGCAGGCAGCTGAGTTGTTGTG
TTCTGATAAGAGTCAGAGGTAACTCCCGTTGCGGTGCTGTTAACGGTGGAGGGCAGTGTAGTCTGAGCAGTACTC
GTTGCTGCCGCGCGCGCCACCAGACATAATAGCTGACAGACTAACAGACTGTTCCTTTCCATGGGTCTTTTCTGC
AG
ECMV IRES Sequence
(SEQ ID NO: 50)
CCCCCCTCTCCCTCCCCCCCCCCTAACGTTACTGGCCGAAGCCGCTTGGAATAAGGCCGGTGTGCGTTTGTCTAT
ATGTTATTTTCCACCATATTGCCGTCTTTTGGCAATGTGAGGGCCCGGAAACCTGGCCCTGTCTTCTTGACGAGC
ATTCCTAGGGGTCTTTCCCCTCTCGCCAAAGGAATGCAAGGTCTGTTGAATGTCGTGAAGGAAGCAGTTCCTCTG
GAAGCTTCTTGAAGACAAACAACGTCTGTAGCGACCCTTTGCAGGCAGCGGAACCCCCCACCTGGCGACAGGTGC
CTCTGCGGCCAAAAGCCACGTGTATAAGATACACCTGCAAAGGCGGCACAACCCCAGTGCCACGTTGTGAGTTGG
ATAGTTGTGGAAAGAGTCAAATGGCTCTCCTCAAGCGTATTCAACAAGGGGCTGAAGGATGCCCAGAAGGTACCC
CATTGTATGGGATCTGATCTGGGGCCTCGGTGCACATGCTTTACATGTGTTTAGTCGAGGTTAAAAAACGTCTAG
GCCCCCCGAACCACGGGGACGTGGTTTTCCTTTGAAAAACACGATTGCTCGAATCACC
FMDV IRES
(SEQ ID NO: 51)
AGCAGGTTTCCCCAACTGACACAAAACGTGCAACTTGAAACTCCGCCTGGTCTTTCCAGGTCTAGAGGGGTAACA
CTTTGTACTGTGTTTGGCTCCACGCTCGATCCACTGGCGAGTGTTAGTAACAGCACTGTTGCTTCGTAGCGGAGC
ATGACGGCCGTGGGAACTCCTCCTTGGTAACAAGGACCCACGGGGCCAAAAGCCACGCCCACACGGGCCCGTCAT
GTGTGCAACCCCAGCACGGCGACTTTACTGCGAAACCCACTTTAAAGTGACATTGAAACTGGTACCCACACACTG
GTGACAGGCTAAGGATGCCCTTCAGGTACCCCGAGGTAACACGCGACACTCGGGATCTGAGAAGGGGACTGGGGC
TTCTATAAAAGCGCTCGGTTTAAAAAGCTTCTATGCCTGAATAGGTGACCGGAGGTCGGCACCTTTCCTTTACAA
TTAATGACCCT

### Example 14—CHO and Mouse Stable Site 1 Sequences—U.S. Pat. No. 7,771,997

211> 6473

### <212> DNA

<213> Cricetulus griseus
<400> 1

(SEQ ID NO: 52)
tctagaaaca aaaccaaaaa tattaagtca ggcttggctt caggtgctgg ggtggagtgc 60
tgacaaaaat acacaaattc ctggctttct aaggcttttt cggggattca ggtattgggt 120
gatggtagaa taaaaatctg aaacataggt gatgtatctg ccatactgca tgggtgtgta 180
tgtgtgtgta tgtgtgtctg tgtgtgtgcc cagacagaaa taccatgaag gaaaaaaaca 240
cttcaaagac aggagagaag agtgacctgg gaaggactcc ccaatgagat gagaactgag 300
cacatgccag aggaggtgag gactgaacca ttcaacacaa gtggtgaata gtcctgcaga 360
cacagagagg gccagaagca ctcagaactc cagggggtca ggagtggttc tctggaggct 420
tctgcccttg gaggttcctg aggaggaggc ttccatattg aaaatgtagt tagtggccgt 480
ttccattagt acagtgacta gagagagctg agggaccact ggactgaggc ctagatgctc 540
agtcagatgg ccatgaaagc ctagacaagc acttccgggt ggaaaggaaa cagcaggtgt 600
gaggggtcag gggcaagtta gtgggagagg tcttccagat gaagtagcag gaacggagac 660
gcactggatg gccccacttg tcaaccagca aaagcttgga tcttgttcta agaggccagg 720
gacatgacaa gggtgatctc ggtttttaaa aggctttgtg ttacctaatc acttctatta 780
gtcagatact ttgtaacaca aatgagtact tggcctgtat tttagaaact tctgggatcc 840
tgaaaaaaca caatgacatt ctggctgcaa cacctggaga ctcccagcca ggccctggac 900
ccgggtccat tcatgcaaat actcagggac agattcttca ctaggtactg atgagctgtc 960
ttggatgcaa atgtggcctc ttcattttac tacaagtcac catgagtcag gaggtgctgt 1020
ttgcacagtg tgactaagtg atggagtgtt gactgcagcc attcccggcc ccagcttgtg 1080
agagagatcc ttttaaattg aaagtaagct caaagttacc acgaagccac acatgtataa 1140
actgtgtgaa taatctgtgc acatacacaa accatgtgaa taatctgtgt acatgtataa 1200
actgtgtgaa taatctgtgt gcagcctttc cttacctact accttccagt gatcaggttt 1260
ggactgcctg tgtgctactg gaccctgaat gtccccaccg ctgtcccctg tcttttacga 1320
ttctgacatt tttaataaat tcagcggctt cccctctgct ctgtgcctag ctataccttg 1380
gtactctgca ttttggtttc tgtgacattt ctctgtgact ctgctacatt ctcagatgac 1440
atgtgacaca gaaggtgttc cctctggaga catgtgatgt ccctgtcatt agtggaatca 1500
gatgccccca aactgttgtc cagtgtttgg gaaagtgaca cgtgaaggag gatcaggaaa 1560
agaggggtgg aaatcaagat gtgtctgagt atctcatgtc cctgagtggt ccaggctgct 1620
gacttcactc ccccaagtga gggaggccat ggtgagtaca cacacctcac acatactata 1680
tccaacacac acacacacac acacacacac acgcacgcac gcacgcacgc acgcacacat 1740
gcacacacac gaactacatt tcacaaacca catacgcata ttacacccca aacgtatcac 1800
ctatacatac cacacataca cacccctcca cacatcacac acataccaca cccacacaca 1860
gcacacacat acataggcac acattcacac accacacata tacatttgtg tatgcataca 1920
tgcatacaca cacaggcaca cagacaccac acacatgcat tgtgtacgca cacatgcata 1980
cacacacata ggcacacatt gagcacacac atacatttgt gtacgcacac tacatagaca 2040
tatatgcatt tgtatatgca cacatgcatg cacacataca taggcacaca tagagcacac 2100
acatacattt gtgtatgcac acatgcacac accaatcaca tgggaagact caggttcttc 2160
actaaggttc acatgaactt agcagttcct ggttatctcg tgaaacttgg aagattgctg 2220
tggagaagag gaagcgttgg cttgagccct ggcagcaatt aaccccgccc agaagaagta 2280
ggtttaaaaa tgagagggtc tcaatgtgga acccgcaggg cgccagttca gagaagagac 2340
ctacccaagc caactgagag caaaggcaga gggatgaacc tgggatgtag tttgaacctc 2400
tgtaccagct gggcttcatg ctattttgtt atatctttat taaatattct tttagtttta 2460
tgtgcgtgaa taccttgctt gcataaatgt atgggcactg tatgtgttct tggtgccggt 2520
ggaggccagg agagggcatg gatcctccgg agctggcgtt tgagacagtt gtgacccaca 2580
gtgtggggtc tgggaactgg gtcttagtgt tccgcaagtg cagctggggc tcttaacctc 2640
tgagccatcc ctccagcttc aagaaactta ttttcttagg acatggggga agggatccag 2700
ggctttaggc ttgtttgttc agcaaatact cttttcgtgt attttgaatt ttattttatt 2760
ttactttttt gggatagaat cacattctgc agctcaggct gggcctgaac tcatcaaaat 2820
cctcctgtct cagtctacca ggtgataaga ttactgatgt gagcctggct ttgacaagca 2880
ctttagagtc cccagccctt ctggacactt gttccaagta taatatatat atatatatat 2940
atatatatat atatatatat atatattgtg tgtgtgtgtt tgtgtgtgta tgagacactt 3000
gctctaaggg tatcatatat atccttgatt tgcttttaat ttatttttta attaaaaatg 3060
attagctaca tgtcacctgt atgcgtctgt atcatctata tatccttcct tccttctctc 3120
tctttctctc ttcttcttct cacccccaag catctatttt caaatccttg tgccgaggag 3180
atgccaagag tctcgttggg ggagatggtg agggggcgat acaggggaag agcaggagga 3240
aagggggaca gactggtgtg ggtctttgga gagctcagga gaatagcagc gatcttccct 3300
gtccctggtg tcacctctta cagccaacac cattttgtgg cctggcagaa gagttgtcaa 3360
gctggtcgca ggtctgccac acaaccccaa tctggcccca agaaaaggca cctgtgtgtg 3420
actctggggt taaaggcgct gcctggtcgt ctccagctgg acttgaaact cccgtttaat 3480
aaagagttct gcaaaataat acccgcagag tcacagtgcc aggttcccgt gctttcctga 3540
agcgccaggc acgggttccc taggaaatgg ggccttgctt gccaagctcc cacggcttgc 3600
cctgcaaacg gcctgaatga tctggcactc tgcgttgcca ctgggatgaa atggaaaaaa 3660
gaaaaagaag aagtgtctct ggaagcgggc gcgctcacac aaacccgcaa cgattgtgta 3720
aacactctcc attgagaatc tggagtgcgg ttgccctcta ctggggagct gaagacagct 3780
agtgggggcg gggggaggac cgtgctagca tccttccacg gtgctcgctg gctgtggtgc 3840
atgccgggaa ccgaaacgcg gaactaaagt caagtcttgc tttggtggaa ctgacaatca 3900
acgaaatcac ttcgattgtt ttcctctttt tactggaatt cttggatttg atagatgggg 3960
gaggatcaga gggggagggg aggggcgggg agacggaggg aggaggggag gaggggagga 4020
ggggaggagg ggaggagggg aagggatgga ggaaaatact aacttttcta attcaacatg 4080
acaaagattc ggagaaagtg caccgctagt gaccgggagg aggaatgccc tattgggcat 4140
tatattccct gtcgtctaat ggaatcaaac tcttggttcc agcaccaagg attctgagcc 4200
tatcctattc aagacagtaa ctacagccca cacggaagag gctatacaac tgaagaaata 4260
aaattttcac tttatttcat ttctgtgact gcatgttcac atgtagagag ccacctgtgt 4320
ctaggggctg atgtgctggg cagtagagtt ctgagcccgt taactggaac aacccagaac 4380
tcccaccaca gttagagctt gctgagagag ggaggccctt ggtgagattt ctttgtgtat 4440
ttatttagag acagggtctc atactgtagt ccaagctagc ctccagctca cagaaattct 4500
cctgttccgg tttccaaagt actggagtta tgagtgtgtg ttaattgaac gctaagaatt 4560
tgctgattga agaaaacctc aagtgggttt ggctaatccc cacgacccca gaggctgagg 4620
caggaggaat gagagaattc aaggtttgcc agagccacag ggtgagctca atgtggagac 4680
tgtgagggtg agctcaatgt ggagactgtg agggtgagct caatgtggag actgtgaggg 4740
tgagctcaat gtggagactg tgagggtgag ctcaatgtgg agactgtgag ggtgagctca 4800
atgtggagac ctgtatcaag ataataatag tagtagtaac aatgcaggcg agggtgtggt 4860
tgagtggtag agcagttagt tgatttgaca tgcttgaggt ctcccggtcc atctgtggcc 4920
ctgcaacagg aagggaggga ggaagggggg gaacgagaga gaggaaagag agacagaagc 4980
taagataggg aatgagagag gaaggaagaa acgggaagaa attcagactc cttcctgagt 5040
tccgccaacg cctagtgaca tcctgtgcac accctaaggt ggcctttgtg tggcactggc 5100
ttgggtggtc gggaaaggca ttttcagctt gttgcagaac tgccacagta gcatgctggg 5160
tccgtgaaag tttctgcccg ttaacaagaa gtctctacta cttgtgacct caccagtgaa 5220
aatttcttta attgtctcct ggtgttctgg gttttgcatt tttgtttcta aggatacatt 5280
cctgggtgat gtcatgaagt ccccaaagac acagtggggc tgtgttggat tgggaaagat 5340
gatttatctg gggtgtcaaa aggaaaagaa gggaaacagg cacttgggaa aatgtcctcc 5400
cgcccacccg aattttggct tggcaaccgt ggtggaggag caagaaacac gtggacgttt 5460
gaggaggcat ggggtcctag gaggacagga agcagaagga gagagctggg ctgacagcct 5520
gcaggcattg cacagtttca gaaggagatt acagcatgac tgagttttta gggatccaac 5580
agggacctgg gtagagattc tgtgggctct gaggcaactt gacctcagcc agatggtatt 5640
tgaataacct gctcttagag ggaaaacaga catagcaaac agagccacgt ttagtgatga 5700
aactctcact ttgcctgagt catgtgcggc catgcccagg ggtcaggctg acactcaact 5760
caaaaacaag tgagaaattg aagacaatcc gtggtggcag ctactggaag ggccaccaca 5820
tccccagaaa gagtggagct gctaaaaagc catttgtgat aggcacagtt atcttgaatg 5880
catggagcag agattacgga aaaatcgaga atgttaatga ggcaacattc gagttgagtc 5940
attcagtgtg ggaaacccag acgcttccat cccctaaaag gaacatcttg ctctcagtca 6000
aaatggaaat aaaaattggg gcttgaattt ggcaaatgat tcagaactct gtgtaggtat 6060
tttcacacgc acagtggata attttcatgt tggagtttat ttgtgctaaa aggcagaaaa 6120
gggtaaaaag cacatcttaa gagttatgag gttctacgaa taaaaataat gttacttaca 6180
gctattcctt aattagtacc cccttccacc tgtggtaatt tcctgagata gtcagtgggg 6240
aaaagatctc tccttctctt ctttctcccc ctcccctcct ctccctccct ccctccctcc 6300
ctccctcctc tccctccctc cccctttcct tctttctttg ctccttctcc tctgcctcct 6360
tctccctttc ttcttcattt attctaagta gcttttaaca gcacaccaat tacctgtgta 6420
taacgggaaa acacaggctc aagcagctta gagaagattg atctgtgttc act 6473

<211> 7045

### <212> DNA

<213> Cricetulus griseus
<400> 2

(SEQ ID NO: 53)
actagcgtgc aattcagagg tgggtgaaga taaaaggcaa acatttgagg ccatttcctt 60
atttggcacg gcacttagga agtggaacat gcctaatcta ctggtttgta ccacctttcc 120
ctataatgga ctgtttggga agctcctggg caaccgattc tggcatctca ttggtcagag 180
gcctgttaaa tggtactctt atttgcaaag aaggctgtaa cttgtagctt taaaagcctc 240
tcctcaagaa agaagggaga aaggatatgg ctagacatat ctaatagact taaccactgt 300
gaaaagcctt agtatgaatc agatagaacc tatttttaac tcagttttga aaaaaataat 360
ctttatattt atttgtgtgt gtgtgtgtgt gtgtgtgtgt gtgtgtgtgt gtgtgtgtgt 420
gaaccacatg tagcaggtgc tggaggaggc cagaagaggg caccagatct cctggaactg 480
acaccacaca tggttatgag ctgcctgatg tgggtgctgg gaactgaact ctcgtgttct 540
gcaagagcag caactgttct cttaactgat gagccatctc tccagccccc cccataattt 600
taattgttca ttttagtaaa ttttattcat aatcaattat cacagtataa aacaatgatt 660
ttatatatat catatacata tcaaggatga cagtgagggg gatatgtgtg tgtgtgtgtg 720
tgtgtgtgtg tgtgtgtgtg tgtgttattt gtgtgtgtgc tttttaagaa ggtgccatag 780
tcactgcatt tctctgaagg atttcaaagg aatgagacat gtctgtctgc caggaaccct 840
atcttcctct ttgggaatct gacccaaatg aggtattctg aggaactgaa tgaagagctc 900
aagtagcagt gtcttaaacc caaatgtgct gtctagagaa agtcaacgtc atcagtgagc 960
tgaggagaga tttactgagc ggaagacaag cgctctttga tttaagtggc tcgaacagtc 1020
acggctgtgg agtggagcct gtgctcaggt ctgaggcagt ctttgctagc cagctgtgat 1080
gagcagtgaa gaaagggtgg agatggaggc agggtgggag cagggctatg gttcagacta 1140
ggtatcgtga gcacaccagc tggttgactt gtggtctgtg ggtcaggcgt tgtaaacgcc 1200
ctcagggtca ggcagtcaca ttgcttgaag ctgaatgggt gaggcaacac agagagtgca 1260
aagaaggcaa agtaccacct cttccccgac ccaggtcact tctgggttat agctgagact 1320
ccggacagca tgcaaccagc tggttagagc ttcagggaaa acttgatgtc tgcatgttgc 1380
tatgaaatgt gattcggtac atctggagaa aatttataat gctggctcag tcaagcactg 1440
aacaaaggta ccttggcttt gggagctaca tgacattgac ttgtaggcag actttttttt 1500
ttctgcccgc caattcccag ataaccaata tggaggctca atattaatta taaatgctcg 1560
gctgatagct caggcttgtt actagctaac tcttccaact taaatgaacc catttctatt 1620
atctacattc tgccacgtga ctttaccttg tacttcctgt ttcctctcct tgtctgactc 1680
tgcccttctg cttcccagag tccttagtct ggttctcctg cctaacctta tcctgcccag 1740
ctgctgacca agcatttata attaatatta agtctcccag tgagactctc atccagggag 1800
gacttgggtg ctcccccctc ctcattgcca tccgtgtctt cctcttccct cgcttccccc 1860
tcctcttcct gctcttcctc ctccacccct cctttcatag tattgatggc aagggtgttc 1920
tagaatggag gagtgcccat aggcatgcaa agaaaccagt taggatgctc tgtgaggggt 1980
tgtaatcata agcgatggac acaattcaag ccacagagtg aagacggaag gatgcactgt 2040
gctctagagc aacttctggg gcagaatcac agggtgagtt tctgacttga gggcgaagag 2100
gccacgagga agggagtgag tttgtctgag ctagaagcta cggcccacct cttggtagca 2160
gacctgccca caagcatgct ttgttaatca tgtgggatct gattttcctc taaatctatg 2220
ttcaactctt aagaaaatgt gaattctcac attaaaattt agatatacgt cttttggtgg 2280
ggggggtgta aaaaatcctc aagaatatgg atttctgggg gccggagaga tggctcagag 2340
gttaagagaa ctggttgctc ttctagacat tctgagttca attcccagca accacatggt 2400
ggctcacaac catctgtaat gcgacctggt gccatcttct gacatgcatg gatacatgca 2460
ggcagaaagc tgtatacata gtaaattgat aaatcttttt ttaaaaagag tatggattct 2520
gccgggtgtt ggtggcgcac gcctttaatc ccagcactct ggaggcagag gcaggtggat 2580
ctctgtgagt tcgagaccag cctggtctat aagagctagt tccaggacag cctccaaagc 2640
cacagagaaa ccctgtctcg aaaaaccaaa aaaaaaaaaa aaaaaaaaaa aaaaaaaaga 2700
gtatggattc taagaaagcc gtaacagctg gagctgtgta cggagttcag cgtggtacta 2760
gaagaacaga cattcatgat gaaacacccc aggattttta cttagtatct agtttccatt 2820
gttgttttga gaccggctct tatgctctcc aggctggcct caaactgctg atcttcccgc 2880
ctctacctct caagtcctgg gactacttgg ctcataaaac agtttttgtc gggctccctg 2940
aagttatggt tgtacaaacc gtgggggtca atatactcac ttgggcagag agagaaggtc 3000
tgaatcccag acaatgactg catctcagga cagttgggaa gaggacaatg gcagaaggac 3060
ttagaaaaga tagactggag ggtggaaaag cagcaggaac agagaaacaa aacaggaagc 3120
ttgctatcca gggccactct ggagtcctgt ggcaagatgg aagcgggcta ggggaataca 3180
tttgtgctac tgtgtgtgtg tgtgtgtgtg tgtgtgtgtg tgtgtgtgat caatgcctat 3240
caatgttgaa ggggaaatat gtataccaca ttgattctgg gagcaattct cagtatctgg 3300
cctagagaaa ggaatggccc ctgcagaata gacagagtga atggtgccct ttatcatttg 3360
ctaaagtgaa ggagaaataa acatccttcc atagagtttc aggtaaatga accccacagt 3420
tcatctgtgc cgtggtggag gcctggccaa cagttaaaaa gattagacac ggacaaagtc 3480
tgaaggaaac acctcgaata ggaagaggag agccacctca ttctgtaact ttcctcaagg 3540
ggaagatgtt ccaagagtgg gaataaatgg tcaaaggggg gatttttaat taggaaaacg 3600
atttcctgta tcacttgtga aactggaggt tgatttgggg cataggacaa tagatttgat 3660
gctttgcaaa aagctgtttc aaagcagaga aatggaatag agacaattat gtagcgagga 3720
gggagggtgg ggcgaagatg gagacagaga agtggaagct gactttaggg aagaggaaca 3780
tagaccacag gggcggggcg gggggcaggg gcggggggcg gggctcaaag gaggcagtgg 3840
gaacgttgct agtgttcgca gcgtaagcgt gaatgtgcaa gcgtctttgt ggtgtgtgac 3900
caggagtagc gtggctggct tgtgtgctgc ttgtaatccc agtctttgag gtttccacac 3960
tgttccacag tgggtgtgat tttccctcgg agagcatgag ggctctgctt tccccacatc 4020
ctccccagcg ttcgttggta tttgtttcca agatgttagt gggtgagaca aagcctctct 4080
gttgatttgc ctttaacagg tgacaaaaaa agctcaacca ggagacattt ttgccttctt 4140
ggaaggtaat gctcccatgt agagcaatgg gacccatctc taaggtgagg ctactcttgc 4200
agtttgcacc cagctcttct gatgcaggaa ggaagttggt gggcaagcaa gactgtttgc 4260
ttcttgcgat ggacacattc tgcacacaaa ggctcaggag gggagaaggc tgtttgatgt 4320
ttagcactca ggaaggcccc tgatgcatct gtgattagct gtctccatct gtggagcaga 4380
cacggactaa ctaaaaacca gtgtttttaa attgtcaagc ctttaaggtg aggaaattga 4440
cttattgtgc tgggccatac gtagagcaag tgctctgcat tgggccaacc cccggctctg 4500
gtttctaggc accagaatgg cctagaacta actcacaatc ctcccattcc aggtctcagg 4560
tgctagaatg aaccactata ccagcctgcc tgcctgccta cctgccttcc taaattttaa 4620
atcatgggga gtaggggaga atacacttat cttagttagg gtttctattg ctgtgaagag 4680
acaccatgag catggcaact cttataaagg aaaacattta gttgggtggc agtttcagag 4740
gttttagtac attgtcatca tggctgggaa catgatggca tgcagacaga catggtgctg 4800
gagaaaggga tgagagtcct acatcttgca ggcaacagga cctcagctga gacactggct 4860
ggtaccctga gcataggaaa cctcacagcc caccctcaca gtgacatatt tccttcaaca 4920
aagccatacc tcctaatagt gccactccct atgagatgac agggccaatt acattcaaac 4980
tgctataaca ctttaaagta ttttattttt attattgtaa attatgtatg tagctgggtg 5040
gtggcagccg aggtgcacgc ctttaatccc agcacttggg aggcagaggc agatggatct 5100
ctgtgagttc aagaccagcc tggtctataa gagctagttg caaggaagga tatacaaaga 5160
acagttctag gatagccttc aaagccacag agaagtgctg tcttgaaaac caaaaattgt 5220
gctgggacct gtctctgctt tggttgcttc ccactccccc agagctggac tcttggtcaa 5280
cactgaatca gctgcaaaat aaactcctgg attcctctct tgtaacagga gcccgaagtc 5340
aggcgcccac ttgtcttctc gcaggattgc catagacttt ttctgtgtgc ccaccattcc 5400
agactgaagt agagatggca gtggcagaga ctgggaaggc tgcaacgaaa acaggaagtt 5460
attgcaccct gggaatagtc tggaaatgaa gcttcaaaac ttgcttcatg ttcagttgta 5520
cacagactca ctcccaggtt gactcacacg tgtaaatatt cctgactatg tctgcactgc 5580
ttttatctga tgcttccttc ccaaaatgcc aagtgtacaa ggtgagggaa tcacccttgg 5640
attcagagcc cagggtcgtc ctccttaacc tggacttgtc tttctccggc agcctctgac 5700
acccctcccc ccattttctc tatcagaagg tctgagcaga gttggggcac gctcatgtcc 5760
tgatacactc cttgtcttcc tgaagatcta acttctgacc cagaaagatg gctaaggtgg 5820
tgaagtgttt gacatgaaga cttggtctta agaactggag caggggaaaa aagtcggatg 5880
tggcagcatg tacccgaaat cccagaactg gggaggtaga gacggatgag tgcccggggc 5940
tagctggctg ctcagccagc ctagctgaat tgccaaattc caactcctat tgaaaaacct 6000
ttaccaaaca aacaaacaaa caaataataa caacaacaac aacaacaaac taccccatac 6060
aaggtgggcg gctcttggct cttgaggaat gactcaccca aacccaaagc ttgccacagc 6120
tgttctctgg cctaaatggg gtgggggtgg ggcagagaca gagacagaga gagacatgac 6180
ttcctgggct gggctgtgtg ctctaggcca ccaggaactt tcctgtcttg ctctctgtct 6240
ggcacagcca gagcaccagc acccagcagg tgcacacacc tccctccgtg cttcttgagc 6300
aaacacaggt gccttggtct gtctattgaa ccggagtaag ttcttgcaga tgtatgcatg 6360
gaaacaacat tgtcctggtt ttatttctac tgttgtgata aaaaccgggg aactccagga 6420
agcagctgag gcagaggcaa atgcaaggaa tgctgcctcc tagcttgctc cccatggctt 6480
gccgggcctg ctttctgcaa gcccttctct ccccattggc atgcctgaca tgaacagcgt 6540
ttgaaatgct ctcaaatgtc actttcaaag aaggcttctc tgatcttgct aactaaatca 6600
gaccatgttt caccgtgcat tatctttctg ctgtctgtct gtctgtctgt ctgtctatct 6660
gtctatcatc tatcaatcat ctatctatct atcttctatt tatctaccta tcattcaatc 6720
atctatcttc taactagtta tcatttattt atttgtttac ttactttttt tatttgagac 6780
agtatttctc tgagtgacag ccttggctgt cctggaaccc attctgtaac caggctgtcc 6840
tcaaactcac agagatccaa ctgcctctgc ctctctggtg ctggggttaa agacgtgcac 6900
caccaacgcc ccgctctatc atctatttat gtacttatta ttcagtcatt atctatcctc 6960
taactatcca tcatctgtct atccatcatc tatctatcta tctatctatc tatctatcta 7020
tctatcatcc atctataatc aattg 7045

<211> 6473

### <212> DNA

<213> Cricetulus griseus
<400> 3

(SEQ ID NO: 54)
agtgaacaca gatcaatctt ctctaagctg cttgagcctg tgttttcccg ttatacacag 60
gtaattggtg tgctgttaaa agctacttag aataaatgaa gaagaaaggg agaaggaggc 120
agaggagaag gagcaaagaa agaaggaaag ggggagggag ggagaggagg gagggaggga 180
gggagggagg gagaggaggg gagggggaga aagaagagaa ggagagatct tttccccact 240
gactatctca ggaaattacc acaggtggaa gggggtacta attaaggaat agctgtaagt 300
aacattattt ttattcgtag aacctcataa ctcttaagat gtgcttttta cccttttctg 360
ccttttagca caaataaact ccaacatgaa aattatccac tgtgcgtgtg aaaataccta 420
cacagagttc tgaatcattt gccaaattca agccccaatt tttatttcca ttttgactga 480
gagcaagatg ttccttttag gggatggaag cgtctgggtt tcccacactg aatgactcaa 540
ctcgaatgtt gcctcattaa cattctcgat ttttccgtaa tctctgctcc atgcattcaa 600
gataactgtg cctatcacaa atggcttttt agcagctcca ctctttctgg ggatgtggtg 660
gcccttccag tagctgccac cacggattgt cttcaatttc tcacttgttt ttgagttgag 720
tgtcagcctg acccctgggc atggccgcac atgactcagg caaagtgaga gtttcatcac 780
taaacgtggc tctgtttgct atgtctgttt tccctctaag agcaggttat tcaaatacca 840
tctggctgag gtcaagttgc ctcagagccc acagaatctc tacccaggtc cctgttggat 900
ccctaaaaac tcagtcatgc tgtaatctcc ttctgaaact gtgcaatgcc tgcaggctgt 960
cagcccagct ctctccttct gcttcctgtc ctcctaggac cccatgcctc ctcaaacgtc 1020
cacgtgtttc ttgctcctcc accacggttg ccaagccaaa attcgggtgg gcgggaggac 1080
attttcccaa gtgcctgttt cccttctttt ccttttgaca ccccagataa atcatctttc 1140
ccaatccaac acagccccac tgtgtctttg gggacttcat gacatcaccc aggaatgtat 1200
ccttagaaac aaaaatgcaa aacccagaac accaggagac aattaaagaa attttcactg 1260
gtgaggtcac aagtagtaga gacttcttgt taacgggcag aaactttcac ggacccagca 1320
tgctactgtg gcagttctgc aacaagctga aaatgccttt cccgaccacc caagccagtg 1380
ccacacaaag gccaccttag ggtgtgcaca ggatgtcact aggcgttggc ggaactcagg 1440
aaggagtctg aatttcttcc cgtttcttcc ttcctctctc attccctatc ttagcttctg 1500
tctctctttc ctctctctcg ttccccccct tcctccctcc cttcctgttg cagggccaca 1560
gatggaccgg gagacctcaa gcatgtcaaa tcaactaact gctctaccac tcaaccacac 1620
cctcgcctgc attgttacta ctactattat tatcttgata caggtctcca cattgagctc 1680
accctcacag tctccacatt gagctcaccc tcacagtctc cacattgagc tcaccctcac 1740
agtctccaca ttgagctcac cctcacagtc tccacattga gctcaccctc acagtctcca 1800
cattgagctc accctgtggc tctggcaaac cttgaattct ctcattcctc ctgcctcagc 1860
ctctggggtc gtggggatta gccaaaccca cttgaggttt tcttcaatca gcaaattctt 1920
agcgttcaat taacacacac tcataactcc agtactttgg aaaccggaac aggagaattt 1980
ctgtgagctg gaggctagct tggactacag tatgagaccc tgtctctaaa taaatacaca 2040
aagaaatctc accaagggcc tccctctctc agcaagctct aactgtggtg ggagttctgg 2100
gttgttccag ttaacgggct cagaactcta ctgcccagca catcagcccc tagacacagg 2160
tggctctcta catgtgaaca tgcagtcaca gaaatgaaat aaagtgaaaa ttttatttct 2220
tcagttgtat agcctcttcc gtgtgggctg tagttactgt cttgaatagg ataggctcag 2280
aatccttggt gctggaacca agagtttgat tccattagac gacagggaat ataatgccca 2340
atagggcatt cctcctcccg gtcactagcg gtgcactttc tccgaatctt tgtcatgttg 2400
aattagaaaa gttagtattt tcctccatcc cttcccctcc tcccctcctc ccctcctccc 2460
ctcctcccct cctccctccg tctccccgcc cctcccctcc ccctctgatc ctcccccatc 2520
tatcaaatcc aagaattcca gtaaaaagag gaaaacaatc gaagtgattt cgttgattgt 2580
cagttccacc aaagcaagac ttgactttag ttccgcgttt cggttcccgg catgcaccac 2640
agccagcgag caccgtggaa ggatgctagc acggtcctcc ccccgccccc actagctgtc 2700
ttcagctccc cagtagaggg caaccgcact ccagattctc aatggagagt gtttacacaa 2760
tcgttgcggg tttgtgtgag cgcgcccgct tccagagaca cttcttcttt ttcttttttc 2820
catttcatcc cagtggcaac gcagagtgcc agatcattca ggccgtttgc agggcaagcc 2880
gtgggagctt ggcaagcaag gccccatttc ctagggaacc cgtgcctggc gcttcaggaa 2940
agcacgggaa cctggcactg tgactctgcg ggtattattt tgcagaactc tttattaaac 3000
gggagtttca agtccagctg gagacgacca ggcagcgcct ttaaccccag agtcacacac 3060
aggtgccttt tcttggggcc agattggggt tgtgtggcag acctgcgacc agcttgacaa 3120
ctcttctgcc aggccacaaa atggtgttgg ctgtaagagg tgacaccagg gacagggaag 3180
atcgctgcta ttctcctgag ctctccaaag acccacacca gtctgtcccc ctttcctcct 3240
gctcttcccc tgtatcgccc cctcaccatc tcccccaacg agactcttgg catctcctcg 3300
gcacaaggat ttgaaaatag atgcttgggg gtgagaagaa gaagagagaa agagagagaa 3360
ggaaggaagg atatatagat gatacagacg catacaggtg acatgtagct aatcattttt 3420
aattaaaaaa taaattaaaa gcaaatcaag gatatatatg atacccttag agcaagtgtc 3480
tcatacacac acaaacacac acacacaata tatatatata tatatatata tatatatata 3540
tatatatata ttatacttgg aacaagtgtc cagaagggct ggggactcta aagtgcttgt 3600
caaagccagg ctcacatcag taatcttatc acctggtaga ctgagacagg aggattttga 3660
tgagttcagg cccagcctga gctgcagaat gtgattctat cccaaaaaag taaaataaaa 3720
taaaattcaa aatacacgaa aagagtattt gctgaacaaa caagcctaaa gccctggatc 3780
ccttccccca tgtcctaaga aaataagttt cttgaagctg gagggatggc tcagaggtta 3840
agagccccag ctgcacttgc ggaacactaa gacccagttc ccagacccca cactgtgggt 3900
cacaactgtc tcaaacgcca gctccggagg atccatgccc tctcctggcc tccaccggca 3960
ccaagaacac atacagtgcc catacattta tgcaagcaag gtattcacgc acataaaact 4020
aaaagaatat ttaataaaga tataacaaaa tagcatgaag cccagctggt acagaggttc 4080
aaactacatc ccaggttcat ccctctgcct ttgctctcag ttggcttggg taggtctctt 4140
ctctgaactg gcgccctgcg ggttccacat tgagaccctc tcatttttaa acctacttct 4200
tctgggcggg gttaattgct gccagggctc aagccaacgc ttcctcttct ccacagcaat 4260
cttccaagtt tcacgagata accaggaact gctaagttca tgtgaacctt agtgaagaac 4320
ctgagtcttc ccatgtgatt ggtgtgtgca tgtgtgcata cacaaatgta tgtgtgtgct 4380
ctatgtgtgc ctatgtatgt gtgcatgcat gtgtgcatat acaaatgcat atatgtctat 4440
gtagtgtgcg tacacaaatg tatgtgtgtg ctcaatgtgt gcctatgtgt gtgtatgcat 4500
gtgtgcgtac acaatgcatg tgtgtggtgt ctgtgtgcct gtgtgtgtat gcatgtatgc 4560
atacacaaat gtatatgtgt ggtgtgtgaa tgtgtgccta tgtatgtgtg tgctgtgtgt 4620
gggtgtggta tgtgtgtgat gtgtggaggg gtgtgtatgt gtggtatgta taggtgatac 4680
gtttggggtg taatatgcgt atgtggtttg tgaaatgtag ttcgtgtgtg tgcatgtgtg 4740
cgtgcgtgcg tgcgtgcgtg cgtgtgtgtg tgtgtgtgtg tgtgtgtgtt ggatatagta 4800
tgtgtgaggt gtgtgtactc accatggcct ccctcacttg ggggagtgaa gtcagcagcc 4860
tggaccactc agggacatga gatactcaga cacatcttga tttccacccc tcttttcctg 4920
atcctccttc acgtgtcact ttcccaaaca ctggacaaca gtttgggggc atctgattcc 4980
actaatgaca gggacatcac atgtctccag agggaacacc ttctgtgtca catgtcatct 5040
gagaatgtag cagagtcaca gagaaatgtc acagaaacca aaatgcagag taccaaggta 5100
tagctaggca cagagcagag gggaagccgc tgaatttatt aaaaatgtca gaatcgtaaa 5160
agacagggga cagcggtggg gacattcagg gtccagtagc acacaggcag tccaaacctg 5220
atcactggaa ggtagtaggt aaggaaaggc tgcacacaga ttattcacac agtttataca 5280
tgtacacaga ttattcacat ggtttgtgta tgtgcacaga ttattcacac agtttataca 5340
tgtgtggctt cgtggtaact ttgagcttac tttcaattta aaaggatctc tctcacaagc 5400
tggggccggg aatggctgca gtcaacactc catcacttag tcacactgtg caaacagcac 5460
ctcctgactc atggtgactt gtagtaaaat gaagaggcca catttgcatc caagacagct 5520
catcagtacc tagtgaagaa tctgtccctg agtatttgca tgaatggacc cgggtccagg 5580
gcctggctgg gagtctccag gtgttgcagc cagaatgtca ttgtgttttt tcaggatccc 5640
agaagtttct aaaatacagg ccaagtactc atttgtgtta caaagtatct gactaataga 5700
agtgattagg taacacaaag ccttttaaaa accgagatca cccttgtcat gtccctggcc 5760
tcttagaaca agatccaagc ttttgctggt tgacaagtgg ggccatccag tgcgtctccg 5820
ttcctgctac ttcatctgga agacctctcc cactaacttg cccctgaccc ctcacacctg 5880
ctgtttcctt tccacccgga agtgcttgtc taggctttca tggccatctg actgagcatc 5940
taggcctcag tccagtggtc cctcagctct ctctagtcac tgtactaatg gaaacggcca 6000
ctaactacat tttcaatatg gaagcctcct cctcaggaac ctccaagggc agaagcctcc 6060
agagaaccac tcctgacccc ctggagttct gagtgcttct ggccctctct gtgtctgcag 6120
gactattcac cacttgtgtt gaatggttca gtcctcacct cctctggcat gtgctcagtt 6180
ctcatctcat tggggagtcc ttcccaggtc actcttctct cctgtctttg aagtgttttt 6240
ttccttcatg gtatttctgt ctgggcacac acacagacac acatacacac acatacacac 6300
ccatgcagta tggcagatac atcacctatg tttcagattt ttattctacc atcacccaat 6360
acctgaatcc ccgaaaaagc cttagaaagc caggaatttg tgtatttttg tcagcactcc 6420
accccagcac ctgaagccaa gcctgactta atatttttgg ttttgtttct aga 6473

<211> 7045

### <212> DNA

<213> Cricetulus griseus
<400> 4

(SEQ ID NO: 55)
caattgatta tagatggatg atagatagat agatagatag atagatagat agatagatga 60
tggatagaca gatgatggat agttagagga tagataatga ctgaataata agtacataaa 120
tagatgatag agcggggcgt tggtggtgca cgtctttaac cccagcacca gagaggcaga 180
ggcagttgga tctctgtgag tttgaggaca gcctggttac agaatgggtt ccaggacagc 240
caaggctgtc actcagagaa atactgtctc aaataaaaaa agtaagtaaa caaataaata 300
aatgataact agttagaaga tagatgattg aatgataggt agataaatag aagatagata 360
gatagatgat tgatagatga tagacagata gacagacaga cagacagaca gacagcagaa 420
agataatgca cggtgaaaca tggtctgatt tagttagcaa gatcagagaa gccttctttg 480
aaagtgacat ttgagagcat ttcaaacgct gttcatgtca ggcatgccaa tggggagaga 540
agggcttgca gaaagcaggc ccggcaagcc atggggagca agctaggagg cagcattcct 600
tgcatttgcc tctgcctcag ctgcttcctg gagttccccg gtttttatca caacagtaga 660
aataaaacca ggacaatgtt gtttccatgc atacatctgc aagaacttac tccggttcaa 720
tagacagacc aaggcacctg tgtttgctca agaagcacgg agggaggtgt gtgcacctgc 780
tgggtgctgg tgctctggct gtgccagaca gagagcaaga caggaaagtt cctggtggcc 840
tagagcacac agcccagccc aggaagtcat gtctctctct gtctctgtct ctgccccacc 900
cccaccccat ttaggccaga gaacagctgt ggcaagcttt gggtttgggt gagtcattcc 960
tcaagagcca agagccgccc accttgtatg gggtagtttg ttgttgttgt tgttgttatt 1020
atttgtttgt ttgtttgttt ggtaaaggtt tttcaatagg agttggaatt tggcaattca 1080
gctaggctgg ctgagcagcc agctagcccc gggcactcat ccgtctctac ctccccagtt 1140
ctgggatttc gggtacatgc tgccacatcc gacttttttc ccctgctcca gttcttaaga 1200
ccaagtcttc atgtcaaaca cttcaccacc ttagccatct ttctgggtca gaagttagat 1260
cttcaggaag acaaggagtg tatcaggaca tgagcgtgcc ccaactctgc tcagaccttc 1320
tgatagagaa aatgggggga ggggtgtcag aggctgccgg agaaagacaa gtccaggtta 1380
aggaggacga ccctgggctc tgaatccaag ggtgattccc tcaccttgta cacttggcat 1440
tttgggaagg aagcatcaga taaaagcagt gcagacatag tcaggaatat ttacacgtgt 1500
gagtcaacct gggagtgagt ctgtgtacaa ctgaacatga agcaagtttt gaagcttcat 1560
ttccagacta ttcccagggt gcaataactt cctgttttcg ttgcagcctt cccagtctct 1620
gccactgcca tctctacttc agtctggaat ggtgggcaca cagaaaaagt ctatggcaat 1680
cctgcgagaa gacaagtggg cgcctgactt cgggctcctg ttacaagaga ggaatccagg 1740
agtttatttt gcagctgatt cagtgttgac caagagtcca gctctggggg agtgggaagc 1800
aaccaaagca gagacaggtc ccagcacaat ttttggtttt caagacagca cttctctgtg 1860
gctttgaagg ctatcctaga actgttcttt gtatatcctt ccttgcaact agctcttata 1920
gaccaggctg gtcttgaact cacagagatc catctgcctc tgcctcccaa gtgctgggat 1980
taaaggcgtg cacctcggct gccaccaccc agctacatac ataatttaca ataataaaaa 2040
taaaatactt taaagtgtta tagcagtttg aatgtaattg gccctgtcat ctcataggga 2100
gtggcactat taggaggtat ggctttgttg aaggaaatat gtcactgtga gggtgggctg 2160
tgaggtttcc tatgctcagg gtaccagcca gtgtctcagc tgaggtcctg ttgcctgcaa 2220
gatgtaggac tctcatccct ttctccagca ccatgtctgt ctgcatgcca tcatgttccc 2280
agccatgatg acaatgtact aaaacctctg aaactgccac ccaactaaat gttttccttt 2340
ataagagttg ccatgctcat ggtgtctctt cacagcaata gaaaccctaa ctaagataag 2400
tgtattctcc cctactcccc atgatttaaa atttaggaag gcaggtaggc aggcaggcag 2460
gctggtatag tggttcattc tagcacctga gacctggaat gggaggattg tgagttagtt 2520
ctaggccatt ctggtgccta gaaaccagag ccgggggttg gcccaatgca gagcacttgc 2580
tctacgtatg gcccagcaca ataagtcaat ttcctcacct taaaggcttg acaatttaaa 2640
aacactggtt tttagttagt ccgtgtctgc tccacagatg gagacagcta atcacagatg 2700
catcaggggc cttcctgagt gctaaacatc aaacagcctt ctcccctcct gagcctttgt 2760
gtgcagaatg tgtccatcgc aagaagcaaa cagtcttgct tgcccaccaa cttccttcct 2820
gcatcagaag agctgggtgc aaactgcaag agtagcctca ccttagagat gggtcccatt 2880
gctctacatg ggagcattac cttccaagaa ggcaaaaatg tctcctggtt gagctttttt 2940
tgtcacctgt taaaggcaaa tcaacagaga ggctttgtct cacccactaa catcttggaa 3000
acaaatacca acgaacgctg gggaggatgt ggggaaagca gagccctcat gctctccgag 3060
ggaaaatcac acccactgtg gaacagtgtg gaaacctcaa agactgggat tacaagcagc 3120
acacaagcca gccacgctac tcctggtcac acaccacaaa gacgcttgca cattcacgct 3180
tacgctgcga acactagcaa cgttcccact gcctcctttg agccccgccc cccgcccctg 3240
ccccccgccc cgcccctgtg gtctatgttc ctcttcccta aagtcagctt ccacttctct 3300
gtctccatct tcgccccacc ctccctcctc gctacataat tgtctctatt ccatttctct 3360
gctttgaaac agctttttgc aaagcatcaa atctattgtc ctatgcccca aatcaacctc 3420
cagtttcaca agtgatacag gaaatcgttt tcctaattaa aaatcccccc tttgaccatt 3480
tattcccact cttggaacat cttccccttg aggaaagtta cagaatgagg tggctctcct 3540
cttcctattc gaggtgtttc cttcagactt tgtccgtgtc taatcttttt aactgttggc 3600
caggcctcca ccacggcaca gatgaactgt ggggttcatt tacctgaaac tctatggaag 3660
gatgtttatt tctccttcac tttagcaaat gataaagggc accattcact ctgtctattc 3720
tgcaggggcc attcctttct ctaggccaga tactgagaat tgctcccaga atcaatgtgg 3780
tatacatatt tccccttcaa cattgatagg cattgatcac acacacacac acacacacac 3840
acacacacac acacagtagc acaaatgtat tcccctagcc cgcttccatc ttgccacagg 3900
actccagagt ggccctggat agcaagcttc ctgttttgtt tctctgttcc tgctgctttt 3960
ccaccctcca gtctatcttt tctaagtcct tctgccattg tcctcttccc aactgtcctg 4020
agatgcagtc attgtctggg attcagacct tctctctctg cccaagtgag tatattgacc 4080
cccacggttt gtacaaccat aacttcaggg agcccgacaa aaactgtttt atgagccaag 4140
tagtcccagg acttgagagg tagaggcggg aagatcagca gtttgaggcc agcctggaga 4200
gcataagagc cggtctcaaa acaacaatgg aaactagata ctaagtaaaa atcctggggt 4260
gtttcatcat gaatgtctgt tcttctagta ccacgctgaa ctccgtacac agctccagct 4320
gttacggctt tcttagaatc catactcttt tttttttttt tttttttttt ttttttttgg 4380
tttttcgaga cagggtttct ctgtggcttt ggaggctgtc ctggaactag ctcttataga 4440
ccaggctggt ctcgaactca cagagatcca cctgcctctg cctccagagt gctgggatta 4500
aaggcgtgcg ccaccaacac ccggcagaat ccatactctt tttaaaaaaa gatttatcaa 4560
tttactatgt atacagcttt ctgcctgcat gtatccatgc atgtcagaag atggcaccag 4620
gtcgcattac agatggttgt gagccaccat gtggttgctg ggaattgaac tcagaatgtc 4680
tagaagagca accagttctc ttaacctctg agccatctct ccggccccca gaaatccata 4740
ttcttgagga ttttttacac cccccccacc aaaagacgta tatctaaatt ttaatgtgag 4800
aattcacatt ttcttaagag ttgaacatag atttagagga aaatcagatc ccacatgatt 4860
aacaaagcat gcttgtgggc aggtctgcta ccaagaggtg ggccgtagct tctagctcag 4920
acaaactcac tcccttcctc gtggcctctt cgccctcaag tcagaaactc accctgtgat 4980
tctgccccag aagttgctct agagcacagt gcatccttcc gtcttcactc tgtggcttga 5040
attgtgtcca tcgcttatga ttacaacccc tcacagagca tcctaactgg tttctttgca 5100
tgcctatggg cactcctcca ttctagaaca cccttgccat caatactatg aaaggagggg 5160
tggaggagga agagcaggaa gaggaggggg aagcgaggga agaggaagac acggatggca 5220
atgaggaggg gggagcaccc aagtcctccc tggatgagag tctcactggg agacttaata 5280
ttaattataa atgcttggtc agcagctggg caggataagg ttaggcagga gaaccagact 5340
aaggactctg ggaagcagaa gggcagagtc agacaaggag aggaaacagg aagtacaagg 5400
taaagtcacg tggcagaatg tagataatag aaatgggttc atttaagttg gaagagttag 5460
ctagtaacaa gcctgagcta tcagccgagc atttataatt aatattgagc ctccatattg 5520
gttatctggg aattggcggg cagaaaaaaa aaagtctgcc tacaagtcaa tgtcatgtag 5580
ctcccaaagc caaggtacct ttgttcagtg cttgactgag ccagcattat aaattttctc 5640
cagatgtacc gaatcacatt tcatagcaac atgcagacat caagttttcc ctgaagctct 5700
aaccagctgg ttgcatgctg tccggagtct cagctataac ccagaagtga cctgggtcgg 5760
ggaagaggtg gtactttgcc ttctttgcac tctctgtgtt gcctcaccca ttcagcttca 5820
agcaatgtga ctgcctgacc ctgagggcgt ttacaacgcc tgacccacag accacaagtc 5880
aaccagctgg tgtgctcacg atacctagtc tgaaccatag ccctgctccc accctgcctc 5940
catctccacc ctttcttcac tgctcatcac agctggctag caaagactgc ctcagacctg 6000
agcacaggct ccactccaca gccgtgactg ttcgagccac ttaaatcaaa gagcgcttgt 6060
cttccgctca gtaaatctct cctcagctca ctgatgacgt tgactttctc tagacagcac 6120
atttgggttt aagacactgc tacttgagct cttcattcag ttcctcagaa tacctcattt 6180
gggtcagatt cccaaagagg aagatagggt tcctggcaga cagacatgtc tcattccttt 6240
gaaatccttc agagaaatgc agtgactatg gcaccttctt aaaaagcaca cacacaaata 6300
acacacacac acacacacac acacacacac acacacacac atatccccct cactgtcatc 6360
cttgatatgt atatgatata tataaaatca ttgttttata ctgtgataat tgattatgaa 6420
taaaatttac taaaatgaac aattaaaatt atgggggggg ctggagagat ggctcatcag 6480
ttaagagaac agttgctgct cttgcagaac acgagagttc agttcccagc acccacatca 6540
ggcagctcat aaccatgtgt ggtgtcagtt ccaggagatc tggtgccctc ttctggcctc 6600
ctccagcacc tgctacatgt ggttcacaca cacacacaca cacacacaca cacacacaca 6660
cacacacaca caaataaata taaagattat ttttttcaaa actgagttaa aaataggttc 6720
tatctgattc atactaaggc ttttcacagt ggttaagtct attagatatg tctagccata 6780
tcctttctcc cttctttctt gaggagaggc ttttaaagct acaagttaca gccttctttg 6840
caaataagag taccatttaa caggcctctg accaatgaga tgccagaatc ggttgcccag 6900
gagcttccca aacagtccat tatagggaaa ggtggtacaa accagtagat taggcatgtt 6960
ccacttccta agtgccgtgc caaataagga aatggcctca aatgtttgcc ttttatcttc 7020
acccacctct gaattgcacg ctagt 7045

<211> 13515

### <212> DNA

<213> Cricetulus griseus
<400> 5

(SEQ ID NO: 56)
tctagaaaca aaaccaaaaa tattaagtca ggcttggctt caggtgctgg ggtggagtgc 60
tgacaaaaat acacaaattc ctggctttct aaggcttttt cggggattca ggtattgggt 120
gatggtagaa taaaaatctg aaacataggt gatgtatctg ccatactgca tgggtgtgta 180
tgtgtgtgta tgtgtgtctg tgtgtgtgcc cagacagaaa taccatgaag gaaaaaaaca 240
cttcaaagac aggagagaag agtgacctgg gaaggactcc ccaatgagat gagaactgag 300
cacatgccag aggaggtgag gactgaacca ttcaacacaa gtggtgaata gtcctgcaga 360
cacagagagg gccagaagca ctcagaactc cagggggtca ggagtggttc tctggaggct 420
tctgcccttg gaggttcctg aggaggaggc ttccatattg aaaatgtagt tagtggccgt 480
ttccattagt acagtgacta gagagagctg agggaccact ggactgaggc ctagatgctc 540
agtcagatgg ccatgaaagc ctagacaagc acttccgggt ggaaaggaaa cagcaggtgt 600
gaggggtcag gggcaagtta gtgggagagg tcttccagat gaagtagcag gaacggagac 660
gcactggatg gccccacttg tcaaccagca aaagcttgga tcttgttcta agaggccagg 720
gacatgacaa gggtgatctc ggtttttaaa aggctttgtg ttacctaatc acttctatta 780
gtcagatact ttgtaacaca aatgagtact tggcctgtat tttagaaact tctgggatcc 840
tgaaaaaaca caatgacatt ctggctgcaa cacctggaga ctcccagcca ggccctggac 900
ccgggtccat tcatgcaaat actcagggac agattcttca ctaggtactg atgagctgtc 960
ttggatgcaa atgtggcctc ttcattttac tacaagtcac catgagtcag gaggtgctgt 1020
ttgcacagtg tgactaagtg atggagtgtt gactgcagcc attcccggcc ccagcttgtg 1080
agagagatcc ttttaaattg aaagtaagct caaagttacc acgaagccac acatgtataa 1140
actgtgtgaa taatctgtgc acatacacaa accatgtgaa taatctgtgt acatgtataa 1200
actgtgtgaa taatctgtgt gcagcctttc cttacctact accttccagt gatcaggttt 1260
ggactgcctg tgtgctactg gaccctgaat gtccccaccg ctgtcccctg tcttttacga 1320
ttctgacatt tttaataaat tcagcggctt cccctctgct ctgtgcctag ctataccttg 1380
gtactctgca ttttggtttc tgtgacattt ctctgtgact ctgctacatt ctcagatgac 1440
atgtgacaca gaaggtgttc cctctggaga catgtgatgt ccctgtcatt agtggaatca 1500
gatgccccca aactgttgtc cagtgtttgg gaaagtgaca cgtgaaggag gatcaggaaa 1560
agaggggtgg aaatcaagat gtgtctgagt atctcatgtc cctgagtggt ccaggctgct 1620
gacttcactc ccccaagtga gggaggccat ggtgagtaca cacacctcac acatactata 1680
tccaacacac acacacacac acacacacac acgcacgcac gcacgcacgc acgcacacat 1740
gcacacacac gaactacatt tcacaaacca catacgcata ttacacccca aacgtatcac 1800
ctatacatac cacacataca cacccctcca cacatcacac acataccaca cccacacaca 1860
gcacacacat acataggcac acattcacac accacacata tacatttgtg tatgcataca 1920
tgcatacaca cacaggcaca cagacaccac acacatgcat tgtgtacgca cacatgcata 1980
cacacacata ggcacacatt gagcacacac atacatttgt gtacgcacac tacatagaca 2040
tatatgcatt tgtatatgca cacatgcatg cacacataca taggcacaca tagagcacac 2100
acatacattt gtgtatgcac acatgcacac accaatcaca tgggaagact caggttcttc 2160
actaaggttc acatgaactt agcagttcct ggttatctcg tgaaacttgg aagattgctg 2220
tggagaagag gaagcgttgg cttgagccct ggcagcaatt aaccccgccc agaagaagta 2280
ggtttaaaaa tgagagggtc tcaatgtgga acccgcaggg cgccagttca gagaagagac 2340
ctacccaagc caactgagag caaaggcaga gggatgaacc tgggatgtag tttgaacctc 2400
tgtaccagct gggcttcatg ctattttgtt atatctttat taaatattct tttagtttta 2460
tgtgcgtgaa taccttgctt gcataaatgt atgggcactg tatgtgttct tggtgccggt 2520
ggaggccagg agagggcatg gatcctccgg agctggcgtt tgagacagtt gtgacccaca 2580
gtgtggggtc tgggaactgg gtcttagtgt tccgcaagtg cagctggggc tcttaacctc 2640
tgagccatcc ctccagcttc aagaaactta ttttcttagg acatggggga agggatccag 2700
ggctttaggc ttgtttgttc agcaaatact cttttcgtgt attttgaatt ttattttatt 2760
ttactttttt gggatagaat cacattctgc agctcaggct gggcctgaac tcatcaaaat 2820
cctcctgtct cagtctacca ggtgataaga ttactgatgt gagcctggct ttgacaagca 2880
ctttagagtc cccagccctt ctggacactt gttccaagta taatatatat atatatatat 2940
atatatatat atatatatat atatattgtg tgtgtgtgtt tgtgtgtgta tgagacactt 3000
gctctaaggg tatcatatat atccttgatt tgcttttaat ttatttttta attaaaaatg 3060
attagctaca tgtcacctgt atgcgtctgt atcatctata tatccttcct tccttctctc 3120
tctttctctc ttcttcttct cacccccaag catctatttt caaatccttg tgccgaggag 3180
atgccaagag tctcgttggg ggagatggtg agggggcgat acaggggaag agcaggagga 3240
aagggggaca gactggtgtg ggtctttgga gagctcagga gaatagcagc gatcttccct 3300
gtccctggtg tcacctctta cagccaacac cattttgtgg cctggcagaa gagttgtcaa 3360
gctggtcgca ggtctgccac acaaccccaa tctggcccca agaaaaggca cctgtgtgtg 3420
actctggggt taaaggcgct gcctggtcgt ctccagctgg acttgaaact cccgtttaat 3480
aaagagttct gcaaaataat acccgcagag tcacagtgcc aggttcccgt gctttcctga 3540
agcgccaggc acgggttccc taggaaatgg ggccttgctt gccaagctcc cacggcttgc 3600
cctgcaaacg gcctgaatga tctggcactc tgcgttgcca ctgggatgaa atggaaaaaa 3660
gaaaaagaag aagtgtctct ggaagcgggc gcgctcacac aaacccgcaa cgattgtgta 3720
aacactctcc attgagaatc tggagtgcgg ttgccctcta ctggggagct gaagacagct 3780
agtgggggcg gggggaggac cgtgctagca tccttccacg gtgctcgctg gctgtggtgc 3840
atgccgggaa ccgaaacgcg gaactaaagt caagtcttgc tttggtggaa ctgacaatca 3900
acgaaatcac ttcgattgtt ttcctctttt tactggaatt cttggatttg atagatgggg 3960
gaggatcaga gggggagggg aggggcgggg agacggaggg aggaggggag gaggggagga 4020
ggggaggagg ggaggagggg aagggatgga ggaaaatact aacttttcta attcaacatg 4080
acaaagattc ggagaaagtg caccgctagt gaccgggagg aggaatgccc tattgggcat 4140
tatattccct gtcgtctaat ggaatcaaac tcttggttcc agcaccaagg attctgagcc 4200
tatcctattc aagacagtaa ctacagccca cacggaagag gctatacaac tgaagaaata 4260
aaattttcac tttatttcat ttctgtgact gcatgttcac atgtagagag ccacctgtgt 4320
ctaggggctg atgtgctggg cagtagagtt ctgagcccgt taactggaac aacccagaac 4380
tcccaccaca gttagagctt gctgagagag ggaggccctt ggtgagattt ctttgtgtat 4440
ttatttagag acagggtctc atactgtagt ccaagctagc ctccagctca cagaaattct 4500
cctgttccgg tttccaaagt actggagtta tgagtgtgtg ttaattgaac gctaagaatt 4560
tgctgattga agaaaacctc aagtgggttt ggctaatccc cacgacccca gaggctgagg 4620
caggaggaat gagagaattc aaggtttgcc agagccacag ggtgagctca atgtggagac 4680
tgtgagggtg agctcaatgt ggagactgtg agggtgagct caatgtggag actgtgaggg 4740
tgagctcaat gtggagactg tgagggtgag ctcaatgtgg agactgtgag ggtgagctca 4800
atgtggagac ctgtatcaag ataataatag tagtagtaac aatgcaggcg agggtgtggt 4860
tgagtggtag agcagttagt tgatttgaca tgcttgaggt ctcccggtcc atctgtggcc 4920
ctgcaacagg aagggaggga ggaagggggg gaacgagaga gaggaaagag agacagaagc 4980
taagataggg aatgagagag gaaggaagaa acgggaagaa attcagactc cttcctgagt 5040
tccgccaacg cctagtgaca tcctgtgcac accctaaggt ggcctttgtg tggcactggc 5100
ttgggtggtc gggaaaggca ttttcagctt gttgcagaac tgccacagta gcatgctggg 5160
tccgtgaaag tttctgcccg ttaacaagaa gtctctacta cttgtgacct caccagtgaa 5220
aatttcttta attgtctcct ggtgttctgg gttttgcatt tttgtttcta aggatacatt 5280
cctgggtgat gtcatgaagt ccccaaagac acagtggggc tgtgttggat tgggaaagat 5340
gatttatctg gggtgtcaaa aggaaaagaa gggaaacagg cacttgggaa aatgtcctcc 5400
cgcccacccg aattttggct tggcaaccgt ggtggaggag caagaaacac gtggacgttt 5460
gaggaggcat ggggtcctag gaggacagga agcagaagga gagagctggg ctgacagcct 5520
gcaggcattg cacagtttca gaaggagatt acagcatgac tgagttttta gggatccaac 5580
agggacctgg gtagagattc tgtgggctct gaggcaactt gacctcagcc agatggtatt 5640
tgaataacct gctcttagag ggaaaacaga catagcaaac agagccacgt ttagtgatga 5700
aactctcact ttgcctgagt catgtgcggc catgcccagg ggtcaggctg acactcaact 5760
caaaaacaag tgagaaattg aagacaatcc gtggtggcag ctactggaag ggccaccaca 5820
tccccagaaa gagtggagct gctaaaaagc catttgtgat aggcacagtt atcttgaatg 5880
catggagcag agattacgga aaaatcgaga atgttaatga ggcaacattc gagttgagtc 5940
attcagtgtg ggaaacccag acgcttccat cccctaaaag gaacatcttg ctctcagtca 6000
aaatggaaat aaaaattggg gcttgaattt ggcaaatgat tcagaactct gtgtaggtat 6060
tttcacacgc acagtggata attttcatgt tggagtttat ttgtgctaaa aggcagaaaa 6120
gggtaaaaag cacatcttaa gagttatgag gttctacgaa taaaaataat gttacttaca 6180
gctattcctt aattagtacc cccttccacc tgtggtaatt tcctgagata gtcagtgggg 6240
aaaagatctc tccttctctt ctttctcccc ctcccctcct ctccctccct ccctccctcc 6300
ctccctcctc tccctccctc cccctttcct tctttctttg ctccttctcc tctgcctcct 6360
tctccctttc ttcttcattt attctaagta gcttttaaca gcacaccaat tacctgtgta 6420
taacgggaaa acacaggctc aagcagctta gagaagattg atctgtgttc actagcgtgc 6480
aattcagagg tgggtgaaga taaaaggcaa acatttgagg ccatttcctt atttggcacg 6540
gcacttagga agtggaacat gcctaatcta ctggtttgta ccacctttcc ctataatgga 6600
ctgtttggga agctcctggg caaccgattc tggcatctca ttggtcagag gcctgttaaa 6660
tggtactctt atttgcaaag aaggctgtaa cttgtagctt taaaagcctc tcctcaagaa 6720
agaagggaga aaggatatgg ctagacatat ctaatagact taaccactgt gaaaagcctt 6780
agtatgaatc agatagaacc tatttttaac tcagttttga aaaaaataat ctttatattt 6840
atttgtgtgt gtgtgtgtgt gtgtgtgtgt gtgtgtgtgt gtgtgtgtgt gaaccacatg 6900
tagcaggtgc tggaggaggc cagaagaggg caccagatct cctggaactg acaccacaca 6960
tggttatgag ctgcctgatg tgggtgctgg gaactgaact ctcgtgttct gcaagagcag 7020
caactgttct cttaactgat gagccatctc tccagccccc cccataattt taattgttca 7080
ttttagtaaa ttttattcat aatcaattat cacagtataa aacaatgatt ttatatatat 7140
catatacata tcaaggatga cagtgagggg gatatgtgtg tgtgtgtgtg tgtgtgtgtg 7200
tgtgtgtgtg tgtgttattt gtgtgtgtgc tttttaagaa ggtgccatag tcactgcatt 7260
tctctgaagg atttcaaagg aatgagacat gtctgtctgc caggaaccct atcttcctct 7320
ttgggaatct gacccaaatg aggtattctg aggaactgaa tgaagagctc aagtagcagt 7380
gtcttaaacc caaatgtgct gtctagagaa agtcaacgtc atcagtgagc tgaggagaga 7440
tttactgagc ggaagacaag cgctctttga tttaagtggc tcgaacagtc acggctgtgg 7500
agtggagcct gtgctcaggt ctgaggcagt ctttgctagc cagctgtgat gagcagtgaa 7560
gaaagggtgg agatggaggc agggtgggag cagggctatg gttcagacta ggtatcgtga 7620
gcacaccagc tggttgactt gtggtctgtg ggtcaggcgt tgtaaacgcc ctcagggtca 7680
ggcagtcaca ttgcttgaag ctgaatgggt gaggcaacac agagagtgca aagaaggcaa 7740
agtaccacct cttccccgac ccaggtcact tctgggttat agctgagact ccggacagca 7800
tgcaaccagc tggttagagc ttcagggaaa acttgatgtc tgcatgttgc tatgaaatgt 7860
gattcggtac atctggagaa aatttataat gctggctcag tcaagcactg aacaaaggta 7920
ccttggcttt gggagctaca tgacattgac ttgtaggcag actttttttt ttctgcccgc 7980
caattcccag ataaccaata tggaggctca atattaatta taaatgctcg gctgatagct 8040
caggcttgtt actagctaac tcttccaact taaatgaacc catttctatt atctacattc 8100
tgccacgtga ctttaccttg tacttcctgt ttcctctcct tgtctgactc tgcccttctg 8160
cttcccagag tccttagtct ggttctcctg cctaacctta tcctgcccag ctgctgacca 8220
agcatttata attaatatta agtctcccag tgagactctc atccagggag gacttgggtg 8280
ctcccccctc ctcattgcca tccgtgtctt cctcttccct cgcttccccc tcctcttcct 8340
gctcttcctc ctccacccct cctttcatag tattgatggc aagggtgttc tagaatggag 8400
gagtgcccat aggcatgcaa agaaaccagt taggatgctc tgtgaggggt tgtaatcata 8460
agcgatggac acaattcaag ccacagagtg aagacggaag gatgcactgt gctctagagc 8520
aacttctggg gcagaatcac agggtgagtt tctgacttga gggcgaagag gccacgagga 8580
agggagtgag tttgtctgag ctagaagcta cggcccacct cttggtagca gacctgccca 8640
caagcatgct ttgttaatca tgtgggatct gattttcctc taaatctatg ttcaactctt 8700
aagaaaatgt gaattctcac attaaaattt agatatacgt cttttggtgg ggggggtgta 8760
aaaaatcctc aagaatatgg atttctgggg gccggagaga tggctcagag gttaagagaa 8820
ctggttgctc ttctagacat tctgagttca attcccagca accacatggt ggctcacaac 8880
catctgtaat gcgacctggt gccatcttct gacatgcatg gatacatgca ggcagaaagc 8940
tgtatacata gtaaattgat aaatcttttt ttaaaaagag tatggattct gccgggtgtt 9000
ggtggcgcac gcctttaatc ccagcactct ggaggcagag gcaggtggat ctctgtgagt 9060
tcgagaccag cctggtctat aagagctagt tccaggacag cctccaaagc cacagagaaa 9120
ccctgtctcg aaaaaccaaa aaaaaaaaaa aaaaaaaaaa aaaaaaaaga gtatggattc 9180
taagaaagcc gtaacagctg gagctgtgta cggagttcag cgtggtacta gaagaacaga 9240
cattcatgat gaaacacccc aggattttta cttagtatct agtttccatt gttgttttga 9300
gaccggctct tatgctctcc aggctggcct caaactgctg atcttcccgc ctctacctct 9360
caagtcctgg gactacttgg ctcataaaac agtttttgtc gggctccctg aagttatggt 9420
tgtacaaacc gtgggggtca atatactcac ttgggcagag agagaaggtc tgaatcccag 9480
acaatgactg catctcagga cagttgggaa gaggacaatg gcagaaggac ttagaaaaga 9540
tagactggag ggtggaaaag cagcaggaac agagaaacaa aacaggaagc ttgctatcca 9600
gggccactct ggagtcctgt ggcaagatgg aagcgggcta ggggaataca tttgtgctac 9660
tgtgtgtgtg tgtgtgtgtg tgtgtgtgtg tgtgtgtgat caatgcctat caatgttgaa 9720
ggggaaatat gtataccaca ttgattctgg gagcaattct cagtatctgg cctagagaaa 9780
ggaatggccc ctgcagaata gacagagtga atggtgccct ttatcatttg ctaaagtgaa 9840
ggagaaataa acatccttcc atagagtttc aggtaaatga accccacagt tcatctgtgc 9900
cgtggtggag gcctggccaa cagttaaaaa gattagacac ggacaaagtc tgaaggaaac 9960
acctcgaata ggaagaggag agccacctca ttctgtaact ttcctcaagg ggaagatgtt 10020
ccaagagtgg gaataaatgg tcaaaggggg gatttttaat taggaaaacg atttcctgta 10080
tcacttgtga aactggaggt tgatttgggg cataggacaa tagatttgat gctttgcaaa 10140
aagctgtttc aaagcagaga aatggaatag agacaattat gtagcgagga gggagggtgg 10200
ggcgaagatg gagacagaga agtggaagct gactttaggg aagaggaaca tagaccacag 10260
gggcggggcg gggggcaggg gcggggggcg gggctcaaag gaggcagtgg gaacgttgct 10320
agtgttcgca gcgtaagcgt gaatgtgcaa gcgtctttgt ggtgtgtgac caggagtagc 10380
gtggctggct tgtgtgctgc ttgtaatccc agtctttgag gtttccacac tgttccacag 10440
tgggtgtgat tttccctcgg agagcatgag ggctctgctt tccccacatc ctccccagcg 10500
ttcgttggta tttgtttcca agatgttagt gggtgagaca aagcctctct gttgatttgc 10560
ctttaacagg tgacaaaaaa agctcaacca ggagacattt ttgccttctt ggaaggtaat 10620
gctcccatgt agagcaatgg gacccatctc taaggtgagg ctactcttgc agtttgcacc 10680
cagctcttct gatgcaggaa ggaagttggt gggcaagcaa gactgtttgc ttcttgcgat 10740
ggacacattc tgcacacaaa ggctcaggag gggagaaggc tgtttgatgt ttagcactca 10800
ggaaggcccc tgatgcatct gtgattagct gtctccatct gtggagcaga cacggactaa 10860
ctaaaaacca gtgtttttaa attgtcaagc ctttaaggtg aggaaattga cttattgtgc 10920
tgggccatac gtagagcaag tgctctgcat tgggccaacc cccggctctg gtttctaggc 10980
accagaatgg cctagaacta actcacaatc ctcccattcc aggtctcagg tgctagaatg 11040
aaccactata ccagcctgcc tgcctgccta cctgccttcc taaattttaa atcatgggga 11100
gtaggggaga atacacttat cttagttagg gtttctattg ctgtgaagag acaccatgag 11160
catggcaact cttataaagg aaaacattta gttgggtggc agtttcagag gttttagtac 11220
attgtcatca tggctgggaa catgatggca tgcagacaga catggtgctg gagaaaggga 11280
tgagagtcct acatcttgca ggcaacagga cctcagctga gacactggct ggtaccctga 11340
gcataggaaa cctcacagcc caccctcaca gtgacatatt tccttcaaca aagccatacc 11400
tcctaatagt gccactccct atgagatgac agggccaatt acattcaaac tgctataaca 11460
ctttaaagta ttttattttt attattgtaa attatgtatg tagctgggtg gtggcagccg 11520
aggtgcacgc ctttaatccc agcacttggg aggcagaggc agatggatct ctgtgagttc 11580
aagaccagcc tggtctataa gagctagttg caaggaagga tatacaaaga acagttctag 11640
gatagccttc aaagccacag agaagtgctg tcttgaaaac caaaaattgt gctgggacct 11700
gtctctgctt tggttgcttc ccactccccc agagctggac tcttggtcaa cactgaatca 11760
gctgcaaaat aaactcctgg attcctctct tgtaacagga gcccgaagtc aggcgcccac 11820
ttgtcttctc gcaggattgc catagacttt ttctgtgtgc ccaccattcc agactgaagt 11880
agagatggca gtggcagaga ctgggaaggc tgcaacgaaa acaggaagtt attgcaccct 11940
gggaatagtc tggaaatgaa gcttcaaaac ttgcttcatg ttcagttgta cacagactca 12000
ctcccaggtt gactcacacg tgtaaatatt cctgactatg tctgcactgc ttttatctga 12060
tgcttccttc ccaaaatgcc aagtgtacaa ggtgagggaa tcacccttgg attcagagcc 12120
cagggtcgtc ctccttaacc tggacttgtc tttctccggc agcctctgac acccctcccc 12180
ccattttctc tatcagaagg tctgagcaga gttggggcac gctcatgtcc tgatacactc 12240
cttgtcttcc tgaagatcta acttctgacc cagaaagatg gctaaggtgg tgaagtgttt 12300
gacatgaaga cttggtctta agaactggag caggggaaaa aagtcggatg tggcagcatg 12360
tacccgaaat cccagaactg gggaggtaga gacggatgag tgcccggggc tagctggctg 12420
ctcagccagc ctagctgaat tgccaaattc caactcctat tgaaaaacct ttaccaaaca 12480
aacaaacaaa caaataataa caacaacaac aacaacaaac taccccatac aaggtgggcg 12540
gctcttggct cttgaggaat gactcaccca aacccaaagc ttgccacagc tgttctctgg 12600
cctaaatggg gtgggggtgg ggcagagaca gagacagaga gagacatgac ttcctgggct 12660
gggctgtgtg ctctaggcca ccaggaactt tcctgtcttg ctctctgtct ggcacagcca 12720
gagcaccagc acccagcagg tgcacacacc tccctccgtg cttcttgagc aaacacaggt 12780
gccttggtct gtctattgaa ccggagtaag ttcttgcaga tgtatgcatg gaaacaacat 12840
tgtcctggtt ttatttctac tgttgtgata aaaaccgggg aactccagga agcagctgag 12900
gcagaggcaa atgcaaggaa tgctgcctcc tagcttgctc cccatggctt gccgggcctg 12960
ctttctgcaa gcccttctct ccccattggc atgcctgaca tgaacagcgt ttgaaatgct 13020
ctcaaatgtc actttcaaag aaggcttctc tgatcttgct aactaaatca gaccatgttt 13080
caccgtgcat tatctttctg ctgtctgtct gtctgtctgt ctgtctatct gtctatcatc 13140
tatcaatcat ctatctatct atcttctatt tatctaccta tcattcaatc atctatcttc 13200
taactagtta tcatttattt atttgtttac ttactttttt tatttgagac agtatttctc 13260
tgagtgacag ccttggctgt cctggaaccc attctgtaac caggctgtcc tcaaactcac 13320
agagatccaa ctgcctctgc ctctctggtg ctggggttaa agacgtgcac caccaacgcc 13380
ccgctctatc atctatttat gtacttatta ttcagtcatt atctatcctc taactatcca 13440
tcatctgtct atccatcatc tatctatcta tctatctatc tatctatcta tctatcatcc 13500
atctataatc aattg 13515

<211> 14553

### <212> DNA

<213> Mus musculus
<400> 6

(SEQ ID NO: 57)
cttgaagaac acatgttttc caagagggag cacccatgtt ggaatgacaa tgtagttagt 60
gctcctctcc tgtaggttag tgctcctttg ctataggtaa gtgctcctct cctataggtc 120
agtgctcctc tcctataggt tagtgctcct ctcctatagg ttagtgctcc tctcctacag 180
gttagtgctc ctctgctcta ggttagtcct gctctcctat agtacctaga gagctagggc 240
aaatgggcta ggcccgaagt gcagagacaa acagctatgg aagactgggt aagcacttcc 300
aagctacgaa agagcagtgt gaagggtcag ggcttgtgca gttagtaggg gagatcttcc 360
agttgaagaa acagaagaac tgagagccac tgggtatcat cctcctgcgc catgccttcc 420
tggatactgc catgctccca ccttgatgat aatggaatga acctctgaac ctgtaagcca 480
gccccaatga aatattgttt ttatgagagt tgccttggtc atgctgtctg ttcacagcag 540
taaaacccta aataaggcag aagttggtac cagtattgct gtgatagacc tgaccatgct 600
ttcctttgaa agaatgtgga tttggtgact ttggatttgc aacacagtgg aatgctttaa 660
atggagatta atgggtcatc aattcctagt aggaatatgg aagactttgt tgctgggagt 720
atttgaactg tgttgacctg gcctaagaga tttcaaagga gaagaatttc agaatgtggc 780
ataaagacag tttttgtggt attttggtga agaatgtggc tactttttgc ccttgtctga 840
aaagtctgcc tgagactaaa gtgaagagaa tcagattaat tgcattgaca agggaagttt 900
gtggctgcgc tatctggaaa cttacagcca gcctcttgga cctcgggtga cttacgcaaa 960
tactcaggga cagagatgct tgactctgta ctgatgagtt gtcttggatg caaatatggg 1020
ctcttcattt gactacatgt cacgatgagt caggagctgc tctctccaga gtgtgacaaa 1080
gcgaggggat gctgacggta gctgttctag ctttgaaggt aagcctgcac ttatgctaaa 1140
gtcacacata cacgagccgg gtggagaacc tgtctgtgtg gagacacctt tcattacctg 1200
tggcatccag cctctcaagc ttggactgcc tgtgtgctcc tggactctgg aggtcccact 1260
gctctgtcct ctgctgctta tgatactgac attttaaaag aatccagtgg ttcccccctg 1320
tactcggtgt ctacttctac ctggatgttc ctcatttatg ttctgtgaca cttctctgtg 1380
actctgctgc attcctgggt gacatgtgga caccctgtcc ctttgcagac catgatgtca 1440
ctgtcactag tggaatcaga tgccccaagt gttgtcctgt gtttgggaac gtgacaggca 1500
gtacagaagc agaagaggaa gggtgaaaac ggaaatgtca cagcagcatc tgatgtgtgc 1560
ctcagtcacg catgctgctg attggaacta ctcagcatga gagagggcca tggtgaatac 1620
acaaccctat acacactgtg tccatttctc tctctctctt acacagagag agagggagga 1680
gggggagggg gaggcggagg gggaggggga gggagaggga gtgggagagg gagagggaga 1740
gggagaggga gagggagagg gagagggaga gggagagttt aatgtctgtg aagagatacc 1800
atgaccaaag caactcttat aaaggacaac atttaattgg ggctggctta caggttcaga 1860
aattcagtcc attctcacca tggtgggaag catgcaggta gatgtggtgc tggaggaacc 1920
aagagttcta tatcctgatc tgaaggcagc caggagaaga ctgcctcttc tgcacagggc 1980
agagcttgag catagaacat caaagccctt ccccacactt cctccaacaa ggtcatacat 2040
acttcaacaa agacacacct cctaacggtg ccactccctg tggaccaacc atttaaacgc 2100
atgagtctat gagggtcaaa gctcttcaaa ccaccacact catgtacaca cacacacaca 2160
cacacacaca ctctcataca cacacacaca cacactcaca cacacacaca cacacacaca 2220
cacacacaca ccacacacac acacacacac agagttctat tttgcactgt ttcactgtca 2280
caaggttcta cttatctcag acacactgcc aggaattgtg tgggaagact ttcagtttct 2340
ttgggttcac atggacttag cagttcttgg tgatcctgaa agatttctgc agaaagaagc 2400
caaagtgttg agcccaaggc ctggccacac attagtcctg tctagatgaa caggggttta 2460
aaaataaggg ggcatcaagg tgaagccagc aggggctgac ttagagagga gacccaccca 2520
agccaactgc tcgaagtcaa aagcgatgaa tccccatatc cagctgtgcc cggtgctgtc 2580
ttgctacatc tttagtaaat gttcttttag ttgtatgcgt atgaatattt tgcttgcata 2640
tatttgtgta caccataggt gttcctaggg cctatggagg ccagaagagg gcatcagatc 2700
ctttggaact ggaattatag acacttgtta cccatagagt agattgtggg aaatgagcct 2760
ttagtcttcg agagcggcca gtgctcttaa cctttggtcg tttctccagg tctttgagac 2820
tttattttct tggacatcag gacaggatcc agggctttga gcttgtttct tcagccagct 2880
ttcttttcat gtatattaaa ttttatgtta ttttgctttc tttttcccca agacagaatc 2940
acactctata tagctcaggc tgggtttgaa ttcagtttcc ctgtctcagt ctaccgggta 3000
atatgattac agatgtgagt ctgactttgg tatcaaagtc cccagccctt ctggatatgt 3060
gttttaagga tatcagatat atccttgatt tgctttgaat tttcttttta gttacaacat 3120
aattagttcc gtgtcacctg aatatgtgta tgtcacctac atagtcttcc ttcttctctt 3180
cttccctctc ccaccttccc aggtacctgt ctgtcttcat atccttgtgc tgagagtctt 3240
gttgagggag atgatgaccg agacagagcc actggggaag ggagatgggc tagtgcaggt 3300
cttcagagag gagctcgtga atattgtagc ccctttagtc cctggcatgt cctcttgtat 3360
agccaccgcc atgctgtggc ctggcagaag tgaataagtt gtccagctgt tgacaggcct 3420
gccctccaga cccagtctga tcccaagaaa gggcatctgt gtctgtctct gaggccgtaa 3480
gtgctgcctg gttgtctcca gcttgacttg acactccctc cttaataaga gtaccacaga 3540
acagggtctg cagagtccct gggccaggtc cctgtgctgt cctggaatgc caggcgtgaa 3600
tttcctgtga agtaggactt tgctcgccaa gctcccacgg cttgcccttc agatagccag 3660
aattatctgg taccctgcat tgccgttcaa tacgcagagt atcactggaa gcgcgcgcgc 3720
gcacacacac acacacacac acacacacac acacacacac acacgcccac tccatcttta 3780
aaccccaccc cccagcaacg gcggtgtaaa cactctccat caggaagctg aaacgcagtt 3840
gccctctgct ggggagatga aggcagcttg ctgggggcga ggaccgtgct agcaaccttc 3900
cctggtgcac acgggctctg gtgcatgacg ggaacggaaa cgcggaacta aagtcagtcc 3960
tgcttttttt tttttttttt tttttttttt tttttttttt tttttttttt ggcgttggtg 4020
gtggactgag tgacaatcag tgaaatcact taggttgttt ttctcttctt cgttgggttt 4080
gatagacggt gggagagggt cagaggagaa ggggagggat ggggagagag ggaggaggga 4140
ggggcgggag gcggggggcg aggaaaacgt gctaacttct ccaatcctac aagacaaagg 4200
tttggagaaa gccgcactga gtgacccagc agaaggaatc caggaatgtc cgctggaatc 4260
tgactgttga ttccagcgcc atgcagagaa tctaggctgg taggaacatt ctttgtccta 4320
tccgacataa taactccaac caacacggaa aagaaaggct atacaagtga agaaatggca 4380
ttttcacttt catgactata caatcacttc caggtagtaa cacgtgtcta gcacagcggt 4440
tctcaacctg ggggtcacga tcccccactt ttctgcatat cagacatttt tacgttgtta 4500
ttcataacag tagcaaaatt gcagctatga agtaacaatg aaatgcattt atggtgcgtg 4560
tgtgtgtgtg tgggggggta tcaccttaac atttactgta agaaggttga gaatactgct 4620
ccagcagcta gtgtgttgga cttaggttct gggtatatta ttagcaatag ccaaccagaa 4680
tccccaccca ccacagcatt gaggccccat gcagggcttg ctgggagagg cactgataag 4740
acttctttat gtatttattt agagacgaat actcattagg taggccaagc tagcgtcaaa 4800
ctcatggcaa ttctcctcct ccagtttcct aagtactgga ctcaggagtg tgttgccatc 4860
atatacagta aggatttatt gactgaagaa aatctcaagt ggctttggtt aatccctact 4920
acgccagagg ctgaggcagg aggcgcgcaa ggtcaaggct tgcctgggct acatatagag 4980
tgagctcaat tttgacactt ggtgcggtgt tagtagtaat agtaaagatg aaggtgtggc 5040
tcaggtgggg ccggtgattg gacacacttg gggtctcctg gtccatctgc agctgtgcaa 5100
caggaagagc ggagaatgag aggaaagaga gaaaagacag aatgagagag agggaggaag 5160
agagaaaaag gaaaagagag aggaaaggaa aaaggaaaat gaggaaagcg agaaagaaga 5220
aatgagaaag aggaaaggga gaaagaaatg agagagagaa aagaaaagac agaatgcgag 5280
agagggagga agagagaaaa aggaaaagag agaggaaagg aaaaaggaaa atgaggaaag 5340
cgagaaagaa gaaatgagaa agaggaaagg gagaaagaaa tgagagagag aaaagaaaag 5400
acagaatgcg agagagggag gaagagagaa aaaggaaaag agagaggaag ggaaaaagga 5460
aaatgaggaa agcgagaaag aagaaatgag aaagaggaaa gggagaaaga aatgagagag 5520
agaaaagaaa agacagaatg cgagagaggg aggaagagag aaaaaggaaa agagagagga 5580
agggaaaaag gaaaatgagg aaagcgagaa agaagaaatg agaaagagga aagggagaaa 5640
gaaatgagag agagaaaaga aaagacagaa tgcgagagag ggaggaagag agaaaaagga 5700
aaagagagag gaagggaaaa tggaaaatga ggaaagcgag aaagaagaaa tgagaaagag 5760
gaaagggaga aagaaatgag cgagataaaa gacagaattt gagagaggga ggaagaaata 5820
ggaaaagaga ggaaaggatg gagaaaagag agaaagaaag agagatgaaa gagagaaagg 5880
agaaatgaaa tgagagagag agagagacac aaagagccag agagagaaga aaaaagggga 5940
aagagaaaga gaaagaggaa ggctcctctt ggacacatct tcctttatct ttccctgggg 6000
accgccaaag cctggtggca tactgtacat tctgtacact gttcattcaa aacaggctct 6060
gtcttaaaga tggtctgagc ggtcagaaaa gggtattgtt aacttgtttg caaaactgcc 6120
tcaggagagt gctgagtgcg tgaaagttgc tgcccgttaa ggagaagtct ctactacttg 6180
tgatctcacc atcgaaaatt tctttaattg tctcctggtg ttctgggttt tgcagttttg 6240
tttctaagga tacattcttg ggtgatgtca caaagtcccc aaagacacgg tggagctgtg 6300
ttagatgggg aaagacagtc tgctgaggat ttatctggaa ctgtcagaag gaaaagaagg 6360
taaatggggc acttgggaaa gtggcctcta gtttgacttc tggcttagca aaggttgtgg 6420
ggagataagg catacacagt agttagcagg aggcaacagg gtcctgggag gacgcgaggc 6480
agaaggagag gctgggctga cagcatgcaa tcattgcata gtctccaaag gagattgcaa 6540
catggctgag ttttcagagg tcctacagag cccgtggtag agattctgtg ggttctgaga 6600
caacttgact ttagccagat ggtatttgag taatctggga gagagaaaac agctacagca 6660
aacagggcca catttagtga cgaaactctc actttgactg ttgagtcatt tgcagtgggc 6720
cctgaggtca ggctggccct cagctcaaaa acaagcgagg aactgaagca attactcaga 6780
taatccacag ccacagccac tggaaagggc cacatcccca gagacagcac agcaggggtg 6840
ggggtggggc tatgagaaag ttagtgattg tagcagttat ctagaatgtg cggagcagag 6900
gaggttacac aaaaacctag aatgtcattc aatgtgggaa accgagaggc tcccaagccc 6960
taaaaggaac agtttgcttt cagccaaaat ggaaataaaa tttggggctt aaatctggca 7020
aatgattcag accttctgtg taggtgtctt taaatgcaca gcagattgat tttcatgttg 7080
gagtttattt gaactaaaag acagaaatgg tgaaaagcac acctgaagaa attgagatgc 7140
tatgaataaa atcatttact tacagctatc acttaattag tacctccttc caccttgctg 7200
atttattggg ctagtcaagg aagaaaagat cttccctcct ccttctctcc tcctccccct 7260
cctctcctcc tcccctcccc tccttgacct tcctctcctc cttttccctc ctccccctct 7320
tcttctcttc accccctcct cccctcccct cctctgtact cctccccttt cctcccaatc 7380
tcttttttct cccccttctt ctctttctcc cccctcctct tccctcctct tcctccctcc 7440
ctccctcctc ctcctcatcc tcctcttcct cttcatcctc ttctccttcc tccctctcct 7500
cctcctcctt ttccagccct acctaccttc cctttcttct tcatttattc aaagtagctt 7560
tgaacagcac tactcggttt agttgtgtat aaaaggaaaa tgcaggtcca agcagcttgg 7620
ggaagattgc tttttgctct ctggaggcag atgatgacag ttcaagatca ttccttttgc 7680
tccatgtcac aggaaggggg acatgccgaa tctaccagtt tgcagccacc tacacaggat 7740
ccaccttcac ttctaaggaa atgtttggga agctacctac caaccacttc tggcatctca 7800
tgggctagag gactcttaaa tggcactctt atttgtttaa taaaggaggt tgtgacgtgt 7860
agttttaaat cccttccaca caacaattgc tactctctga ccaaaaaaga agggagacag 7920
gatacggcta ggtgtctagt agactttacc actttgaaaa gccttaatat aaatcaggta 7980
gatacatctt tttaacttat tcttgtaaag acaaaaacaa aactttattt ttatttgtgt 8040
gtatgcttgt gtgtgtgtgc ctgtgtgtat accacatgtc gctggtgccg gagaacacca 8100
gaagagggga cctgatctcc tggagctaaa gctatccatg gttctgagct gcctgatgtg 8160
ggtgctggga acagaactct ggtcttctgc aagagcaaca agcctcctct taactacgaa 8220
tctcctcccc atccccccaa atacatttaa ttattcattt tagcagcttt atttcgtaac 8280
tacttatcac agcataaaac aaggatttta tatatattac atgcaatcga ggataagagt 8340
tgaggggaga tgcgtgtgct ccttctgggt gtctgtgctt ttgaagaatg taagcagtgc 8400
acaagggacc gaggcgtgcc tgtctgccag gagctgtctt cttcccttgg actctgagct 8460
gagtgcagtg ctccgaagaa gtaaaagacg acctcatgaa gcaatgtctt caacccaaac 8520
atgctgtcca gacaaagtcc agcttcatta gtgctctgag gagagactta ctgagcctca 8580
ggaaagcccc cctcagcatg gcgaaagtcc actttgattg aagtgactcg aaagccatgg 8640
cagtgcggcg gcggccgcgt ggagcttgtg ctcgagtcgg aagcggcatc tttgtcaggc 8700
ggctgtgatt agcacgggga ggcaggactg gagtgaagga agagttgggg gcggggctta 8760
gcgctctggt ctcctaagct gtagtcagcg cctcaagatt tgtaacctgc cttctgcctt 8820
cccagccagg cagtcaagtg gctccaagct gaagactgca aagtgcccct aaccttttgg 8880
ttatagcgag gctgaagaca ccgtgctctt tcatgaaagc cggatgtctg aaatccgatt 8940
tgataaatat ggataaaacg tataacgctc gatcaatcga atcgaaggag ctcacgattg 9000
gcaccacggc tttggggaca acagagtact gactcgttgg gaggacttgg atacttcccc 9060
tcctcttcca tctcttcccc tttcctcact tcctcctcct tccttctcca ttttctccct 9120
cttcactgtt tcttactatt tttacaaaag attttattta tttatttatt tatttattta 9180
tttatttatt tatttattta tttatttaat gtatgcgagt acactgtagc tgtcttcaga 9240
cacaccagaa gagggcgtca agttccatta gagatggttt cgagccacca tgtggttgct 9300
ggggcctctg gaaggaccgc cagtgctctt aacccctgag ccatttctcc agtacccttc 9360
tcaccgtttc tcttcaatct tcttcctctt ccttctccac tttccttgtc ttcttggttt 9420
cattatcttt ctccctttct tcctcttctc cccttcttcc tcctccactg tagttttcct 9480
tccctactct tttcctgcct ccctcctcct cccctctcat tccccctcct ctttcctcct 9540
tctccctcct cctccttcct tctccctctc ccctctcccc tctcccttct cccttctccc 9600
cctcctcttc ctctttctcc ttctccaccc ctcctgtcac agtatcaatg gcaagggtgt 9660
tctagaatgg aggagtgtcc cctaggcact aacgaaagcc agttaggatg ctctgagacg 9720
ggtacaattc agggagggcc gtggggatgg aagggttgtg ctgcgattca ttctggagca 9780
acccccaggc agaatcatga ggttggttcc ggattcgcag ggcacaattc agaagaggaa 9840
ggtttcagga aggacgagtt tgtctgagat aggagttaca tctgatgtct tggcagcaga 9900
gccactgtac aagcgtgctt tattaaccac gtgggattaa atcttctttt aaatttattt 9960
tcaactctta aggaaacgtg aactttcaca ttcaaattta gacttgcagc tcttatgggg 10020
aaaaaaaggg gatcttaaga atattaagca taggcggctg gagagatggc tcagcggtta 10080
agagcactct ctgctctccc agaggtcctg agttcaattc ctagcaacca cataatagtt 10140
aacaacagtc tttaatgaat tctaatgccc tcttctggtg tgtctgaaga cagttacagt 10200
gtactcatat aaataaaata aagaaattta aaaaaatgaa tattaggcat agattcctgg 10260
atcctaagaa agccatcaga gctggagcca tgtgtgggat cctgcttggt gctggagggg 10320
cagagttcat gcccccgggg tttttactta ttatcacatt ttcatcgttg ttttgaaaca 10380
gggtcttgtg tggtccaggc tggccttgaa ctcatctttc agcctctacc tcacaggttc 10440
tgggattact tggttcctaa aagtatctcc gtcaagctcc ctggtgttat ggctgtgcca 10500
accaggaggg tctatacact cgctcaggta gagggagaag atccgaatct ctgacaggga 10560
ctgctgcctc tcggggcaaa tggagtgaag gacagcggca gaaggattta ggaaagatgg 10620
acgggagagt ggaaatgctg cagaagccag aaaacaaagc aggaagcctg ctgtccagtg 10680
gggctcaaga gcggagggat gcgagggggc tgcgcaggaa catttagcgt ctgcgtctat 10740
gggggtaggg gcggggtgcc agcacctagt cacctgaagg ggaaatgctt gcccagggag 10800
caggtctcag tagctgacct agagaaagga gcggccccta cagaggagac acgggtcact 10860
gtttgttaaa gtgaaggaga aataaatatt ctttcaaaga atcttaggtg agcccagttc 10920
atctgcgctg tggaggcctg gggaacagtt aaaaagaccc tgacacacac ccaaggcaaa 10980
caagcaacac acggctcctt ccgtaagggt ccatgattct ctgaagaatc agccccggaa 11040
tcagccccgg aatcaggtag tccgtaaaca caatgagtgt tttactctgc agaagtccag 11100
cctgctggcg tctcccatta ccaaaataga gggatagtca cgtgagctca ccggctcgat 11160
ttaaggcacg tggttttcca gggtagatga gctttggctt ctggaaccat tatggggcac 11220
gaaggatgga gccaggattt tttttttttt tttttttttc tattagcaat tgatttgctt 11280
gggcttggct ggacttgccc agttcttagg cccagtcttc ttaactgccg atctgaagtc 11340
tgtcatggag tcagcctagc cttctcactt cccttcagct cgaataggaa gaggaggtgc 11400
acaccagatg gtctgagagc agggataaat ggtgtgcctt tgtctttcag tatttcgtta 11460
ttttaagtag gaagatgctt ttctgtatta cattgcttgt gaaaccggaa gttgattcgg 11520
ggcacaggac aatggatttg gtgttttgca aggactgttt cagaagagag aggagtggaa 11580
gggtggttag agtgaggagt ggggtgggac gggatggggg aagagaagga agggccagac 11640
aggctaggta gggctgagag gaggcggtgg gaacttcttg agttagcgca gcagtaaact 11700
tggatgtgcg tgtatctttg tgatatatga cccggagccg tgtagctggc tccgatagta 11760
ctgctaatgt cagtgtcggg gggggggggt cccatactgt tccacagggg ctgcacattc 11820
ccatcgagag caggagggct cctctctcca tacatcctcg ccagcattcc ttgttgtttc 11880
tgtgatgaca gggggtggga tgaaatctct ctgttggttt gagagaccgt gaagaagctc 11940
aaccccagga cattttgcag tcttggaagg cagtgcctcc atgtggagcc gtggagccca 12000
tctctgagtc caggtcactc ttgcagttcg cactcagctc ttcagatgca ggagagacgt 12060
tggtgggaaa gcaagattgt ttgcttgttg agatagacac attctccaca caaaggctca 12120
cgtggggcaa aggctgattg acgtacagcg ttcaggaacg cctgtggtag agctatgatt 12180
agctgtctcc atctatgaag cagacaaaga gttataaaaa aaatcaatgt tttcaaattg 12240
tcaaactttt aacccgacag caagcgctct gtccctgggc taatccctag ccctggtttc 12300
ttgagatggg gtcttttgtg cactagactg gcctagaact cacgatctta gtgttccagc 12360
ctcccagctg ctgggatgag ccgctataac cagtctgcct gccttcctaa attttaagtg 12420
atgggaagtg ggggagaata cagtttaaag tatgcagatc tgagagcagg aacctggcaa 12480
agccaagggg ccggagttac aggcggctaa catgggtgct gggaactgac ccaggtcctt 12540
gagaggagca gtgtgtactc ttgaccaaac aggtccgtct ctccagtccc cgtagtatta 12600
aaaataggta ctacgggcat ggtggtgcac acctttaatc ccagcactag ggaggcagag 12660
gcaggtggat ttctgagttt gaggccagcc tggtctacaa aatgagttcc aggacagcca 12720
cggctataca gagaaaccct gtcttgaaaa caaaacaaca acaaaatagg tactacaaag 12780
cgatgtaatt gtgctcaaac atgcaaaccg aggggactgt atgcataaga aagagaaaga 12840
cggccacact ggttctatct gggtgacagg aaatcagtat ttttattttt cacattcatt 12900
tttttgttgt tgttgttgac acagtgattt ttctatcaaa aacattattt cttttatagt 12960
tcccctgagg agctgttttt aaagccgtgc tttgaaaaac cattgaagga gcagaggcag 13020
ggagactcct gtgtggcagt cggtgaagca ggccctctgc aggcaggctg gccctggact 13080
tgggagtctc tttccctccc tcctgtgctc aaatagcaaa tgtcaggctt caatgtagct 13140
agaaggttct agaatgatta agtttccaag gctgaagagc ttccctgttt gcctttcact 13200
tccctggaga ggtcgttgtg tgttccggag tctgcaaggt gcctttggtg atgcgggtgg 13260
ttcatctcgg gagattccgc ctggaggacc caagttcaag ccctgcctga gctacagagt 13320
gactttcagg tcttctgcgc aattcagtga gacccagtct acaaataaaa agtaaaaaga 13380
aggctgtgga tggaactcgg tggtagagtt ctgggtttac tccctagagg aggggagaag 13440
gaggaggagg gaggaggaag aggaagaaag aagaagagaa gggaagagga gaaggaaggg 13500
agggaagggg ctgacaagaa gagagaagag ggagggaggg gagggaaagg aaggggaaag 13560
gaagggaggg aaggggctga caagaagaga gaagagggag ggaggggagg gaaaggaagg 13620
ggaaagaaga gaagggtaag aagaaactgt tccaatggtc tgggccacag agtgatggcc 13680
ttttgtggtg atcagctgta atccttgatt tgacacaacc tagaatctgg gaagcgagtt 13740
tctgtgaagg agcattcaca ctggctggcc tgtgggcgtg catgtgggag actgtcataa 13800
ttaggttcat taatacagga agtcccagcc cactacaaat ggcttcgttc catacccaag 13860
agatgctaac tgtagacggt tggagaaagc aagcaagctg tggatacccc acgctctttc 13920
acctcggctc ctggggggtg ggtgcactgt gtctcttggt attttaaagt cctgccttga 13980
cgtccctgct gtgacagact gtaactggaa ttgtgagctt tagtccttta gttttctacg 14040
ttggtttttc tcaggatatt ttatcgcagt aacagaaaca agaccaggac acttgatctc 14100
ctctgatcaa cactgaagag ttacaaaaca ggctgaggaa acaaactttc ttctccctct 14160
cccccttctg tccctcccct tccttctcgc tccctccctt gccccctctc tccctgtctc 14220
tgtctctgtc tctgtctctg tctctgtctc tgtctctgcc tctcccctcc cctcccctcc 14280
ctctgtctct gtctctgtct ctgtctctgt ctctgtctct gtctctgtcc ctttctcctc 14340
tatctcctaa atggctggag gccatgctag ctcaatgttg aactttgaac acgtatttag 14400
gaaatctttg ttcttaacag ttctgaagtg ctgaagtggt ggtttagtct ctcggcctga 14460
caagctcact tcctctcact ctgtcttaat gaccaaatct gccatttccc taaaacagca 14520
caggctccag ctccaggttg ctccggagcg gag 14553

### Example 15—CHO Stable Site 2 Sequences—U.S. Pat. No. 9,816,110

<211> 4001

### <212> DNA

<213> Cricetulus griseus
<400> 1

(SEQ ID NO: 58)
ccaagatgcc catcaactga ttaatagatg ataaaattat tgtacatttc agtgtaatat 60
tattcagttt ttaagaaaaa tgaaattatg taataagcat gtaaatggat atatcttgaa 120
acaaccattc cccattatat tacctaaaca ttgaaagtcc aaaatcatat gatcttttta 180
gtggatctac taatcttttg ctatatgtat tttattgaac tacccatgga tgtgagataa 240
ttggtaacaa cagcacatgg gagagcatgg gatcattcaa ggaagattag agagaatgca 300
ttttttagga gataatggag gagcaataga aaggattaaa tgaggttact gatgaaagtg 360
atggttagag aaggcaatat gaggagggat aactagcact tagggccttt tgaaaaagac 420
atagagaaaa tactattgta gaaacttcct ataattggtg tatagttata tacaccaaag 480
agctcagatg gagttaccct ataatggaaa tattaactac tttttatcac tgtgataaaa 540
catcctgaac agagcaacat agattgggaa gcatttactt tggcttacag ttctaacggg 600
ataaaaattc atgatgaaag aatgaatatg tcagcaaaca gcagtagcaa tggcctgaga 660
agcaggtgag agctcacatc ttgaagtgta agaatgtagc agagagaaca aactgcaaat 720
gaccagaaaa tgcttttgga tcagagccca tacccctctg actgacttct ccagaaattc 780
tgaacaaata aaactcccca aacagagcca taactgaagg tccagtgtct gagactacta 840
ggggtatttc ttattcaaac cactacaatg gggtgggggg agcaatcctc caagtaggca 900
ctacacacag acaaataaaa actctagtaa ctggaatgga ttgacttatt tgaattactt 960
gccagtggag ctacatagag cacaattatt gtatttaaat taccctttat gatcttacaa 1020
aacttgacag taagatcata ttgctaaaga aaccacatat ttgaatcagg gaacatggtg 1080
atatctagtt gttcttcaac tggaaacttc atgctttctg cccagcattc atgttgctgg 1140
aaagagcaat gtacactacc agtgtagaaa ttaaatcatc aatcttatca agatgtggat 1200
cctataagtt acaataaaaa ttagcctgat aagatatccc caccagaaga atattcacat 1260
aaatgctatg ggagcaacaa gctattttct aaattagctt taatcctatt ctacaagaga 1320
gaatccatat ctagaatagt tatagggatc aagaacccat ggcttgattg gtcataggcc 1380
caatgggaga tcctaatatt attgttctac aaaatgaaaa taactcctaa tgacttgttg 1440
ctgcagtaat aagttagtat gttgctcaac tctcacaaga gaagttttgt cttacaataa 1500
atggcaatta aagcagcccc acaagattta tatcataccg atctcctcat ggcctatgca 1560
tctagaagct aggaaacaaa gaggacccta agagagacat acatggtccc cctggagaag 1620
gggaaggggg caagacctcc aaagctaatt gggagcatgg gggaggggag agggagttag 1680
aagaaagaga aggggataaa aggagggaga ggaggacaag agagagaagg aagatctagt 1740
caagagaaga tagaggagag caagaaaaga gataccatag tagagggagc cttgtatgtt 1800
taaatagaaa actggcacta gggaattgtc caaagatcca caaggtccaa ctaataatct 1860
aagcaatagt cgagaggcta ccttaaaagc ctttctctga taatgagatt gatgactacc 1920
ttatatacca tcctagagcc ttcatccagt agctgatgga agcagaagca gacatctaca 1980
gctaaacact gagctagttg cagacaggga ggagtgatga gcaaagtcaa gaccaggctg 2040
gagaaacaca cagaaacagc agacctgaaa aaaatgttgc acatggaccc cagactgata 2100
gctgggagtc cagcatagga cttttctaga aaccctgaat gaggatatca gtttggaggt 2160
ctggttaatc tatggggaca ctggtagtgg atcaatattt atccctagtt catgactgga 2220
atttgggtac ccattccaca tggaggaatt ctctgtcagc ctagacacat gggggaggtt 2280
ctaggtcctg ctccaaataa tgtgttagac tttgaagaac tcccttgaga agactcaccc 2340
tccctgggga gcagaaaggg gatgggatga gggttggtga gggacaggag aggaggggag 2400
ggtgagggaa ctgggattga caagtaaatg atgcttgttt ctaatttaaa tgaataaagg 2460
aaaagtaaaa gaagaaaaga aaacaggcca aaagattata aaagacagag gtggtgggtg 2520
actataaaga aacactatta tctaaataaa aacatgtcag aagcacacat gaacttatag 2580
tgtttatgaa agtatgtata ataactacat aatctcaagc caagaaaaaa atatcatctt 2640
tcagtgatga aggtgatttt atttctccca gaattaaagc caaagaccta atgaaagtaa 2700
ttatcttcaa aaggttgaaa atacatactt tgcaatacac agatctgcct agaaatctca 2760
tgttcacaat acacatgatg ctcaattgaa ttccattcaa tgttacagtt tagataaaca 2820
gtttgtagat aaactcacaa tgtatcattt ctttttattt tttgaccaaa cagcttctca 2880
tctgttattc agaataattc ctcgatggca ggatatccat cccaattggg ggaaggggag 2940
aatttgaaga aaacctagac cacatacata tttgccattg ggaaacaaag tctaaaatga 3000
tgttgttcac atcttctcta ctagtcctct ccccgtccca aagaaccttg gtatatgtgc 3060
ctcattttac agagagagga aagcaggaac tgagcatccc ttacttgcca tcctcaaccc 3120
aaaatttgca tcattgctca gctctgccct tctcatatga cagttacaag tcaaggcttc 3180
caaagtccct ctgtcatgtt tggtgtcaat agtttataca gatgacttca tgtcttcata 3240
tctaatgtct tatatagatt aatattaaac aatgttattt ctctaaccac attttaaatt 3300
aatttaaaaa tccattaatt gtgtctataa aatgcagaca gagtgctgag acacaatata 3360
agcctgatga tctgaatttg aaactcacac ccaccacatg gagaatcaac ttccaaaaat 3420
tttcctatta cttccacact tacaccattg tacaaacaca ataataatga acaaaatgaa 3480
atgaaataaa aaattaagtc tctgtaggta atgctactgt gcagcaaaag taaaaatggc 3540
agcttaagct tgctttatgg ttacacttta ccatcttcca ttaattataa ggacttcaat 3600
catggcagaa ctatgctgtt attgtctcag tgtaacctaa ccaggtgttc cagatgttct 3660
taatgtggac acctaaacta tttgatattt gggttaagat ctttccctct ttcagaagaa 3720
acctcaggac agagggaatc ttgtctttta attttgagtc tgtagacttt ttccatttca 3780
aatatacatg aaacaagtga tgaagaaaat taatcaaaag gtgggaattg caatgatatt 3840
aggttcaata ttaagcttca atattatcat ggaatcgcct gttatacact gagtgtttgg 3900
caataaggga tttttagaag aaggagtttt tattctcaac aggttcctta agtttagctc 3960
aaataaatct aagcaatcca ctctagaatt aaatagtttc c 4001

<211> 14931

### <212> DNA

<213> Cricetulus griseus
<220>
<221> misc_feature
<222> (2176) . . . (2239)
<223> n is a, c, g, t or nucleotide is missing
<400> 4

(SEQ ID NO: 59)
catgtacact tatgcaagta tgatatggcc caacacagta ttttacacca atttttatct 60
ataaaatata catgtacatc aaaatatatt attaataata acatcattat tctttctttc 120
caagtaataa acacatacac tgaaattttg gttcttgtgg ataattttaa tgaaacagga 180
aatgcaaatt tatcttagca tgtttacttc actttctttg catagataac cagtaatcac 240
attgatggat catgtagtga aatgtatttt taggtatcta aggaattttg gcttcgtttt 300
gtgcttgttg acactgaatt ctattcctaa caacagtgtg taaggattct gtctgatttc 360
ttttaccagt atttgtccat ttgcattttc tttattattc atggctgctg ttctagaaag 420
tggaaggtag tgtgtcaagt ctgtttaaca tgtttccctg atgatcagtg tcttaacacc 480
tctctgagta catgttggcc aatgtcgttt ctagacccat ctattcttgc ttgacttatc 540
ctggtacatg cctgccaaga aatttctcct catcctttct gtctcttcac tgatttactt 600
gatgtgtgga tttcacattg atcatatgga aatagaagat acaattttct ttattcacag 660
tttggaagac tttcaatctc atagatcatc attatttttt gctactgttc cctatgctat 720
ggtgaaattt ccatttgaat aattgcttaa acaattaaca agaaagaatc tatttttact 780
tgcaataact tccatttcag aacatttact acactgttac tatatccaaa aactagtttt 840
atatatcatg tgagaaatga ctaattcata atttggccat gacatttttt tcagaaacag 900
aaaaagtgac caatacatac acaatgctat aaatattaag acttcagcaa attaaatatt 960
tattcatgat atcacataaa attcatttat tatgttttat ttaaatgtgt ttttaaaaca 1020
gtggtatcac taaatattaa gttagatgtg tttatgtgct taatgaattt atattttaga 1080
atgttataag ttgtatatag tcaaatatgt aataaatttt attttttagg tctttctcat 1140
taaggtattt taattttggg tcccttttcc agagtgactc tagctcatga tgagttgaca 1200
taaaaactaa acagtacaaa atgtacattg cattcagtat tgcacttgat ctttgcactg 1260
aagtttgagt cagttcatac atttagtact tgggaagtac attaagctaa ctttcattgc 1320
tctggcaaaa tgctcgataa gataagagtc tattgtggaa agccatggca gcaggaaagt 1380
aagactgctg atgatgttta atccatagtc aagacgcaga aggagatgaa tgctggtatc 1440
caacattttt tgctgttcat tttctctaga accctagtcc ataaagatgt atgacttgca 1500
ttcaaaatgc gtccccttca gttgttcaac ttttctgtaa atatcctttc aggcatgtct 1560
agaagattgt ttcgcaaata cttctcaatc cattcaagtt gatagtgcag attaatcact 1620
gcagaataaa agcctgtaac ttggctcacg tgccaaggaa tatgcacact cctgacacat 1680
caataagtaa atcaaagtgt agcttttgcc tttaacattg ccagacttat gtaatgttct 1740
gcacgttctt cctccatcac tttttattct aatggtgttt ccttgacatt gaatcacgct 1800
gtggaagctg cttagaatta acattgaaat ctactgatat atttatgatg cagcaattta 1860
gatttactat tttacttaga attttttata attgagagaa tataatattt tcacagttat 1920
ctatctgctg taaatagagg attttaaaaa aaatctctat aacttttttt tacaacacac 1980
agtaaaatta agttaaaatt taataaagtc actatgttga tttcaaagtg tgctacgccc 2040
acggtggtca cgcaggtgta gcagaagatg ccactaaggt gggctaaggc cgatgggttg 2100
gggtctgcgc tccctggaga tgagccccag gcggttccct ggcaatcagc tgcgatcatg 2160
atgcccgatg agccannnnn nnnnnnnnnn nnnnnnnnnn nnnnnnnnnn nnnnnnnnnn 2220
nnnnnnnnnn nnnnnnnnnc tgggtgactt tatggaaaga atttgataga tttcatgatg 2280
tagaagaatt ttattaggct tattttacag gagactaaga ccctgggacc taaagatatc 2340
tgggtcctga gaatcaggaa atgggtagag acgtggttga tggtatgaga cagattttag 2400
agaactctta gatcatgggc aatgaccgca atctgatgct tagaatagat catctataaa 2460
caattatgct gttctttttc tttctgttgt atgatctgat gatgtagccc ccttgccaag 2520
ttccctgatc ccccttgcca agttccctga ttgtaacagt atataagcat tgcttgagag 2580
catattcaac tacattgagt gtgtctgtct gtcatttcct cgccgattcc tgatttctcc 2640
ttgagccttt tcccttgttc tccctcggtc ggtggtctcc acgagaggcg gtccgtggca 2700
aaagtgtata aatgttctaa aacatttgaa ctctaaaaca tgcaaaatga aaaattaaaa 2760
taaataaaca tgaaaattaa aatatattag ctgctaaaag ttaaacaata ctatataata 2820
ttttgttatt agaattcaaa atcacattag ttggatttaa tttgaacatt gcattctttc 2880
aataataatt tcaataaaaa aagtttcccc atgatagtag aaaataataa catatgtatc 2940
tatctattta tttaactaca catatatagc atttgtttca actaaaataa atgaatgagc 3000
aaagcaccta agtaattggt gtctattata tttatgaagc caatagtttc aaataaatta 3060
tcatgcataa ggaggtattg caaatgttaa accttttttg aaacagatat tcccagttac 3120
agaaattata atttctaatc tttcctataa gtagaatgat gataattaat ataggccatt 3180
tgtaaataat gttcagatta aaatattctc tatttcacta gagaagaatg atattaaatg 3240
tattatattt tatttcccat tttgtttgca ccactattct atatccctca gcagtttaaa 3300
tttgtttcac catatgtgtg tgtgtttgta tcttaaatat ggcactaaaa ttagaataat 3360
ttaatataaa tctttaggag aaaagatatt gaattatttt atgttgatag gaaaatatct 3420
tttaattgtc caagaatact ttttcttcta ttttaggact gatcagaccc aggactaata 3480
ttttatatgt actaattcta tgtaccaaaa tatgttatta tctcatgaat tctgtctcaa 3540
tattgaggta ataaaaatag tccatcatga actttaaaat taaaataatg attaattaat 3600
ttttattcat attttgtttg tatgaatggt tatacatcac atgtgtgcct ggtgactgtg 3660
aatgtcagga gaaggtatga aagccactgg aattggaata agagataata tttgagatgt 3720
tatgtgggtg ctgagaatta gacgcaagcc atcttcaaga atagccagca tactatacca 3780
ctgagtaatc cattcatccc tcaataatta tctttgtaga cagtaaatat atttctaaac 3840
tataaatgac cagaaaaatt aatgtattat taatgaagac attcatctca tgtgacacac 3900
ttcacctgtc taaatcagta acactctctc cactaattaa gattttctaa gtgcatgaca 3960
cttactattt ctaaagctgt ccaatggggg ccagtcccca gtcagcaccc agtgagataa 4020
tccatgaatg catttatatc ttaggaaaaa ttcttatcta tgtagtattt agaacatttt 4080
catgtgaggg gataaacaag gaagcacaga tgctttctga tagaaacttt ctctttaatt 4140
catctagaaa aaaaaaacct ctcaggaaaa tctctcttgc tctcctccca atgctctatt 4200
cagcatcttc tccctactta attctagatc tttttctcta tgcctccttg ctgctgccct 4260
gctggctctg ctctatgcct ccccatgtca cttttctttg ctatctcacc gttaccttct 4320
ctgcctcact ctctgccttc ttctctgctt ctcacatggc caggctctgg acaattatag 4380
ttatatgtta cattctcata acacatgata tgtcacatag tttctctcag gctagggata 4440
tcacaatgac tggccaatga gcaagtggcc ttgcatgtag ctctaagttg gtgatggttc 4500
ccagacagta agtagccatt tggttgaaat ttgaggttgg gtagtacatg aagactgaat 4560
tttcttcaaa ctctggcctt gaaatagtaa aacaacacct atgaaaatga cgacctgtat 4620
ttgtctttag aggcaaccac atattgtctg cagggcctgc tttgaatttg ctctgaagtt 4680
agcttgtttg tgtaaaagga agaatcctat atcagcctga gaaatgtaaa atatcctagc 4740
atttcaagtc atcaaaatta tatggagagt ataaatcatc cttctgacta ttcatagtca 4800
tatttgtgtc caccaagtat aaaacacact accaaagggc tgtggaaaaa atcgccataa 4860
ctgttcttat tagggaggca tagcagtggt acctgaggaa gttacagcaa caaccagtca 4920
tccagtcaat aaccccatgg ctttgccact tggaggtacc caataatgtt tggctttgcc 4980
gagtaggact ccaacaaatt cagagggtca atttttaaat gctggttgtc actgctgaac 5040
agtcccattg ccctctgcat aattccacaa tggaaagctt tttacactga ttgccaatca 5100
ttaaacagcc tactcagcat aaacaggtat gatattattc tgcattttgt tacattacta 5160
gatgaattcc tatttcttcc tacaatagtg gaactgaaaa aagatacaca atcatactac 5220
ccctctacta atcttatgac ttatatcatt tcaattttca gaccataatg caaactattg 5280
accaaaacat gtgaagatga aaaatagaaa tgtagaataa tattacatat aaaaagaaaa 5340
ggcggactta ttttgtttta tttcttagca tgcatagcaa tacatgattt gaggtttata 5400
taataaaggg acaataaatc ttcaagaaac ttacccctac tgaattaaaa tattaaagaa 5460
ggtcacacat ttactcaaat atattagact actgggcaaa tagacatgaa aagtagagtt 5520
aatattgagg taggccttct gtgaaatgtc taaggaaatt atgtttcata cagtgtgtaa 5580
ccaagtggga atcatatcag aaagcagtca aaagcttata ttacaagtaa cagatgcttg 5640
gttatatgac ctcccagagc ttgactgtct atacacaaaa agtggtgtta ataaaactgt 5700
aatttgggct atgttttttt aaatggcttc accaacatga aaggaaggga atgagcatgt 5760
catggatgct tagagattat gcttccagca agaagaattg agctttggct cttattacag 5820
aaacatgaca aggtgtgagt tttatttatt agaaattata taatatttta agctggggac 5880
taaaaatttt attgaaacaa acaggcaagg gataggcatg tactagaagc aaaaatagga 5940
tgtcaatgct gtaatgttat tttttggacc aaaatagtat ttcctataga aatgacaatg 6000
atcttaggtt attattcttc ataaagatga caagttcaca agatatccta gttcattaaa 6060
atcgttttag tcatttaata gagtgctgtg atagattaca caaaggaaag cacttacgat 6120
gagaaataat gatatccaca attattttct taattcttag aaacattcta ttgttatatc 6180
tcaatctcag aagccactta ttgctttatt attgaaacat atgaaattgt aagttatata 6240
ttgtctatgg tgacatttca aagaacatgt gacgtacagt gtagcacaga taaagaacat 6300
aactgcagct gaatcagtaa ctaaacttac atacattaaa tctgccatgt tggcaacagt 6360
gtgtgcacta ccaaaggatg tactaatgct cacgacactc ccctatgtca ccctttgttc 6420
atcattacat cataggtcta ttttgtttgc ttttgaaatc tagaccaagt cttttgtgtc 6480
tttccaagca cagagctcat taatttacct catagacttg ttaaacttct tctggttcat 6540
caattgaata gaaatactca ctactaatta tgtgagaccc tgccagtacc atagcacatg 6600
gataattttt acataaaaca tgcatacaag taagattatt cagactgaac atgaatttta 6660
gagaaatcag gaaggagtat atgggagtgg ttggagtgag actagagaaa tgtaattaaa 6720
ctataatctc aatacaaaga tctactaagc aaaaaacatg aaacattgtc attcaagtga 6780
aacatcagtc ttcaaattgg aaagatattt ttactaggaa aatgtctggt agatggttat 6840
tatctagaaa acacaaaaat tagaaaacgg taaactttaa taaaaagaat aatacaatga 6900
gactacatga aaagttctta actaatgaaa caaatatctt gaaacttttt tcttaaaagt 6960
ttaatatcaa taaccatcat ggaaattcaa attaaaacta tttacatatt acccctgaaa 7020
taataactaa tacccaataa aaataatata aacaaaaaat ggcaatgcat gccatcatgg 7080
atttgggaga gagaatgttc attgcagttc tgaatggata ctggtgccac cacggtgaaa 7140
atctctgtat aggtccttcc aaaagctgaa aatagacata tcacaagacc tgccacacat 7200
ttttcaagca aatacccaaa ggactctacc tgactgcaga gacactttct cataaaatat 7260
tattgttgat ctattcataa tatctggaaa atagaaacag ccaagatgcc catcaactga 7320
ttaatagatg ataaaattat tgtacatttc agtgtaatat tattcagttt ttaagaaaaa 7380
tgaaattatg taataagcat gtaaatggat atatcttgaa acaaccattc cccattatat 7440
tacctaaaca ttgaaagtcc aaaatcatat gatcttttta gtggatctac taatcttttg 7500
ctatatgtat tttattgaac tacccatgga tgtgagataa ttggtaacaa cagcacatgg 7560
gagagcatgg gatcattcaa ggaagattag agagaatgca ttttttagga gataatggag 7620
gagcaataga aaggattaaa tgaggttact gatgaaagtg atggttagag aaggcaatat 7680
gaggagggat aactagcact tagggccttt tgaaaaagac atagagaaaa tactattgta 7740
gaaacttcct ataattggtg tatagttata tacaccaaag agctcagatg gagttaccct 7800
ataatggaaa tattaactac tttttatcac tgtgataaaa catcctgaac agagcaacat 7860
agattgggaa gcatttactt tggcttacag ttctaacggg ataaaaattc atgatgaaag 7920
aatgaatatg tcagcaaaca gcagtagcaa tggcctgaga agcaggtgag agctcacatc 7980
ttgaagtgta agaatgtagc agagagaaca aactgcaaat gaccagaaaa tgcttttgga 8040
tcagagccca tacccctctg actgacttct ccagaaattc tgaacaaata aaactcccca 8100
aacagagcca taactgaagg tccagtgtct gagactacta ggggtatttc ttattcaaac 8160
cactacaatg gggtgggggg agcaatcctc caagtaggca ctacacacag acaaataaaa 8220
actctagtaa ctggaatgga ttgacttatt tgaattactt gccagtggag ctacatagag 8280
cacaattatt gtatttaaat taccctttat gatcttacaa aacttgacag taagatcata 8340
ttgctaaaga aaccacatat ttgaatcagg gaacatggtg atatctagtt gttcttcaac 8400
tggaaacttc atgctttctg cccagcattc atgttgctgg aaagagcaat gtacactacc 8460
agtgtagaaa ttaaatcatc aatcttatca agatgtggat cctataagtt acaataaaaa 8520
ttagcctgat aagatatccc caccagaaga atattcacat aaatgctatg ggagcaacaa 8580
gctattttct aaattagctt taatcctatt ctacaagaga gaatccatat ctagaatagt 8640
tatagggatc aagaacccat ggcttgattg gtcataggcc caatgggaga tcctaatatt 8700
attgttctac aaaatgaaaa taactcctaa tgacttgttg ctgcagtaat aagttagtat 8760
gttgctcaac tctcacaaga gaagttttgt cttacaataa atggcaatta aagcagcccc 8820
acaagattta tatcataccg atctcctcat ggcctatgca tctagaagct aggaaacaaa 8880
gaggacccta agagagacat acatggtccc cctggagaag gggaaggggg caagacctcc 8940
aaagctaatt gggagcatgg gggaggggag agggagttag aagaaagaga aggggataaa 9000
aggagggaga ggaggacaag agagagaagg aagatctagt caagagaaga tagaggagag 9060
caagaaaaga gataccatag tagagggagc cttgtatgtt taaatagaaa actggcacta 9120
gggaattgtc caaagatcca caaggtccaa ctaataatct aagcaatagt cgagaggcta 9180
ccttaaaagc ctttctctga taatgagatt gatgactacc ttatatacca tcctagagcc 9240
ttcatccagt agctgatgga agcagaagca gacatctaca gctaaacact gagctagttg 9300
cagacaggga ggagtgatga gcaaagtcaa gaccaggctg gagaaacaca cagaaacagc 9360
agacctgaaa aaaatgttgc acatggaccc cagactgata gctgggagtc cagcatagga 9420
cttttctaga aaccctgaat gaggatatca gtttggaggt ctggttaatc tatggggaca 9480
ctggtagtgg atcaatattt atccctagtt catgactgga atttgggtac ccattccaca 9540
tggaggaatt ctctgtcagc ctagacacat gggggaggtt ctaggtcctg ctccaaataa 9600
tgtgttagac tttgaagaac tcccttgaga agactcaccc tccctgggga gcagaaaggg 9660
gatgggatga gggttggtga gggacaggag aggaggggag ggtgagggaa ctgggattga 9720
caagtaaatg atgcttgttt ctaatttaaa tgaataaagg aaaagtaaaa gaagaaaaga 9780
aaacaggcca aaagattata aaagacagag gtggtgggtg actataaaga aacactatta 9840
tctaaataaa aatatgtcag aagcacacat gaacttatag tgtttatgaa agtatgtata 9900
ataactacat aatctcaagc caagaaaaaa atatcatctt tcagtgatga aggtgatttt 9960
atttctccca gaattaaagc caaagaccta atgaaagtaa ttatcttcaa aaggttgaaa 10020
atacatactt tgcaatacac agatctgcct agaaatctca tgttcacaat acacatgatg 10080
ctcaattgaa ttccattcaa tgttacagtt tagataaaca gtttgtagat aaactcacaa 10140
tgtatcattt ctttttattt tttgaccaaa cagcttctca tctgttattc agaataattc 10200
ctcgatggca ggatatccat cccaattggg ggaaggggag aatttgaaga aaacctagac 10260
cacatacata tttgccattg ggaaacaaag tctaaaatga tgttgttcac atcttctcta 10320
ctagtcctct ccccgtccca aagaaccttg gtatatgtgc ctcattttac agagagagga 10380
aagcaggaac tgagcatccc ttacttgcca tcctcaaccc aaaatttgca tcattgctca 10440
gctctgccct tctcatatga cagttacaag tcaaggcttc caaagtccct ctgtcatgtt 10500
tggtgtcaat agtttataca gatgacttca tgtcttcata tctaatgtct tatatagatt 10560
aatattaaac aatgttattt ctctaaccac attttaaatt aatttaaaaa tccattaatt 10620
gtgtctataa aatgcagaca gagtgctgag acacaatata agcctgatga tctgaatttg 10680
aaactcacac ccaccacatg gagaatcaac ttccaaaaat tttcctatta cttccacact 10740
tacaccattg tacaaacaca ataataatga acaaaatgaa atgaaataaa aaattaagtc 10800
tctgtaggta atgctactgt gcagcaaaag taaaaatggc agcttaagct tgctttatgg 10860
ttacacttta ccatcttcca ttaattataa ggacttcaat catggcagaa ctatgctgtt 10920
attgtctcag tgtaacctaa ccaggtgttc cagatgttct taatgtggac acctaaacta 10980
tttgatattt gggttaagat ctttccctct ttcagaagaa acctcaggac agagggaatc 11040
ttgtctttta attttgagtc tgtagacttt ttccatttca aatatacatg aaacaagtga 11100
tgaagaaaat taatcaaaag gtgggaattg caatgatatt aggttcaata ttaagcttca 11160
atattatcat ggaatcgcct gttatacact gagtgtttgg caataaggga tttttagaag 11220
aaggagtttt tattctcaac aggttcctta agtttagctc aaataaatct aagcaatcca 11280
ctctagaatt aaatagtttc ctaagggcac agctatgaat agagctcaat ttacatataa 11340
aattttgttc accatttatg tcattccagt tttcattagt acaaggaaaa tacaaaatat 11400
ttagatgtca atatcaagtg aatagttcat ctcctttttt aatatatatc acctaaatca 11460
ccattttctc agaaaaatct ggcctgaagt tctgtctgga acttcaacat gaaaaatatg 11520
cacagcttgc tattataaat cctagttgat ttttaagatt catgtctggt gtctgactca 11580
gaggggccag aggctagaca aatatttttt gaatcttcat tgtgaagatt tttaatgatt 11640
attttaatat aaataacaaa gatgatggat aatgtaactt tgtacagttc atagacgctg 11700
aactactttg tgcttaaaat gttagttccc tatcataaat gataggtgat aagtgtatgt 11760
ttaatacttt ccctctgagc tatattcatg tactagagaa ttattttaaa catgaaaaga 11820
ctgtgtttat agtctcagct cctgagaact ggtccaacct taggcaggtg aatgccagga 11880
gcaacgtttt tcttctacag aggatgcttt gctgccaagc aacctggttg tgtggaaatg 11940
ttcctttttt aatcaagttt aaagggtctt catcatgctg ttgctccaca tattttcagg 12000
ttagagcttg gtccttggag tattatcttt taccagaaaa ttcatagtat tctttcaata 12060
actaacaact aaacttttcg ataaaaaaga attggaattt caattttaaa gcctgagtaa 12120
aattcttgtg aatcaggata ttttatttta agtcttatct tttaaaaagt tattttattt 12180
tttaaaaaat tataatatac tttcataatt tccctccttc acttttcttt acaaacactt 12240
ctatagatca ccatgtgttt ttttttttac atttatggcc tctttctgtt cattgttatt 12300
acatacaaat agtcttgcct atagaagaac accacaattt gttacctgat aacaaattat 12360
caacccttaa aacctacaaa ctattgatat tactgaaaag actatactta tagatgtaaa 12420
gatatatgtg tgtgcacata tatagataca catatatgta ggatttttaa ttttagattt 12480
tagacatcaa aattatttat atgactgaga aactagacac tataaatgag cattcagtat 12540
tcaacaccgt gattttagat attgtcacaa tgacagaaaa ttttcttata gaaaatttta 12600
agttttgtga ttgctctgtg cacttagtga agtctcacag aaaaagaatc atagtatttt 12660
tagtttataa taaaaagtac atataattaa aatggttggc acaaaacaac atttgagcat 12720
ttttcctatt tactatcaag tagtatcatt ttgaaataat aatttgacta gtttcaaaaa 12780
tgaaaacaaa atttaaacta aatgcctaat ctagcctgat aacattttta tgaatgaaat 12840
tattcaatag tgttatcaat taggggccca aaacttttcc taaaataaaa cttttaattt 12900
ttttccattt ttatttaaat tagaaacaaa attgttttac atgtaaatca gagtttcctc 12960
accctcccct tctccctgtc cctcactaac accctacttg tcccatacca tttctgctcc 13020
ccagggaggg tgaggccttc catggggaaa cttcagagtc tgtctatcct ttcggatagg 13080
gcctaggccc tcacccattt gtctaggcta aggctcacaa agtttactcc tatgctagtg 13140
ataagtactg atctactaca agagacacca tagatttcct aggcttcctc actgacaccc 13200
atgttcatgg ggtctggaac aatcatatgc tagtttccta ggtatcagtc tggggaccat 13260
gagctccccc ttgttcaggt caactgtttc tgtgggtttc accaccctgg tcttgactgc 13320
tttgctcatc actcctccct ttctgtaact gggttccagt acaattccgt gtttagctgt 13380
gggtgtctac ttctactttc atcagcttct gggatggagc ctctaggata gcatacaatt 13440
agtcatcatc tcattatcag ggaagggcat ttaaagtagc ctctccattg ttgcttggat 13500
tgttagttgg tgtcatcttt gtagatctct ggacatttcc ctagtgccag atatctcttt 13560
aaacctacaa gactacctct attatggtat ctcttttctt gctctcgtct attcttccag 13620
acaaaatctt cctgctccct tatattttcc tctcccctcc tcttctcccc ttctcattct 13680
cctagatcca tcttcccttc ccccatgctc ccaagagaga tgttgctcag gagatcttgt 13740
tccttaaccc ttttcttggg gatctgtctc tcttagggtt gtccttgttt cctagcttct 13800
ctggaagtgt ggattgtaag ctggtaatca tttgctccat gtctaaaatc catatatgag 13860
tgatgtttgt ctttttgtga ctgggttacc tcactcaaaa tggtttcttc catatgtctg 13920
tggatttcaa tagcacaaac aacatacagt atcttggggc aacactaacc aaacaagtga 13980
aagaccagta tagcaagaac tttgagttta aagaaagaaa ttaaagaaga taccagaaaa 14040
tggaaagatc tcccatgctc tttgataggc agaatcaaca tagtaaaaat ggcaatcttg 14100
ccaaaatcca tctacagact caatgcaatc cccattaaat accagcacac ttcttcacag 14160
acctgaaaga ataatactta actttatatg gagaaacaaa agacccagga taggccaaac 14220
aaccctgtac aatgaaggca cttccagagg catccccatc cctgacttca agctctatta 14280
tagagtaata atcctgaaaa cagcttggta atggcacaaa aatagacagg tagaccaatg 14340
gaattgagtt gaaaaccctg atattaaccc acatatctat gaacacctga ctttgacaaa 14400
gaagctaagg ttatacaatg taagaaagaa agcatcttca acaaatcgtg ctggcataac 14460
tggatgctgg catgtagaag actgcagata gatccatgtc taatgccatg cacaaaactt 14520
aagtccaaat ggatcaaaaa cctcaacata aatccagcca cactgaacct catagaagag 14580
aaagtgggaa gtatccttga ataaattggt acaggagacc acatcttgaa cttaacacca 14640
gtagcacaga caatcagatc aataatcaat aaatgggacc tcctgaaact gagaagcttc 14700
tgtaaggcaa tggataagtc aacaggacaa aatggcagcc cacggaatgg gaaaagatat 14760
tcaccaatcc tatatctgac agagggctgc tctctatttg caaagaacac aataagctag 14820
tttttaaaac accaattaat ccgattataa agttgggtag agaactaaat aaagaattgt 14880
taacagagca atctaacttg gcagaaagac acataagaaa gtgctcacca t 14931

It is to be understood that the description, specific examples and data, while indicating exemplary embodiments, are given by way of illustration and are not intended to limit the present inventions. Various changes and modifications within the present invention, including combining embodiments in whole and in part, will become apparent to the skilled artisan from the discussion, disclosure and data contained herein, and thus are considered part of the inventions.

1-81. (canceled)

82. A polynucleotide comprising in order (i) a promoter, (ii) an intron, (iii) a first internal ribosome entry site, (iv) a first AAV Cap gene, (v) a second internal ribosome entry site, (vi) a second AAV Cap gene, and (vii) a polyadenylation site.

83. The polynucleotide according to claim 82, wherein the polynucleotide is integrated into a eukaryotic cell genome.

84. The polynucleotide according to claim 83, wherein the eukaryotic cell is a CHO cell or a BHK cell.

85. The polynucleotide according to claim 83, wherein the eukaryotic cell is a HEK 293 cell, HeLa cell, or a human amniotic cell.

86. The polynucleotide according to claim 83, further comprising an operator.

87. The polynucleotide according to claim 82, wherein the promoter is a CMV promoter.

88. The polynucleotide according to claim 86, wherein the operator is a Tet operator.

89. A method of producing recombinant adeno-associated virus (AAV) comprising a gene of interest, wherein the method comprises the steps of:
(a) providing the cell culture that comprises eukaryotic cells, wherein a eukaryotic cell of the cell culture comprises a polynucleotide comprising in order (i) a promoter, (ii) an intron, (iii) a first internal ribosome entry site, (iv) a first AAV Cap gene, (v) a second internal ribosome entry site, (vi) a second AAV Cap gene, and (vii) a polyadenylation site,
wherein the eukaryotic cell further comprises an AAV Rep gene, adenovirus polynucleotides that act in a helper capacity, and the gene of interest flanked by AAV inverted terminal repeats; and
(b) culturing the eukaryotic cells in a culture medium to allow the eukaryotic cells to produce a recombinant AAV comprising the gene of interest.

90. The method according to claim 89, wherein the polynucleotide comprising in order (i) a promoter, (ii) an intron, (iii) a first internal ribosome entry site, (iv) a first AAV Cap gene, (v) a second internal ribosome entry site, (vi) a second AAV Cap gene, and (vii) a polyadenylation site is integrated into a eukaryotic cell genome.

91. The method according to claim 90, wherein the eukaryotic cell is a CHO cell or a BHK cell.

92. The method according to claim 90, wherein the eukaryotic cell is a HEK 293 cell, HeLa cell, or a human amniotic cell.

93. The method according to claim 89, wherein the adenovirus polynucleotides comprise at least one selected from the group consisting of E2A or E2A orf, E4 or E4 orf 6, and VA RNA.

94. The method according to claim 89, wherein the adenovirus polynucleotides comprise E2A or E2A orf, E4 or E4 orf 6, and VA RNA.

95. The method according to claim 93, wherein the adenovirus polynucleotides further comprise at least one of E1A or E1B.

96. The method according to claim 95, wherein the adenovirus polynucleotides further comprise E1A and E1B.

97. The method according to claim 89, wherein polynucleotide further comprises an operator.

98. The method according to claim 89, wherein the promoter of the polynucleotide is a CMV promoter.

99. The method according to claim 93, wherein the operator is a Tet operator.


SEQUENCE LISTING (59 sequences)

SEQ ID NO: 1 - DNA, 2211 bp, Adeno-associated virus - 1
  misc_feature 1..2211: note = CapVP1
  source 1..2211: mol_type = genomic DNA
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 2 - DNA, 1872 bp, Adeno-associated virus - 1
  misc_feature 1..1872: note = Rep78
  source 1..1872: mol_type = genomic DNA
atgccgggcttctacgagatcgtgatcaaggtgccgagcgacctggacgagcacctgccgggcatttctgactcgtttgtgagctgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcctgtttgaacctggccgagcgcaaacggctcgtggcgcagcacctgacccacgtcagccagacccaggagcagaacaaggagaatctgaaccccaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcttccaactcgcggtcccagatcaaggccgctctggacaatgccggcaagatcatggcgctgaccaaatccgcgcccgactacctggtaggccccgctccgcccgcggacattaaaaccaaccgcatctaccgcatcctggagctgaacggctacgaacctgcctacgccggctccgtctttctcggctgggcccagaaaaggttcgggaagcgcaacaccatctggctgtttgggccggccaccacgggcaagaccaacatcgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaaccggtcgtcagaaagaggacgtatcggaaactctgtgccattcatcatctgctggggcgggctcccgagattgcttgctcggcctgcgatctggtcaacgtggacctggatgactgtgtttctgagcaataa

SEQ ID NO: 3 - DNA, 1866 bp, Adeno-associated virus - 2
  misc_feature 1..1866: note = Rep78
  source 1..1866: mol_type = genomic DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 4 - DNA, 1194 bp, Adeno-associated virus - 2
  misc_feature 1..1194: note = Rep52
  source 1..1194: mol_type = genomic DNA
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 5 - DNA, 2208 bp, Adeno-associated virus - 2
  misc_feature 1..2208: note = CapVP1
  source 1..2208: mol_type = genomic DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 6 - DNA, 1797 bp, Adeno-associated virus - 2
  misc_feature 1..1797: note = CapVP2
  source 1..1797: mol_type = genomic DNA
acggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 7 - DNA, 1602 bp, Adeno-associated virus - 2
  misc_feature 1..1602: note = CapVP3
  source 1..1602: mol_type = genomic DNA
atggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - DNA, 615 bp, Adeno-associated virus - 2
  misc_feature 1..615: note = CapAAP
  source 1..615: mol_type = genomic DNA
ctggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctga

SEQ ID NO: 9 - DNA, 1875 bp, Adeno-associated virus - 3
  misc_feature 1..1875: note = Rep78
  source 1..1875: mol_type = genomic DNA
atgccggggttctacgagattgtcctgaaggtcccgagtgacctggacgagcgcctgccgggcatttctaactcgtttgttaactgggtggccgagaaggaatgggacgtgccgccggattctgacatggatccgaatctgattgagcaggcacccctgaccgtggccgaaaagcttcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaaaacgcgaaatggcgccgggggcgggaacaaggtggtggacgactgctacatccccaactacctgctccccaagacccagcccgagctccagtgggcgtggactaacatggaccagtatttaagcgcctgtttgaatctcgcggagcgtaaacggctggtggcgcagcatctgacgcacgtgtcgcagacgcaggagcagaacaaagagaatcagaaccccaattctgacgcgccggtcatcaggtcaaaaacctcagccaggtacatggagctggtcgggtggctggtggaccgcgggatcacgtcagaaaagcaatggattcaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtaaactggaccaatgagaactttcccttcaacgattgcgtcgacaagatggtgatctggtgggaggagggcaagatgacggccaaggtcgtggagagcgccaaggccattctgggcggaagcaaggtgcgcgtggaccaaaagtgcaagtcatcggcccagatcgaacccactcccgtgatcgtcacctccaacaccaacatgtgcgccgtgattgacgggaacagcaccaccttcgagcatcagcagccgctgcaggaccggatgtttgaatttgaacttacccgccgtttggaccatgactttgggaaggtcaccaaacaggaagtaaaggactttttccggtgggcttccgatcacgtgactgacgtggctcatgagttctacgtcagaaagggtggagctaagaaacgccccgcctccaatgacgcggatgtaagcgagccaaaacgggagtgcacgtcacttgcgcagccgacaacgtcagacgcggaagcaccggcggactacgcggacaggtaccaaaacaaatgttctcgtcacgtgggcatgaatctgatgctttttccctgtaaaacatgcgagagaatgaatcaaatttccaatgtctgttttacgcatggtcaaagagactgtggggaatgcttccctggaatgtcagaatctcaacccgtttctgtcgtcaaaaagaagacttatcagaaactgtgtccaattcatcatatcctgggaagggcacccgagattgcctgttcggcctgcgatttggccaatgtggacttggatgactgtgtttctgagcaataa

SEQ ID NO: 10 - DNA, 2211 bp, Adeno-associated virus - 3
  misc_feature 1..2211: note = CapVP1
  source 1..2211: mol_type = genomic DNA
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 11 - DNA, 1872 bp, Adeno-associated virus - 4
  misc_feature 1..1872: note = Rep78
  source 1..1872: mol_type = genomic DNA
atgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataa

SEQ ID NO: 12 - DNA, 2205 bp, Adeno-associated virus - 4
  misc_feature 1..2205: note = CapVP1
  source 1..2205: mol_type = genomic DNA
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaa

SEQ ID NO: 13 - DNA, 1833 bp, Adeno-associated virus - 5
  misc_feature 1..1833: note = Rep78
  source 1..1833: mol_type = genomic DNA
atggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaa

SEQ ID NO: 14 - DNA, 2175 bp, Adeno-associated virus - 5
  misc_feature 1..2175: note = CapVP1
  source 1..2175: mol_type = genomic DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 15 - DNA, 1872 bp, Adeno-associated virus - 6
  misc_feature 1..1872: note = Rep78
  source 1..1872: mol_type = genomic DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataa

SEQ ID NO: 16 - DNA, 2211 bp, Adeno-associated virus - 6
  misc_feature 1..2211: note = CapVP1
  source 1..2211: mol_type = genomic DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 17 - DNA, 1872 bp, Adeno-associated virus - 7
  misc_feature 1..1872: note = Rep78
  source 1..1872: mol_type = genomic DNA
atgccgggtttctacgagatcgtgatcaaggtgccgagcgacctggacgagcacctgccgggcatttctgactcgtttgtgaactgggtggccgagaaggaatgggagctgcccccggattctgacatggatctgaatctgatcgagcaggcacccctgaccgtggccgagaagctgcagcgcgacttcctggtccaatggcgccgcgtgagtaaggccccggaggccctgttctttgttcagttcgagaagggcgagagctacttccaccttcacgttctggtggagaccacgggggtcaagtccatggtgctaggccgcttcctgagtcagattcgggagaagctggtccagaccatctaccgcggggtcgagcccacgctgcccaactggttcgcggtgaccaagacgcgtaatggcgccggcggggggaacaaggtggtggacgagtgctacatccccaactacctcctgcccaagacccagcccgagctgcagtgggcgtggactaacatggaggagtatataagcgcgtgtttgaacctggccgaacgcaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattaaaaccaaccgcatctaccgcatcctggagctgaacgggtacgatcctgcctacgccggctccgtctttctcggctgggcccagaaaaagttcgggaagcgcaacaccatctggctgtttgggcccgccaccaccggcaagaccaacattgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcacgactttggcaaggtgacgaagcaggaagtcaaagagttcttccgctgggccagtgatcacgtgaccgaggtggcgcatgagttctacgtcagaaagggcggagccagcaaaagacccgcccccgatgacgcggatataagcgagcccaagcgggcctgcccctcagtcgcggatccatcgacgtcagacgcggaaggagctccggtggactttgccgacaggtaccaaaacaaatgttctcgtcacgcgggcatgattcagatgctgtttccctgcaaaacgtgcgagagaatgaatcagaatttcaacatttgcttcacacacggggtcagagactgtttagagtgtttccccggcgtgtcagaatctcaaccggtcgtcagaaaaaagacgtatcggaaactctgcgcgattcatcatctgctggggcgggcgcccgagattgcttgctcggcctgcgacctggtcaacgtggacctggacgactgcgtttctgagcaataa

SEQ ID NO: 18 - DNA, 2214 bp, Adeno-associated virus - 7
  misc_feature 1..2214: note = CapVP1
  source 1..2214: mol_type = genomic DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 19 - DNA, 1878 bp, Adeno-associated virus - 8
  misc_feature 1..1878: note = Rep78
  source 1..1878: mol_type = genomic DNA
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataa

SEQ ID NO: 20 - DNA, 2217 bp, Adeno-associated virus - 8
  misc_feature 1..2217: note = CapVP1
  source 1..2217: mol_type = genomic DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 21 - DNA, 2211 bp, Adeno-associated virus - 9
  misc_feature 1..2211: note = CapVP1
  source 1..2211: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 22 - DNA, 1869 bp, Adeno-associated virus - 10
  misc_feature 1..1869: note = Rep78
  source 1..1869: mol_type = genomic DNA
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagtcctactttcacctgcacgttctggtcgagaccacgggggtcaagtccatggtcctgggccgcttcctgagtcagatcagagacaggctggtgcagaccatctaccgcggggtagagcccacgctgcccaactggttcgcggtgaccaagacgcgaaatggcgccggcggggggaacaaggtggtggacgagtgctacatccccaactacctcctgcccaagacgcagcccgagctgcagtgggcgtggactaacatggaggagtatataagcgcgtgtctgaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggaaagatcatggcgctgaccaaatccgcgcccgactacctggtaggcccgtccttacccgcggacattaaggccaaccgcatctaccgcatcctggagctcaacggctacgaccccgcctacgccggctccgtcttcctgggctgggcgcagaaaaagttcggtaaaaggaatacaatttggctgttcgggcccgccaccaccggcaagaccaacatcgcggaagccatcgcccacgccgtgcccttctacggctgcgtcaactggaccaatgagaactttcccttcaacgattgcgtcgacaagatggtgatctggtgggaggagggcaagatgaccgccaaggtcgtggagtccgccaaggccattctgggcggaagcaaggtgcgcgtcgaccaaaagtgcaagtcctcggcccagatcgaccccacgcccgtgatcgtcacctccaacaccaacatgtgcgccgtgatcgacgggaacagcaccaccttcgagcaccagcagcccctgcaggaccgcatgttcaagttcgagctcacccgccgtctggagcacgactttggcaaggtgaccaagcaggaagtcaaagagttcttccgctgggctcaggatcacgtgactgaggtgacgcatgagttctacgtcagaaagggcggagccaccaaaagacccgcccccagtgacgcggatataagcgagcccaagcgggcctgcccctcagttgcggagccatcgacgtcagacgcggaagcaccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgcgtcagaatctcaacctgtcgtcagaaaaaagacgtatcagaaactgtgcgcgattcatcatctgctggggcgggcacccgagattgcgtgttcggcctgcgatctcgtcaacgtggacttggatgactgtgtttctgagcaataa

SEQ ID NO: 23 - DNA, 2217 bp, Adeno-associated virus - 10
  misc_feature 1..2217: note = CapVP1
  source 1..2217: mol_type = genomic DNA
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttgggcagactggcgagtcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaacgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggaagagactctctggtgaatcccggtgtcgccatggcaacccacaaggacgacgaggaacgcttcttcccgtcgagcggagtcctgatgtttggaaaacagggtgctggaagagacaatgtggactacagcagcgttatgctaacaagcgaagaagaaattaaaaccactaaccctgtagccacagaacaatacggcgtggtggctgacaacttgcagcaagccaatacagggcctattgtgggaaatgtcaacagccaaggagccttacctggcatggtctggcagaaccgagacgtgtacctgcagggtcccatctgggccaagattcctcacacggacggcaactttcacccgtctcctctgatgggcggctttggacttaaacacccgcctccacagatcctgatcaagaacacgccggtacctgcggatcctccaacaacgttcagccaggcgaaattggcttccttcatcacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttatctgacacgtaatctgtaa

SEQ ID NO: 24 - DNA, 1869 bp, Adeno-associated virus - 11
  misc_feature 1..1869: note = Rep78
  source 1..1869: mol_type = genomic DNA
atgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccactggcgccgcgtgagtaaggccccggaggccctcttctttgttcagttcgagaagggcgagtcctacttccacctccacgttctcgtcgagaccacgggggtcaagtccatggtcctgggccgcttcctgagtcagatcagagacaggctggtgcagaccatctaccgcggggtcgagcccacgctgcccaactggttcgcggtgaccaagacgcgaaatggcgccggcggggggaacaaggtggtggacgagtgctacatccccaactacctcctgcccaagacccagcccgagctgcagtgggcgtggactaacatggaggagtatataagcgcgtgtctaaacctcgcggagcgtaaacggctcgtggcgcagcacctgacccacgtcagccagacgcaggagcagaacaaggagaatctgaacccgaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggaaagatcatggcgctgaccaaatccgcgcccgactacctggtaggcccgtccttacccgcggacattaaggccaaccgcatctaccgcatcctggagctcaacggctacgaccccgcctacgccggctccgtcttcctgggctgggcgcagaaaaagttcggtaaacgcaacaccatctggctgtttgggcccgccaccaccggcaagaccaacatcgcggaagccatagcccacgccgtgcccttctacggctgcgtgaactggaccaatgagaactttcccttcaacgattgcgtcgacaagatggtgatctggtgggaggagggcaagatgaccgccaaggtcgtggagtccgccaaggccattctgggcggaagcaaggtgcgcgtggaccaaaagtgcaagtcctcggcccagatcgaccccacgcccgtgatcgtcacctccaacaccaacatgtgcgccgtgatcgacgggaacagcaccaccttcgagcaccagcagccgctgcaggaccgcatgttcaagttcgagctcacccgccgtctggagcacgactttggcaaggtgaccaagcaggaagtcaaagagttcttccgctgggctcaggatcacgtgactgaggtggcgcatgagttctacgtcagaaagggcggagccaccaaaagacccgcccccagtgacgcggatataagcgagcccaagcgggcctgcccctcagttccggagccatcgacgtcagacgcggaagcaccggtggactttgcggacaggtaccaaaacaaatgttctcgtcacgcgggcatgcttcagatgctgtttccctgcaagacatgcgagagaatgaatcagaatttcaacgtctgcttcacgcacggggtcagagactgctcagagtgcttccccggcgcgtcagaatctcaacccgtcgtcagaaaaaagacgtatcagaaactgtgcgcgattcatcatctgctggggcgggcacccgagattgcgtgttcggcctgcgatctcgtcaacgtggacttggatgactgtgtttctgagcaataa

SEQ ID NO: 25 - DNA, 2202 bp, Adeno-associated virus - 11
  misc_feature 1..2202: note = CapVP1
  source 1..2202: mol_type = genomic DNA
atggctgctgacggttatcttccagattggctcgaggacaacctctctgagggcattcgcgagtggtgggacctgaaacctggagccccgaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaaaacggctcctggaaagaagagaccgttagagtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacttgtacctgcgtctcggaacaacatcaagcagcaacacctacaacggattctccaccccctggggatattttgacttcaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagaggggagcctgcctcctttccccaatgacgtgttcatggtgcctcaatatggctactgtggcatcgtgactggcgagaatcagaaccaaacggacagaaacgctttctactgcctggagtattttccttcgcaaatgttgagaactggcaacaactttgaaatggcttacaactttgagaaggtgccgttccactcaatgtatgctcacagccagagcctggacagactgatgaatcccctcctggaccagtacctgtggcacttacagtcgactacctctggagagactctgaatcaaggcaatgcagcaaccacatttggaaaaatcaggagtggagactttgccttttacagaaagaactggctgcctgggccttgtgttaaacagcagagattctcaaaaactgccagtcaaaattacaagattcctgccagcgggggcaacgctctgttaaagtatgacacccactataccttaaacaaccgctggagcaacatcgcgcccggacctccaatggccacagccggaccttcggatggggacttcagtaacgcccagcttatattccctggaccatctgttaccggaaatacaacaacttcagccaacaatctgttgtttacatcagaagaagaaattgctgccaccaacccaagagacacggacatgtttggccagattgctgacaataatcagaatgctacaactgctcccataaccggcaacgtgactgctatgggagtgctgcctggcatggtgtggcaaaacagagacatttactaccaagggccaatttgggccaagatcccacacgcggacggacattttcatccttcaccgctgattggtgggtttggactgaaacacccgcctccccagatattcatcaagaacactcccgtacctgccaatcctgcgacaaccttcactgcagccagagtggactctttcatcacacaatacagcaccggccaggtcgctgttcagattgaatgggaaattgaaaaggaacgctccaaacgctggaatcctgaagtgcagtttacttcaaactatgggaaccagtcttctatgttgtgggctcctgatacaactgggaagtatacagagccgcgggttattggctctcgttatttgactaatcatttgtaa

SEQ ID NO: 26 - DNA, 1866 bp, Adeno-associated virus - 12
  misc_feature 1..1866: note = Rep78
  source 1..1866: mol_type = genomic DNA
atgccggggttctacgaggtggtgatcaaggtgcccagcgacctggacgagcacctgcccggcatttctgactcctttgtgaactgggtggccgagaaggaatgggagttgcccccggattctgacatggatcagaatctgattgagcaggcacccctgaccgtggccgagaagctgcagcgcgagttcctggtggaatggcgccgagtgagtaaatttctggaggccaagttttttgtgcagtttgaaaagggggactcgtactttcatttgcatattctgattgaaattaccggcgtgaaatccatggtggtgggccgctacgtgagtcagattagggataaactgatccagcgcatctaccgcggggtcgagccccagctgcccaactggttcgcggtcacaaagacccgaaatggcgccggaggcgggaacaaggtggtggacgagtgctacatccccaactacctgctccccaaggtccagcccgagcttcagtgggcgtggactaacatggaggagtatataagcgcctgtttgaacctcgcggagcgtaaacggctcgtggcgcagcacctgacgcacgtctcccagacccaggagggcgacaaggagaatctgaacccgaattctgacgcgccggtgatccggtcaaaaacctccgccaggtacatggagctggtcgggtggctggtggacaagggcatcacgtccgagaagcagtggatccaggaggaccaggcctcgtacatctccttcaacgcggcctccaactcccggtcgcagatcaaggcggccctggacaatgcctccaaaatcatgagcctcaccaaaacggctccggactatctcatcgggcagcagcccgtgggggacattaccaccaaccggatctacaaaatcctggaactgaacgggtacgacccccagtacgccgcctccgtctttctcggctgggcccagaaaaagtttggaaagcgcaacaccatctggctgtttgggcccgccaccaccggcaagaccaacatcgcggaagccatcgcccacgcggtccccttctacggctgcgtcaactggaccaatgagaactttcccttcaacgactgcgtcgacaaaatggtgatttggtgggaggagggcaagatgaccgccaaggtcgtagagtccgccaaggccattctgggcggcagcaaggtgcgcgtggaccaaaaatgcaaggcctctgcgcagatcgaccccacccccgtgatcgtcacctccaacaccaacatgtgcgccgtgattgacgggaacagcaccaccttcgagcaccagcagcccctgcaggaccggatgttcaagtttgaactcacccgccgcctcgaccacgactttggcaaggtcaccaagcaggaagtcaaggactttttccggtgggcggctgatcacgtgactgacgtggctcatgagttttacgtcacaaagggtggagctaagaaaaggcccgccccctctgacgaggatataagcgagcccaagcggccgcgcgtgtcatttgcgcagccggagacgtcagacgcggaagctcccggagacttcgccgacaggtaccaaaacaaatgttctcgtcacgcgggtatgctgcagatgctctttccctgcaagacgtgcgagagaatgaatcagaattccaacgtctgcttcacgcacggtcagaaagattgcggggagtgctttcccgggtcagaatctcaaccggtttctgtcgtcagaaaaacgtatcagaaactgtgcatccttcatcagctccggggggcacccgagatcgcctgctctgcttgcgaccaactcaaccccgatttggacgattgccaatttgagcaataa

SEQ ID NO: 27 - DNA, 2229 bp, Adeno-associated virus - 12
  misc_feature 1..2229: note = CapVP1
  source 1..2229: mol_type = genomic DNA
atggctgctgacggttatcttccagattggctcgaggacaacctctctgaaggcattcgcgagtggtgggcgctgaaacctggagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtatctcaagtacaaccacgccgacgccgagttccagcagcgcttggcgaccgacacctcttttgggggcaacctcgggcgagcagtcttccaggccaaaaagaggattctcgagcctctgggtctggttgaagagggcgttaaaacggctcctggaaagaaacgcccattagaaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgagatgcgtgcggcgccaggcggaaatgctgtcgaggcgggacaaggtgccgatggagtgggtaatgcctccggtgattggcattgcgattccacctggtcagagggccgagtcaccaccaccagcacccgaacctgggtcctacccacgtacaacaaccacctgtacctgcgaatcggaacaacggccaacagcaacacctacaacggattctccaccccctggggatactttgactttaaccgcttccactgccacttttccccacgcgactggcagcgactcatcaacaacaactggggactcaggccgaaatcgatgcgtgttaaaatcttcaacatacaggtcaaggaggtcacgacgtcaaacggcgagactacggtcgctaataaccttaccagcacggttcagatctttgcggattcgacgtatgaactcccatacgtgatggacgccggtcaggaggggagctttcctccgtttcccaacgacgtctttatggttccccaatacggatactgcggagttgtcactggaaaaaaccagaaccagacagacagaaatgccttttactgcctggaatactttccatcccaaatgctaagaactggcaacaattttgaagtcagttaccaatttgaaaaagttcctttccattcaatgtacgcgcacagccagagcctggacagaatgatgaatcctttactggatcagtacctgtggcatctgcaatcgaccactaccggaaattcccttaatcaaggaacagctaccaccacgtacgggaaaattaccactggagactttgcctactacaggaaaaactggttgcctggagcctgcattaaacaacaaaaattttcaaagaatgccaatcaaaactacaagattcccgccagcgggggagacgcccttttaaagtatgacacgcataccactctaaatgggcgatggagtaacatggctcctggacctccaatggcaaccgcaggtgccggggactcggattttagcaacagccagctgatctttgccggacccaatccgagcggtaacacgaccacatcttcaaacaatttgttgtttacctcagaagaggagattgccacaacaaacccacgagacacggacatgtttggacagattgcagataataatcaaaatgccaccaccgcccctcacatcgctaacctggacgctatgggaattgttcccggaatggtctggcaaaacagagacatctactaccagggccctatttgggccaaggtccctcacacggacggacactttcacccttcgccgctgatgggaggatttggactgaaacacccgcctccacagattttcatcaaaaacacccccgtacccgccaatcccaatactacctttagcgctgcaaggattaattcttttctgacgcagtacagcaccggacaagttgccgttcagatcgactgggaaattcagaaggagcattccaaacgctggaatcccgaagttcaatttacttcaaactacggcactcaaaattctatgctgtgggctcccgacaatgctggcaactaccacgaactccgggctattgggtcccgtttcctcacccaccacttgtaa

SEQ ID NO: 28 - DNA, 1872 bp, Adeno-associated virus - 13
  misc_feature 1..1872: note = Rep78
  source 1..1872: mol_type = genomic DNA
atgccgggattctacgagattgtcctgaaggtgcccagcgacctggacgagcacctgcctggcatttctgactcttttgtaaactgggtggcggagaaggaatgggagctgccgccggattctgacatggatctgaatctgattgagcaggcacccctaaccgtggccgaaaagctgcaacgcgaattcctggtcgagtggcgccgcgtgagtaaggccccggaggccctcttctttgttcagttcgagaagggggacagctacttccacctacacattctggtggagaccgtgggcgtgaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaatatggaccagtatttaagcgcctgtttgaatctcgcggagcgtaaacggctggtggcgcagcatctgacgcacgtgtcgcagacgcaggagcagaacaaagagaaccagaatcccaattctgacgcgccggtgatcagatcaaaaacctccgcgaggtacatggagctggtcgggtggctggtggaccgcgggatcacgtcagaaaagcaatggatccaggaggaccaggcctcttacatctccttcaacgccgcctccaactcgcggtcacaaatcaaggccgcactggacaatgcctccaaatttatgagcctgacaaaaacggctccggactacctggtgggaaacaacccgccggaggacattaccagcaaccggatctacaaaatcctcgagatgaacgggtacgatccgcagtacgcggcctccgtcttcctgggctgggcgcaaaagaagttcgggaagaggaacaccatctggctctttgggccggccacgacgggtaaaaccaacatcgctgaagctatcgcccacgccgtgcccttttacggctgcgtgaactggaccaatgagaactttccgttcaacgattgcgtcgacaagatggtgatctggtgggaggagggcaagatgacggccaaggtcgtggagtccgccaaggccattctgggcggaagcaaggtgcgcgtggaccaaaagtgcaagtcatcggcccagatcgacccaactcccgtcatcgtcacctccaacaccaacatgtgcgcggtcatcgacggaaattccaccaccttcgagcaccaacaaccactccaagaccggatgttcaagttcgagctcaccaagcgcctggagcacgactttggcaaggtcaccaagcaggaagtcaaggactttttccggtgggcgtcagatcacgtgactgaggtgtctcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggcatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacatatcagaaactgtgtccgattcatcacatcatggggagggcgcccgaggtggcttgttcggcctgcgatctggccaatgtggacttggatgactgtgacatggagcaataa

SEQ ID NO: 29 - DNA, 2202 bp, Adeno-associated virus - 13
  misc_feature 1..2202: note = CapVP1
  source 1..2202: mol_type = genomic DNA
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggacttgacaagggggaacccgtcaacgcagcggacgcggcagccctcgaacacgacaaggcctacgaccagcagctcaaggccggtgacaacccctacctcaagtacaaccacgccgacgccgagtttcaggagcgtcttcaagaagatacgtcttttgggggcaacctcggacgagcagtcttccaggccaaaaagaggatccttgagcctctgggtctggttgaggaagcggctaagacggctcctggaaaaaagagacctgtagagcaatctccagcagaaccggactcctcttcgggcatcggcaaatcaggccagcagcccgctagaaaaagactgaattttggtcagactggcgacacagagtcagtcccagaccctcaaccactcggacaacctcccgcagccccctctggtgtgggatctactacaatggcttcaggcggtggcgcaccaatggcagacaataacgagggtgccgatggagtgggtaattcctcaggaaattggcattgcgattcccaatggctgggcgacagagtcatcaccaccagcacccgcacctgggccctgcccacctacaacaatcacctctacaagcaaatctccagccaatcaggagccaccaacgacaaccactactttggctacagcaccccctgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactccgagtaccagctcccgtacgtcctcggctcggcgcatcagggatgcctcccgccgttcccagcagacgtcttcatggtcccacagtatggatacctcaccctgaacaacgggagtcaggcggtaggacgctcttccttttactgcctggagtactttccttctcagatgctgcgtactggaaacaactttcagtttagctacacttttgaagacgtgcctttccacagcagctacgctcacagccaaagtctggaccgtctcatgaatcctctgatcgaccagtacctgtactatctgaacaggacacaaacagccagtggaactcagcagtctcggctactgtttagccaagctggacccaccagtatgtctcttcaagctaaaaactggctgcctggaccttgctacagacagcagcgtctgtcaaagcaggcaaacgacaacaacaacagcaactttccctggactggtgccaccaaatatcatctgaatggccgggactcattggtgaacccgggccctgctatggccagtcacaaggatgacaaagaaaagtttttccccatgcatggaaccctgatatttggtaaagaaggaacaaatgccaacaacgcggatttggaaaatgtcatgattacagatgaagaagaaatccgcaccaccaatcccgtggctacggagcagtacgggactgtgtcaaataatttgcaaaactcaaacgctggtccaactactggaactgtcaatcaccaaggagcgttacctggtatggtgtggcaggatcgagacgtgtacctgcagggacccatttgggccaagattcctcacaccgatggacactttcatccttctccactgatgggaggttttgggctcaaacacccgcctcctcagatcatgatcaaaaacactcccgttccagccaatcctcccacaaactttagtgcggcaaagtttgcttccttcatcacacagtactccacggggcaggtcagcgtggagatcgagtgggagctgcagaaggagaacagcaaacgctggaatcccgaaattcagtacacttccaactacaacaaatctgttaatgtggactttactgtggacactaatggtgtgtattcagagcctcgccccattggcaccagatacctgactcgtaatctgtaa

SEQ ID NO: 30 - DNA, 141 bp, Adeno-associated virus
  misc_feature 1..141: note = ITR Sequence
  source 1..141: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 31 - DNA, 1866 bp, Adeno-associated virus; identical to SEQ ID NO: 3
  misc_feature 1..1866: note = Rep2 Sequence Contains Rep78 and Rep52
  source 1..1866: mol_type = genomic DNA

SEQ ID NO: 32 - DNA, 2208 bp, Adeno-associated virus; identical to SEQ ID NO: 5
  misc_feature 1..2208: note = Cap2 Sequence contains sequentially VP1, VP2, AAP, VP3
  source 1..2208: mol_type = genomic DNA

SEQ ID NO: 33 - DNA, 2175 bp, Adeno-associated virus
  misc_feature 1..2175: note = Cap5 Sequence contains sequentially VP1, VP2, AAP, VP3
  source 1..2175: mol_type = genomic DNA
atggcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaagcaccagacctatcggaacccgataccttacccgacccctttaa

SEQ ID NO: 34 - DNA, 5336 bp, Adeno-associated virus
  misc_feature 1..5336: note = E2A Full Sequence
  source 1..5336: mol_type = genomic DNA
cgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttcgaggagaattcggtttcggtgggcgcggattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacgacaggtgctggcgccgggtgtggccgctggagatgacgtagttttcgcgcttaaatttgagaaagggcgcgaaactagtccttaagagtcagcgcgcagtatttgctgaagagagcctccgcgtcttccagcgtgcgccgaagctgatcttcgcttttgtgatacaggcagctgcgggtgagggagcgcagagacctgttttttattttcagctcttgttcttggcccctgctttgttgaaatatagcatacagagtgggaaaaatcctatttctaagctcgcgggtcgatacgggttcgttgggcgccagacgcagcgctcctcctcctgctgctgccgccgctgtggatttcttgggctttgtcagagtcttgctatccggtcgcctttgcttctgtgtgaccgctgctgttgctgccgctgccgctgccgccggtgcagtaggggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccacgagcggtagccttggcgctgttgttgctcttgggctaacggcggcggctgcttagacttaccggccctggttccagtggtgtcccatctacggttgggtcggcgaacaggcagtgccggcggcgcctgaggagcggaggttgtagcgatgctgggaacggttgccaatttctggggcgccggcgaggggaatgcgaccgagggtgacggtgtttcgtctgacacctcttcggcctcggaagcttcgtctaggctgtcccagtcttccatcatctcctcctcctcgtccaaaacctcctctgcctgactgtcccagtattcctcctcgtccgtgggtggcggcggcggcagctgcagcttctttttgggtgccatcctgggaagcaagggcccgcggctgctgatagggctgcggcggcggggggattgggttgagctcctcgccggactgggggtccaggtaaaccccccgtccctttcgtagcagaaactcttggcgggctttgttgatggcttgcaattggccaaggatgtggccctgggtaatgacgcaggcggtaagctccgcatttggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgctaagcagttgcgaattgcagaccagggagcggtgcggggtgcataggttgcagcgacagtgacactccagtaggccgtcaccgctcacgtcttccatgatgtcggagtggtaggcaaggtagttggctagctgcagaaggtagcagtgaccccaaagcggcggagggcattcacggtacttaatgggcacaaagtcgctaggaagcgcacagcaggtggcgggcagaattcctgaacgctctaggataaagttcctaaagttttgcaacatgctttgactggtgaagtctggcagaccctgttgcagggttttaagcaggcgttcggggaagataatgtccgccaggtgcgcggccacggagcgctcgttgaaggccgtccataggtccttcaagttttgctttagcagcttctgcagctcctttaggttgcgctcctccaggcattgctgccacacgcccatggccgtttgccaggtgtagcacagaaataagtaaacgcagtcgcggacgtagtcgcggcgcgcctcgcccttgagcgtggaatgaagcacgttttgcccgaggcggttttcgtgcaaaattccaaggtaggagaccaggttgcagagctccacgttggaaattttgcaggcctggcgcacgtagccctggcgaaaggtgtagtgcaacgtttcctctagcttgcgctgcatctccgggtcagcaaagaaccgctgcatgcactcaagctccacggtaacaagcactgcggccatcattagcttgcgtcgctcctccaagtcggcaggctcgcgcgtctcaagccagcgcgccagctgctcatcgccaactgcgggtaggccctcctcggtttgttcttgcaagtttgcatccctctccaggggtcgtgcacggcgcacgatcagctcgctcatgactgtgctcataaccttggggggtaggttaagtgccgggtaggcaaagtgggtgacctcgatgctgcgtttcagcacggctaggcgcgcgttgtcaccctcaagttccaccagcactccacagtgactttcattttcgctgttttcttgttgcagagcgtttgccgcgcgtttctcgtcgcgtccaagaccctcaaagatttttggcacttcgtcgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaacaggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttggtccccccgcccgacttgttcctcgtttgcctctgcgtcgtcctggtcttgctttttatcctctgttggtactgagcgatcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcaaagggggcggttaggctgtcctccttctcgactgactccatgatctttttctgcctataggagaaggaaatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtggggggtaaataatcacccgagagtgtacaaataaaaacatttgcctttattgaaagtgtctcctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttaagcatggagttgggtacc

SEQ ID NO: 35 - DNA, 1590 bp, unidentified
  misc_feature 1..1590: note = Adenovirus - E2A ORF Sequence
  source 1..1590: mol_type = unassigned DNA
atggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaa

SEQ ID NO: 36 - DNA, 3201 bp, unidentified
  misc_feature 1..3201: note = Adenovirus - E4 Full Sequence
  source 1..3201: mol_type = unassigned DNA
cccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaa

SEQ ID NO: 37 - DNA, 885 bp, unidentified
  misc_feature 1..885: note = Adenovirus - E4 Orf6 Sequence
  source 1..885: mol_type = unassigned DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtag

SEQ ID NO: 38 - DNA, 743 bp, unidentified
  misc_feature 1..743: note = Adenovirus - VA Sequence
  source 1..743: mol_type = unassigned DNA
cgtaatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatcctacatcga

SEQ ID NO: 39 - DNA, 870 bp, Human mastadenovirus C
  misc_feature 1..870: note = Ad5 E1A
  source 1..870: mol_type = genomic DNA
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataa

SEQ ID NO: 40 - DNA, 732 bp, Human mastadenovirus C
  misc_feature 1..732: note = Ad5 E1A
  source 1..732: mol_type = genomic DNA
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataa

SEQ ID NO: 41 - DNA, 531 bp, Human mastadenovirus C
  misc_feature 1..531: note = Ad5 E1B_19K
  source 1..531: mol_type = genomic DNA
atggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatga

SEQ ID NO: 42 - DNA, 1491 bp, Human mastadenovirus C
  misc_feature 1..1491: note = Ad5 E1B_55K
  source 1..1491: mol_type = genomic DNA
atggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattga

SEQ ID NO: 43 - DNA, 1590 bp, Human mastadenovirus C; identical to SEQ ID NO: 35
  misc_feature 1..1590: note = Ad5 E2A orf
  source 1..1590: mol_type = genomic DNA

SEQ ID NO: 44 - DNA, 453 bp, Human mastadenovirus C
  misc_feature 1..453: note = Ad5 E4A
  source 1..453: mol_type = genomic DNA
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtga

SEQ ID NO: 45 - DNA, 885 bp, Human mastadenovirus C; identical to SEQ ID NO: 37
  misc_feature 1..885: note = Ad5 E4A
  source 1..885: mol_type = genomic DNA

SEQ ID NO: 46 - DNA, 743 bp, synthetic construct
  misc_feature 1..743: note = Synthetic: Ad5 VA
  source 1..743: mol_type = other DNA
tcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacg

SEQ ID NO: 47 - DNA, 588 bp, synthetic construct
  misc_feature 1..588: note = Synthetic: CMV Promoter
  source 1..588: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtcgacgtttagtgaaccg

SEQ ID NO: 48 - DNA, 40 bp, synthetic construct
  misc_feature 1..40: note = Synthetic: 2xTet Operator Sequence
  source 1..40: mol_type = other DNA
tccctatcagtgatagagatctccctatcagtgatagaga

SEQ ID NO: 49 - DNA, 827 bp, synthetic construct
  misc_feature 1..827: note = Synthetic: hCMV Intron Sequence
  source 1..827: mol_type = other DNA
gtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatggtctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcag

SEQ ID NO: 50 - DNA, 583 bp, synthetic construct
  misc_feature 1..583: note = Synthetic: ECMV IRES Sequence
  source 1..583: mol_type = other DNA
cccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattgctcgaatcacc

SEQ ID NO: 51 - DNA, 461 bp, synthetic construct
  misc_feature 1..461: note = Synthetic: FMDV IRES
  source 1..461: mol_type = other DNA
agcaggtttccccaactgacacaaaacgtgcaacttgaaactccgcctggtctttccaggtctagaggggtaacactttgtactgtgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttacaattaatgaccct

SEQ ID NO: 52 - DNA, 6473 bp, Cricetulus griseus
  source 1..6473: mol_type = genomic DNA
tctagaaacaaaaccaaaaatattaagtcaggcttggcttcaggtgctggggtggagtgctgacaaaaatacacaaattcctggctttctaaggctttttcggggattcaggtattgggtgatggtagaataaaaatctgaaacataggtgatgtatctgccatactgcatgggtgtgtatgtgtgtgtatgtgtgtctgtgtgtgtgcccagacagaaataccatgaaggaaaaaaacacttcaaagacaggagagaagagtgacctgggaaggactccccaatgagatgagaactgagcacatgccagaggaggtgaggactgaaccattcaacacaagtggtgaatagtcctgcagacacagagagggccagaagcactcagaactccagggggtcaggagtggttctctggaggcttctgcccttggaggttcctgaggaggaggcttccatattgaaaatgtagttagtggccgtttccattagtacagtgactagagagagctgagggaccactggactgaggcctagatgctcagtcagatggccatgaaagcctagacaagcacttccgggtggaaaggaaacagcaggtgtgaggggtcaggggcaagttagtgggagaggtcttccagatgaagtagcaggaacggagacgcactggatggccccacttgtcaaccagcaaaagcttggatcttgttctaagaggccagggacatgacaagggtgatctcggtttttaaaaggctttgtgttacctaatcacttctattagtcagatactttgtaacacaaatgagtacttggcctgtattttagaaacttctgggatcctgaaaaaacacaatgacattctggctgcaacacctggagactcccagccaggccctggacccgggtccattcatgcaaatactcagggacagattcttcactaggtactgatgagctgtcttggatgcaaatgtggcctcttcattttactacaagtcaccatgagtcaggaggtgctgtttgcacagtgtgactaagtgatggagtgttgactgcagccattcccggccccagcttgtgagagagatccttttaaattgaaagtaagctcaaagttaccacgaagccacacatgtataaactgtgtgaataatctgtgcacatacacaaaccatgtgaataatctgtgtacatgtataaactgtgtgaataatctgtgtgcagcctttccttacctactaccttccagtgatcaggtttggactgcctgtgtgctactggaccctgaatgtccccaccgctgtcccctgtcttttacgattctgacatttttaataaattcagcggcttcccctctgctctgtgcctagctataccttggtactctgcattttggtttctgtgacatttctctgtgactctgctacattctcagatgacatgtgacacagaaggtgttccctctggagacatgtgatgtccctgtcattagtggaatcagatgcccccaaactgttgtccagtgtttgggaaagtgacacgtgaaggaggatcaggaaaagaggggtggaaatcaagatgtgtctgagtatctcatgtccctgagtggtccaggctgctgacttcactcccccaagtgagggaggccatggtgagtacacacacctcacacatactatatccaacacacacacacacacacacacacacacgcacgcacgcacgcacgcacgcacacatgcacacacacgaactacatttcacaaaccacatacgcatattacaccccaaacgtatcacctatacataccacacatacacacccctccacacatcacacacataccacacccacacacagcacacacatacataggcacacattcacacaccacacatatacatttgtgtatgcatacatgcatacacacacaggcacacagacaccacacacatgcattgtgtacgcacacatgcatacacacacataggcacacattgagcacacacatacatttgtgtacgcacactacatagacatatatgcatttgtatatgcacacatgcatgcacacatacataggcacacatagagcacacacatacatttgtgtatgcacacatgcacacaccaatcacatgggaagactcaggttcttcactaaggttcacatgaacttagcagttcctggttatctcgtgaaacttggaagattgctgtggagaagaggaagcgttggcttgagccctggcagcaattaaccccgcccagaagaagtaggtttaaaaatgagagggtctcaatgtggaacccgcagggcgccagttcagagaagagacctacccaagccaactgagagcaaaggcagagggatgaacctgggatgtagtttgaacctctgtaccagctgggcttcatgctattttgttatatctttattaaatattcttttagttttatgtgcgtgaataccttgcttgcataaatgtatgggcactgtatgtgttcttggtgccggtggaggccaggagagggcatggatcctccggagctggcgtttgagacagttgtgacccacagtgtggggtctgggaactgggtcttagtgttccgcaagtgcagctggggctcttaacctctgagccatccctccagcttcaagaaacttattttcttaggacatgggggaagggatccagggctttaggcttgtttgttcagcaaatactcttttcgtgtattttgaattttattttattttacttttttgggatagaatcacattctgcagctcaggctgggcctgaactcatcaaaatcctcctgtctcagtctaccaggtgataagattactgatgtgagcctggctttgacaagcactttagagtccccagcccttctggacacttgttccaagtataatatatatatatatatatatatatatatatatatatatatatattgtgtgtgtgtgtttgtgtgtgtatgagacacttgctctaagggtatcatatatatccttgatttgcttttaatttattttttaattaaaaatgattagctacatgtcacctgtatgcgtctgtatcatctatatatccttccttccttctctctctttctctcttcttcttctcacccccaagcatctattttcaaatccttgtgccgaggagatgccaagagtctcgttgggggagatggtgagggggcgatacaggggaagagcaggaggaaagggggacagactggtgtgggtctttggagagctcaggagaatagcagcgatcttccctgtccctggtgtcacctcttacagccaacaccattttgtggcctggcagaagagttgtcaagctggtcgcaggtctgccacacaaccccaatctggccccaagaaaaggcacctgtgtgtgactctggggttaaaggcgctgcctggtcgtctccagctggacttgaaactcccgtttaataaagagttctgcaaaataatacccgcagagtcacagtgccaggttcccgtgctttcctgaagcgccaggcacgggttccctaggaaatggggccttgcttgccaagctcccacggcttgccctgcaaacggcctgaatgatctggcactctgcgttgccactgggatgaaatggaaaaaagaaaaagaagaagtgtctctggaagcgggcgcgctcacacaaacccgcaacgattgtgtaaacactctccattgagaatctggagtgcggttgccctctactggggagctgaagacagctagtgggggcggggggaggaccgtgctagcatccttccacggtgctcgctggctgtggtgcatgccgggaaccgaaacgcggaactaaagtcaagtcttgctttggtggaactgacaatcaacgaaatcacttcgattgttttcctctttttactggaattcttggatttgatagatgggggaggatcagagggggaggggaggggcggggagacggagggaggaggggaggaggggaggaggggaggaggggaggaggggaagggatggaggaaaatactaacttttctaattcaacatgacaaagattcggagaaagtgcaccgctagtgaccgggaggaggaatgccctattgggcattatattccctgtcgtctaatggaatcaaactcttggttccagcaccaaggattctgagcctatcctattcaagacagtaactacagcccacacggaagaggctatacaactgaagaaataaaattttcactttatttcatttctgtgactgcatgttcacatgtagagagccacctgtgtctaggggctgatgtgctgggcagtagagttctgagcccgttaactggaacaacccagaactcccaccacagttagagcttgctgagagagggaggcccttggtgagatttctttgtgtatttatttagagacagggtctcatactgtagtccaagctagcctccagctcacagaaattctcctgttccggtttccaaagtactggagttatgagtgtgtgttaattgaacgctaagaatttgctgattgaagaaaacctcaagtgggtttggctaatccccacgaccccagaggctgaggcaggaggaatgagagaattcaaggtttgccagagccacagggtgagctcaatgtggagactgtgagggtgagctcaatgtggagactgtgagggtgagctcaatgtggagactgtgagggtgagctcaatgtggagactgtgagggtgagctcaatgtggagactgtgagggtgagctcaatgtggagacctgtatcaagataataatagtagtagtaacaatgcaggcgagggtgtggttgagtggtagagcagttagttgatttgacatgcttgaggtctcccggtccatctgtggccctgcaacaggaagggagggaggaagggggggaacgagagagaggaaagagagacagaagctaagatagggaatgagagaggaaggaagaaacgggaagaaattcagactccttcctgagttccgccaacgcctagtgacatcctgtgcacaccctaaggtggcctttgtgtggcactggcttgggtggtcgggaaaggcattttcagcttgttgcagaactgccacagtagcatgctgggtccgtgaaagtttctgcccgttaacaagaagtctctactacttgtgacctcaccagtgaaaatttctttaattgtctcctggtgttctgggttttgcatttttgtttctaaggatacattcctgggtgatgtcatgaagtccccaaagacacagtggggctgtgttggattgggaaagatgatttatctggggtgtcaaaaggaaaagaagggaaacaggcacttgggaaaatgtcctcccgcccacccgaattttggcttggcaaccgtggtggaggagcaagaaacacgtggacgtttgaggaggcatggggtcctaggaggacaggaagcagaaggagagagctgggctgacagcctgcaggcattgcacagtttcagaaggagattacagcatgactgagtttttagggatccaacagggacctgggtagagattctgtgggctctgaggcaacttgacctcagccagatggtatttgaataacctgctcttagagggaaaacagacatagcaaacagagccacgtttagtgatgaaactctcactttgcctgagtcatgtgcggccatgcccaggggtcaggctgacactcaactcaaaaacaagtgagaaattgaagacaatccgtggtggcagctactggaagggccaccacatccccagaaagagtggagctgctaaaaagccatttgtgataggcacagttatcttgaatgcatggagcagagattacggaaaaatcgagaatgttaatgaggcaacattcgagttgagtcattcagtgtgggaaacccagacgcttccatcccctaaaaggaacatcttgctctcagtcaaaatggaaataaaaattggggcttgaatttggcaaatgattcagaactctgtgtaggtattttcacacgcacagtggataattttcatgttggagtttatttgtgctaaaaggcagaaaagggtaaaaagcacatcttaagagttatgaggttctacgaataaaaataatgttacttacagctattccttaattagtacccccttccacctgtggtaatttcctgagatagtcagtggggaaaagatctctccttctcttctttctccccctcccctcctctccctccctccctccctccctccctcctctccctccctccccctttccttctttctttgctccttctcctctgcctccttctccctttcttcttcatttattctaagtagcttttaacagcacaccaattacctgtgtataacgggaaaacacaggctcaagcagcttagagaagattgatctgtgttcact

SEQ ID NO: 53 - DNA, 7045 bp, Cricetulus griseus
  source 1..7045: mol_type = genomic DNA
actagcgtgcaattcagaggtgggtgaagataaaaggcaaacatttgaggccatttccttatttggcacggcacttaggaagtggaacatgcctaatctactggtttgtaccacctttccctataatggactgtttgggaagctcctgggcaaccgattctggcatctcattggtcagaggcctgttaaatggtactcttatttgcaaagaaggctgtaacttgtagctttaaaagcctctcctcaagaaagaagggagaaaggatatggctagacatatctaatagacttaaccactgtgaaaagccttagtatgaatcagatagaacctatttttaactcagttttgaaaaaaataatctttatatttatttgtgtgtgtgtgtgtgtgtgtgtgtgtgtgtgtgtgtgtgtgtgtgtgaaccacatgtagcaggtgctggaggaggccagaagagggcaccagatctcctggaactgacaccacacatggttatgagctgcctgatgtgggtgctgggaactgaactctcgtgttctgcaagagcagcaactgttctcttaactgatgagccatctctccagccccccccataattttaattgttcattttagtaaattttattcataatcaattatcacagtataaaacaatgattttatatatatcatatacatatcaaggatgacagtgagggggatatgtgtgtgtgtgtgtgtgtgtgtgtgtgtgtgtgtgtgtgttatttgtgtgtgtgctttttaagaaggtgccatagtcactgcatttctctgaaggatttcaaaggaatgagacatgtctgtctgccaggaaccctatcttcctctttgggaatctgacccaaatgaggtattctgaggaactgaatgaagagctcaagtagcagtgtcttaaacccaaatgtgctgtctagagaaagtcaacgtcatcagtgagctgaggagagatttactgagcggaagacaagcgctctttgatttaagtggctcgaacagtcacggctgtggagtggagcctgtgctcaggtctgaggcagtctttgctagccagctgtgatgagcagtgaagaaagggtggagatggaggcagggtgggagcagggctatggttcagactaggtatcgtgagcacaccagctggttgacttgtggtctgtgggtcaggcgttgtaaacgccctcagggtcaggcagtcacattgcttgaagctgaatgggtgaggcaacacagagagtgcaaagaaggcaaagtaccacctcttccccgacccaggtcacttctgggttatagctgagactccggacagcatgcaaccagctggttagagcttcagggaaaacttgatgtctgcatgttgctatgaaatgtgattcggtacatctggagaaaatttataatgctggctcagtcaagcactgaacaaaggtaccttggctttgggagctacatgacattgacttgtaggcagacttttttttttctgcccgccaattcccagataaccaatatggaggctcaatattaattataaatgctcggctgatagctcaggcttgttactagctaactcttccaacttaaatgaacccatttctattatctacattctgccacgtgactttaccttgtacttcctgtttcctctccttgtctgactctgcccttctgcttcccagagtccttagtctggttctcctgcctaaccttatcctgcccagctgctgaccaagcatttataattaatattaagtctcccagtgagactctcatccagggaggacttgggtgctcccccctcctcattgccatccgtgtcttcctcttccctcgcttccccctcctcttcctgctcttcctcctccacccctcctttcatagtattgatggcaagggtgttctagaatggaggagtgcccataggcatgcaaagaaaccagttaggatgctctgtgaggggttgtaatcataagcgatggacacaattcaagccacagagtgaagacggaaggatgcactgtgctctagagcaacttctggggcagaatcacagggtgagtttctgacttgagggcgaagaggccacgaggaagggagtgagtttgtctgagctagaagctacggcccacctcttggtagcagacctgcccacaagcatgctttgttaatcatgtgggatctgattttcctctaaatctatgttcaactcttaagaaaatgtgaattctcacattaaaatttagatatacgtcttttggtggggggggtgtaaaaaatcctcaagaatatggatttctgggggccggagagatggctcagaggttaagagaactggttgctcttctagacattctgagttcaattcccagcaaccacatggtggctcacaaccatctgtaatgcgacctggtgccatcttctgacatgcatggatacatgcaggcagaaagctgtatacatagtaaattgataaatctttttttaaaaagagtatggattctgccgggtgttggtggcgcacgcctttaatcccagcactctggaggcagaggcaggtggatctctgtgagttcgagaccagcctggtctataagagctagttccaggacagcctccaaagccacagagaaaccctgtctcgaaaaaccaaaaaaaaaaaaaaaaaaaaaaaaaaaaaaagagtatggattctaagaaagccgtaacagctggagctgtgtacggagttcagcgtggtactagaagaacagacattcatgatgaaacaccccaggatttttacttagtatctagtttccattgttgttttgagaccggctcttatgctctccaggctggcctcaaactgctgatcttcccgcctctacctctcaagtcctgggactacttggctcataaaacagtttttgtcgggctccctgaagttatggttgtacaaaccgtgggggtcaatatactcacttgggcagagagagaaggtctgaatcccagacaatgactgcatctcaggacagttgggaagaggacaatggcagaaggacttagaaaagatagactggagggtggaaaagcagcaggaacagagaaacaaaacaggaagcttgctatccagggccactctggagtcctgtggcaagatggaagcgggctaggggaatacatttgtgctactgtgtgtgtgtgtgtgtgtgtgtgtgtgtgtgtgtgtgatcaatgcctatcaatgttgaaggggaaatatgtataccacattgattctgggagcaattctcagtatctggcctagagaaaggaatggcccctgcagaatagacagagtgaatggtgccctttatcatttgctaaagtgaaggagaaataaacatccttccatagagtttcaggtaaatgaaccccacagttcatctgtgccgtggtggaggcctggccaacagttaaaaagattagacacggacaaagtctgaaggaaacacctcgaataggaagaggagagccacctcattctgtaactttcctcaaggggaagatgttccaagagtgggaataaatggtcaaaggggggatttttaattaggaaaacgatttcctgtatcacttgtgaaactggaggttgatttggggcataggacaatagatttgatgctttgcaaaaagctgtttcaaagcagagaaatggaatagagacaattatgtagcgaggagggagggtggggcgaagatggagacagagaagtggaagctgactttagggaagaggaacatagaccacaggggcggggcggggggcaggggcggggggcggggctcaaaggaggcagtgggaacgttgctagtgttcgcagcgtaagcgtgaatgtgcaagcgtctttgtggtgtgtgaccaggagtagcgtggctggcttgtgtgctgcttgtaatcccagtctttgaggtttccacactgttccacagtgggtgtgattttccctcggagagcatgagggctctgctttccccacatcctccccagcgttcgttggtatttgtttccaagatgttagtgggtgagacaaagcctctctgttgatttgcctttaacaggtgacaaaaaaagctcaaccaggagacatttttgccttcttggaaggtaatgctcccatgtagagcaatgggacccatctctaaggtgaggctactcttgcagtttgcacccagctcttctgatgcaggaaggaagttggtgggcaagcaagactgtttgcttcttgcgatggacacattctgcacacaaaggctcaggaggggagaaggctgtttgatgtttagcactcaggaaggcccctgatgcatctgtgattagctgtctccatctgtggagcagacacggactaactaaaaaccagtgtttttaaattgtcaagcctttaaggtgaggaaattgacttattgtgctgggccatacgtagagcaagtgctctgcattgggccaacccccggctctggtttctaggcaccagaatggcctagaactaactcacaatcctcccattccaggtctcaggtgctagaatgaaccactataccagcctgcctgcctgcctacctgccttcctaaattttaaatcatggggagtaggggagaatacacttatcttagttagggtttctattgctgtgaagagacaccatgagcatggcaactcttataaaggaaaacatttagttgggtggcagtttcagaggttttagtacattgtcatcatggctgggaacatgatggcatgcagacagacatggtgctggagaaagggatgagagtcctacatcttgcaggcaacaggacctcagctgagacactggctggtaccctgagcataggaaacctcacagcccaccctcacagtgacatatttccttcaacaaagccatacctcctaatagtgccactccctatgagatgacagggccaattacattcaaactgctataacactttaaagtattttatttttattattgtaaattatgtatgtagctgggtggtggcagccgaggtgcacgcctttaatcccagcacttgggaggcagaggcagatggatctctgtgagttcaagaccagcctggtctataagagctagttgcaaggaaggatatacaaagaacagttctaggatagccttcaaagccacagagaagtgctgtcttgaaaaccaaaaattgtgctgggacctgtctctgctttggttgcttcccactcccccagagctggactcttggtcaacactgaatcagctgcaaaataaactcctggattcctctcttgtaacaggagcccgaagtcaggcgcccacttgtcttctcgcaggattgccatagactttttctgtgtgcccaccattccagactgaagtagagatggcagtggcagagactgggaaggctgcaacgaaaacaggaagttattgcaccctgggaatagtctggaaatgaagcttcaaaacttgcttcatgttcagttgtacacagactcactcccaggttgactcacacgtgtaaatattcctgactatgtctgcactgcttttatctgatgcttccttcccaaaatgccaagtgtacaaggtgagggaatcacccttggattcagagcccagggtcgtcctccttaacctggacttgtctttctccggcagcctctgacacccctccccccattttctctatcagaaggtctgagcagagttggggcacgctcatgtcctgatacactccttgtcttcctgaagatctaacttctgacccagaaagatggctaaggtggtgaagtgtttgacatgaagacttggtcttaagaactggagcaggggaaaaaagtcggatgtggcagcatgtacccgaaatcccagaactggggaggtagagacggatgagtgcccggggctagctggctgctcagccagcctagctgaattgccaaattccaactcctattgaaaaacctttaccaaacaaacaaacaaacaaataataacaacaacaacaacaacaaactaccccatacaaggtgggcggctcttggctcttgaggaatgactcacccaaacccaaagcttgccacagctgttctctggcctaaatggggtgggggtggggcagagacagagacagagagagacatgacttcctgggctgggctgtgtgctctaggccaccaggaactttcctgtcttgctctctgtctggcacagccagagcaccagcacccagcaggtgcacacacctccctccgtgcttcttgagcaaacacaggtgccttggtctgtctattgaaccggagtaagttcttgcagatgtatgcatggaaacaacattgtcctggttttatttctactgttgtgataaaaaccggggaactccaggaagcagctgaggcagaggcaaatgcaaggaatgctgcctcctagcttgctccccatggcttgccgggcctgctttctgcaagcccttctctccccattggcatgcctgacatgaacagcgtttgaaatgctctcaaatgtcactttcaaagaaggcttctctgatcttgctaactaaatcagaccatgtttcaccgtgcattatctttctgctgtctgtctgtctgtctgtctgtctatctgtctatcatctatcaatcatctatctatctatcttctatttatctacctatcattcaatcatctatcttctaactagttatcatttatttatttgtttacttactttttttatttgagacagtatttctctgagtgacagccttggctgtcctggaacccattctgtaaccaggctgtcctcaaactcacagagatccaactgcctctgcctctctggtgctggggttaaagacgtgcaccaccaacgccccgctctatcatctatttatgtacttattattcagtcattatctatcctctaactatccatcatctgtctatccatcatctatctatctatctatctatctatctatctatctatcatccatctataatcaattg

SEQ ID NO: 54 - DNA, 6473 bp, Cricetulus griseus
  source 1..6473: mol_type = genomic DNA
agtgaacacagatcaatcttctctaagctgcttgagcctgtgttttcccgttatacacaggtaattggtgtgctgttaaaagctacttagaataaatgaagaagaaagggagaaggaggcagaggagaaggagcaaagaaagaaggaaagggggagggagggagaggagggagggagggagggagggagggagaggaggggagggggagaaagaagagaaggagagatcttttccccactgactatctcaggaaattaccacaggtggaagggggtactaattaaggaatagctgtaagtaacattatttttattcgtagaacctcataactcttaagatgtgctttttacccttttctgccttttagcacaaataaactccaacatgaaaattatccactgtgcgtgtgaaaatacctacacagagttctgaatcatttgccaaattcaagccccaatttttatttccattttgactgagagcaagatgttccttttaggggatggaagcgtctgggtttcccacactgaatgactcaactcgaatgttgcctcattaacattctcgatttttccgtaatctctgctccatgcattcaagataactgtgcctatcacaaatggctttttagcagctccactctttctggggatgtggtggcccttccagtagctgccaccacggattgtcttcaatttctcacttgtttttgagttgagtgtcagcctgacccctgggcatggccgcacatgactcaggcaaagtgagagtttcatcactaaacgtggctctgtttgctatgtctgttttccctctaagagcaggttattcaaataccatctggctgaggtcaagttgcctcagagcccacagaatctctacccaggtccctgttggatccctaaaaactcagtcatgctgtaatctccttctgaaactgtgcaatgcctgcaggctgtcagcccagctctctccttctgcttcctgtcctcctaggaccccatgcctcctcaaacgtccacgtgtttcttgctcctccaccacggttgccaagccaaaattcgggtgggcgggaggacattttcccaagtgcctgtttcccttcttttccttttgacaccccagataaatcatctttcccaatccaacacagccccactgtgtctttggggacttcatgacatcacccaggaatgtatccttagaaacaaaaatgcaaaacccagaacaccaggagacaattaaagaaattttcactggtgaggtcacaagtagtagagacttcttgttaacgggcagaaactttcacggacccagcatgctactgtggcagttctgcaacaagctgaaaatgcctttcccgaccacccaagccagtgccacacaaaggccaccttagggtgtgcacaggatgtcactaggcgttggcggaactcaggaaggagtctgaatttcttcccgtttcttccttcctctctcattccctatcttagcttctgtctctctttcctctctctcgttcccccccttcctccctcccttcctgttgcagggccacagatggaccgggagacctcaagcatgtcaaatcaactaactgctctaccactcaaccacaccctcgcctgcattgttactactactattattatcttgatacaggtctccacattgagctcaccctcacagtctccacattgagctcaccctcacagtctccacattgagctcaccctcacagtctccacattgagctcaccctcacagtctccacattgagctcaccctcacagtctccacattgagctcaccctgtggctctggcaaaccttgaattctctcattcctcctgcctcagcctctggggtcgtggggattagccaaacccacttgaggttttcttcaatcagcaaattcttagcgttcaattaacacacactcataactccagtactttggaaaccggaacaggagaatttctgtgagctggaggctagcttggactacagtatgagaccctgtctctaaataaatacacaaagaaatctcaccaagggcctccctctctcagcaagctctaactgtggtgggagttctgggttgttccagttaacgggctcagaactctactgcccagcacatcagcccctagacacaggtggctctctacatgtgaacatgcagtcacagaaatgaaataaagtgaaaattttatttcttcagttgtatagcctcttccgtgtgggctgtagttactgtcttgaataggataggctcagaatccttggtgctggaaccaagagtttgattccattagacgacagggaatataatgcccaatagggcattcctcctcccggtcactagcggtgcactttctccgaatctttgtcatgttgaattagaaaagttagtattttcctccatcccttcccctcctcccctcctcccctcctcccctcctcccctcctccctccgtctccccgcccctcccctccccctctgatcctcccccatctatcaaatccaagaattccagtaaaaagaggaaaacaatcgaagtgatttcgttgattgtcagttccaccaaagcaagacttgactttagttccgcgtttcggttcccggcatgcaccacagccagcgagcaccgtggaaggatgctagcacggtcctccccccgcccccactagctgtcttcagctccccagtagagggcaaccgcactccagattctcaatggagagtgtttacacaatcgttgcgggtttgtgtgagcgcgcccgcttccagagacacttcttctttttcttttttccatttcatcccagtggcaacgcagagtgccagatcattcaggccgtttgcagggcaagccgtgggagcttggcaagcaaggccccatttcctagggaacccgtgcctggcgcttcaggaaagcacgggaacctggcactgtgactctgcgggtattattttgcagaactctttattaaacgggagtttcaagtccagctggagacgaccaggcagcgcctttaaccccagagtcacacacaggtgccttttcttggggccagattggggttgtgtggcagacctgcgaccagcttgacaactcttctgccaggccacaaaatggtgttggctgtaagaggtgacaccagggacagggaagatcgctgctattctcctgagctctccaaagacccacaccagtctgtccccctttcctcctgctcttcccctgtatcgccccctcaccatctcccccaacgagactcttggcatctcctcggcacaaggatttgaaaatagatgcttgggggtgagaagaagaagagagaaagagagagaaggaaggaaggatatatagatgatacagacgcatacaggtgacatgtagctaatcatttttaattaaaaaataaattaaaagcaaatcaaggatatatatgatacccttagagcaagtgtctcatacacacacaaacacacacacacaatatatatatatatatatatatatatatatatatatatatatattatacttggaacaagtgtccagaagggctggggactctaaagtgcttgtcaaagccaggctcacatcagtaatcttatcacctggtagactgagacaggaggattttgatgagttcaggcccagcctgagctgcagaatgtgattctatcccaaaaaagtaaaataaaataaaattcaaaatacacgaaaagagtatttgctgaacaaacaagcctaaagccctggatcccttcccccatgtcctaagaaaataagtttcttgaagctggagggatggctcagaggttaagagccccagctgcacttgcggaacactaagacccagttcccagaccccacactgtgggtcacaactgtctcaaacgccagctccggaggatccatgccctctcctggcctccaccggcaccaagaacacatacagtgcccatacatttatgcaagcaaggtattcacgcacataaaactaaaagaatatttaataaagatataacaaaatagcatgaagcccagctggtacagaggttcaaactacatcccaggttcatccctctgcctttgctctcagttggcttgggtaggtctcttctctgaactggcgccctgcgggttccacattgagaccctctcatttttaaacctacttcttctgggcggggttaattgctgccagggctcaagccaacgcttcctcttctccacagcaatcttccaagtttcacgagataaccaggaactgctaagttcatgtgaaccttagtgaagaacctgagtcttcccatgtgattggtgtgtgcatgtgtgcatacacaaatgtatgtgtgtgctctatgtgtgcctatgtatgtgtgcatgcatgtgtgcatatacaaatgcatatatgtctatgtagtgtgcgtacacaaatgtatgtgtgtgctcaatgtgtgcctatgtgtgtgtatgcatgtgtgcgtacacaatgcatgtgtgtggtgtctgtgtgcctgtgtgtgtatgcatgtatgcatacacaaatgtatatgtgtggtgtgtgaatgtgtgcctatgtatgtgtgtgctgtgtgtgggtgtggtatgtgtgtgatgtgtggaggggtgtgtatgtgtggtatgtataggtgatacgtttggggtgtaatatgcgtatgtggtttgtgaaatgtagttcgtgtgtgtgcatgtgtgcgtgcgtgcgtgcgtgcgtgcgtgtgtgtgtgtgtgtgtgtgtgtgtgttggatatagtatgtgtgaggtgtgtgtactcaccatggcctccctcacttgggggagtgaagtcagcagcctggaccactcagggacatgagatactcagacacatcttgatttccacccctcttttcctgatcctccttcacgtgtcactttcccaaacactggacaacagtttgggggcatctgattccactaatgacagggacatcacatgtctccagagggaacaccttctgtgtcacatgtcatctgagaatgtagcagagtcacagagaaatgtcacagaaaccaaaatgcagagtaccaaggtatagctaggcacagagcagaggggaagccgctgaatttattaaaaatgtcagaatcgtaaaagacaggggacagcggtggggacattcagggtccagtagcacacaggcagtccaaacctgatcactggaaggtagtaggtaaggaaaggctgcacacagattattcacacagtttatacatgtacacagattattcacatggtttgtgtatgtgcacagattattcacacagtttatacatgtgtggcttcgtggtaactttgagcttactttcaatttaaaaggatctctctcacaagctggggccgggaatggctgcagtcaacactccatcacttagtcacactgtgcaaacagcacctcctgactcatggtgacttgtagtaaaatgaagaggccacatttgcatccaagacagctcatcagtacctagtgaagaatctgtccctgagtatttgcatgaatggacccgggtccagggcctggctgggagtctccaggtgttgcagccagaatgtcattgtgttttttcaggatcccagaagtttctaaaatacaggccaagtactcatttgtgttacaaagtatctgactaatagaagtgattaggtaacacaaagccttttaaaaaccgagatcacccttgtcatgtccctggcctcttagaacaagatccaagcttttgctggttgacaagtggggccatccagtgcgtctccgttcctgctacttcatctggaagacctctcccactaacttgcccctgacccctcacacctgctgtttcctttccacccggaagtgcttgtctaggctttcatggccatctgactgagcatctaggcctcagtccagtggtccctcagctctctctagtcactgtactaatggaaacggccactaactacattttcaatatggaagcctcctcctcaggaacctccaagggcagaagcctccagagaaccactcctgaccccctggagttctgagtgcttctggccctctctgtgtctgcaggactattcaccacttgtgttgaatggttcagtcctcacctcctctggcatgtgctcagttctcatctcattggggagtccttcccaggtcactcttctctcctgtctttgaagtgtttttttccttcatggtatttctgtctgggcacacacacagacacacatacacacacatacacacccatgcagtatggcagatacatcacctatgtttcagatttttattctaccatcacccaatacctgaatccccgaaaaagccttagaaagccaggaatttgtgtatttttgtcagcactccaccccagcacctgaagccaagcctgacttaatatttttggttttgtttctaga

SEQ ID NO: 55 - DNA, 7045 bp, Cricetulus griseus
  source 1..7045: mol_type = genomic DNA
caattgattatagatggatgatagatagatagatagatagatagatagatagatagatgatggatagacagatgatggatagttagaggatagataatgactgaataataagtacataaatagatgatagagcggggcgttggtggtgcacgtctttaaccccagcaccagagaggcagaggcagttggatctctgtgagtttgaggacagcctggttacagaatgggttccaggacagccaaggctgtcactcagagaaatactgtctcaaataaaaaaagtaagtaaacaaataaataaatgataactagttagaagatagatgattgaatgataggtagataaatagaagatagatagatagatgattgatagatgatagacagatagacagacagacagacagacagacagcagaaagataatgcacggtgaaacatggtctgatttagttagcaagatcagagaagccttctttgaaagtgacatttgagagcatttcaaacgctgttcatgtcaggcatgccaatggggagagaagggcttgcagaaagcaggcccggcaagccatggggagcaagctaggaggcagcattccttgcatttgcctctgcctcagctgcttcctggagttccccggtttttatcacaacagtagaaataaaaccaggacaatgttgtttccatgcatacatctgcaagaacttactccggttcaatagacagaccaaggcacctgtgtttgctcaagaagcacggagggaggtgtgtgcacctgctgggtgctggtgctctggctgtgccagacagagagcaagacaggaaagttcctggtggcctagagcacacagcccagcccaggaagtcatgtctctctctgtctctgtctctgccccacccccaccccatttaggccagagaacagctgtggcaagctttgggtttgggtgagtcattcctcaagagccaagagccgcccaccttgtatggggtagtttgttgttgttgttgttgttattatttgtttgtttgtttgtttggtaaaggtttttcaataggagttggaatttggcaattcagctaggctggctgagcagccagctagccccgggcactcatccgtctctacctccccagttctgggatttcgggtacatgctgccacatccgacttttttcccctgctccagttcttaagaccaagtcttcatgtcaaacacttcaccaccttagccatctttctgggtcagaagttagatcttcaggaagacaaggagtgtatcaggacatgagcgtgccccaactctgctcagaccttctgatagagaaaatggggggaggggtgtcagaggctgccggagaaagacaagtccaggttaaggaggacgaccctgggctctgaatccaagggtgattccctcaccttgtacacttggcattttgggaaggaagcatcagataaaagcagtgcagacatagtcaggaatatttacacgtgtgagtcaacctgggagtgagtctgtgtacaactgaacatgaagcaagttttgaagcttcatttccagactattcccagggtgcaataacttcctgttttcgttgcagccttcccagtctctgccactgccatctctacttcagtctggaatggtgggcacacagaaaaagtctatggcaatcctgcgagaagacaagtgggcgcctgacttcgggctcctgttacaagagaggaatccaggagtttattttgcagctgattcagtgttgaccaagagtccagctctgggggagtgggaagcaaccaaagcagagacaggtcccagcacaatttttggttttcaagacagcacttctctgtggctttgaaggctatcctagaactgttctttgtatatccttccttgcaactagctcttatagaccaggctggtcttgaactcacagagatccatctgcctctgcctcccaagtgctgggattaaaggcgtgcacctcggctgccaccacccagctacatacataatttacaataataaaaataaaatactttaaagtgttatagcagtttgaatgtaattggccctgtcatctcatagggagtggcactattaggaggtatggctttgttgaaggaaatatgtcactgtgagggtgggctgtgaggtttcctatgctcagggtaccagccagtgtctcagctgaggtcctgttgcctgcaagatgtaggactctcatccctttctccagcaccatgtctgtctgcatgccatcatgttcccagccatgatgacaatgtactaaaacctctgaaactgccacccaactaaatgttttcctttataagagttgccatgctcatggtgtctcttcacagcaatagaaaccctaactaagataagtgtattctcccctactccccatgatttaaaatttaggaaggcaggtaggcaggcaggcaggctggtatagtggttcattctagcacctgagacctggaatgggaggattgtgagttagttctaggccattctggtgcctagaaaccagagccgggggttggcccaatgcagagcacttgctctacgtatggcccagcacaataagtcaatttcctcaccttaaaggcttgacaatttaaaaacactggtttttagttagtccgtgtctgctccacagatggagacagctaatcacagatgcatcaggggccttcctgagtgctaaacatcaaacagccttctcccctcctgagcctttgtgtgcagaatgtgtccatcgcaagaagcaaacagtcttgcttgcccaccaacttccttcctgcatcagaagagctgggtgcaaactgcaagagtagcctcaccttagagatgggtcccattgctctacatgggagcattaccttccaagaaggcaaaaatgtctcctggttgagctttttttgtcacctgttaaaggcaaatcaacagagaggctttgtctcacccactaacatcttggaaacaaataccaacgaacgctggggaggatgtggggaaagcagagccctcatgctctccgagggaaaatcacacccactgtggaacagtgtggaaacctcaaagactgggattacaagcagcacacaagccagccacgctactcctggtcacacaccacaaagacgcttgcacattcacgcttacgctgcgaacactagcaacgttcccactgcctcctttgagccccgccccccgcccctgccccccgccccgcccctgtggtctatgttcctcttccctaaagtcagcttccacttctctgtctccatcttcgccccaccctccctcctcgctacataattgtctctattccatttctctgctttgaaacagctttttgcaaagcatcaaatctattgtcctatgccccaaatcaacctccagtttcacaagtgatacaggaaatcgttttcctaattaaaaatcccccctttgaccatttattcccactcttggaacatcttccccttgaggaaagttacagaatgaggtggctctcctcttcctattcgaggtgtttccttcagactttgtccgtgtctaatctttttaactgttggccaggcctccaccacggcacagatgaactgtggggttcatttacctgaaactctatggaaggatgtttatttctccttcactttagcaaatgataaagggcaccattcactctgtctattctgcaggggccattcctttctctaggccagatactgagaattgctcccagaatcaatgtggtatacatatttccccttcaacattgataggcattgatcacacacacacacacacacacacacacacacacacacagtagcacaaatgtattcccctagcccgcttccatcttgccacaggactccagagtggccctggatagcaagcttcctgttttgtttctctgttcctgctgcttttccaccctccagtctatcttttctaagtccttctgccattgtcctcttcccaactgtcctgagatgcagtcattgtctgggattcagaccttctctctctgcccaagtgagtatattgacccccacggtttgtacaaccataacttcagggagcccgacaaaaactgttttatgagccaagtagtcccaggacttgagaggtagaggcgggaagatcagcagtttgaggccagcctggagagcataagagccggtctcaaaacaacaatggaaactagatactaagtaaaaatcctggggtgtttcatcatgaatgtctgttcttctagtaccacgctgaactccgtacacagctccagctgttacggctttcttagaatccatactctttttttttttttttttttttttttttttttggtttttcgagacagggtttctctgtggctttggaggctgtcctggaactagctcttatagaccaggctggtctcgaactcacagagatccacctgcctctgcctccagagtgctgggattaaaggcgtgcgccaccaacacccggcagaatccatactctttttaaaaaaagatttatcaatttactatgtatacagctttctgcctgcatgtatccatgcatgtcagaagatggcaccaggtcgcattacagatggttgtgagccaccatgtggttgctgggaattgaactcagaatgtctagaagagcaaccagttctcttaacctctgagccatctctccggcccccagaaatccatattcttgaggattttttacaccccccccaccaaaagacgtatatctaaattttaatgtgagaattcacattttcttaagagttgaacatagatttagaggaaaatcagatcccacatgattaacaaagcatgcttgtgggcaggtctgctaccaagaggtgggccgtagcttctagctcagacaaactcactcccttcctcgtggcctcttcgccctcaagtcagaaactcaccctgtgattctgccccagaagttgctctagagcacagtgcatccttccgtcttcactctgtggcttgaattgtgtccatcgcttatgattacaacccctcacagagcatcctaactggtttctttgcatgcctatgggcactcctccattctagaacacccttgccatcaatactatgaaaggaggggtggaggaggaagagcaggaagaggagggggaagcgagggaagaggaagacacggatggcaatgaggaggggggagcacccaagtcctccctggatgagagtctcactgggagacttaatattaattataaatgcttggtcagcagctgggcaggataaggttaggcaggagaaccagactaaggactctgggaagcagaagggcagagtcagacaaggagaggaaacaggaagtacaaggtaaagtcacgtggcagaatgtagataatagaaatgggttcatttaagttggaagagttagctagtaacaagcctgagctatcagccgagcatttataattaatattgagcctccatattggttatctgggaattggcgggcagaaaaaaaaaagtctgcctacaagtcaatgtcatgtagctcccaaagccaaggtacctttgttcagtgcttgactgagccagcattataaattttctccagatgtaccgaatcacatttcatagcaacatgcagacatcaagttttccctgaagctctaaccagctggttgcatgctgtccggagtctcagctataacccagaagtgacctgggtcggggaagaggtggtactttgccttctttgcactctctgtgttgcctcacccattcagcttcaagcaatgtgactgcctgaccctgagggcgtttacaacgcctgacccacagaccacaagtcaaccagctggtgtgctcacgatacctagtctgaaccatagccctgctcccaccctgcctccatctccaccctttcttcactgctcatcacagctggctagcaaagactgcctcagacctgagcacaggctccactccacagccgtgactgttcgagccacttaaatcaaagagcgcttgtcttccgctcagtaaatctctcctcagctcactgatgacgttgactttctctagacagcacatttgggtttaagacactgctacttgagctcttcattcagttcctcagaatacctcatttgggtcagattcccaaagaggaagatagggttcctggcagacagacatgtctcattcctttgaaatccttcagagaaatgcagtgactatggcaccttcttaaaaagcacacacacaaataacacacacacacacacacacacacacacacacacacacacatatccccctcactgtcatccttgatatgtatatgatatatataaaatcattgttttatactgtgataattgattatgaataaaatttactaaaatgaacaattaaaattatggggggggctggagagatggctcatcagttaagagaacagttgctgctcttgcagaacacgagagttcagttcccagcacccacatcaggcagctcataaccatgtgtggtgtcagttccaggagatctggtgccctcttctggcctcctccagcacctgctacatgtggttcacacacacacacacacacacacacacacacacacacacacacacacaaataaatataaagattatttttttcaaaactgagttaaaaataggttctatctgattcatactaaggcttttcacagtggttaagtctattagatatgtctagccatatcctttctcccttctttcttgaggagaggcttttaaagctacaagttacagccttctttgcaaataagagtaccatttaacaggcctctgaccaatgagatgccagaatcggttgcccaggagcttcccaaacagtccattatagggaaaggtggtacaaaccagtagattaggcatgttccacttcctaagtgccgtgccaaataaggaaatggcctcaaatgtttgccttttatcttcacccacctctgaattgcacgctagt

SEQ ID NO: 56 - DNA, 13515 bp, Cricetulus griseus
  source 1..13515: mol_type = genomic DNA
tctagaaacaaaaccaaaaatattaagtcaggcttggcttcaggtgctggggtggagtgctgacaaaaatacacaaattcctggctttctaaggctttttcggggattcaggtattgggtgatggtagaataaaaatctgaaacataggtgatgtatctgccatactgcatgggtgtgtatgtgtgtgtatgtgtgtctgtgtgtgtgcccagacagaaataccatgaaggaaaaaaacacttcaaagacaggagagaagagtgacctgggaaggactccccaatgagatgagaactgagcacatgccagaggaggtgaggactgaaccattcaacacaagtggtgaatagtcctgcagacacagagagggccagaagcactcagaactccagggggtcaggagtggttctctggaggcttctgcccttggaggttcctgaggaggaggcttccatattgaaaatgtagttagtggccgtttccattagtacagtgactagagagagctgagggaccactggactgaggcctagatgctcagtcagatggccatgaaagcctagacaagcacttccgggtggaaaggaaacagcaggtgtgaggggtcaggggcaagttagtgggagaggtcttccagatgaagtagcaggaacggagacgcactggatggccccacttgtcaaccagcaaaagcttggatcttgttctaagaggccagggacatgacaagggtgatctcggtttttaaaaggctttgtgttacctaatcacttctattagtcagatactttgtaacacaaatgagtacttggcctgtattttagaaacttctgggatcctgaaaaaacacaatgacattctggctgcaacacctggagactcccagccaggccctggacccgggtccattcatgcaaatactcagggacagattcttcactaggtactgatgagctgtcttggatgcaaatgtggcctcttcattttactacaagtcaccatgagtcaggaggtgctgtttgcacagtgtgactaagtgatggagtgttgactgcagccattcccggccccagcttgtgagagagatccttttaaattgaaagtaagctcaaagttaccacgaagccacacatgtataaactgtgtgaataatctgtgcacatacacaaaccatgtgaataatctgtgtacatgtataaactgtgtgaataatctgtgtgcagcctttccttacctactaccttccagtgatcaggtttggactgcctgtgtgctactggaccctgaatgtccccaccgctgtcccctgtcttttacgattctgacatttttaataaattcagcggcttcccctctgctctgtgcctagctataccttggtactctgcattttggtttctgtgacatttctctgtgactctgctacattctcagatgacatgtgacacagaaggtgttccctctggagacatgtgatgtccctgtcattagtggaatcagatgcccccaaactgttgtccagtgtttgggaaagtgacacgtgaaggaggatcaggaaaagaggggtggaaatcaagatgtgtctgagtatctcatgtccctgagtggtccaggctgctgacttcactcccccaagtgagggaggccatggtgagtacacacacctcacacatactatatccaacacacacacacacacacacacacacacgcacgcacgcacgcacgcacgcacacatgcacacacacgaactacatttcacaaaccacatacgcatattacaccccaaacgtatcacctatacataccacacatacacacccctccacacatcacacacataccacacccacacacagcacacacatacataggcacacattcacacaccacacatatacatttgtgtatgcatacatgcatacacacacaggcacacagacaccacacacatgcattgtgtacgcacacatgcatacacacacataggcacacattgagcacacacatacatttgtgtacgcacactacatagacatatatgcatttgtatatgcacacatgcatgcacacatacataggcacacatagagcacacacatacatttgtgtatgcacacatgcacacaccaatcacatgggaagactcaggttcttcactaaggttcacatgaacttagcagttcctggttatctcgtgaaacttggaagattgctgtggagaagaggaagcgttggcttgagccctggcagcaattaaccccgcccagaagaagtaggtttaaaaatgagagggtctcaatgtggaacccgcagggcgccagttcagagaagagacctacccaagccaactgagagcaaaggcagagggatgaacctgggatgtagtttgaacctctgtaccagctgggcttcatgctattttgttatatctttattaaatattcttttagttttatgtgcgtgaataccttgcttgcataaatgtatgggcactgtatgtgttcttggtgccggtggaggccaggagagggcatggatcctccggagctggcgtttgagacagttgtgacccacagtgtggggtctgggaactgggtcttagtgttccgcaagtgcagctggggctcttaacctctgagccatccctccagcttcaagaaacttattttcttaggacatgggggaagggatccagggctttaggcttgtttgttcagcaaatactcttttcgtgtattttgaattttattttattttacttttttgggatagaatcacattctgcagctcaggctgggcctgaactcatcaaaatcctcctgtctcagtctaccaggtgataagattactgatgtgagcctggctttgacaagcactttagagtccccagcccttctggacacttgttccaagtataatatatatatatatatatatatatatatatatatatatatatattgtgtgtgtgtgtttgtgtgtgtatgagacacttgctctaagggtatcatatatatccttgatttgcttttaatttattttttaattaaaaatgattagctacatgtcacctgtatgcgtctgtatcatctatatatccttccttccttctctctctttctctcttcttcttctcacccccaagcatctattttcaaatccttgtgccgaggagatgccaagagtctcgttgggggagatggtgagggggcgatacaggggaagagcaggaggaaagggggacagactggtgtgggtctttggagagctcaggagaatagcagcgatcttccctgtccctggtgtcacctcttacagccaacaccattttgtggcctggcagaagagttgtcaagctggtcgcaggtctgccacacaaccccaatctggccccaagaaaaggcacctgtgtgtgactctggggttaaaggcgctgcctggtcgtctccagctggacttgaaactcccgtttaataaagagttctgcaaaataatacccgcagagtcacagtgccaggttcccgtgctttcctgaagcgccaggcacgggttccctaggaaatggggccttgcttgccaagctcccacggcttgccctgcaaacggcctgaatgatctggcactctgcgttgccactgggatgaaatggaaaaaagaaaaagaagaagtgtctctggaagcgggcgcgctcacacaaacccgcaacgattgtgtaaacactctccattgagaatctggagtgcggttgccctctactggggagctgaagacagctagtgggggcggggggaggaccgtgctagcatccttccacggtgctcgctggctgtggtgcatgccgggaaccgaaacgcggaactaaagtcaagtcttgctttggtggaactgacaatcaacgaaatcacttcgattgttttcctctttttactggaattcttggatttgatagatgggggaggatcagagggggaggggaggggcggggagacggagggaggaggggaggaggggaggaggggaggaggggaggaggggaagggatggaggaaaatactaacttttctaattcaacatgacaaagattcggagaaagtgcaccgctagtgaccgggaggaggaatgccctattgggcattatattccctgtcgtctaatggaatcaaactcttggttccagcaccaaggattctgagcctatcctattcaagacagtaactacagcccacacggaagaggctatacaactgaagaaataaaattttcactttatttcatttctgtgactgcatgttcacatgtagagagccacctgtgtctaggggctgatgtgctgggcagtagagttctgagcccgttaactggaacaacccagaactcccaccacagttagagcttgctgagagagggaggcccttggtgagatttctttgtgtatttatttagagacagggtctcatactgtagtccaagctagcctccagctcacagaaattctcctgttccggtttccaaagtactggagttatgagtgtgtgttaattgaacgctaagaatttgctgattgaagaaaacctcaagtgggtttggctaatccccacgaccccagaggctgaggcaggaggaatgagagaattcaaggtttgccagagccacagggtgagctcaatgtggagactgtgagggtgagctcaatgtggagactgtgagggtgagctcaatgtggagactgtgagggtgagctcaatgtggagactgtgagggtgagctcaatgtggagactgtgagggtgagctcaatgtggagacctgtatcaagataataatagtagtagtaacaatgcaggcgagggtgtggttgagtggtagagcagttagttgatttgacatgcttgaggtctcccggtccatctgtggccctgcaacaggaagggagggaggaagggggggaacgagagagaggaaagagagacagaagctaagatagggaatgagagaggaaggaagaaacgggaagaaattcagactccttcctgagttccgccaacgcctagtgacatcctgtgcacaccctaaggtggcctttgtgtggcactggcttgggtggtcgggaaaggcattttcagcttgttgcagaactgccacagtagcatgctgggtccgtgaaagtttctgcccgttaacaagaagtctctactacttgtgacctcaccagtgaaaatttctttaattgtctcctggtgttctgggttttgcatttttgtttctaaggatacattcctgggtgatgtcatgaagtccccaaagacacagtggggctgtgttggattgggaaagatgatttatctggggtgtcaaaaggaaaagaagggaaacaggcacttgggaaaatgtcctcccgcccacccgaattttggcttggcaaccgtggtggaggagcaagaaacacgtggacgtttgaggaggcatggggtcctaggaggacaggaagcagaaggagagagctgggctgacagcctgcaggcattgcacagtttcagaaggagattacagcatgactgagtttttagggatccaacagggacctgggtagagattctgtgggctctgaggcaacttgacctcagccagatggtatttgaataacctgctcttagagggaaaacagacatagcaaacagagccacgtttagtgatgaaactctcactttgcctgagtcatgtgcggccatgcccaggggtcaggctgacactcaactcaaaaacaagtgagaaattgaagacaatccgtggtggcagctactggaagggccaccacatccccagaaagagtggagctgctaaaaagccatttgtgataggcacagttatcttgaatgcatggagcagagattacggaaaaatcgagaatgttaatgaggcaacattcgagttgagtcattcagtgtgggaaacccagacgcttccatcccctaaaaggaacatcttgctctcagtcaaaatggaaataaaaattggggcttgaatttggcaaatgattcagaactctgtgtaggtattttcacacgcacagtggataattttcatgttggagtttatttgtgctaaaaggcagaaaagggtaaaaagcacatcttaagagttatgaggttctacgaataaaaataatgttacttacagctattccttaattagtacccccttccacctgtggtaatttcctgagatagtcagtggggaaaagatctctccttctcttctttctccccctcccctcctctccctccctccctccctccctccctcctctccctccctccccctttccttctttctttgctccttctcctctgcctccttctccctttcttcttcatttattctaagtagcttttaacagcacaccaattacctgtgtataacgggaaaacacaggctcaagcagcttagagaagattgatctgtgttcactagcgtgcaattcagaggtgggtgaagataaaaggcaaacatttgaggccatttccttatttggcacggcacttaggaagtggaacatgcctaatctactggtttgtaccacctttccctataatggactgtttgggaagctcctgggcaaccgattctggcatctcattggtcagaggcctgttaaatggtactcttatttgcaaagaaggctgtaacttgtagctttaaaagcctctcctcaagaaagaagggagaaaggatatggctagacatatctaatagacttaaccactgtgaaaagccttagtatgaatcagatagaacctatttttaactcagttttgaaaaaaataatctttatatttatttgtgtgtgtgtgtgtgtgtgtgtgtgtgtgtgtgtgtgtgtgtgtgtgaaccacatgtagcaggtgctggaggaggccagaagagggcaccagatctcctggaactgacaccacacatggttatgagctgcctgatgtgggtgctgggaactgaactctcgtgttctgcaagagcagcaactgttctcttaactgatgagccatctctccagccccccccataattttaattgttcattttagtaaattttattcataatcaattatcacagtataaaacaatgattttatatatatcatatacatatcaaggatgacagtgagggggatatgtgtgtgtgtgtgtgtgtgtgtgtgtgtgtgtgtgtgtgttatttgtgtgtgtgctttttaagaaggtgccatagtcactgcatttctctgaaggatttcaaaggaatgagacatgtctgtctgccaggaaccctatcttcctctttgggaatctgacccaaatgaggtattctgaggaactgaatgaagagctcaagtagcagtgtcttaaacccaaatgtgctgtctagagaaagtcaacgtcatcagtgagctgaggagagatttactgagcggaagacaagcgctctttgatttaagtggctcgaacagtcacggctgtggagtggagcctgtgctcaggtctgaggcagtctttgctagccagctgtgatgagcagtgaagaaagggtggagatggaggcagggtgggagcagggctatggttcagactaggtatcgtgagcacaccagctggttgacttgtggtctgtgggtcaggcgttgtaaacgccctcagggtcaggcagtcacattgcttgaagctgaatgggtgaggcaacacagagagtgcaaagaaggcaaagtaccacctcttccccgacccaggtcacttctgggttatagctgagactccggacagcatgcaaccagctggttagagcttcagggaaaacttgatgtctgcatgttgctatgaaatgtgattcggtacatctggagaaaatttataatgctggctcagtcaagcactgaacaaaggtaccttggctttgggagctacatgacattgacttgtaggcagacttttttttttctgcccgccaattcccagataaccaatatggaggctcaatattaattataaatgctcggctgatagctcaggcttgttactagctaactcttccaacttaaatgaacccatttctattatctacattctgccacgtgactttaccttgtacttcctgtttcctctccttgtctgactctgcccttctgcttcccagagtccttagtctggttctcctgcctaaccttatcctgcccagctgctgaccaagcatttataattaatattaagtctcccagtgagactctcatccagggaggacttgggtgctcccccctcctcattgccatccgtgtcttcctcttccctcgcttccccctcctcttcctgctcttcctcctccacccctcctttcatagtattgatggcaagggtgttctagaatggaggagtgcccataggcatgcaaagaaaccagttaggatgctctgtgaggggttgtaatcataagcgatggacacaattcaagccacagagtgaagacggaaggatgcactgtgctctagagcaacttctggggcagaatcacagggtgagtttctgacttgagggcgaagaggccacgaggaagggagtgagtttgtctgagctagaagctacggcccacctcttggtagcagacctgcccacaagcatgctttgttaatcatgtgggatctgattttcctctaaatctatgttcaactcttaagaaaatgtgaattctcacattaaaatttagatatacgtcttttggtggggggggtgtaaaaaatcctcaagaatatggatttctgggggccggagagatggctcagaggttaagagaactggttgctcttctagacattctgagttcaattcccagcaaccacatggtggctcacaaccatctgtaatgcgacctggtgccatcttctgacatgcatggatacatgcaggcagaaagctgtatacatagtaaattgataaatctttttttaaaaagagtatggattctgccgggtgttggtggcgcacgcctttaatcccagcactctggaggcagaggcaggtggatctctgtgagttcgagaccagcctggtctataagagctagttccaggacagcctccaaagccacagagaaaccctgtctcgaaaaaccaaaaaaaaaaaaaaaaaaaaaaaaaaaaaaagagtatggattctaagaaagccgtaacagctggagctgtgtacggagttcagcgtggtactagaagaacagacattcatgatgaaacaccccaggatttttacttagtatctagtttccattgttgttttgagaccggctcttatgctctccaggctggcctcaaactgctgatcttcccgcctctacctctcaagtcctgggactacttggctcataaaacagtttttgtcgggctccctgaagttatggttgtacaaaccgtgggggtcaatatactcacttgggcagagagagaaggtctgaatcccagacaatgactgcatctcaggacagttgggaagaggacaatggcagaaggacttagaaaagatagactggagggtggaaaagcagcaggaacagagaaacaaaacaggaagcttgctatccagggccactctggagtcctgtggcaagatggaagcgggctaggggaatacatttgtgctactgtgtgtgtgtgtgtgtgtgtgtgtgtgtgtgtgtgtgatcaatgcctatcaatgttgaaggggaaatatgtataccacattgattctgggagcaattctcagtatctggcctagagaaaggaatggcccctgcagaatagacagagtgaatggtgccctttatcatttgctaaagtgaaggagaaataaacatccttccatagagtttcaggtaaatgaaccccacagttcatctgtgccgtggtggaggcctggccaacagttaaaaagattagacacggacaaagtctgaaggaaacacctcgaataggaagaggagagccacctcattctgtaactttcctcaaggggaagatgttccaagagtgggaataaatggtcaaaggggggatttttaattaggaaaacgatttcctgtatcacttgtgaaactggaggttgatttggggcataggacaatagatttgatgctttgcaaaaagctgtttcaaagcagagaaatggaatagagacaattatgtagcgaggagggagggtggggcgaagatggagacagagaagtggaagctgactttagggaagaggaacatagaccacaggggcggggcggggggcaggggcggggggcggggctcaaaggaggcagtgggaacgttgctagtgttcgcagcgtaagcgtgaatgtgcaagcgtctttgtggtgtgtgaccaggagtagcgtggctggcttgtgtgctgcttgtaatcccagtctttgaggtttccacactgttccacagtgggtgtgattttccctcggagagcatgagggctctgctttccccacatcctccccagcgttcgttggtatttgtttccaagatgttagtgggtgagacaaagcctctctgttgatttgcctttaacaggtgacaaaaaaagctcaaccaggagacatttttgccttcttggaaggtaatgctcccatgtagagcaatgggacccatctctaaggtgaggctactcttgcagtttgcacccagctcttctgatgcaggaaggaagttggtgggcaagcaagactgtttgcttcttgcgatggacacattctgcacacaaaggctcaggaggggagaaggctgtttgatgtttagcactcaggaaggcccctgatgcatctgtgattagctgtctccatctgtggagcagacacggactaactaaaaaccagtgtttttaaattgtcaagcctttaaggtgaggaaattgacttattgtgctgggccatacgtagagcaagtgctctgcattgggccaacccccggctctggtttctaggcaccagaatggcctagaactaactcacaatcctcccattccaggtctcaggtgctagaatgaaccactataccagcctgcctgcctgcctacctgccttcctaaattttaaatcatggggagtaggggagaatacacttatcttagttagggtttctattgctgtgaagagacaccatgagcatggcaactcttataaaggaaaacatttagttgggtggcagtttcagaggttttagtacattgtcatcatggctgggaacatgatggcatgcagacagacatggtgctggagaaagggatgagagtcctacatcttgcaggcaacaggacctcagctgagacactggctggtaccctgagcataggaaacctcacagcccaccctcacagtgacatatttccttcaacaaagccatacctcctaatagtgccactccctatgagatgacagggccaattacattcaaactgctataacactttaaagtattttatttttattattgtaaattatgtatgtagctgggtggtggcagccgaggtgcacgcctttaatcccagcacttgggaggcagaggcagatggatctctgtgagttcaagaccagcctggtctataagagctagttgcaaggaaggatatacaaagaacagttctaggatagccttcaaagccacagagaagtgctgtcttgaaaaccaaaaattgtgctgggacctgtctctgctttggttgcttcccactcccccagagctggactcttggtcaacactgaatcagctgcaaaataaactcctggattcctctcttgtaacaggagcccgaagtcaggcgcccacttgtcttctcgcaggattgccatagactttttctgtgtgcccaccattccagactgaagtagagatggcagtggcagagactgggaaggctgcaacgaaaacaggaagttattgcaccctgggaatagtctggaaatgaagcttcaaaacttgcttcatgttcagttgtacacagactcactcccaggttgactcacacgtgtaaatattcctgactatgtctgcactgcttttatctgatgcttccttcccaaaatgccaagtgtacaaggtgagggaatcacccttggattcagagcccagggtcgtcctccttaacctggacttgtctttctccggcagcctctgacacccctccccccattttctctatcagaaggtctgagcagagttggggcacgctcatgtcctgatacactccttgtcttcctgaagatctaacttctgacccagaaagatggctaaggtggtgaagtgtttgacatgaagacttggtcttaagaactggagcaggggaaaaaagtcggatgtggcagcatgtacccgaaatcccagaactggggaggtagagacggatgagtgcccggggctagctggctgctcagccagcctagctgaattgccaaattccaactcctattgaaaaacctttaccaaacaaacaaacaaacaaataataacaacaacaacaacaacaaactaccccatacaaggtgggcggctcttggctcttgaggaatgactcacccaaacccaaagcttgccacagctgttctctggcctaaatggggtgggggtggggcagagacagagacagagagagacatgacttcctgggctgggctgtgtgctctaggccaccaggaactttcctgtcttgctctctgtctggcacagccagagcaccagcacccagcaggtgcacacacctccctccgtgcttcttgagcaaacacaggtgccttggtctgtctattgaaccggagtaagttcttgcagatgtatgcatggaaacaacattgtcctggttttatttctactgttgtgataaaaaccggggaactccaggaagcagctgaggcagaggcaaatgcaaggaatgctgcctcctagcttgctccccatggcttgccgggcctgctttctgcaagcccttctctccccattggcatgcctgacatgaacagcgtttgaaatgctctcaaatgtcactttcaaagaaggcttctctgatcttgctaactaaatcagaccatgtttcaccgtgcattatctttctgctgtctgtctgtctgtctgtctgtctatctgtctatcatctatcaatcatctatctatctatcttctatttatctacctatcattcaatcatctatcttctaactagttatcatttatttatttgtttacttactttttttatttgagacagtatttctctgagtgacagccttggctgtcctggaacccattctgtaaccaggctgtcctcaaactcacagagatccaactgcctctgcctctctggtgctggggttaaagacgtgcaccaccaacgccccgctctatcatctatttatgtacttattattcagtcattatctatcctctaactatccatcatctgtctatccatcatctatctatctatctatctatctatctatctatctatcatccatctataatcaattg

SEQ ID NO: 57 - DNA, 14553 bp, Mus musculus
  source 1..14553: mol_type = genomic DNA
cttgaagaacacatgttttccaagagggagcacccatgttggaatgacaatgtagttagtgctcctctcctgtaggttagtgctcctttgctataggtaagtgctcctctcctataggtcagtgctcctctcctataggttagtgctcctctcctataggttagtgctcctctcctacaggttagtgctcctctgctctaggttagtcctgctctcctatagtacctagagagctagggcaaatgggctaggcccgaagtgcagagacaaacagctatggaagactgggtaagcacttccaagctacgaaagagcagtgtgaagggtcagggcttgtgcagttagtaggggagatcttccagttgaagaaacagaagaactgagagccactgggtatcatcctcctgcgccatgccttcctggatactgccatgctcccaccttgatgataatggaatgaacctctgaacctgtaagccagccccaatgaaatattgtttttatgagagttgccttggtcatgctgtctgttcacagcagtaaaaccctaaataaggcagaagttggtaccagtattgctgtgatagacctgaccatgctttcctttgaaagaatgtggatttggtgactttggatttgcaacacagtggaatgctttaaatggagattaatgggtcatcaattcctagtaggaatatggaagactttgttgctgggagtatttgaactgtgttgacctggcctaagagatttcaaaggagaagaatttcagaatgtggcataaagacagtttttgtggtattttggtgaagaatgtggctactttttgcccttgtctgaaaagtctgcctgagactaaagtgaagagaatcagattaattgcattgacaagggaagtttgtggctgcgctatctggaaacttacagccagcctcttggacctcgggtgacttacgcaaatactcagggacagagatgcttgactctgtactgatgagttgtcttggatgcaaatatgggctcttcatttgactacatgtcacgatgagtcaggagctgctctctccagagtgtgacaaagcgaggggatgctgacggtagctgttctagctttgaaggtaagcctgcacttatgctaaagtcacacatacacgagccgggtggagaacctgtctgtgtggagacacctttcattacctgtggcatccagcctctcaagcttggactgcctgtgtgctcctggactctggaggtcccactgctctgtcctctgctgcttatgatactgacattttaaaagaatccagtggttcccccctgtactcggtgtctacttctacctggatgttcctcatttatgttctgtgacacttctctgtgactctgctgcattcctgggtgacatgtggacaccctgtccctttgcagaccatgatgtcactgtcactagtggaatcagatgccccaagtgttgtcctgtgtttgggaacgtgacaggcagtacagaagcagaagaggaagggtgaaaacggaaatgtcacagcagcatctgatgtgtgcctcagtcacgcatgctgctgattggaactactcagcatgagagagggccatggtgaatacacaaccctatacacactgtgtccatttctctctctctcttacacagagagagagggaggagggggagggggaggcggagggggagggggagggagagggagtgggagagggagagggagagggagagggagagggagagggagagggagagggagagtttaatgtctgtgaagagataccatgaccaaagcaactcttataaaggacaacatttaattggggctggcttacaggttcagaaattcagtccattctcaccatggtgggaagcatgcaggtagatgtggtgctggaggaaccaagagttctatatcctgatctgaaggcagccaggagaagactgcctcttctgcacagggcagagcttgagcatagaacatcaaagcccttccccacacttcctccaacaaggtcatacatacttcaacaaagacacacctcctaacggtgccactccctgtggaccaaccatttaaacgcatgagtctatgagggtcaaagctcttcaaaccaccacactcatgtacacacacacacacacacacacacactctcatacacacacacacacacactcacacacacacacacacacacacacacacacacaccacacacacacacacacacagagttctattttgcactgtttcactgtcacaaggttctacttatctcagacacactgccaggaattgtgtgggaagactttcagtttctttgggttcacatggacttagcagttcttggtgatcctgaaagatttctgcagaaagaagccaaagtgttgagcccaaggcctggccacacattagtcctgtctagatgaacaggggtttaaaaataagggggcatcaaggtgaagccagcaggggctgacttagagaggagacccacccaagccaactgctcgaagtcaaaagcgatgaatccccatatccagctgtgcccggtgctgtcttgctacatctttagtaaatgttcttttagttgtatgcgtatgaatattttgcttgcatatatttgtgtacaccataggtgttcctagggcctatggaggccagaagagggcatcagatcctttggaactggaattatagacacttgttacccatagagtagattgtgggaaatgagcctttagtcttcgagagcggccagtgctcttaacctttggtcgtttctccaggtctttgagactttattttcttggacatcaggacaggatccagggctttgagcttgtttcttcagccagctttcttttcatgtatattaaattttatgttattttgctttctttttccccaagacagaatcacactctatatagctcaggctgggtttgaattcagtttccctgtctcagtctaccgggtaatatgattacagatgtgagtctgactttggtatcaaagtccccagcccttctggatatgtgttttaaggatatcagatatatccttgatttgctttgaattttctttttagttacaacataattagttccgtgtcacctgaatatgtgtatgtcacctacatagtcttccttcttctcttcttccctctcccaccttcccaggtacctgtctgtcttcatatccttgtgctgagagtcttgttgagggagatgatgaccgagacagagccactggggaagggagatgggctagtgcaggtcttcagagaggagctcgtgaatattgtagcccctttagtccctggcatgtcctcttgtatagccaccgccatgctgtggcctggcagaagtgaataagttgtccagctgttgacaggcctgccctccagacccagtctgatcccaagaaagggcatctgtgtctgtctctgaggccgtaagtgctgcctggttgtctccagcttgacttgacactccctccttaataagagtaccacagaacagggtctgcagagtccctgggccaggtccctgtgctgtcctggaatgccaggcgtgaatttcctgtgaagtaggactttgctcgccaagctcccacggcttgcccttcagatagccagaattatctggtaccctgcattgccgttcaatacgcagagtatcactggaagcgcgcgcgcgcacacacacacacacacacacacacacacacacacacacacacgcccactccatctttaaaccccaccccccagcaacggcggtgtaaacactctccatcaggaagctgaaacgcagttgccctctgctggggagatgaaggcagcttgctgggggcgaggaccgtgctagcaaccttccctggtgcacacgggctctggtgcatgacgggaacggaaacgcggaactaaagtcagtcctgctttttttttttttttttttttttttttttttttttttttttttttttggcgttggtggtggactgagtgacaatcagtgaaatcacttaggttgtttttctcttcttcgttgggtttgatagacggtgggagagggtcagaggagaaggggagggatggggagagagggaggagggaggggcgggaggcggggggcgaggaaaacgtgctaacttctccaatcctacaagacaaaggtttggagaaagccgcactgagtgacccagcagaaggaatccaggaatgtccgctggaatctgactgttgattccagcgccatgcagagaatctaggctggtaggaacattctttgtcctatccgacataataactccaaccaacacggaaaagaaaggctatacaagtgaagaaatggcattttcactttcatgactatacaatcacttccaggtagtaacacgtgtctagcacagcggttctcaacctgggggtcacgatcccccacttttctgcatatcagacatttttacgttgttattcataacagtagcaaaattgcagctatgaagtaacaatgaaatgcatttatggtgcgtgtgtgtgtgtgtgggggggtatcaccttaacatttactgtaagaaggttgagaatactgctccagcagctagtgtgttggacttaggttctgggtatattattagcaatagccaaccagaatccccacccaccacagcattgaggccccatgcagggcttgctgggagaggcactgataagacttctttatgtatttatttagagacgaatactcattaggtaggccaagctagcgtcaaactcatggcaattctcctcctccagtttcctaagtactggactcaggagtgtgttgccatcatatacagtaaggatttattgactgaagaaaatctcaagtggctttggttaatccctactacgccagaggctgaggcaggaggcgcgcaaggtcaaggcttgcctgggctacatatagagtgagctcaattttgacacttggtgcggtgttagtagtaatagtaaagatgaaggtgtggctcaggtggggccggtgattggacacacttggggtctcctggtccatctgcagctgtgcaacaggaagagcggagaatgagaggaaagagagaaaagacagaatgagagagagggaggaagagagaaaaaggaaaagagagaggaaaggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaaaggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaagggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaagggaaaaaggaaaatgaggaaagcgagaaagaagaaatgagaaagaggaaagggagaaagaaatgagagagagaaaagaaaagacagaatgcgagagagggaggaagagagaaaaaggaaaagagagaggaagggaaaatggaaaatgaggaaagcgagaaagaagaaatgagaaagaggaaagggagaaagaaatgagcgagataaaagacagaatttgagagagggaggaagaaataggaaaagagaggaaaggatggagaaaagagagaaagaaagagagatgaaagagagaaaggagaaatgaaatgagagagagagagagacacaaagagccagagagagaagaaaaaaggggaaagagaaagagaaagaggaaggctcctcttggacacatcttcctttatctttccctggggaccgccaaagcctggtggcatactgtacattctgtacactgttcattcaaaacaggctctgtcttaaagatggtctgagcggtcagaaaagggtattgttaacttgtttgcaaaactgcctcaggagagtgctgagtgcgtgaaagttgctgcccgttaaggagaagtctctactacttgtgatctcaccatcgaaaatttctttaattgtctcctggtgttctgggttttgcagttttgtttctaaggatacattcttgggtgatgtcacaaagtccccaaagacacggtggagctgtgttagatggggaaagacagtctgctgaggatttatctggaactgtcagaaggaaaagaaggtaaatggggcacttgggaaagtggcctctagtttgacttctggcttagcaaaggttgtggggagataaggcatacacagtagttagcaggaggcaacagggtcctgggaggacgcgaggcagaaggagaggctgggctgacagcatgcaatcattgcatagtctccaaaggagattgcaacatggctgagttttcagaggtcctacagagcccgtggtagagattctgtgggttctgagacaacttgactttagccagatggtatttgagtaatctgggagagagaaaacagctacagcaaacagggccacatttagtgacgaaactctcactttgactgttgagtcatttgcagtgggccctgaggtcaggctggccctcagctcaaaaacaagcgaggaactgaagcaattactcagataatccacagccacagccactggaaagggccacatccccagagacagcacagcaggggtgggggtggggctatgagaaagttagtgattgtagcagttatctagaatgtgcggagcagaggaggttacacaaaaacctagaatgtcattcaatgtgggaaaccgagaggctcccaagccctaaaaggaacagtttgctttcagccaaaatggaaataaaatttggggcttaaatctggcaaatgattcagaccttctgtgtaggtgtctttaaatgcacagcagattgattttcatgttggagtttatttgaactaaaagacagaaatggtgaaaagcacacctgaagaaattgagatgctatgaataaaatcatttacttacagctatcacttaattagtacctccttccaccttgctgatttattgggctagtcaaggaagaaaagatcttccctcctccttctctcctcctccccctcctctcctcctcccctcccctccttgaccttcctctcctccttttccctcctccccctcttcttctcttcaccccctcctcccctcccctcctctgtactcctcccctttcctcccaatctcttttttctcccccttcttctctttctcccccctcctcttccctcctcttcctccctccctccctcctcctcctcatcctcctcttcctcttcatcctcttctccttcctccctctcctcctcctccttttccagccctacctaccttccctttcttcttcatttattcaaagtagctttgaacagcactactcggtttagttgtgtataaaaggaaaatgcaggtccaagcagcttggggaagattgctttttgctctctggaggcagatgatgacagttcaagatcattccttttgctccatgtcacaggaagggggacatgccgaatctaccagtttgcagccacctacacaggatccaccttcacttctaaggaaatgtttgggaagctacctaccaaccacttctggcatctcatgggctagaggactcttaaatggcactcttatttgtttaataaaggaggttgtgacgtgtagttttaaatcccttccacacaacaattgctactctctgaccaaaaaagaagggagacaggatacggctaggtgtctagtagactttaccactttgaaaagccttaatataaatcaggtagatacatctttttaacttattcttgtaaagacaaaaacaaaactttatttttatttgtgtgtatgcttgtgtgtgtgtgcctgtgtgtataccacatgtcgctggtgccggagaacaccagaagaggggacctgatctcctggagctaaagctatccatggttctgagctgcctgatgtgggtgctgggaacagaactctggtcttctgcaagagcaacaagcctcctcttaactacgaatctcctccccatccccccaaatacatttaattattcattttagcagctttatttcgtaactacttatcacagcataaaacaaggattttatatatattacatgcaatcgaggataagagttgaggggagatgcgtgtgctccttctgggtgtctgtgcttttgaagaatgtaagcagtgcacaagggaccgaggcgtgcctgtctgccaggagctgtcttcttcccttggactctgagctgagtgcagtgctccgaagaagtaaaagacgacctcatgaagcaatgtcttcaacccaaacatgctgtccagacaaagtccagcttcattagtgctctgaggagagacttactgagcctcaggaaagcccccctcagcatggcgaaagtccactttgattgaagtgactcgaaagccatggcagtgcggcggcggccgcgtggagcttgtgctcgagtcggaagcggcatctttgtcaggcggctgtgattagcacggggaggcaggactggagtgaaggaagagttgggggcggggcttagcgctctggtctcctaagctgtagtcagcgcctcaagatttgtaacctgccttctgccttcccagccaggcagtcaagtggctccaagctgaagactgcaaagtgcccctaaccttttggttatagcgaggctgaagacaccgtgctctttcatgaaagccggatgtctgaaatccgatttgataaatatggataaaacgtataacgctcgatcaatcgaatcgaaggagctcacgattggcaccacggctttggggacaacagagtactgactcgttgggaggacttggatacttcccctcctcttccatctcttcccctttcctcacttcctcctccttccttctccattttctccctcttcactgtttcttactatttttacaaaagattttatttatttatttatttatttatttatttatttatttatttatttatttatttaatgtatgcgagtacactgtagctgtcttcagacacaccagaagagggcgtcaagttccattagagatggtttcgagccaccatgtggttgctggggcctctggaaggaccgccagtgctcttaacccctgagccatttctccagtacccttctcaccgtttctcttcaatcttcttcctcttccttctccactttccttgtcttcttggtttcattatctttctccctttcttcctcttctccccttcttcctcctccactgtagttttccttccctactcttttcctgcctccctcctcctcccctctcattccccctcctctttcctccttctccctcctcctccttccttctccctctcccctctcccctctcccttctcccttctccccctcctcttcctctttctccttctccacccctcctgtcacagtatcaatggcaagggtgttctagaatggaggagtgtcccctaggcactaacgaaagccagttaggatgctctgagacgggtacaattcagggagggccgtggggatggaagggttgtgctgcgattcattctggagcaacccccaggcagaatcatgaggttggttccggattcgcagggcacaattcagaagaggaaggtttcaggaaggacgagtttgtctgagataggagttacatctgatgtcttggcagcagagccactgtacaagcgtgctttattaaccacgtgggattaaatcttcttttaaatttattttcaactcttaaggaaacgtgaactttcacattcaaatttagacttgcagctcttatggggaaaaaaaggggatcttaagaatattaagcataggcggctggagagatggctcagcggttaagagcactctctgctctcccagaggtcctgagttcaattcctagcaaccacataatagttaacaacagtctttaatgaattctaatgccctcttctggtgtgtctgaagacagttacagtgtactcatataaataaaataaagaaatttaaaaaaatgaatattaggcatagattcctggatcctaagaaagccatcagagctggagccatgtgtgggatcctgcttggtgctggaggggcagagttcatgcccccggggtttttacttattatcacattttcatcgttgttttgaaacagggtcttgtgtggtccaggctggccttgaactcatctttcagcctctacctcacaggttctgggattacttggttcctaaaagtatctccgtcaagctccctggtgttatggctgtgccaaccaggagggtctatacactcgctcaggtagagggagaagatccgaatctctgacagggactgctgcctctcggggcaaatggagtgaaggacagcggcagaaggatttaggaaagatggacgggagagtggaaatgctgcagaagccagaaaacaaagcaggaagcctgctgtccagtggggctcaagagcggagggatgcgagggggctgcgcaggaacatttagcgtctgcgtctatgggggtaggggcggggtgccagcacctagtcacctgaaggggaaatgcttgcccagggagcaggtctcagtagctgacctagagaaaggagcggcccctacagaggagacacgggtcactgtttgttaaagtgaaggagaaataaatattctttcaaagaatcttaggtgagcccagttcatctgcgctgtggaggcctggggaacagttaaaaagaccctgacacacacccaaggcaaacaagcaacacacggctccttccgtaagggtccatgattctctgaagaatcagccccggaatcagccccggaatcaggtagtccgtaaacacaatgagtgttttactctgcagaagtccagcctgctggcgtctcccattaccaaaatagagggatagtcacgtgagctcaccggctcgatttaaggcacgtggttttccagggtagatgagctttggcttctggaaccattatggggcacgaaggatggagccaggattttttttttttttttttttttctattagcaattgatttgcttgggcttggctggacttgcccagttcttaggcccagtcttcttaactgccgatctgaagtctgtcatggagtcagcctagccttctcacttcccttcagctcgaataggaagaggaggtgcacaccagatggtctgagagcagggataaatggtgtgcctttgtctttcagtatttcgttattttaagtaggaagatgcttttctgtattacattgcttgtgaaaccggaagttgattcggggcacaggacaatggatttggtgttttgcaaggactgtttcagaagagagaggagtggaagggtggttagagtgaggagtggggtgggacgggatgggggaagagaaggaagggccagacaggctaggtagggctgagaggaggcggtgggaacttcttgagttagcgcagcagtaaacttggatgtgcgtgtatctttgtgatatatgacccggagccgtgtagctggctccgatagtactgctaatgtcagtgtcggggggggggggtcccatactgttccacaggggctgcacattcccatcgagagcaggagggctcctctctccatacatcctcgccagcattccttgttgtttctgtgatgacagggggtgggatgaaatctctctgttggtttgagagaccgtgaagaagctcaaccccaggacattttgcagtcttggaaggcagtgcctccatgtggagccgtggagcccatctctgagtccaggtcactcttgcagttcgcactcagctcttcagatgcaggagagacgttggtgggaaagcaagattgtttgcttgttgagatagacacattctccacacaaaggctcacgtggggcaaaggctgattgacgtacagcgttcaggaacgcctgtggtagagctatgattagctgtctccatctatgaagcagacaaagagttataaaaaaaatcaatgttttcaaattgtcaaacttttaacccgacagcaagcgctctgtccctgggctaatccctagccctggtttcttgagatggggtcttttgtgcactagactggcctagaactcacgatcttagtgttccagcctcccagctgctgggatgagccgctataaccagtctgcctgccttcctaaattttaagtgatgggaagtgggggagaatacagtttaaagtatgcagatctgagagcaggaacctggcaaagccaaggggccggagttacaggcggctaacatgggtgctgggaactgacccaggtccttgagaggagcagtgtgtactcttgaccaaacaggtccgtctctccagtccccgtagtattaaaaataggtactacgggcatggtggtgcacacctttaatcccagcactagggaggcagaggcaggtggatttctgagtttgaggccagcctggtctacaaaatgagttccaggacagccacggctatacagagaaaccctgtcttgaaaacaaaacaacaacaaaataggtactacaaagcgatgtaattgtgctcaaacatgcaaaccgaggggactgtatgcataagaaagagaaagacggccacactggttctatctgggtgacaggaaatcagtatttttatttttcacattcatttttttgttgttgttgttgacacagtgatttttctatcaaaaacattatttcttttatagttcccctgaggagctgtttttaaagccgtgctttgaaaaaccattgaaggagcagaggcagggagactcctgtgtggcagtcggtgaagcaggccctctgcaggcaggctggccctggacttgggagtctctttccctccctcctgtgctcaaatagcaaatgtcaggcttcaatgtagctagaaggttctagaatgattaagtttccaaggctgaagagcttccctgtttgcctttcacttccctggagaggtcgttgtgtgttccggagtctgcaaggtgcctttggtgatgcgggtggttcatctcgggagattccgcctggaggacccaagttcaagccctgcctgagctacagagtgactttcaggtcttctgcgcaattcagtgagacccagtctacaaataaaaagtaaaaagaaggctgtggatggaactcggtggtagagttctgggtttactccctagaggaggggagaaggaggaggagggaggaggaagaggaagaaagaagaagagaagggaagaggagaaggaagggagggaaggggctgacaagaagagagaagagggagggaggggagggaaaggaaggggaaaggaagggagggaaggggctgacaagaagagagaagagggagggaggggagggaaaggaaggggaaagaagagaagggtaagaagaaactgttccaatggtctgggccacagagtgatggccttttgtggtgatcagctgtaatccttgatttgacacaacctagaatctgggaagcgagtttctgtgaaggagcattcacactggctggcctgtgggcgtgcatgtgggagactgtcataattaggttcattaatacaggaagtcccagcccactacaaatggcttcgttccatacccaagagatgctaactgtagacggttggagaaagcaagcaagctgtggataccccacgctctttcacctcggctcctggggggtgggtgcactgtgtctcttggtattttaaagtcctgccttgacgtccctgctgtgacagactgtaactggaattgtgagctttagtcctttagttttctacgttggtttttctcaggatattttatcgcagtaacagaaacaagaccaggacacttgatctcctctgatcaacactgaagagttacaaaacaggctgaggaaacaaactttcttctccctctcccccttctgtccctccccttccttctcgctccctcccttgccccctctctccctgtctctgtctctgtctctgtctctgtctctgtctctgtctctgcctctcccctcccctcccctccctctgtctctgtctctgtctctgtctctgtctctgtctctgtctctgtccctttctcctctatctcctaaatggctggaggccatgctagctcaatgttgaactttgaacacgtatttaggaaatctttgttcttaacagttctgaagtgctgaagtggtggtttagtctctcggcctgacaagctcacttcctctcactctgtcttaatgaccaaatctgccatttccctaaaacagcacaggctccagctccaggttgctccggagcggag

SEQ ID NO: 58 - DNA, 4001 bp, Cricetulus griseus
  source 1..4001: mol_type = genomic DNA
ccaagatgcccatcaactgattaatagatgataaaattattgtacatttcagtgtaatattattcagtttttaagaaaaatgaaattatgtaataagcatgtaaatggatatatcttgaaacaaccattccccattatattacctaaacattgaaagtccaaaatcatatgatctttttagtggatctactaatcttttgctatatgtattttattgaactacccatggatgtgagataattggtaacaacagcacatgggagagcatgggatcattcaaggaagattagagagaatgcattttttaggagataatggaggagcaatagaaaggattaaatgaggttactgatgaaagtgatggttagagaaggcaatatgaggagggataactagcacttagggccttttgaaaaagacatagagaaaatactattgtagaaacttcctataattggtgtatagttatatacaccaaagagctcagatggagttaccctataatggaaatattaactactttttatcactgtgataaaacatcctgaacagagcaacatagattgggaagcatttactttggcttacagttctaacgggataaaaattcatgatgaaagaatgaatatgtcagcaaacagcagtagcaatggcctgagaagcaggtgagagctcacatcttgaagtgtaagaatgtagcagagagaacaaactgcaaatgaccagaaaatgcttttggatcagagcccatacccctctgactgacttctccagaaattctgaacaaataaaactccccaaacagagccataactgaaggtccagtgtctgagactactaggggtatttcttattcaaaccactacaatggggtggggggagcaatcctccaagtaggcactacacacagacaaataaaaactctagtaactggaatggattgacttatttgaattacttgccagtggagctacatagagcacaattattgtatttaaattaccctttatgatcttacaaaacttgacagtaagatcatattgctaaagaaaccacatatttgaatcagggaacatggtgatatctagttgttcttcaactggaaacttcatgctttctgcccagcattcatgttgctggaaagagcaatgtacactaccagtgtagaaattaaatcatcaatcttatcaagatgtggatcctataagttacaataaaaattagcctgataagatatccccaccagaagaatattcacataaatgctatgggagcaacaagctattttctaaattagctttaatcctattctacaagagagaatccatatctagaatagttatagggatcaagaacccatggcttgattggtcataggcccaatgggagatcctaatattattgttctacaaaatgaaaataactcctaatgacttgttgctgcagtaataagttagtatgttgctcaactctcacaagagaagttttgtcttacaataaatggcaattaaagcagccccacaagatttatatcataccgatctcctcatggcctatgcatctagaagctaggaaacaaagaggaccctaagagagacatacatggtccccctggagaaggggaagggggcaagacctccaaagctaattgggagcatgggggaggggagagggagttagaagaaagagaaggggataaaaggagggagaggaggacaagagagagaaggaagatctagtcaagagaagatagaggagagcaagaaaagagataccatagtagagggagccttgtatgtttaaatagaaaactggcactagggaattgtccaaagatccacaaggtccaactaataatctaagcaatagtcgagaggctaccttaaaagcctttctctgataatgagattgatgactaccttatataccatcctagagccttcatccagtagctgatggaagcagaagcagacatctacagctaaacactgagctagttgcagacagggaggagtgatgagcaaagtcaagaccaggctggagaaacacacagaaacagcagacctgaaaaaaatgttgcacatggaccccagactgatagctgggagtccagcataggacttttctagaaaccctgaatgaggatatcagtttggaggtctggttaatctatggggacactggtagtggatcaatatttatccctagttcatgactggaatttgggtacccattccacatggaggaattctctgtcagcctagacacatgggggaggttctaggtcctgctccaaataatgtgttagactttgaagaactcccttgagaagactcaccctccctggggagcagaaaggggatgggatgagggttggtgagggacaggagaggaggggagggtgagggaactgggattgacaagtaaatgatgcttgtttctaatttaaatgaataaaggaaaagtaaaagaagaaaagaaaacaggccaaaagattataaaagacagaggtggtgggtgactataaagaaacactattatctaaataaaaacatgtcagaagcacacatgaacttatagtgtttatgaaagtatgtataataactacataatctcaagccaagaaaaaaatatcatctttcagtgatgaaggtgattttatttctcccagaattaaagccaaagacctaatgaaagtaattatcttcaaaaggttgaaaatacatactttgcaatacacagatctgcctagaaatctcatgttcacaatacacatgatgctcaattgaattccattcaatgttacagtttagataaacagtttgtagataaactcacaatgtatcatttctttttattttttgaccaaacagcttctcatctgttattcagaataattcctcgatggcaggatatccatcccaattgggggaaggggagaatttgaagaaaacctagaccacatacatatttgccattgggaaacaaagtctaaaatgatgttgttcacatcttctctactagtcctctccccgtcccaaagaaccttggtatatgtgcctcattttacagagagaggaaagcaggaactgagcatcccttacttgccatcctcaacccaaaatttgcatcattgctcagctctgcccttctcatatgacagttacaagtcaaggcttccaaagtccctctgtcatgtttggtgtcaatagtttatacagatgacttcatgtcttcatatctaatgtcttatatagattaatattaaacaatgttatttctctaaccacattttaaattaatttaaaaatccattaattgtgtctataaaatgcagacagagtgctgagacacaatataagcctgatgatctgaatttgaaactcacacccaccacatggagaatcaacttccaaaaattttcctattacttccacacttacaccattgtacaaacacaataataatgaacaaaatgaaatgaaataaaaaattaagtctctgtaggtaatgctactgtgcagcaaaagtaaaaatggcagcttaagcttgctttatggttacactttaccatcttccattaattataaggacttcaatcatggcagaactatgctgttattgtctcagtgtaacctaaccaggtgttccagatgttcttaatgtggacacctaaactatttgatatttgggttaagatctttccctctttcagaagaaacctcaggacagagggaatcttgtcttttaattttgagtctgtagactttttccatttcaaatatacatgaaacaagtgatgaagaaaattaatcaaaaggtgggaattgcaatgatattaggttcaatattaagcttcaatattatcatggaatcgcctgttatacactgagtgtttggcaataagggatttttagaagaaggagtttttattctcaacaggttccttaagtttagctcaaataaatctaagcaatccactctagaattaaatagtttcc

SEQ ID NO: 59 - DNA, 14931 bp, Cricetulus griseus
  source 1..14931: mol_type = genomic DNA
catgtacacttatgcaagtatgatatggcccaacacagtattttacaccaatttttatctataaaatatacatgtacatcaaaatatattattaataataacatcattattctttctttccaagtaataaacacatacactgaaattttggttcttgtggataattttaatgaaacaggaaatgcaaatttatcttagcatgtttacttcactttctttgcatagataaccagtaatcacattgatggatcatgtagtgaaatgtatttttaggtatctaaggaattttggcttcgttttgtgcttgttgacactgaattctattcctaacaacagtgtgtaaggattctgtctgatttcttttaccagtatttgtccatttgcattttctttattattcatggctgctgttctagaaagtggaaggtagtgtgtcaagtctgtttaacatgtttccctgatgatcagtgtcttaacacctctctgagtacatgttggccaatgtcgtttctagacccatctattcttgcttgacttatcctggtacatgcctgccaagaaatttctcctcatcctttctgtctcttcactgatttacttgatgtgtggatttcacattgatcatatggaaatagaagatacaattttctttattcacagtttggaagactttcaatctcatagatcatcattattttttgctactgttccctatgctatggtgaaatttccatttgaataattgcttaaacaattaacaagaaagaatctatttttacttgcaataacttccatttcagaacatttactacactgttactatatccaaaaactagttttatatatcatgtgagaaatgactaattcataatttggccatgacatttttttcagaaacagaaaaagtgaccaatacatacacaatgctataaatattaagacttcagcaaattaaatatttattcatgatatcacataaaattcatttattatgttttatttaaatgtgtttttaaaacagtggtatcactaaatattaagttagatgtgtttatgtgcttaatgaatttatattttagaatgttataagttgtatatagtcaaatatgtaataaattttattttttaggtctttctcattaaggtattttaattttgggtcccttttccagagtgactctagctcatgatgagttgacataaaaactaaacagtacaaaatgtacattgcattcagtattgcacttgatctttgcactgaagtttgagtcagttcatacatttagtacttgggaagtacattaagctaactttcattgctctggcaaaatgctcgataagataagagtctattgtggaaagccatggcagcaggaaagtaagactgctgatgatgtttaatccatagtcaagacgcagaaggagatgaatgctggtatccaacattttttgctgttcattttctctagaaccctagtccataaagatgtatgacttgcattcaaaatgcgtccccttcagttgttcaacttttctgtaaatatcctttcaggcatgtctagaagattgtttcgcaaatacttctcaatccattcaagttgatagtgcagattaatcactgcagaataaaagcctgtaacttggctcacgtgccaaggaatatgcacactcctgacacatcaataagtaaatcaaagtgtagcttttgcctttaacattgccagacttatgtaatgttctgcacgttcttcctccatcactttttattctaatggtgtttccttgacattgaatcacgctgtggaagctgcttagaattaacattgaaatctactgatatatttatgatgcagcaatttagatttactattttacttagaattttttataattgagagaatataatattttcacagttatctatctgctgtaaatagaggattttaaaaaaaatctctataacttttttttacaacacacagtaaaattaagttaaaatttaataaagtcactatgttgatttcaaagtgtgctacgcccacggtggtcacgcaggtgtagcagaagatgccactaaggtgggctaaggccgatgggttggggtctgcgctccctggagatgagccccaggcggttccctggcaatcagctgcgatcatgatgcccgatgagccannnnnnnnnnnnnnnnnnnnnnnnnnnnnnnnnnnnnnnnnnnnnnnnnnnnnnnnnnnnnnnnctgggtgactttatggaaagaatttgatagatttcatgatgtagaagaattttattaggcttattttacaggagactaagaccctgggacctaaagatatctgggtcctgagaatcaggaaatgggtagagacgtggttgatggtatgagacagattttagagaactcttagatcatgggcaatgaccgcaatctgatgcttagaatagatcatctataaacaattatgctgttctttttctttctgttgtatgatctgatgatgtagcccccttgccaagttccctgatcccccttgccaagttccctgattgtaacagtatataagcattgcttgagagcatattcaactacattgagtgtgtctgtctgtcatttcctcgccgattcctgatttctccttgagccttttcccttgttctccctcggtcggtggtctccacgagaggcggtccgtggcaaaagtgtataaatgttctaaaacatttgaactctaaaacatgcaaaatgaaaaattaaaataaataaacatgaaaattaaaatatattagctgctaaaagttaaacaatactatataatattttgttattagaattcaaaatcacattagttggatttaatttgaacattgcattctttcaataataatttcaataaaaaaagtttccccatgatagtagaaaataataacatatgtatctatctatttatttaactacacatatatagcatttgtttcaactaaaataaatgaatgagcaaagcacctaagtaattggtgtctattatatttatgaagccaatagtttcaaataaattatcatgcataaggaggtattgcaaatgttaaaccttttttgaaacagatattcccagttacagaaattataatttctaatctttcctataagtagaatgatgataattaatataggccatttgtaaataatgttcagattaaaatattctctatttcactagagaagaatgatattaaatgtattatattttatttcccattttgtttgcaccactattctatatccctcagcagtttaaatttgtttcaccatatgtgtgtgtgtttgtatcttaaatatggcactaaaattagaataatttaatataaatctttaggagaaaagatattgaattattttatgttgataggaaaatatcttttaattgtccaagaatactttttcttctattttaggactgatcagacccaggactaatattttatatgtactaattctatgtaccaaaatatgttattatctcatgaattctgtctcaatattgaggtaataaaaatagtccatcatgaactttaaaattaaaataatgattaattaatttttattcatattttgtttgtatgaatggttatacatcacatgtgtgcctggtgactgtgaatgtcaggagaaggtatgaaagccactggaattggaataagagataatatttgagatgttatgtgggtgctgagaattagacgcaagccatcttcaagaatagccagcatactataccactgagtaatccattcatccctcaataattatctttgtagacagtaaatatatttctaaactataaatgaccagaaaaattaatgtattattaatgaagacattcatctcatgtgacacacttcacctgtctaaatcagtaacactctctccactaattaagattttctaagtgcatgacacttactatttctaaagctgtccaatgggggccagtccccagtcagcacccagtgagataatccatgaatgcatttatatcttaggaaaaattcttatctatgtagtatttagaacattttcatgtgaggggataaacaaggaagcacagatgctttctgatagaaactttctctttaattcatctagaaaaaaaaaacctctcaggaaaatctctcttgctctcctcccaatgctctattcagcatcttctccctacttaattctagatctttttctctatgcctccttgctgctgccctgctggctctgctctatgcctccccatgtcacttttctttgctatctcaccgttaccttctctgcctcactctctgccttcttctctgcttctcacatggccaggctctggacaattatagttatatgttacattctcataacacatgatatgtcacatagtttctctcaggctagggatatcacaatgactggccaatgagcaagtggccttgcatgtagctctaagttggtgatggttcccagacagtaagtagccatttggttgaaatttgaggttgggtagtacatgaagactgaattttcttcaaactctggccttgaaatagtaaaacaacacctatgaaaatgacgacctgtatttgtctttagaggcaaccacatattgtctgcagggcctgctttgaatttgctctgaagttagcttgtttgtgtaaaaggaagaatcctatatcagcctgagaaatgtaaaatatcctagcatttcaagtcatcaaaattatatggagagtataaatcatccttctgactattcatagtcatatttgtgtccaccaagtataaaacacactaccaaagggctgtggaaaaaatcgccataactgttcttattagggaggcatagcagtggtacctgaggaagttacagcaacaaccagtcatccagtcaataaccccatggctttgccacttggaggtacccaataatgtttggctttgccgagtaggactccaacaaattcagagggtcaatttttaaatgctggttgtcactgctgaacagtcccattgccctctgcataattccacaatggaaagctttttacactgattgccaatcattaaacagcctactcagcataaacaggtatgatattattctgcattttgttacattactagatgaattcctatttcttcctacaatagtggaactgaaaaaagatacacaatcatactacccctctactaatcttatgacttatatcatttcaattttcagaccataatgcaaactattgaccaaaacatgtgaagatgaaaaatagaaatgtagaataatattacatataaaaagaaaaggcggacttattttgttttatttcttagcatgcatagcaatacatgatttgaggtttatataataaagggacaataaatcttcaagaaacttacccctactgaattaaaatattaaagaaggtcacacatttactcaaatatattagactactgggcaaatagacatgaaaagtagagttaatattgaggtaggccttctgtgaaatgtctaaggaaattatgtttcatacagtgtgtaaccaagtgggaatcatatcagaaagcagtcaaaagcttatattacaagtaacagatgcttggttatatgacctcccagagcttgactgtctatacacaaaaagtggtgttaataaaactgtaatttgggctatgtttttttaaatggcttcaccaacatgaaaggaagggaatgagcatgtcatggatgcttagagattatgcttccagcaagaagaattgagctttggctcttattacagaaacatgacaaggtgtgagttttatttattagaaattatataatattttaagctggggactaaaaattttattgaaacaaacaggcaagggataggcatgtactagaagcaaaaataggatgtcaatgctgtaatgttattttttggaccaaaatagtatttcctatagaaatgacaatgatcttaggttattattcttcataaagatgacaagttcacaagatatcctagttcattaaaatcgttttagtcatttaatagagtgctgtgatagattacacaaaggaaagcacttacgatgagaaataatgatatccacaattattttcttaattcttagaaacattctattgttatatctcaatctcagaagccacttattgctttattattgaaacatatgaaattgtaagttatatattgtctatggtgacatttcaaagaacatgtgacgtacagtgtagcacagataaagaacataactgcagctgaatcagtaactaaacttacatacattaaatctgccatgttggcaacagtgtgtgcactaccaaaggatgtactaatgctcacgacactcccctatgtcaccctttgttcatcattacatcataggtctattttgtttgcttttgaaatctagaccaagtcttttgtgtctttccaagcacagagctcattaatttacctcatagacttgttaaacttcttctggttcatcaattgaatagaaatactcactactaattatgtgagaccctgccagtaccatagcacatggataatttttacataaaacatgcatacaagtaagattattcagactgaacatgaattttagagaaatcaggaaggagtatatgggagtggttggagtgagactagagaaatgtaattaaactataatctcaatacaaagatctactaagcaaaaaacatgaaacattgtcattcaagtgaaacatcagtcttcaaattggaaagatatttttactaggaaaatgtctggtagatggttattatctagaaaacacaaaaattagaaaacggtaaactttaataaaaagaataatacaatgagactacatgaaaagttcttaactaatgaaacaaatatcttgaaacttttttcttaaaagtttaatatcaataaccatcatggaaattcaaattaaaactatttacatattacccctgaaataataactaatacccaataaaaataatataaacaaaaaatggcaatgcatgccatcatggatttgggagagagaatgttcattgcagttctgaatggatactggtgccaccacggtgaaaatctctgtataggtccttccaaaagctgaaaatagacatatcacaagacctgccacacatttttcaagcaaatacccaaaggactctacctgactgcagagacactttctcataaaatattattgttgatctattcataatatctggaaaatagaaacagccaagatgcccatcaactgattaatagatgataaaattattgtacatttcagtgtaatattattcagtttttaagaaaaatgaaattatgtaataagcatgtaaatggatatatcttgaaacaaccattccccattatattacctaaacattgaaagtccaaaatcatatgatctttttagtggatctactaatcttttgctatatgtattttattgaactacccatggatgtgagataattggtaacaacagcacatgggagagcatgggatcattcaaggaagattagagagaatgcattttttaggagataatggaggagcaatagaaaggattaaatgaggttactgatgaaagtgatggttagagaaggcaatatgaggagggataactagcacttagggccttttgaaaaagacatagagaaaatactattgtagaaacttcctataattggtgtatagttatatacaccaaagagctcagatggagttaccctataatggaaatattaactactttttatcactgtgataaaacatcctgaacagagcaacatagattgggaagcatttactttggcttacagttctaacgggataaaaattcatgatgaaagaatgaatatgtcagcaaacagcagtagcaatggcctgagaagcaggtgagagctcacatcttgaagtgtaagaatgtagcagagagaacaaactgcaaatgaccagaaaatgcttttggatcagagcccatacccctctgactgacttctccagaaattctgaacaaataaaactccccaaacagagccataactgaaggtccagtgtctgagactactaggggtatttcttattcaaaccactacaatggggtggggggagcaatcctccaagtaggcactacacacagacaaataaaaactctagtaactggaatggattgacttatttgaattacttgccagtggagctacatagagcacaattattgtatttaaattaccctttatgatcttacaaaacttgacagtaagatcatattgctaaagaaaccacatatttgaatcagggaacatggtgatatctagttgttcttcaactggaaacttcatgctttctgcccagcattcatgttgctggaaagagcaatgtacactaccagtgtagaaattaaatcatcaatcttatcaagatgtggatcctataagttacaataaaaattagcctgataagatatccccaccagaagaatattcacataaatgctatgggagcaacaagctattttctaaattagctttaatcctattctacaagagagaatccatatctagaatagttatagggatcaagaacccatggcttgattggtcataggcccaatgggagatcctaatattattgttctacaaaatgaaaataactcctaatgacttgttgctgcagtaataagttagtatgttgctcaactctcacaagagaagttttgtcttacaataaatggcaattaaagcagccccacaagatttatatcataccgatctcctcatggcctatgcatctagaagctaggaaacaaagaggaccctaagagagacatacatggtccccctggagaaggggaagggggcaagacctccaaagctaattgggagcatgggggaggggagagggagttagaagaaagagaaggggataaaaggagggagaggaggacaagagagagaaggaagatctagtcaagagaagatagaggagagcaagaaaagagataccatagtagagggagccttgtatgtttaaatagaaaactggcactagggaattgtccaaagatccacaaggtccaactaataatctaagcaatagtcgagaggctaccttaaaagcctttctctgataatgagattgatgactaccttatataccatcctagagccttcatccagtagctgatggaagcagaagcagacatctacagctaaacactgagctagttgcagacagggaggagtgatgagcaaagtcaagaccaggctggagaaacacacagaaacagcagacctgaaaaaaatgttgcacatggaccccagactgatagctgggagtccagcataggacttttctagaaaccctgaatgaggatatcagtttggaggtctggttaatctatggggacactggtagtggatcaatatttatccctagttcatgactggaatttgggtacccattccacatggaggaattctctgtcagcctagacacatgggggaggttctaggtcctgctccaaataatgtgttagactttgaagaactcccttgagaagactcaccctccctggggagcagaaaggggatgggatgagggttggtgagggacaggagaggaggggagggtgagggaactgggattgacaagtaaatgatgcttgtttctaatttaaatgaataaaggaaaagtaaaagaagaaaagaaaacaggccaaaagattataaaagacagaggtggtgggtgactataaagaaacactattatctaaataaaaatatgtcagaagcacacatgaacttatagtgtttatgaaagtatgtataataactacataatctcaagccaagaaaaaaatatcatctttcagtgatgaaggtgattttatttctcccagaattaaagccaaagacctaatgaaagtaattatcttcaaaaggttgaaaatacatactttgcaatacacagatctgcctagaaatctcatgttcacaatacacatgatgctcaattgaattccattcaatgttacagtttagataaacagtttgtagataaactcacaatgtatcatttctttttattttttgaccaaacagcttctcatctgttattcagaataattcctcgatggcaggatatccatcccaattgggggaaggggagaatttgaagaaaacctagaccacatacatatttgccattgggaaacaaagtctaaaatgatgttgttcacatcttctctactagtcctctccccgtcccaaagaaccttggtatatgtgcctcattttacagagagaggaaagcaggaactgagcatcccttacttgccatcctcaacccaaaatttgcatcattgctcagctctgcccttctcatatgacagttacaagtcaaggcttccaaagtccctctgtcatgtttggtgtcaatagtttatacagatgacttcatgtcttcatatctaatgtcttatatagattaatattaaacaatgttatttctctaaccacattttaaattaatttaaaaatccattaattgtgtctataaaatgcagacagagtgctgagacacaatataagcctgatgatctgaatttgaaactcacacccaccacatggagaatcaacttccaaaaattttcctattacttccacacttacaccattgtacaaacacaataataatgaacaaaatgaaatgaaataaaaaattaagtctctgtaggtaatgctactgtgcagcaaaagtaaaaatggcagcttaagcttgctttatggttacactttaccatcttccattaattataaggacttcaatcatggcagaactatgctgttattgtctcagtgtaacctaaccaggtgttccagatgttcttaatgtggacacctaaactatttgatatttgggttaagatctttccctctttcagaagaaacctcaggacagagggaatcttgtcttttaattttgagtctgtagactttttccatttcaaatatacatgaaacaagtgatgaagaaaattaatcaaaaggtgggaattgcaatgatattaggttcaatattaagcttcaatattatcatggaatcgcctgttatacactgagtgtttggcaataagggatttttagaagaaggagtttttattctcaacaggttccttaagtttagctcaaataaatctaagcaatccactctagaattaaatagtttcctaagggcacagctatgaatagagctcaatttacatataaaattttgttcaccatttatgtcattccagttttcattagtacaaggaaaatacaaaatatttagatgtcaatatcaagtgaatagttcatctccttttttaatatatatcacctaaatcaccattttctcagaaaaatctggcctgaagttctgtctggaacttcaacatgaaaaatatgcacagcttgctattataaatcctagttgatttttaagattcatgtctggtgtctgactcagaggggccagaggctagacaaatattttttgaatcttcattgtgaagatttttaatgattattttaatataaataacaaagatgatggataatgtaactttgtacagttcatagacgctgaactactttgtgcttaaaatgttagttccctatcataaatgataggtgataagtgtatgtttaatactttccctctgagctatattcatgtactagagaattattttaaacatgaaaagactgtgtttatagtctcagctcctgagaactggtccaaccttaggcaggtgaatgccaggagcaacgtttttcttctacagaggatgctttgctgccaagcaacctggttgtgtggaaatgttccttttttaatcaagtttaaagggtcttcatcatgctgttgctccacatattttcaggttagagcttggtccttggagtattatcttttaccagaaaattcatagtattctttcaataactaacaactaaacttttcgataaaaaagaattggaatttcaattttaaagcctgagtaaaattcttgtgaatcaggatattttattttaagtcttatcttttaaaaagttattttattttttaaaaaattataatatactttcataatttccctccttcacttttctttacaaacacttctatagatcaccatgtgtttttttttttacatttatggcctctttctgttcattgttattacatacaaatagtcttgcctatagaagaacaccacaatttgttacctgataacaaattatcaacccttaaaacctacaaactattgatattactgaaaagactatacttatagatgtaaagatatatgtgtgtgcacatatatagatacacatatatgtaggatttttaattttagattttagacatcaaaattatttatatgactgagaaactagacactataaatgagcattcagtattcaacaccgtgattttagatattgtcacaatgacagaaaattttcttatagaaaattttaagttttgtgattgctctgtgcacttagtgaagtctcacagaaaaagaatcatagtatttttagtttataataaaaagtacatataattaaaatggttggcacaaaacaacatttgagcatttttcctatttactatcaagtagtatcattttgaaataataatttgactagtttcaaaaatgaaaacaaaatttaaactaaatgcctaatctagcctgataacatttttatgaatgaaattattcaatagtgttatcaattaggggcccaaaacttttcctaaaataaaacttttaatttttttccatttttatttaaattagaaacaaaattgttttacatgtaaatcagagtttcctcaccctccccttctccctgtccctcactaacaccctacttgtcccataccatttctgctccccagggagggtgaggccttccatggggaaacttcagagtctgtctatcctttcggatagggcctaggccctcacccatttgtctaggctaaggctcacaaagtttactcctatgctagtgataagtactgatctactacaagagacaccatagatttcctaggcttcctcactgacacccatgttcatggggtctggaacaatcatatgctagtttcctaggtatcagtctggggaccatgagctcccccttgttcaggtcaactgtttctgtgggtttcaccaccctggtcttgactgctttgctcatcactcctccctttctgtaactgggttccagtacaattccgtgtttagctgtgggtgtctacttctactttcatcagcttctgggatggagcctctaggatagcatacaattagtcatcatctcattatcagggaagggcatttaaagtagcctctccattgttgcttggattgttagttggtgtcatctttgtagatctctggacatttccctagtgccagatatctctttaaacctacaagactacctctattatggtatctcttttcttgctctcgtctattcttccagacaaaatcttcctgctcccttatattttcctctcccctcctcttctccccttctcattctcctagatccatcttcccttcccccatgctcccaagagagatgttgctcaggagatcttgttccttaacccttttcttggggatctgtctctcttagggttgtccttgtttcctagcttctctggaagtgtggattgtaagctggtaatcatttgctccatgtctaaaatccatatatgagtgatgtttgtctttttgtgactgggttacctcactcaaaatggtttcttccatatgtctgtggatttcaatagcacaaacaacatacagtatcttggggcaacactaaccaaacaagtgaaagaccagtatagcaagaactttgagtttaaagaaagaaattaaagaagataccagaaaatggaaagatctcccatgctctttgataggcagaatcaacatagtaaaaatggcaatcttgccaaaatccatctacagactcaatgcaatccccattaaataccagcacacttcttcacagacctgaaagaataatacttaactttatatggagaaacaaaagacccaggataggccaaacaaccctgtacaatgaaggcacttccagaggcatccccatccctgacttcaagctctattatagagtaataatcctgaaaacagcttggtaatggcacaaaaatagacaggtagaccaatggaattgagttgaaaaccctgatattaacccacatatctatgaacacctgactttgacaaagaagctaaggttatacaatgtaagaaagaaagcatcttcaacaaatcgtgctggcataactggatgctggcatgtagaagactgcagatagatccatgtctaatgccatgcacaaaacttaagtccaaatggatcaaaaacctcaacataaatccagccacactgaacctcatagaagagaaagtgggaagtatccttgaataaattggtacaggagaccacatcttgaacttaacaccagtagcacagacaatcagatcaataatcaataaatgggacctcctgaaactgagaagcttctgtaaggcaatggataagtcaacaggacaaaatggcagcccacggaatgggaaaagatattcaccaatcctatatctgacagagggctgctctctatttgcaaagaacacaataagctagtttttaaaacaccaattaatccgattataaagttgggtagagaactaaataaagaattgttaacagagcaatctaacttggcagaaagacacataagaaagtgctcaccat